(19) United States
(12) Patent Application Publication
(10) Pub. No.: US 2019/0055578 A1
(43) Pub. Date: Feb. 21, 2019

(54) DELIVERY OF CENTRAL NERVOUS SYSTEM TARGETING POLYNUCLEOTIDES

(71) Applicant: Voyager Therapeutics, Inc., Cambridge, MA (US)

(72) Inventors: Dinah Wen-Yee SAH, Hopkinton, MA (US); Jinzhao HOU, Belmont, MA (US); Martin GOULET, Weston, MA (US); Adrian Philip KELLS, Arlington, MA (US); Pengcheng ZHOU, Lexington, MA (US); Gregory Robert STEWART, Plymouth, MN (US)

(73) Assignee: Voyager Therapeutics, Inc., Cambridge, MA (US)

(21) Appl. No.: 15/771,376

(22) PCT Filed: Oct. 28, 2016
(86) PCT No.: PCT/US2016/059302

§ 371 Date: Apr. 26, 2018

Related U.S. Application Data
(60) Provisional application No. 62/248,220, filed on Oct. 29, 2015.
(60) Provisional application No. 62/248,223, filed on Oct. 29, 2015.
(60) Provisional application No. 62/279,420, filed on Jan. 15, 2016.

(51) Int. Cl. C12N 15/86; A61K 9/00; C07K 14/47; A61M 5/142; A61M 25/00

(52) U.S. Cl. CPC C12N 15/86; A61K 9/0085; C07K 14/47; C12N 2750/14145; A61M 25/0023; C12N 2750/14143; A61M 5/14276

(57) ABSTRACT

The invention relates to compositions and methods for the preparation, administration, manufacture and therapeutic use of viral particles for the treatment of CNS disorders.

### CROSS REFERENCE TO RELATED APPLICATIONS

This application claims the benefit of U.S. Provisional Application No. 62/248,220 filed Oct. 29, 2015, entitled Central Nervous System Targeting Polynucleotides, U.S. Provisional Application No. 62/248,223 filed Oct. 29, 2015, entitled Methods of Delivery to the Central Nervous System, and U.S. Provisional Application No. 62/279,420 filed Jan. 15, 2016, entitled Central Nervous System Targeting Polynucleotides, the contents of each are herein incorporated by reference in their entirety.

### REFERENCE TO THE SEQUENCE LISTING

The present application is being filed along with a Sequence Listing in electronic format. The Sequence Listing is provided as a file entitled 20571034PCT_SEQLST.txt created on Oct. 27, 2016 which is 3,463,093 bytes in size. The information in the electronic format of the sequence listing is incorporated herein by reference in its entirety.

### FIELD OF THE INVENTION

The present invention relates to compositions, methods and processes for the formulation and for the administration of a therapeutic agent using parvovirus e.g., adeno-associated virus (AAV) to the central nervous system (CNS), CNS tissues, CNS structures or CNS cells.

### BACKGROUND OF THE INVENTION

Use of adeno-associated virus (AAV) to deliver therapeutic agents (i.e., transgenes) to the central nervous system offers a means to achieve a widespread distribution of delivered genes in the CNS. Tissue of the CNS is highly heterogeneous and consists of different cell types including different types of neurons (e.g., excitatory and inhibitory neurons) and glial cells (e.g., oligodendrocytes, astrocytes and microglia). The characterization of different AAV capsid serotypes reveals that different AAV serotypes have different efficiency of transduction to different CNS tissues (e.g., cervical spinal cord and hippocampus) and cells (e.g., neurons or glial cells). Inclusion of different promoters within the AAV serotypes can further enhance transduction to CNS tissues and cells.

Studies, such as those referenced herein examining the targeting of specific tissues and cell types of the CNS by AAV capsids, address one part of the problem of effective clinical treatment of CNS disorders by AAV delivery of therapeutic transgenes. The appropriate expression of the therapeutic transgene encoding the delivered payload, both temporally and spatially within the desired cell type, is critical to achieving the desired ameliorative effect. The properties of regulatory elements that drive expression of exogenous payloads from AAV genomes have not been well characterized.

On this background there remains, however, much work to be done to optimize delivery of therapeutic agents to the central nervous system. A better understanding and optimizing delivery parameters for viral particle distribution, as described herein, will lead to safer and more effective gene therapy. AAVs have emerged as one of the most widely studied and utilized viral particles for gene transfer to mammalian cells. See, e.g., Tratschin et al., Mol. Cell Biol., 5(11):3251-3260 (1985) and Grimm et al., Hum. Gene Ther., 10(15):2445-2450 (1999).

The present invention addresses the need for new technologies by providing AAV-based compositions and complexes which go beyond those of the art by providing for administration and/or delivery of recombinant adeno-associated virus (AAV) particles in the treatment of diseases or disorders of the CNS, CNS tissues and/or CNS structures.

While delivery is exemplified in the AAV context, other viral vectors, non-viral vectors, nanoparticles, or liposomes may be similarly used to deliver the therapeutic transgenes and include, but are not limited to, vector genomes of any of the AAV serotypes or other parvoviral viral delivery vehicles or lentivirus, etc. The observations and teachings may extend to any macromolecular structure, including modified cells, introduced into the CNS in the manner as described herein.

### SUMMARY OF THE INVENTION

The present invention relates to AAV particles comprising AAV capsid serotypes with specific cell tropisms. Methods for delivering the AAV particles are also included in the present invention.

The present invention provides AAV particles and methods of delivering AAV particles to cells and tissues of the central nervous system.

Provided herein are AAV particles comprising a vector genome packaged in a capsid.

Provided herein are methods for increasing the level of a protein in the CNS of a subject by administering a subject an effective amount of an AAV particle.

Provided herein are methods for increasing distribution of AAV particles in the CNS of a subject by administering a subject an effect amount of an AAV particle. Distribution may be increased by a percentage such as, but not limited to, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 60%, 65%, 70%, 75%, 80%, 85%, 90%, 95% or more than 95%.

In one embodiment, the AAV particle may comprise a vector genome packaged in a capsid, and the capsid may be, but is not limited to, AAVrh.10 (AAVrh10), AAV-DJ (AAVDJ), AAV-DJ8 (AAVDJ8), AAV1, AAV2, AAV2G9, AAV3, AAV3a, AAV3b, AAV3-3, AAV4, AAV4-4, AAV5, AAV6, AAV6.1, AAV6.2, AAV6.1.2, AAV7, AAV7.2, AAV8, AAV9, AAV9.11, AAV9.13, AAV9.16, AAV9.24, AAV9.45, AAV9.47, AAV9.61, AAV9.68, AAV9.84, AAV9.9, AAV10, AAV11, AAV12, AAV16.3, AAV24.1, AAV27.3, AAV42.12, AAV42-1b, AAV42-2, AAV42-3a, AAV42-3b, AAV42-4, AAV42-5a, AAV42-5b, AAV42-6b, AAV42-8, AAV42-10, AAV42-11, AAV42-12, AAV42-13, AAV42-15, AAV42-aa, AAV43-1, AAV43-12, AAV43-20, AAV43-21, AAV43-23, AAV43-25, AAV43-5, AAV44.1, AAV44.2, AAV44.5, AAV223.1, AAV223.2, AAV223.4, AAV223.5, AAV223.6, AAV223.7, AAV1-7/rh.48, AAV1-8/rh.49, AAV2-15/rh.62, AAV2-3/rh.61, AAV2-4/rh.50, AAV2-5/rh.51, AAV3.1/hu.6, AAV3.1/hu.9, AAV3-9/rh.52, AAV3-11/rh.53, AAV4-8/r11.64, AAV4-9/rh.54, AAV4-19/rh.55, AAV5-3/rh.57, AAV5-22/rh.58, AAV7.3/hu.7, AAV16.8/hu.10, AAV16.12/hu.11, AAV29.3/bb.1, AAV29.5/bb.2, AAV106.1/hu.37, AAV114.3/hu.40, AAV127.2/hu.41, AAV127.5/hu.42, AAV128.3/hu.44, AAV130.4/hu.48, AAV145.1/hu.53, AAV145.5/hu.54, AAV145.6/hu.55, AAV161.10/hu.60, AAV161.6/hu.61, AAV33.12/hu.17, AAV33.4/hu.15, AAV33.8/hu.16, AAV52/hu.19, AAV52.1/hu.20, AAV58.2/hu.25, AAVA3.3, AAVA3.4, AAVA3.5, AAVA3.7, AAVC1, AAVC2, AAVC5, AAVF3, AAVF5, AAVH2, AAVrh.72, AAVhu.8, AAVrh.68, AAVrh.70, AAVpi.1, AAVpi.3, AAVpi.2, AAVrh.60, AAVrh.44, AAVrh.65, AAVrh.55, AAVrh.47, AAVrh.69, AAVrh.45, AAVrh.59, AAVhu.12, AAVH6, AAVLK03, AAVH-1/hu.1, AAVH-5/hu.3, AAVLG-10/rh.40, AAVLG-4/rh.38, AAVLG-9/hu.39, AAVN721-8/rh.43, AAVCh.5, AAVCh.5R1, AAVcy.2, AAVcy.3, AAVcy.4, AAVcy.5, AAVCy.5R1, AAVCy.5R2, AAVCy.5R3, AAVCy.5R4, AAVcy.6, AAVhu.1, AAVhu.2, AAVhu.3, AAVhu.4, AAVhu.5, AAVhu.6, AAVhu.7, AAVhu.9, AAVhu.10, AAVhu.11, AAVhu.13, AAVhu.15, AAVhu.16, AAVhu.17, AAVhu.18, AAVhu.20, AAVhu.21, AAVhu.22, AAVhu.23.2, AAVhu.24, AAVhu.25, AAVhu.27, AAVhu.28, AAVhu.29, AAVhu.29R, AAVhu.31, AAVhu.32, AAVhu.34, AAVhu.35, AAVhu.37, AAVhu.39, AAVhu.40, AAVhu.41, AAVhu.42, AAVhu.43, AAVhu.44, AAVhu.44R1, AAVhu.44R2, AAVhu.44R3, AAVhu.45, AAVhu.46, AAVhu.47, AAVhu.48, AAVhu.48R1, AAVhu.48R2, AAVhu.48R3, AAVhu.49, AAVhu.51, AAVhu.52, AAVhu.54, AAVhu.55, AAVhu.56, AAVhu.57, AAVhu.58, AAVhu.60, AAVhu.61, AAVhu.63, AAVhu.64, AAVhu.66, AAVhu.67, AAVhu.14/9, AAVhu.t 19, AAVrh.2, AAVrh.2R, AAVrh.8, AAVrh.8R, AAVrh.12, AAVrh.13, AAVrh.13R, AAVrh.14, AAVrh.17, AAVrh.18, AAVrh.19, AAVrh.20, AAVrh.21, AAVrh.22, AAVrh.23, AAVrh.24, AAVrh.25, AAVrh.31, AAVrh.32, AAVrh.33, AAVrh.34, AAVrh.35, AAVrh.36, AAVrh.37, AAVrh.37R2, AAVrh.38, AAVrh.39, AAVrh.40, AAVrh.46, AAVrh.48, AAVrh.48.1, AAVrh.48.1.2, AAVrh.48.2, AAVrh.49, AAVrh.51, AAVrh.52, AAVrh.53, AAVrh.54, AAVrh.56, AAVrh.57, AAVrh.58, AAVrh.61, AAVrh.64, AAVrh.64R1, AAVrh.64R2, AAVrh.67, AAVrh.73, AAVrh.74, AAVrh8R, AAVrh8R A586R mutant, AAVrh8R R533A mutant, AAAV, BAAV, caprine AAV, bovine AAV, AAVhE1.1, AAVhEr1.5, AAVhER1.14, AAVhEr1.8, AAVhEr1.16, AAVhEr1.18, AAVhEr1.35, AAVhEr1.7, AAVhEr1.36, AAVhEr2.29, AAVhEr2.4, AAVhEr2.16, AAVhEr2.30, AAVhEr2.31, AAVhEr2.36, AAVhER1.23, AAVhEr3.1, AAV2.5T, AAV-PAEC, AAV-LK01, AAV-LK02, AAV-LK03, AAV-LK04, AAV-LK05, AAV-LK06, AAV-LK07, AAV-LK08, AAV-LK09, AAV-LK10, AAV-LK11, AAV-LK12, AAV-LK13, AAV-LK14, AAV-LK15, AAV-LK16, AAV-LK17, AAV-LK18, AAV-LK19, AAV-PAEC2, AAV-PAEC4, AAV-PAEC6, AAV-PAEC7, AAV-PAEC8, AAV-PAEC11, AAV-PAEC12, AAV-2-pre-miRNA-101, AAV-8h, AAV-8b, AAV-h, AAV-b, AAV SM 10-2, AAV Shuffle 100-1, AAV Shuffle 100-3, AAV Shuffle 100-7, AAV Shuffle 10-2, AAV Shuffle 10-6, AAV Shuffle 10-8, AAV Shuffle 100-2, AAV SM 10-1, AAV SM 10-8, AAV SM 100-3, AAV SM 100-10, BNP61 AAV, BNP62 AAV, BNP63 AAV, AAVrh.50, AAVrh.43, AAVrh.62, AAVrh.48, AAVhu.19, AAVhu.11, AAVhu.53, AAV4-8/rh.64, AAVLG-9/hu.39, AAV54.5/hu.23, AAV54.2/hu.22, AAV54.7/hu.24, AAV54.1/hu.21, AAV54.4R/hu.27, AAV46.2/hu.28, AAV46.6/hu.29, AAV128.1/hu.43, true type AAV (ttAAV), UPENN AAV 10 and/or Japanese AAV 10 serotypes, and variants thereof.

In one embodiment, the vector genome comprises a promoter. The promoter may be, but is not limited to, CBA, CMV, PGK, FXN, H1, and fragments or variants thereof.

In one embodiment, the AAV particles may be administered by a route such as, but not limited to, intrathecal (IT) administration, intraparenchymal (IPa) administration, and/or intracerebroventricular (ICV) administration

In one embodiment, the AAV particles may be administered by intrathecal (IT) administration. The IT administration may be by bolus or prolonged infusion. The IT administration may occur in at least one location in at least one region of the spine of a subject. The region may be, but is not limited to, the cervical region (C1, C2, C3, C4, C5, C6, and C7), thoracic region (T1, T2, T3, T3, T4, T5, T6, T7, T8, T9, T10, T11, and T12), lumbar region (L1, L2, L3, L4, and L5) and/or sacral region (S1, S2, S3, S4, and S5). In one embodiment, the IT administration may occur in one location such as, but not limited to, C1, C5, T1, L1 or L5. In one embodiment, the IT administration may occur in three locations such as, but not limited to, L1, T1 and C5.

In one embodiment, the volume of IT administration by any of the methods described herein is less than 1 mL.

In one embodiment, the volume of IT administration by any of the methods described herein is between about 0.1 mL to about 120 mL.

In one embodiment, during IT administration a subject may be in a position such as, but not limited to, supine, prone, right lateral recumbent (RLR), left lateral recumbent (LLR), Fowler's, and Trendelenburg.

In one embodiment, during IT administration a subject may be at an angle between approximately horizontal 00 to about vertical 90° for the duration of the administration. The angle may be, but is not limited to, 0°, 1°, 2°, 3°, 4°, 5°, 6°, 7°, 8°, 9°, 10°, 11°, 12°, 13°, 14°, 15°, 16°, 17°, 18°, 19°, 20°, 21°, 22°, 23°, 24°, 25°, 26°, 27°, 28°, 29°, 30°, 31°, 32°, 33°, 34°, 35°, 36°, 37°, 38°, 39°, 40°, 41°, 42°, 43°, 44°, 45°, 46°, 47°, 48°, 49°, 50°, 51°, 52°, 53°, 54°, 55°, 56°, 57°, 58°, 59°, 60°, 61°, 62°, 63°, 64°, 65°, 66°, 67°, 68°, 69°, 70°, 71°, 72°, 73°, 74°, 75°, 76°, 77°, 78°, 79°, 80°, 81°, 82°, 83°, 84°, 85°, 86°, 87°, 88°, 89°, and 90°.

In one embodiment, the administration route in any of the methods described herein is IT administration via prolonged infusion. The volume of prolonged infusion may be a volume such as, but not limited to, more than 1 mL, at least 3 mL, 3 mL, at least 10 mL, and 10 mL. The duration of the prolonged infusion may be, but is not limited to, 0.17, 0.33, 0.5, 0.67, 0.83, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, and 36 hour(s). The prolonged infusion may occur at a rate which is constant, ramped, or complex. In one aspect, the ramped rate increases over the duration of the prolonged infusion. In one aspect, the complex rate alternates between high and low rates over the duration of the prolonged infusion. In one aspect, the rate of prolonged infusion may be, but is not limited to, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 10.1, 10.2, 10.3, 10.4, 10.5, 10.6, 10.7, 10.8, 10.9, 11.0, 11.1, 11.2, 11.3, 11.4, 11.5, 11.6, 11.7, 11.8, 11.9, 12.0, 12.1, 12.2, 12.3, 12.4, 12.5, 12.6, 12.7, 12.8, 12.9, 13.0, 13.1, 13.2, 13.3, 13.4, 13.5, 13.6, 13.7, 13.8, 13.9, 14.0, 14.1, 14.2, 14.3, 14.4, 14.5, 14.6, 14.7, 14.8, 14.9, 15.0, 15.1, 15.2, 15.3, 15.4, 15.5, 15.6, 15.7, 15.8, 15.9, 16.0, 16.1, 16.2, 16.3, 16.4, 16.5, 16.6, 16.7, 16.8, 16.9, 17.0, 17.1, 17.2, 17.3, 17.4, 17.5, 17.6, 17.7, 17.8, 17.9, 18.0, 18.1, 18.2, 18.3, 18.4, 18.5, 18.6, 18.7, 18.8, 18.9, 19.0, 19.1, 19.2, 19.3, 19.4, 19.5, 19.6, 19.7, 19.8, 19.9, 20.0, 20.1, 20.2, 20.3, 20.4, 20.5, 20.6, 20.7, 20.8, 20.9, 21.0, 21.1, 21.2, 21.3, 21.4, 21.5, 21.6, 21.7, 21.8, 21.9, 22.0, 22.1, 22.2, 22.3, 22.4, 22.5, 22.6, 22.7, 22.8, 22.9, 23.0, 23.1, 23.2, 23.3, 23.4, 23.5, 23.6, 23.7, 23.8, 23.9, 24.0, 24.1, 24.2, 24.3, 24.4, 24.5, 24.6, 24.7, 24.8, 24.9, and 25.0 mL/hour. In one aspect, the rate of prolonged infusion is a rate that exceeds the rate of cerebrospinal fluid (CSF) absorption.

In one embodiment, the administration route may be ICV administration. The ICV administration may be to at least one location such as, but not limited to, right lateral ventricle, left lateral ventricle, third ventricle, fourth ventricle, interventricular foramina (also called foramina of Monro), cerebral aqueduct, central canal, median aperture, right lateral aperture, left lateral aperture, and/or perivascular space in the brain.

In one embodiment, the total dose of administration of the AAV particles described herein may be, but is not limited to, between 1×10⁶ VG and about 1×10¹⁶ VG. The total dose may be, but is not limited to, about 1×10⁶, 2×10⁶, 3×10⁶, 4×10⁶, 5×10⁶, 6×10⁶, 7×10⁶, 8×10⁶, 9×10⁶, 1×10⁷, 2×10⁷, 3×10⁷, 4×10⁷, 5×10⁷, 6×10⁷, 7×10⁷, 8×10⁷, 9×10⁷, 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 2×10¹⁰, 3×10¹⁰, 4×10¹⁰, 5×10¹⁰, 6×10¹⁰, 7×10¹⁰, 8×10¹⁰, 9×10¹⁰, 1×10¹¹, 2×10¹¹, 3×10¹¹, 4×10¹¹, 5×10¹¹, 6×10¹¹, 7×10¹¹, 8×10¹¹, 9×10¹¹, 1×10¹², 2×10¹², 3×10¹², 4×10¹² 5×10¹², 6×10¹², 7×10¹², 8×10¹², 9×10¹², 1×10¹³, 2×10¹³, 3×10¹³, 4×10¹³, 5×10¹³, 6×10¹³, 7×10¹³, 8×10¹³, 9×10¹³, 1×10¹⁴, 2×10¹⁴, 3×10¹⁴, 4×10¹⁴, 5×10¹⁴, 6×10¹⁴, 7×10¹⁴, 8×10¹⁴, 9×10¹⁴, 1×10¹⁵, 2×10¹⁵, 3×10¹⁵, 4×10¹⁵, 5×10¹⁵, 6×10¹⁵, 7×10¹⁵, 8×10¹⁵, 9×10¹⁵, and 1×10¹⁶ VG.

In one embodiment, the concentration of the AAV particles described herein delivered to a subject may be, but is not limited to, 1×10⁶, 2×10⁶, 3×10⁶, 4×10⁶, 5×10⁶, 6×10⁶, 7×10⁶, 8×10⁶, 9×10⁶, 1×10⁷, 2×10⁷, 3×10⁷, 4×10⁷, 5×10⁷, 6×10⁷, 7×10⁷, 8×10⁷, 9×10⁷, 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 2×10¹⁰, 3×10¹⁰, 4×10¹⁰, 5×10¹⁰, 6×10¹⁰, 7×10¹⁰, 8×10¹⁰, 9×10¹⁰, 1×10¹¹, 2×10¹¹, 3×10¹¹, 4×10¹¹, 5×10¹¹, 6×10¹¹, 7×10¹¹, 8×10¹¹, 9×10¹¹, 1×10¹², 2×10¹², 3×10¹², 4×10¹² 5×10¹², 6×10¹², 7×10¹², 8×10¹², 9×10¹², 1×10¹³, 2×10¹³, 3×10¹³, 4×10¹³, 5×10¹³, 6×10¹³, 7×10¹³, 8×10¹³, 9×10¹³, 1×10¹⁴, 2×10¹⁴, 3×10¹⁴, 4×10¹⁴, 5×10¹⁴, 6×10¹⁴, 7×10¹⁴, 8×10¹⁴, 9×10¹⁴, 1×10¹⁵, 2×10¹⁵, 3×10¹⁵, 4×10¹⁵, 5×10¹⁵, 6×10¹⁵, 7×10¹⁵, 8×10¹⁵, 9×10¹⁵, and 1×10¹⁶ VG/mL.

In one embodiment, delivery devices may be used to administer the AAV particles using the methods described herein. As a non-limiting example, an infusion pump or device in combination with a catheter may be used during IT administration. The catheter may be a single or multi-port catheter and the catheter may have a flexible, rigid and/or retractable catheter. As another non-limiting example, a head trajectory guide, head trajectory frame, and/or a skull frame is used for ICV administration. Optionally, neuronavigational software may also be used for ICV administration.

The details of one or more embodiments of the invention are set forth in the accompanying description below. Other features, objects and advantages of the invention will be apparent from the description. In the description, the singular forms also include the plural unless the context clearly dictates otherwise. Unless defined otherwise, all technical and scientific terms used herein have the same meaning as commonly understood by one of ordinary skill in the art to which this invention belongs. In the case of conflict, the present description will control.

### DETAILED DESCRIPTION OF THE INVENTION

Described herein are compositions, methods, processes, kits and devices for the design, preparation, manufacture and/or formulation of AAV particles. In some embodiments, payload may be encoded by payload construct and contained within plasmids or vectors or recombinant adeno-associated viruses (AAVs).

The present invention provides AAV capsid serotypes with specific CNS cell type tropism, expression levels and bio-distribution in the CNS. Additionally, the present invention provides regulatory elements and codon optimization of the AAV genome useful in vitro and in vivo in both cell lines and primary CNS cell types. Accordingly, the present invention provides novel AAV particles with novel combinations of capsid and/or payload that target specific cells and/or tissue in a particular anatomic location in the CNS.

The present invention provides administration and/or delivery methods for vectors and viral particles, e.g., AAV particles, for the treatment or amelioration of diseases or disorders of the CNS. Such methods may involve the inhibition of gene expression, gene replacement or gene activation. Such outcomes are achieved by utilizing the methods and compositions taught herein.

The present disclosure provides a method of delivering to a subject, including a mammalian subject, any of the described AAV particles comprising administering to the subject said AAV particle, or administering to the subject a particle comprising said AAV particle, or administering to the subject any of the described compositions, including pharmaceutical compositions.

### Parvoviridae Virus, Viral Particle and Production of Viral Particles

Viruses of the Parvoviridae family are small non-enveloped icosahedral capsid viruses characterized by a single stranded DNA genome. Parvoviridae family viruses consist of two subfamilies: Parvovirinae, which infect vertebrates, and Densovirinae, which infect invertebrates. The parvoviruses and other members of the Parvoviridae family are generally described in Kenneth I. Berns, “Parvoviridae: The Viruses and Their Replication,” Chapter 69 in FIELDS VIROLOGY (3d Ed. 1996), the contents of which is incorporated by reference in its entirety.

The genome of the viruses of the Parvoviridae family may be modified to contain a minimum of components for the assembly of a functional recombinant virus which is loaded with or engineered to express or deliver a desired nucleic acid construct or payload, e.g., a transgene, polypeptide-encoding polynucleotide or modulatory nucleic acid, which may be delivered to a target cell, tissue or organism. As used herein, a “viral particle” refers to a functional recombinant virus.

The Parvoviridae family may be used as a biological tool due to a relatively simple structure that may be manipulated with standard molecular biology techniques.

The Parvoviridae family comprises the Dependovirus genus which includes adeno-associated viruses (AAVs) which are capable of replication in vertebrate hosts including, but not limited to, human, primate, bovine, canine, equine, and ovine species. The naturally occurring AAV Cap gene expresses VP1, VP2, and VP3 capsid proteins are encoded by a single open reading frame of the Cap gene under control of the p40 promoter. In one embodiment, nucleotide sequences encoding VP1, VP2 and VP3 proteins and/or amino acid sequences of AAV VP capsid proteins may be modified for increased efficiency to target to the central nervous system (e.g., CNS tissue tropism). Any of the VP genes of the serotypes selected from, but not limited to, AAV1, AAV2, AAV3, AAV4, AAV5, AAV6, AAV7, AAV8, AAV9, AAV10, and AAV11, AAV12, AAVrh8, AAVrh10, AAV-DJ, and AAV-DJ/8 capsid serotypes, or variants thereof (e.g., AAV3A and AAV3B) may be modified.

In one embodiment, the present invention provides administration and/or delivery methods for viral particles.

In some embodiments, the present invention provides administration and/or delivery methods for viral particles for the treatment and/or amelioration of diseases or disorders of the CNS. As a non-limiting example, the disease or disorder of the CNS is Alzheimer's Diseases (AD), Amyotrophic lateral sclerosis (ALS), Creutzfeldt-Jakob Disease, Huntingtin's disease (HD), Friedreich's ataxia (FA or FRDA), Parkinson Disease (PD), Multiple System Atrophy (MSA), Spinal Muscular Atrophy (SMA), Multiple Sclerosis (MS), Primary progressive aphasia, Progressive supranuclear palsy, Dementia, Brain Cancer, Degenerative Nerve Diseases, Encephalitis, Epilepsy, Genetic Brain Disorders that cause neurodegeneration, Retinitis pigmentosa (RP), Head and Brain Malformations, Hydrocephalus, Stroke, Prion disease, Infantile neuronal ceroid lipofuscinosis (INCL) (a neurodegenerative disease of children caused by a deficiency in the lysosomal enzyme palmitoyl protein thioesterase-1 (PPT1)).

The present disclosure provides a method for the generation of viral particles, by viral genome replication in a viral replication cell comprising contacting the viral replication cell with a payload construct vector and a viral construct vector.

The present disclosure provides a method for producing a viral particle having enhanced (increased, improved) transduction efficiency comprising the steps of: 1) co-transfecting competent bacterial cells with a bacmid vector and either a viral construct vector and/or payload construct vector, 2) isolating the resultant viral construct vector and payload construct vector and separately transfecting viral replication cells, 3) isolating and purifying resultant payload and viral construct particles comprising viral construct vector or payload construct vector, 4) co-infecting a viral replication cell with both the payload construct vector and viral construct vector, 5) harvesting and purifying the viral particle comprising a parvoviral genome. Production methods are further disclosed in commonly owned and co-pending International Publication No. WO2015191508, the contents of which are herein incorporated by reference in their entirety.

In one embodiment, provided are particles comprising nucleic acids and cells (in vivo or in culture) comprising the nucleic acids and/or particles of the invention. Suitable particles include without limitation viral particles (e.g., adenovirus, AAV, herpes virus, vaccinia, poxviruses, baculoviruses, and the like), plasmids, phage, YACs, BACs, and the like as are well known in the art. Such nucleic acids, particles and cells can be used, for example, as reagents (e.g., helper packaging constructs or packaging cells) for the production of modified virus capsids or virus particles as described herein.

The particles of the invention which comprise nucleic acids include any genetic element (vector) which may be delivered to a host cell, e.g., naked DNA, plasmid, phage, transposon, cosmid, episome, a protein in a non-viral delivery vehicle (e.g., a lipid-based carrier), virus, etc., which transfers the sequences carried thereon. The methods used to construct any embodiment of this invention are known to those with skill in nucleic acid manipulation and include genetic engineering, recombinant engineering, and synthetic techniques. See, e.g., Sambrook et al, Molecular Cloning: A Laboratory Manual, Cold Spring Harbor Press, Cold Spring Harbor, N.Y.

The nucleic acid (e.g., transgene or payload) can be carried on any suitable vector, e.g., a plasmid, which is delivered to a host cell. The plasmids useful in this invention may be engineered such that they are suitable for replication and, optionally, integration in prokaryotic cells, mammalian cells, or both. These plasmids may contain sequences permitting replication of the transgene in eukaryotes and/or prokaryotes and selection markers for these systems. Selectable markers or reporter genes may include sequences encoding geneticin, hygromicin or purimycin resistance, among others. The plasmids may also contain certain selectable reporters or marker genes that can be used to signal the presence of the vector in bacterial cells, such as ampicillin resistance. Other components of the plasmid may include an origin of replication and an amplicon, such as the amplicon system employing the Epstein Barr virus nuclear antigen. This amplicon system, or other similar amplicon components permit high copy episomal replication in the cells. Preferably, the molecule carrying the transgene or payload is transfected into the cell, where it may exist transiently. Alternatively, the transgene may be stably integrated into the genome of the host cell, either chromosomally or as an episome. In certain embodiments, the transgene may be present in multiple copies, optionally in head-to-head, head-to-tail, or tail-to-tail concatamers. Suitable transfection techniques are known and may readily be utilized to deliver the transgene to the host cell.

### AAV Particle

In one embodiment, the present invention provides administration and/or delivery methods for AAV particles. As used herein, “AAV particles” refers to a viral particle where the virus is adeno-associated virus (AAV). An AAV particle comprises a viral genome and a capsid. As used herein, “viral genome” is a polynucleotide encoding at least one inverted terminal repeat (ITR), at least one regulatory sequence, and at least one payload.

The AAV particles described herein may be useful in the fields of human disease, antibodies, viruses, veterinary applications and a variety of in vivo and in vitro settings.

In some embodiments, AAV particles described herein are useful in the field of medicine for the treatment, palliation and/or amelioration of conditions or diseases such as, but not limited to, blood, cardiovascular, CNS, and/or genetic disorders.

In some embodiments, AAV particles in accordance with the present invention may be used for the treatment of disorders, and/or conditions, including but not limited to neurological disorders (e.g., Alzheimer's disease, Huntington's disease, autism, Parkinson's disease, Spinal muscular atrophy, Friedreich's ataxia).

In some embodiments, the present invention provides administration and/or delivery methods for AAV particles for the treatment and/or amelioration of diseases or disorders of the CNS. As a non-limiting example, the disease or disorder of the CNS is Alzheimer's Diseases (AD), Amyotrophic lateral sclerosis (ALS), Creutzfeldt-Jakob Disease, Huntingtin's disease (HD), Friedreich's ataxia (FA or FRDA), Parkinson Disease (PD), Multiple System Atrophy (MSA), Spinal Muscular Atrophy (SMA), Multiple Sclerosis (MS), Primary progressive aphasia, Progressive supranuclear palsy, Dementia, Brain Cancer, Degenerative Nerve Diseases, Encephalitis, Epilepsy, Genetic Brain Disorders that cause neurodegeneration, Retinitis pigmentosa (RP), Head and Brain Malformations, Hydrocephalus, Stroke, Prion disease, Infantile neuronal ceroid lipofuscinosis (INCL) (a neurodegenerative disease of children caused by a deficiency in the lysosomal enzyme palmitoyl protein thioesterase-1 (PPT1)).

In some embodiments, AAV particles produced according to the present invention may target to deliver and/or to transfer a payload of interest to specific population of cells in specific anatomical regions (e.g., dopaminergic (DAergic) neurons in the Substantia Nigra (SN)) in the central nervous system).

In one embodiment, the AAV particles of the invention may be a single-stranded AAV (ssAAV) or a self-complementary AAV (scAAV) described herein or known in the art.

### Payload

AAV particles of the present invention may comprise a nucleic acid sequence encoding at least one “payload.” As used herein, a “payload” refers to one or more polynucleotides or polynucleotide regions encoded by or within a viral genome or an expression product of such polynucleotide or polynucleotide region, e.g., a transgene, a polynucleotide encoding a polypeptide or multi-polypeptide or a modulatory nucleic acid or regulatory nucleic acid.

The payload may comprise any nucleic acid known in the art which is useful for modulating the expression in a target cell transduced or contacted with the AAV particle carrying the payload. In one embodiment, modulation may be by supplementation of the payload in a target cell or tissue. In one embodiment, modulation may be gene replacement of the payload in a target cell or tissue. In one embodiment, modulation may be by inhibition using a modulatory nucleic acid of the payload in a target cell or tissue.

In one embodiment, the payload may comprise a combination of coding and non-coding nucleic acid sequences.

mRNA

In one embodiment, a messenger RNA (mRNA) may be encoded by a payload. As used herein, the term “messenger RNA” (mRNA) refers to any polynucleotide which encodes a polypeptide of interest and which is capable of being translated to produce the encoded polypeptide of interest in vitro, in vivo, in situ, or ex vivo. The components of an mRNA include, but are not limited to, a coding region, a 5′UTR, a 3′UTR, a 5′ cap and a poly-A tail. In some embodiments, the encoded mRNA or any portion of the mRNA be codon optimized.

### Polypeptide

In one embodiment, the payload encodes a polypeptide which may be a peptide or protein. A protein encoded by the payload may comprise a secreted protein, an intracellular protein, an extracellular protein, a membrane protein, and/or fragment or variant thereof.

In one embodiment, the encoded proteins may be structural or functional.

In one embodiment, proteins encoded by the payload construct payload construct include, but are not limited to, mammalian proteins.

In one embodiment the protein encoded by the payload is between 50-5000 amino acids in length. In some embodiments the protein encoded is between 50-2000 amino acids in length. In some embodiments the protein encoded is between 50-1000 amino acids in length. In some embodiments the protein encoded is between 50-1500 amino acids in length. In some embodiments the protein encoded is between 50-1000 amino acids in length. In some embodiments the protein encoded is between 50-800 amino acids in length. In some embodiments the protein encoded is between 50-600 amino acids in length. In some embodiments the protein encoded is between 50-400 amino acids in length. In some embodiments the protein encoded is between 50-200 amino acids in length. In some embodiments the protein encoded is between 50-100 amino acids in length.

In some embodiments the peptide encoded by the payload is between 4-50 amino acids in length. In one embodiment, the shortest length of a region of the payload of the present invention encoding a peptide can be the length that is sufficient to encode for a tetrapeptide, a pentapeptide, a hexapeptide, a heptapeptide, an octapeptide, a nonapeptide, or a decapeptide. In another embodiment, the length may be sufficient to encode a peptide of 2-30 amino acids, e.g. 5-30, 10-30, 2-25, 5-25, 10-25, or 10-20 amino acids. The length may be sufficient to encode for a peptide of at least 11, 12, 13, 14, 15, 17, 20, 25 or 30 amino acids, or a peptide that is no longer than 50 amino acids, e.g. no longer than 35, 30, 25, 20, 17, 15, 14, 13, 12, 11 or 10 amino acids.

### Modulatory Nucleic Acids

In one embodiment, an RNA sequence encoded by the payload may be a tRNA, rRNA, tmRNA, miRNA, RNAi, siRNA, piRNA, shRNA antisense RNA, double stranded RNA, snRNA, snoRNA, and/or long non-coding RNA (IncRNA). These RNA sequences along with siRNA, shRNA, antisense molecules and the like may also be referred to as “modulatory nucleic acids”.

In one embodiment, the RNA encoded by the payload is a IncRNA or RNAi construct designed to target IncRNA. Non-limiting examples of such IncRNA molecules and RNAi constructs designed to target such IncRNA are taught in International Publication, WO2012/018881, the contents of which are incorporated by reference in their entirety.

In one embodiment, the payload encodes a microRNA (miRNA) or engineered precursors thereof, as the payload. MicroRNAs (miRNAs) are 19-25 nucleotide RNAs that bind to nucleic acid molecules and down-regulate gene expression either by reducing nucleic acid molecule stability or by inhibiting translation. As a non-limiting example, the payloads described herein may encode one or more microRNA target sequences, microRNA sequences, or microRNA seeds, or any known precursors thereof such as pre- or pri-microRNAs. Such sequences may correspond to any known microRNA such as those taught in US Publication US2005/0261218 and US Publication US2005/0059005, the contents of which are incorporated herein by reference in their entirety.

A microRNA sequence comprises a “seed” region, i.e., a sequence in the region of positions 2-8 of the mature microRNA, which sequence has perfect Watson-Crick complementarity to the miRNA target sequence. A microRNA seed may comprise positions 2-8 or 2-7 of the mature microRNA. In some embodiments, a microRNA seed may comprise 7 nucleotides (e.g., nucleotides 2-8 of the mature microRNA), wherein the seed-complementary site in the corresponding miRNA target is flanked by an adenine (A) opposed to microRNA position 1. In some embodiments, a microRNA seed may comprise 6 nucleotides (e.g., nucleotides 2-7 of the mature microRNA), wherein the seed-complementary site in the corresponding miRNA target is flanked by an adenine (A) opposed to microRNA position 1. See for example, Grimson A, Farh K K, Johnston W K, Garrett-Engele P, Lim L P, Bartel D P; Mol Cell. 2007 Jul. 6; 27(1):91-105; each of which is herein incorporated by reference in their entirety. The bases of the microRNA seed have complete complementarity with the target sequence.

In one embodiment, the payload encodes an RNA sequence that may be processed to produce a siRNA, miRNA or other double stranded (ds) or single stranded (ss) gene modulatory nucleic acids or motifs.

In one embodiment, the siRNA duplexes or dsRNA encoded by the payload can be used to inhibit gene expression in a cell, in particular cells of the CNS. In some aspects, the inhibition of gene expression refers to an inhibition by at least about 20%, 30%, 40%, 50%, 60%, 70%, 80%, 85%, 90%, 95% and 100%. In one aspect, the protein product of the targeted gene may be inhibited by at least about 20%, 30%, 40%, 50%, 60%, 70%, 80%, 85%, 90%, 95% and 100%. The gene can be either a wild type gene or a gene with at least one mutation (mutated gene). The targeted protein may be either a wild type protein or a protein with at least one mutation (mutated protein).

In one embodiment, the present invention provides methods for treating, or ameliorating a disease or condition associated with abnormal gene and/or protein in a subject in need of treatment, the method comprising administering to the subject any effective amount of at least one AAV particle encoding an siRNA duplex targeting the gene, delivering duplex into targeted cells, inhibiting the gene expression and protein production, and ameliorating symptoms of the disease or condition in the subject.

### Gene Replacement or Activation

In one embodiment, the payload encodes an RNA sequence to increase the expression of a gene or replace a gene. As a non-limiting example, AAV particles may comprise a viral genome comprising a payload which encodes a normal gene to replace a mutated, defective or nonfunctional copy of that gene in the recipient.

In some aspects, the increase of gene expression refers to an increase by at least about 20%, 30%, 40%, 50%, 60%, 70%, 80%, 85%, 90%, 95% and 100%. In one aspect, the protein product of the targeted gene may be increased by at least about 20%, 30%, 40%, 50%, 60%, 70%, 80%, 85%, 90%, 95% and 100%.

### Functional Payloads

In one embodiment, a payload may encode polypeptides that are or can be a fusion protein.

In one embodiment, a payload may encode polypeptides that are or can be polypeptides having a desired biological activity.

In one embodiment, a payload may encode polypeptides that are or can be gene products that can complement a genetic defect.

In one embodiment, a payload may encode polypeptides that are or can be RNA molecules.

In one embodiment, a payload may encode polypeptides that are or can be transcription factors.

In one embodiment, a payload may encode polypeptides that are or can be other gene products that are of interest in regulation and/or expression.

In one embodiment, a payload may comprise nucleotide sequences that provide a desired effect or regulatory function (e.g., transposons, transcription factors).

The encoded payload may comprise a gene therapy product. In some embodiments, a gene therapy product may comprise a substitute for a non-functional gene that is absent or mutated.

In one embodiment, a payload may encode polypeptides that are or can be a marker to assess cell transformation and expression.

In one embodiment, a payload may comprise or encode a selectable marker. A selectable marker may comprise a gene sequence or a protein encoded by a gene sequence expressed in a host cell that allows for the identification, selection, and/or purification of the host cell from a population of cells that may or may not express the selectable marker. In one embodiment, the selectable marker provides resistance to survive a selection process that would otherwise kill the host cell, such as treatment with an antibiotic. In another embodiment, an antibiotic selectable marker may comprise one or more antibiotic resistance factors, including but not limited to neomycin resistance (e.g., neo), hygromycin resistance, kanamycin resistance, and/or puromycin resistance.

In some embodiments, a payload may comprise or encode any nucleic acid sequence encoding a polypeptide can be used as a selectable marker comprising recognition by a specific antibody.

In some embodiments, a payload may comprise or encode a selectable marker including, but not limited to, β-lactamase, luciferase, β-galactosidase, or any other reporter gene as that term is understood in the art, including cell-surface markers, such as CD4 or the truncated nerve growth factor (NGFR) (for GFP, see WO 96/23810; Heim et al., Current Biology 2:178-182 (1996); Heim et al., Proc. Natl. Acad. Sci. USA (1995); or Heim et al., Science 373:663-664 (1995); for β-lactamase, see WO 96/30540; the contents of each of which are herein incorporated by reference in its entirety).

In some embodiments, a payload may comprise or encode a selectable marker comprising a fluorescent protein. A fluorescent protein as herein described may comprise any fluorescent marker including but not limited to green, yellow, and/or red fluorescent protein (GFP, YFP, and/or RFP).

### Payload Construct

In one embodiment, the AAV particle may comprise a payload construct. As used herein, “payload construct” refers to one or more polynucleotide regions encoding or comprising a payload that is flanked on one or both sides by an inverted terminal repeat (ITR) sequence.

In one embodiment, the payload construct may comprise more than one payload. As a non-limiting example, a target cell transduced with an AAV particle comprising more than one payload may express each of the payloads in a single cell.

In some embodiments, the payload construct may encode a coding or non-coding RNA.

In one embodiment, a payload construct encoding one or more payloads for expression in a target cell may comprise one or more payload or non-payload nucleotide sequences operably linked to at least one target cell-compatible promoter.

In one embodiment, the ITRs in the AAV particle are derived from the same AAV serotype.

In one embodiment, the ITRs in the AAV particle are derived from different AAV serotypes.

In one embodiment, the ITRs in the AAV particle are the same.

In one embodiment, the ITRs in the AAV particle are different. In one aspect, the ITRs may be derived from the same AAV serotype. In another aspect, the ITRs may be derived from different serotypes.

### Regulatory Sequence

A person skilled in the art may recognize that expression of a payload in a target cell may require a regulatory sequence.

In one embodiment, the viral genome comprises a regulatory sequence efficient for expression of the payload.

In one embodiment, the viral genome comprises a regulatory sequence efficient for driving expression in the cell being targeted.

In one embodiment, the viral genome comprises a regulatory sequence such as, but not limited to, promoters. As a non-limiting example, the promoter may be (1) CMV promoter, (2) CBA promoter, (3) FRDA or FXN promoter, (4) UBC promoter, (5) GUSB promoter, (6) NSE promoter, (7) Synapsin promoter, (8) MeCP2 promoter, (9) GFAP promoter, (10) H1 promoter, (11) U6 promoter, (12) NFL promoter, (13) NFH promoter, (14) SCN8A promoter, or (15) PGK promoter.

### Promoters

A person skilled in the art may recognize that expression of a payload in a target cell may require a specific promoter including, but not limited to, a promoter that is species specific, inducible, tissue-specific, or cell cycle-specific (Parr et al., Nat. Med. 3:1145-9 (1997); the contents of which are herein incorporated by reference in its entirety).

In one embodiment, the viral genome comprises a promoter efficient for expression of the payload.

In one embodiment, the viral genome comprise a promoter efficient for driving expression in the cell being targeted.

In one embodiment, the promoter provides expression of a payload for a period of time in targeted tissues such as, but not limited to, nervous system tissues. Expression of the payload may be for a period of 1 hour, 2, hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 1 day, 2 days, 3 days, 4 days, 5 days, 6 days, 1 week, 8 days, 9 days, 10 days, 11 days, 12 days, 13 days, 2 weeks, 15 days, 16 days, 17 days, 18 days, 19 days, 20 days, 3 weeks, 22 days, 23 days, 24 days, 25 days, 26 days, 27 days, 28 days, 29 days, 30 days, 31 days, 1 month, 2 months, 3 months, 4 months, 5 months, 6 months, 7 months, 8 months, 9 months, 10 months, 11 months, 1 year, 13 months, 14 months, 15 months, 16 months, 17 months, 18 months, 19 months, 20 months, 21 months, 22 months, 23 months, 2 years, 3 years, 4 years, 5 years, 6 years, 7 years, 8 years, 9 years, 10 years, 11 years, 12 years, 13 years, 14 years, 15 years, 16 years, 17 years, 18 years, 19 years, 20 years, 21 years, 22 years, 23 years, 24 years, 25 years, 26 years, 27 years, 28 years, 29 years, 30 years, 31 years, 32 years, 33 years, 34 years, 35 years, 36 years, 37 years, 38 years, 39 years, 40 years, 41 years, 42 years, 43 years, 44 years, 45 years, 46 years, 47 years, 48 years, 49 years, 50 years, 55 years, 60 years, 65 years, or more than 65 years. Expression of the payload may be for 1-5 hours, 1-12 hours, 1-2 days, 1-5 days, 1-2 weeks, 1-3 weeks, 1-4 weeks, 1-2 months, 1-4 months, 1-6 months, 2-6 months, 3-6 months, 3-9 months, 4-8 months, 6-12 months, 1-2 years, 1-5 years, 2-5 years, 3-6 years, 3-8 years, 4-8 years or 5-10 years or 10-15 years, or 15-20 years, or 20-25 years, or 25-30 years, or 30-35 years, or 35-40 years, or 40-45 years, or 45-50 years, or 50-55 years, or 55-60 years, or 60-65 years.

In one embodiment, the viral genome comprises a region located approximately ˜5 kb upstream of the first exon of the encoded payload, more specifically, there is a 17 bp region located approximately 4.9 kb upstream of the first exon of the encoded frataxin gene in order to allow for expression with the FRDA promoter (See e.g., Puspasari et al. Long Range Regulation of Human FXN Gene Expression, PLOS ONE, 2011; the contents of which is herein incorporated by reference in its entirety).

In one embodiment, the promoter is less than 1 kb. The promoter may have a length of 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800 or more than 800. The promoter may have a length between 200-300, 200-400, 200-500, 200-600, 200-700, 200-800, 300-400, 300-500, 300-600, 300-700, 300-800, 400-500, 400-600, 400-700, 400-800, 500-600, 500-700, 500-800, 600-700, 600-800 or 700-800.

In one embodiment, the promoter may be a combination of two or more components, regions or sequences of the same or different promoters such as, but not limited to, CMV and CBA. Each component may have a length of 200, 210, 220, 230, 240, 250, 260, 270, 280, 290, 300, 310, 320, 330, 340, 350, 360, 370, 380, 381, 382, 383, 384, 385, 386, 387, 388, 389, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800 or more than 800. Each component may have a length between 200-300, 200-400, 200-500, 200-600, 200-700, 200-800, 300-400, 300-500, 300-600, 300-700, 300-800, 400-500, 400-600, 400-700, 400-800, 500-600, 500-700, 500-800, 600-700, 600-800 or 700-800.

In one embodiment, the promoter is a combination of a 382 nucleotide CMV-enhancer sequence and a 260 nucleotide CBA-promoter sequence.

In one embodiment, the viral genome comprises a ubiquitous promoter. Non-limiting examples of ubiquitous promoters include CMV, CBA (including derivatives CAG, CBh, etc.), EF-1α, PGK, UBC, GUSB (hGBp), and UCOE (promoter of HNRPA2B1-CBX3).

In one embodiment, any of the promoters taught by Yu, Soderblom, Gill, Husain, Passini, Xu, Drews or Raymond may be used in the present inventions. Yu et al. (Molecular Pain 2011, 7:63; the contents of which are herein incorporated by reference in its entirety) evaluated the expression of eGFP under the CAG, EFIα, PGK and UBC promoters in rat DRG cells and primary DRG cells using lentiviral vectors and found that UBC showed weaker expression than the other 3 promoters and there was only 10-12% glia expression seen for all promoters. Soderblom et al. (E. Neuro 2015; the contents of which are herein incorporated by reference in its entirety) evaluated the expression of eGFP in AAV8 with CMV and UBC promoters and AAV2 with the CMV promoter after injection in the motor cortex. Intranasal administration of a plasmid containing a UBC or EFIa promoter showed a sustained airway expression greater than the expression with the CMV promoter (See e.g., Gill et al., Gene Therapy 2001, Vol. 8, 1539-1546; the contents of which are herein incorporated by reference in its entirety). Husain et al. (Gene Therapy 2009; the contents of which are herein incorporated by reference in its entirety) evaluated a HβH construct with a hGUSB promoter, a HSV-1LAT promoter and a NSE promoter and found that the HβH construct showed weaker expression than NSE in mice brain. Passini and Wolfe (J. Virol. 2001, 12382-12392, the contents of which are herein incorporated by reference in its entirety) evaluated the long term effects of the HβH vector following an intraventricular injection in neonatal mice and found that there was sustained expression for at least 1 year. Low expression in all brain regions was found by Xu et al. (Gene Therapy 2001, 8, 1323-1332; the contents of which are herein incorporated by reference in its entirety) when NF-L and NF-H promoters were used as compared to the CMV-lacZ, CMV-luc, EF, GFAP, hENK, nAChR, PPE, PPE+wpre, NSE (0.3 kb), NSE (1.8 kb) and NSE (1.8 kb+wpre). Xu et al. found that the promoter activity in descending order was NSE (1.8 kb), EF, NSE (0.3 kb), GFAP, CMV, hENK, PPE, NFL and NFH. NFL is a 650 nucleotide promoter and NFH is a 920 nucleotide promoter which are both absent in the liver but NFH is abundant in the sensory proprioceptive neurons, brain and spinal cord and NFH is present in the heart. SCN8A is a 470 nucleotide promoter which expresses throughout the DRG, spinal cord and brain with particularly high expression seen in the hippocampal neurons and cerebellar Purkinje cells, cortex, thalamus and hypothalamus (See e.g., Drews et al. Identification of evolutionary conserved, functional noncoding elements in the promoter region of the sodium channel gene SCN8A, Mamm Genome (2007) 18:723-731; and Raymond et al. Expression of Alternatively Spliced Sodium Channel α-subunit genes, Journal of Biological Chemistry (2004) 279(44) 46234-46241; the contents of each of which are herein incorporated by reference in their entireties).

In one embodiment, the viral genome comprises a promoter which is not cell specific.

In one embodiment, the promoter is a weak promoter (classified according to its affinity and other promoters affinity for RNA polymerase and/or sigma factor) for sustained expression of a payload in nervous tissues. In one embodiment, the promoter is a weak promoter for sustained frataxin expression in nervous system tissue such as, but not limited to, neuronal tissue and glial tissue.

In one embodiment, the Friedreich's Ataxia (FRDA) promoter is used in the viral genomes of the AAV particles described herein.

In one embodiment, the viral genome comprises an ubiquitin c (UBC) promoter. The UBC promoter may have a size of 300-350 nucleotides. As a non-limiting example, the UBC promoter is 332 nucleotides.

In one embodiment, the viral genome comprises a β-glucuronidase (GUSB) promoter. The GUSB promoter may have a size of 350-400 nucleotides. As a non-limiting example, the GUSB promoter is 378 nucleotides. As a non-limiting example, the viral genome may be 5′-promoter-CMV/globin intron-hFXN-RBG-3′, where the viral genome may be self-complementary and the capsid may be the DJ serotype.

In one embodiment, the viral genome comprises a neurofilament (NFL) promoter. The NFL promoter may have a size of 600-700 nucleotides. As a non-limiting example, the NFL promoter is 650 nucleotides. As a non-limiting example, the viral genome may be 5′-promoter-CMV/globin intron-hFXN-RBG-3, where the viral genome may be self-complementary and the capsid may be the DJ serotype.

In one embodiment, the viral genome comprises a neurofilament heavy (NFH) promoter. The NFH promoter may have a size of 900-950 nucleotides. As a non-limiting example, the NFH promoter is 920 nucleotides. As a non-limiting example, the viral genome may be 5′-promoter-CMV/globin intron-hFXN-RBG-3′, where the viral genome may be self-complementary and the capsid may be the DJ serotype.

In one embodiment, the viral genome comprises a SCN8A promoter. The SCN8A promoter may have a size of 450-500 nucleotides. As a non-limiting example, the SCN8A promoter is 470 nucleotides. As a non-limiting example, the viral genome may be d′-promoter-CMV/globin intron-hFXN-RBG-3, where the viral genome may be self-complementary and the capsid may be the DJ serotype.

In one embodiment, the viral genome comprises a frataxin (FXN) promoter.

In one embodiment, the viral genome comprises a phosphoglycerate kinase 1 (PGK) promoter.

In one embodiment, the viral genome comprises a chicken β-actin (CBA) promoter.

In one embodiment, the viral genome comprises an immediate-early cytomegalovirus (CMV) promoter.

In one embodiment, the viral genome comprises a H1 promoter.

In one embodiment, the viral genome comprises a U6 promoter.

In one embodiment, the viral genome comprises a liver or a skeletal muscle promoter. Non-limiting examples of liver promoters include hAAT and TBG. Non-limiting examples of skeletal muscle promoters include Desmin, MCK and C5-12.

In one embodiment, the viral genome comprises a liver or a skeletal muscle promoter. Non-limiting examples of liver promoters include hAAT and TBG. Non-limiting examples of skeletal muscle promoters include Desmin, MCK and C5-12.

In one embodiment, the viral genome comprises an engineered promoter.

### Enhancement Element

In one embodiment, the viral genome may comprise at least one an enhancer and/or expression element. The enhancer or expression element may be used in combination with a regulatory sequence.

In one embodiment, the viral genome comprises an transgene enhancer, a promoter and/or a 5′UTR intron. The transgene enhancer, also referred to herein as an “enhancer,” may be, but is not limited to, a CMV enhancer. The promoter may be, but is not limited to, a CMV, CBA, UBC, GUSB, NSE, Synapsin, MeCP2, and GFAP promoter. The 5′UTR/intron may be, but is not limited to, SV40, and CBA-MVM.

In one embodiment, the viral genome comprises an transgene enhancer, a promoter and/or an intron combination such as, but not limited to, (1) CMV enhancer, CMV promoter, SV40 5′UTR intron; (2) CMV enhancer, CBA promoter, SV 40 5′UTR intron; (3) CMV enhancer, CBA promoter, CBA-MVM 5′UTR intron.

### Transgene Enhancement

In one embodiment, the viral genome comprises at least one transgene enhancer element which can enhance the transgene target specificity and expression (See e.g., Powell et al. Viral Expression Cassette Elements to Enhance Transgene Target Specificity and Expression in Gene Therapy, 2015; the contents of which are herein incorporated by reference in its entirety). Non-limiting examples of transgene enhancer elements to enhance the transgene target specificity and expression include promoters, endogenous miRNAs, post-transcriptional regulatory elements (PREs), polyadenylation (PolyA) signal sequences and upstream enhancers (USEs), CMV enhancers and introns.

In one embodiment, the viral genome comprises at least one transgene enhancer element which is a CMV enhancer.

In one embodiment, the viral genome comprises at least one transgene enhancer element which is a promoter.

In one embodiment, the viral genome comprises at least one transgene enhancer element which is an intron.

In one embodiment, the viral genome comprises at least one transgene enhancer element which is endogenous miRNAs.

In one embodiment, the viral genome comprises at least one transgene enhancer element which is post-transcriptional regulatory elements (PREs).

In one embodiment, the viral genome comprises at least one transgene enhancer element which is polyadenylation (PolyA) signal sequences.

In one embodiment, the viral genome comprises at least one transgene enhancer element which is upstream enhancers (USEs).

### Tissue-Specific Expression

In one embodiment, the vector genome may comprise a tissue-specific expression element to promote expression of the payload in tissues and/or cells. As a non-limiting example, promoters can be tissue-specific expression elements include, but are not limited to, human elongation factor 1α-subunit (EF1α), immediate-early cytomegalovirus (CMV), chicken β-actin (CBA) and its derivative CAG, the β glucuronidase (GUSB), and ubiquitin C (UBC).

In one embodiment, the vector genome may comprise a tissue-specific expression elements which can be used to restrict expression to certain cell types such as, but not limited to, nervous system promoters which can be used to restrict expression to neurons, astrocytes, or oligodendrocytes.

In one embodiment, the vector genome may comprise a tissue-specific expression elements for neurons such as, but not limited to, neuron-specific enolase (NSE), platelet-derived growth factor (PDGF), platelet-derived growth factor B-chain (PDGF-β), the synapsin (Syn), the methyl-CpG binding protein 2 (MeCP2), Ca²⁺/calmodulin-dependent protein kinase II (CaMKII), metabotropic glutamate receptor 2 (mGluR2), NFL, NFH, np32, PPE, Enk and EAAT2 promoters.

In one embodiment, the vector genome may comprise a tissue-specific expression elements for astrocytes such as, but not limited to, the glial fibrillary acidic protein (GFAP) and EAAT2 promoters.

In one embodiment, the vector genome may comprise a tissue-specific expression elements for oligodendrocytes such as, but not limited to, the myelin basic protein (MBP) promoter.

### Introns

In one embodiment, the viral genome comprises at least one element to enhance the transgene expression such as one or more introns or portions thereof.

In one embodiment, the payload construct comprises at least one element to enhance the transgene expression such as one or more introns or portions thereof.

Non-limiting examples of introns include, MVM (67-97 bps), F.IX truncated intron 1 (300 bps), β-globin SD/immunoglobulin heavy chain splice acceptor (250 bps), adenovirus splice donor/immunoglobin splice acceptor (500 bps), SV40 late splice donor/splice acceptor (19S/16S) (180 bps) and hybrid adenovirus splice donor/IgG splice acceptor (230 bps).

In one embodiment, the intron or intron portion may be 100-500 nucleotides in length. The intron may have a length of 80, 90, 100, 110, 120, 130, 140, 150, 160, 170, 171, 172, 173, 174, 175, 176, 177, 178, 179, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490 or 500. The intron may have a length between 80-100, 80-120, 80-140, 80-160, 80-180, 80-200, 80-250, 80-300, 80-350, 80-400, 80-450, 80-500, 200-300, 200-400, 200-500, 300-400, 300-500, or 400-500.

### Capsids and Capsid Serotypes

In some embodiments, AAV particles of the present invention may be packaged in a capsid structure or may be capsid free. Such capsid free viral vector donor and/or acceptor sequences such as AAV, are described in, for example, US Publication 20140107186, the content of which is incorporated by reference in its entirety.

In one embodiment, the present invention, provides nucleic acids encoding the mutated or modified virus capsids and capsid proteins of the invention. In some embodiments the capsids are engineered according to the methods of co-owned and co-pending International Publication No. WO2015191508, the contents of which are herein incorporated by reference in their entirety.

In some embodiments, AAV particles produced according to the present invention may comprise hybrid serotypes with enhanced transduction to specific cell types of interest in the central nervous system, prolonged transgene expression and/or a safety profile. The hybrid serotypes may be generated by transcapsidation, adsorption of bi-specific antibody to capsid surface, mosaic capsid, and chimeric capsid, and/or other capsid protein modifications.

In some embodiments, AAV particles of the present invention may be further modified toward a specific therapeutic application by rational mutagenesis of capsid proteins (see, e.g., Pulicherla et al., Mol Ther, 2011, 19: 1070-1078), incorporation of peptide ligands to the capsid, for example a peptide derived from an NMDA receptor agonist for enhanced retrograde transport (Xu et al., Virology, 2005, 341: 203-214), and directed evolution to produce new AAV variants for increased CNS transduction.

In some embodiments, AAV particles produced according to the present invention may comprise different capsid proteins, either naturally occurring and/or recombinant, including, but not limited to, AAV1, AAV2, AAV3, AAV4, AAV5, AAV6, AAV7, AAV8, AAV9, AAV10, and AAV11, AAV12, AAVrh8, AAVrh10, AAV-DJ, and AAV-DJ/8 capsid serotypes, or variants thereof (e.g., AAV3A and AAV3B). Nucleic acid sequences encoding one or more AAV capsid proteins useful in the present invention are disclosed in the commonly owned International Publication No. WO2015191508, the contents of which are herein incorporated by reference in their entirety.

In some embodiments, AAV particles of the present invention may comprise or be derived from any natural or recombinant AAV serotype. According to the present invention, the AAV particles may utilize or be based on a serotype selected from any of the following AAV1, AAV2, AAV2G9, AAV3, AAV3a, AAV3b, AAV3-3, AAV4, AAV4-4, AAV5, AAV6, AAV6.1, AAV6.2, AAV6.1.2, AAV7, AAV7.2, AAV8, AAV9, AAV9.11, AAV9.13, AAV9.16, AAV9.24, AAV9.45, AAV9.47, AAV9.61, AAV9.68, AAV9.84, AAV9.9, AAV10, AAV11, AAV12, AAV16.3, AAV24.1, AAV27.3, AAV42.12, AAV42-1b, AAV42-2, AAV42-3a, AAV42-3b, AAV42-4, AAV42-5a, AAV42-5b, AAV42-6b, AAV42-8, AAV42-10, AAV42-11, AAV42-12, AAV42-13, AAV42-15, AAV42-aa, AAV43-1, AAV43-12, AAV43-20, AAV43-21, AAV43-23, AAV43-25, AAV43-5, AAV44.1, AAV44.2, AAV44.5, AAV223.1, AAV223.2, AAV223.4, AAV223.5, AAV223.6, AAV223.7, AAV1-7/rh.48, AAV1-8/rh.49, AAV2-15/rh.62, AAV2-3/rh.61, AAV2-4/rh.50, AAV2-5/rh.51, AAV3.1/hu.6, AAV3.1/hu.9, AAV3-9/rh.52, AAV3-11/rh.53, AAV4-8/r11.64, AAV4-9/rh.54, AAV4-19/rh.55, AAV5-3/rh.57, AAV5-22/rh.58, AAV7.3/hu.7, AAV16.8/hu.10, AAV16.12/hu.11, AAV29.3/bb.1, AAV29.5/bb.2, AAV106.1/hu.37, AAV114.3/hu.40, AAV127.2/hu.41, AAV127.5/hu.42, AAV128.3/hu.44, AAV130.4/hu.48, AAV145.1/hu.53, AAV145.5/hu.54, AAV145.6/hu.55, AAV161.10/hu.60, AAV161.6/hu.61, AAV33.12/hu.17, AAV33.4/hu.15, AAV33.8/hu.16, AAV52/hu.19, AAV52.1/hu.20, AAV58.2/hu.25, AAVA3.3, AAVA3.4, AAVA3.5, AAVA3.7, AAVC1, AAVC2, AAVC5, AAV-DJ, AAV-DJ8, AAVF3, AAVF5, AAVH2, AAVrh.72, AAVhu.8, AAVrh.68, AAVrh.70, AAVpi.1, AAVpi.3, AAVpi.2, AAVrh.60, AAVrh.44, AAVrh.65, AAVrh.55, AAVrh.47, AAVrh.69, AAVrh.45, AAVrh.59, AAVhu.12, AAVH6, AAVLK03, AAVH-1/hu.1, AAVH-5/hu.3, AAVLG-10/rh.40, AAVLG-4/rh.38, AAVLG-9/hu.39, AAVN721-8/rh.43, AAVCh.5, AAVCh.5R1, AAVcy.2, AAVcy.3, AAVcy.4, AAVcy.5, AAVCy.5R1, AAVCy.5R2, AAVCy.5R3, AAVCy.5R4, AAVcy.6, AAVhu.1, AAVhu.2, AAVhu.3, AAVhu.4, AAVhu.5, AAVhu.6, AAVhu.7, AAVhu.9, AAVhu.10, AAVhu.11, AAVhu.13, AAVhu.15, AAVhu.16, AAVhu.17, AAVhu.18, AAVhu.20, AAVhu.21, AAVhu.22, AAVhu.23.2, AAVhu.24, AAVhu.25, AAVhu.27, AAVhu.28, AAVhu.29, AAVhu.29R, AAVhu.31, AAVhu.32, AAVhu.34, AAVhu.35, AAVhu.37, AAVhu.39, AAVhu.40, AAVhu.41, AAVhu.42, AAVhu.43, AAVhu.44, AAVhu.44R1, AAVhu.44R2, AAVhu.44R3, AAVhu.45, AAVhu.46, AAVhu.47, AAVhu.48, AAVhu.48R1, AAVhu.48R2, AAVhu.48R3, AAVhu.49, AAVhu.51, AAVhu.52, AAVhu.54, AAVhu.55, AAVhu.56, AAVhu.57, AAVhu.58, AAVhu.60, AAVhu.61, AAVhu.63, AAVhu.64, AAVhu.66, AAVhu.67, AAVhu.14/9, AAVhu.t 19, AAVrh.2, AAVrh.2R, AAVrh.8, AAVrh.8R, AAVrh.10, AAVrh.12, AAVrh.13, AAVrh.13R, AAVrh.14, AAVrh.17, AAVrh.18, AAVrh.19, AAVrh.20, AAVrh.21, AAVrh.22, AAVrh.23, AAVrh.24, AAVrh.25, AAVrh.31, AAVrh.32, AAVrh.33, AAVrh.34, AAVrh.35, AAVrh.36, AAVrh.37, AAVrh.37R2, AAVrh.38, AAVrh.39, AAVrh.40, AAVrh.46, AAVrh.48, AAVrh.48.1, AAVrh.48.1.2, AAVrh.48.2, AAVrh.49, AAVrh.51, AAVrh.52, AAVrh.53, AAVrh.54, AAVrh.56, AAVrh.57, AAVrh.58, AAVrh.61, AAVrh.64, AAVrh.64R1, AAVrh.64R2, AAVrh.67, AAVrh.73, AAVrh.74, AAVrh8R, AAVrh8R A586R mutant, AAVrh8R R533A mutant, AAAV, BAAV, caprine AAV, bovine AAV, AAVhE1.1, AAVhEr1.5, AAVhER1.14, AAVhEr1.8, AAVhEr1.16, AAVhEr1.18, AAVhEr1.35, AAVhEr1.7, AAVhEr1.36, AAVhEr2.29, AAVhEr2.4, AAVhEr2.16, AAVhEr2.30, AAVhEr2.31, AAVhEr2.36, AAVhER1.23, AAVhEr3.1, AAV2.5T, AAV-PAEC, AAV-LK01, AAV-LK02, AAV-LK03, AAV-LK04, AAV-LK05, AAV-LK06, AAV-LK07, AAV-LK08, AAV-LK09, AAV-LK10, AAV-LK11, AAV-LK12, AAV-LK13, AAV-LK14, AAV-LK15, AAV-LK16, AAV-LK17, AAV-LK18, AAV-LK19, AAV-PAEC2, AAV-PAEC4, AAV-PAEC6, AAV-PAEC7, AAV-PAEC8, AAV-PAEC11, AAV-PAEC12, AAV-2-pre-miRNA-101, AAV-8h, AAV-8b, AAV-h, AAV-b, AAV SM 10-2, AAV Shuffle 100-1, AAV Shuffle 100-3, AAV Shuffle 100-7, AAV Shuffle 10-2, AAV Shuffle 10-6, AAV Shuffle 10-8, AAV Shuffle 100-2, AAV SM 10-1, AAV SM 10-8, AAV SM 100-3, AAV SM 100-10, BNP61 AAV, BNP62 AAV, BNP63 AAV, AAVrh.50, AAVrh.43, AAVrh.62, AAVrh.48, AAVhu.19, AAVhu.11, AAVhu.53, AAV4-8/rh.64, AAVLG-9/hu.39, AAV54.5/hu.23, AAV54.2/hu.22, AAV54.7/hu.24, AAV54.1/hu.21, AAV54.4R/hu.27, AAV46.2/hu.28, AAV46.6/hu.29, AAV128.1/hu.43, true type AAV (ttAAV), UPENN AAV 10 and/or Japanese AAV 10 serotypes, and variants thereof. As a non-limiting example, the capsid of the recombinant AAV virus is AAV2. As a non-limiting example, the capsid of the recombinant AAV virus is AAVrh10. As a non-limiting example, the capsid of the recombinant AAV virus is AAV9(hu14). As a non-limiting example, the capsid of the recombinant AAV virus is AAV-DJ. As a non-limiting example, the capsid of the recombinant AAV virus is AAV9.47. As a non-limiting example, the capsid of the recombinant AAV virus is AAV-DJ8.

In some embodiments, the AAV particles of the present invention may comprise or be derived from an AAV serotype which may be, or have, a sequence as described in United States Publication No. US20030138772, the contents of which are herein incorporated by reference in their entirety, such as, but not limited to, AAV1 (SEQ ID NO: 6 and 64 of US20030138772), AAV2 (SEQ ID NO: 7 and 70 of US20030138772), AAV3 (SEQ ID NO: 8 and 71 of US20030138772), AAV4 (SEQ ID NO: 63 of US20030138772), AAV5 (SEQ ID NO: 114 of US20030138772), AAV6 (SEQ ID NO: 65 of US20030138772), AAV7 (SEQ ID NO: 1-3 of US20030138772), AAV8 (SEQ ID NO: 4 and 95 of US20030138772), AAV9 (SEQ ID NO: 5 and 100 of US20030138772), AAV10 (SEQ ID NO: 117 of US20030138772), AAV11 (SEQ ID NO: 118 of US20030138772), AAV12 (SEQ ID NO: 119 of US20030138772), AAVrh10 (amino acids 1 to 738 of SEQ ID NO: 81 of US20030138772), AAV16.3 (US20030138772 SEQ ID NO: 10), AAV29.3/bb.1 (US20030138772 SEQ ID NO: 11), AAV29.4 (US20030138772 SEQ ID NO: 12), AAV29.5/bb.2 (US20030138772 SEQ ID NO: 13), AAV1.3 (US20030138772 SEQ ID NO: 14), AAV13.3 (US20030138772 SEQ ID NO: 15), AAV24.1 (US20030138772 SEQ ID NO: 16), AAV27.3 (US20030138772 SEQ ID NO: 17), AAV7.2 (US20030138772 SEQ ID NO: 18), AAVC1 (US20030138772 SEQ ID NO: 19), AAVC3 (US20030138772 SEQ ID NO: 20), AAVC5 (US20030138772 SEQ ID NO: 21), AAVF1 (US20030138772 SEQ ID NO: 22), AAVF3 (US20030138772 SEQ ID NO: 23), AAVF5 (US20030138772 SEQ ID NO: 24), AAVH6 (US20030138772 SEQ ID NO: 25), AAVH2 (US20030138772 SEQ ID NO: 26), AAV42-8 (US20030138772 SEQ ID NO: 27), AAV42-15 (US20030138772 SEQ ID NO: 28), AAV42-5b (US20030138772 SEQ ID NO: 29), AAV42-1b (US20030138772 SEQ ID NO: 30), AAV42-13 (US20030138772 SEQ ID NO: 31), AAV42-3a (US20030138772 SEQ ID NO: 32), AAV42-4 (US20030138772 SEQ ID NO: 33), AAV42-5a (US20030138772 SEQ ID NO: 34), AAV42-10 (US20030138772 SEQ ID NO: 35), AAV42-3b (US20030138772 SEQ ID NO: 36), AAV42-11 (US20030138772 SEQ ID NO: 37), AAV42-6b (US20030138772 SEQ ID NO: 38), AAV43-1 (US20030138772 SEQ ID NO: 39), AAV43-5 (US20030138772 SEQ ID NO: 40), AAV43-12 (US20030138772 SEQ ID NO: 41), AAV43-20 (US20030138772 SEQ ID NO: 42), AAV43-21 (US20030138772 SEQ ID NO: 43), AAV43-23 (US20030138772 SEQ ID NO: 44), AAV43-25 (US20030138772 SEQ ID NO: 45), AAV44.1 (US20030138772 SEQ ID NO: 46), AAV44.5 (US20030138772 SEQ ID NO: 47), AAV223.1 (US20030138772 SEQ ID NO: 48), AAV223.2 (US20030138772 SEQ ID NO: 49), AAV223.4 (US20030138772 SEQ ID NO: 50), AAV223.5 (US20030138772 SEQ ID NO: 51), AAV223.6 (US20030138772 SEQ ID NO: 52), AAV223.7 (US20030138772 SEQ ID NO: 53), AAVA3.4 (US20030138772 SEQ ID NO: 54), AAVA3.5 (US20030138772 SEQ ID NO: 55), AAVA3.7 (US20030138772 SEQ ID NO: 56), AAVA3.3 (US20030138772 SEQ ID NO: 57), AAV42.12 (US20030138772 SEQ ID NO: 58), AAV44.2 (US20030138772 SEQ ID NO: 59), AAV42-2 (US20030138772 SEQ ID NO: 9), or variants thereof.

In some embodiments, the AAV particles of the present invention may comprise or be derived from AAV serotype which may be, or have, a sequence as described in United States Publication No. US20150159173, the contents of which are herein incorporated by reference in their entirety, such as, but not limited to, AAV2 (SEQ ID NO: 7 and 23 of US20150159173), rh20 (SEQ ID NO: 1 of US20150159173), rh32/33 (SEQ ID NO: 2 of US20150159173), rh39 (SEQ ID NO: 3, 20 and 36 of US20150159173), rh46 (SEQ ID NO: 4 and 22 of US20150159173), rh73 (SEQ ID NO: 5 of US20150159173), rh74 (SEQ ID NO: 6 of US20150159173), AAV6.1 (SEQ ID NO: 29 of US20150159173), rh.8 (SEQ ID NO: 41 of US20150159173), rh.48.1 (SEQ ID NO: 44 of US20150159173), hu.44 (SEQ ID NO: 45 of US20150159173), hu.29 (SEQ ID NO: 42 of US20150159173), hu.48 (SEQ ID NO: 38 of US20150159173), rh54 (SEQ ID NO: 49 of US20150159173), AAV2 (SEQ ID NO: 7 of US20150159173), cy.5 (SEQ ID NO: 8 and 24 of US20150159173), rh.10 (SEQ ID NO: 9 and 25 of US20150159173), rh.13 (SEQ ID NO: 10 and 26 of US20150159173), AAV1 (SEQ ID NO: 11 and 27 of US20150159173), AAV3 (SEQ ID NO: 12 and 28 of US20150159173), AAV6 (SEQ ID NO: 13 and 29 of US20150159173), AAV7 (SEQ ID NO: 14 and 30 of US20150159173), AAV8 (SEQ ID NO: 15 and 31 of US20150159173), hu.13 (SEQ ID NO: 16 and 32 of US20150159173), hu.26 (SEQ ID NO: 17 and 33 of US20150159173), hu.37 (SEQ ID NO: 18 and 34 of US20150159173), hu.53 (SEQ ID NO: 19 and 35 of US20150159173), rh.43 (SEQ ID NO: 21 and 37 of US20150159173), rh2 (SEQ ID NO: 39 of US20150159173), rh.37 (SEQ ID NO: 40 of US20150159173), rh.64 (SEQ ID NO: 43 of US20150159173), rh.48 (SEQ ID NO: 44 of US20150159173), ch.5 (SEQ ID NO 46 of US20150159173), rh.67 (SEQ ID NO: 47 of US20150159173), rh.58 (SEQ ID NO: 48 of US20150159173), or variants thereof including, but not limited to Cy5R1, Cy5R2, Cy5R3, Cy5R4, rh.13R, rh.37R2, rh.2R, rh.8R, rh.48.1, rh.48.2, rh.48.1.2, hu.44R1, hu.44R2, hu.44R3, hu.29R, ch.5R1, rh64R1, rh64R2, AAV6.2, AAV6.1, AAV6.12, hu.48R1, hu.48R2, and hu.48R3.

In some embodiments, the AAV particles of the present invention may comprise or be derived from AAV serotype which may be, or have, a sequence as described in U.S. Pat. No. 7,198,951, the contents of which are herein incorporated by reference in their entirety, such as, but not limited to, AAV9 (SEQ ID NO: 1-3 of U.S. Pat. No. 7,198,951), AAV2 (SEQ ID NO: 4 of U.S. Pat. No. 7,198,951), AAV1 (SEQ ID NO: 5 of U.S. Pat. No. 7,198,951), AAV3 (SEQ ID NO: 6 of U.S. Pat. No. 7,198,951), and AAV8 (SEQ ID NO: 7 of U.S. Pat. No. 7,198,951).

In some embodiments, the AAV particles of the present invention may comprise or be derived from AAV serotype which may be, or have, a mutation in the AAV9 sequence as described by N Pulicherla et al. (Molecular Therapy 19(6): 1070-1078 (2011), herein incorporated by reference in its entirety), such as but not limited to, AAV9.9, AAV9.11, AAV9.13, AAV9.16, AAV9.24, AAV9.45, AAV9.47, AAV9.61, AAV9.68, AAV9.84.

In some embodiments, the AAV particles of the present invention may comprise or be derived from AAV serotype which may be, or have, a sequence as described in U.S. Pat. No. 6,156,303, the contents of which are herein incorporated by reference in their entirety, such as, but not limited to, AAV3B (SEQ ID NO: 1 and 10 of U.S. Pat. No. 6,156,303), AAV6 (SEQ ID NO: 2, 7 and 11 of U.S. Pat. No. 6,156,303), AAV2 (SEQ ID NO: 3 and 8 of U.S. Pat. No. 6,156,303), AAV3A (SEQ ID NO: 4 and 9, of U.S. Pat. No. 6,156,303), or derivatives thereof.

In some embodiments, the AAV particles of the present invention may comprise or be derived from AAV serotype which may be, or have, a sequence as described in United States Publication No. US20140359799, the contents of which are herein incorporated by reference in their entirety, such as, but not limited to, AAV8 (SEQ ID NO: 1 of US20140359799), AAVDJ (SEQ ID NO: 2 and 3 of US20140359799), or variants thereof.

In some embodiments, the AAV particle may comprise a capsid from a serotype such as, but not limited to, AAVDJ or a variant thereof, such as AAVDJ8 (or AAV-DJ8), as described by Grimm et al. (Journal of Virology 82(12): 5887-5911 (2008), herein incorporated by reference in its entirety). The amino acid sequence of AAVDJ8 may comprise two or more mutations in order to remove the heparin binding domain (HBD). As a non-limiting example, the AAV-DJ sequence described as SEQ ID NO: 1 in U.S. Pat. No. 7,588,772, the contents of which are herein incorporated by reference in their entirety, may comprise two mutations: (1) R587Q where arginine (R; Arg) at amino acid 587 is changed to glutamine (Q; Gln) and (2) R590T where arginine (R; Arg) at amino acid 590 is changed to threonine (T; Thr). As another non-limiting example, may comprise three mutations: (1) K406R where lysine (K; Lys) at amino acid 406 is changed to arginine (R; Arg), (2) R587Q where arginine (R; Arg) at amino acid 587 is changed to glutamine (Q; Gln) and (3) R590T where arginine (R; Arg) at amino acid 590 is changed to threonine (T; Thr).

In some embodiments, the AAV particles of the present invention may comprise or be derived from AAV serotype which may be, or have, a sequence of AAV4 as described in International Publication No. WO1998011244, the contents of which are herein incorporated by reference in their entirety, such as, but not limited to AAV4 (SEQ ID NO: 1-20 of WO1998011244).

In some embodiments, the AAV particles of the present invention may comprise or be derived from AAV serotype which may be, or have, a mutation in the AAV2 sequence to generate AAV2G9 as described in International Publication No. WO2014144229 and herein incorporated by reference in its entirety.

In some embodiments, the AAV particles of the present invention may comprise or be derived from AAV serotype which may be, or have, a sequence as described in International Publication No. WO2005033321, the contents of which are herein incorporated by reference in their entirety, such as, but not limited to AAV3-3 (SEQ ID NO: 217 of WO2005033321), AAV1 (SEQ ID NO: 219 and 202 of WO2005033321), AAV106.1/hu.37 (SEQ ID No: 10 of WO2005033321), AAV114.3/hu.40 (SEQ ID No: 11 of WO2005033321), AAV127.2/hu.41 (SEQ ID NO:6 and 8 of WO2005033321), AAV128.3/hu.44 (SEQ ID No: 81 of WO2005033321), AAV130.4/hu.48 (SEQ ID NO: 78 of WO2005033321), AAV145.1/hu.53 (SEQ ID No: 176 and 177 of WO2005033321), AAV145.6/hu.56 (SEQ ID NO: 168 and 192 of WO2005033321), AAV16.12/hu.11 (SEQ ID NO: 153 and 57 of WO2005033321), AAV16.8/hu.10 (SEQ ID NO: 156 and 56 of WO2005033321), AAV161.10/hu.60 (SEQ ID No: 170 of WO2005033321), AAV161.6/hu.61 (SEQ ID No: 174 of WO2005033321), AAV1-7/rh.48 (SEQ ID NO: 32 of WO2005033321), AAV1-8/rh.49 (SEQ ID NOs: 103 and 25 of WO2005033321), AAV2 (SEQ ID NO: 211 and 221 of WO2005033321), AAV2-15/rh.62 (SEQ ID No: 33 and 114 of WO2005033321), AAV2-3/rh.61 (SEQ ID NO: 21 of WO2005033321), AAV2-4/rh.50 (SEQ ID No: 23 and 108 of WO2005033321), AAV2-5/rh.51 (SEQ ID NO: 104 and 22 of WO2005033321), AAV3.1/hu.6 (SEQ ID NO: 5 and 84 of WO2005033321), AAV3.1/hu.9 (SEQ ID NO: 155 and 58 of WO2005033321), AAV3-11/rh.53 (SEQ ID NO: 186 and 176 of WO2005033321), AAV3-3 (SEQ ID NO: 200 of WO2005033321), AAV33.12/hu.17 (SEQ ID NO:4 of WO2005033321), AAV33.4/hu.15 (SEQ ID No: 50 of WO2005033321), AAV33.8/hu.16 (SEQ ID No: 51 of WO2005033321), AAV3-9/rh.52 (SEQ ID NO: 96 and 18 of WO2005033321), AAV4-19/rh.55 (SEQ ID NO: 117 of WO2005033321), AAV4-4 (SEQ ID NO: 201 and 218 of WO2005033321), AAV4-9/rh.54 (SEQ ID NO: 116 of WO2005033321), AAV5 (SEQ ID NO: 199 and 216 of WO2005033321), AAV52.1/hu.20 (SEQ ID NO: 63 of WO2005033321), AAV52/hu.19 (SEQ ID NO: 133 of WO2005033321), AAV5-22/rh.58 (SEQ ID No: 27 of WO2005033321), AAV5-3/rh.57 (SEQ ID NO: 105 of WO2005033321), AAV5-3/rh.57 (SEQ ID No: 26 of WO2005033321), AAV58.2/hu.25 (SEQ ID No: 49 of WO2005033321), AAV6 (SEQ ID NO: 203 and 220 of WO2005033321), AAV7 (SEQ ID NO: 222 and 213 of WO2005033321), AAV7.3/hu.7 (SEQ ID No: 55 of WO2005033321), AAV8 (SEQ ID NO: 223 and 214 of WO2005033321), AAVH-1/hu.1 (SEQ ID No: 46 of WO2005033321), AAVH-5/hu.3 (SEQ ID No: 44 of WO2005033321), AAVhu.1 (SEQ ID NO: 144 of WO2005033321), AAVhu.10 (SEQ ID NO: 156 of WO2005033321), AAVhu.11 (SEQ ID NO: 153 of WO2005033321), AAVhu.12 (WO2005033321 SEQ ID NO: 59), AAVhu.13 (SEQ ID NO: 129 of WO2005033321), AAVhu.14/AAV9 (SEQ ID NO: 123 and 3 of WO2005033321), AAVhu.15 (SEQ ID NO: 147 of WO2005033321), AAVhu.16 (SEQ ID NO: 148 of WO2005033321), AAVhu.17 (SEQ ID NO: 83 of WO2005033321), AAVhu.18 (SEQ ID NO: 149 of WO2005033321), AAVhu.19 (SEQ ID NO: 133 of WO2005033321), AAVhu.2 (SEQ ID NO: 143 of WO2005033321), AAVhu.20 (SEQ ID NO: 134 of WO2005033321), AAVhu.21 (SEQ ID NO: 135 of WO2005033321), AAVhu.22 (SEQ ID NO: 138 of WO2005033321), AAVhu.23.2 (SEQ ID NO: 137 of WO2005033321), AAVhu.24 (SEQ ID NO: 136 of WO2005033321), AAVhu.25 (SEQ ID NO: 146 of WO2005033321), AAVhu.27 (SEQ ID NO: 140 of WO2005033321), AAVhu.29 (SEQ ID NO: 132 of WO2005033321), AAVhu.3 (SEQ ID NO: 145 of WO2005033321), AAVhu.31 (SEQ ID NO: 121 of WO2005033321), AAVhu.32 (SEQ ID NO: 122 of WO2005033321), AAVhu.34 (SEQ ID NO: 125 of WO2005033321), AAVhu.35 (SEQ ID NO: 164 of WO2005033321), AAVhu.37 (SEQ ID NO: 88 of WO2005033321), AAVhu.39 (SEQ ID NO: 102 of WO2005033321), AAVhu.4 (SEQ ID NO: 141 of WO2005033321), AAVhu.40 (SEQ ID NO: 87 of WO2005033321), AAVhu.41 (SEQ ID NO: 91 of WO2005033321), AAVhu.42 (SEQ ID NO: 85 of WO2005033321), AAVhu.43 (SEQ ID NO: 160 of WO2005033321), AAVhu.44 (SEQ ID NO: 144 of WO2005033321), AAVhu.45 (SEQ ID NO: 127 of WO2005033321), AAVhu.46 (SEQ ID NO: 159 of WO2005033321), AAVhu.47 (SEQ ID NO: 128 of WO2005033321), AAVhu.48 (SEQ ID NO: 157 of WO2005033321), AAVhu.49 (SEQ ID NO: 189 of WO2005033321), AAVhu.51 (SEQ ID NO: 190 of WO2005033321), AAVhu.52 (SEQ ID NO: 191 of WO2005033321), AAVhu.53 (SEQ ID NO: 186 of WO2005033321), AAVhu.54 (SEQ ID NO: 188 of WO2005033321), AAVhu.55 (SEQ ID NO: 187 of WO2005033321), AAVhu.56 (SEQ ID NO: 192 of WO2005033321), AAVhu.57 (SEQ ID NO: 193 of WO2005033321), AAVhu.58 (SEQ ID NO: 194 of WO2005033321), AAVhu.6 (SEQ ID NO: 84 of WO2005033321), AAVhu.60 (SEQ ID NO: 184 of WO2005033321), AAVhu.61 (SEQ ID NO: 185 of WO2005033321), AAVhu.63 (SEQ ID NO: 195 of WO2005033321), AAVhu.64 (SEQ ID NO: 196 of WO2005033321), AAVhu.66 (SEQ ID NO: 197 of WO2005033321), AAVhu.67 (SEQ ID NO: 198 of WO2005033321), AAVhu.7 (SEQ ID NO: 150 of WO2005033321), AAVhu.8 (WO2005033321 SEQ ID NO: 12), AAVhu.9 (SEQ ID NO: 155 of WO2005033321), AAVLG-10/rh.40 (SEQ ID No: 14 of WO2005033321), AAVLG-4/rh.38 (SEQ ID NO: 86 of WO2005033321), AAVLG-4/rh.38 (SEQ ID No: 7 of WO2005033321), AAVN721-8/rh.43 (SEQ ID NO: 163 of WO2005033321), AAVN721-8/rh.43 (SEQ ID No: 43 of WO2005033321), AAVpi.1 (WO2005033321 SEQ ID NO: 28), AAVpi.2 (WO2005033321 SEQ ID NO: 30), AAVpi.3 (WO2005033321 SEQ ID NO: 29), AAVrh.38 (SEQ ID NO: 86 of WO2005033321), AAVrh.40 (SEQ ID NO: 92 of WO2005033321), AAVrh.43 (SEQ ID NO: 163 of WO2005033321), AAVrh.44 (WO2005033321 SEQ ID NO: 34), AAVrh.45 (WO2005033321 SEQ ID NO: 41), AAVrh.47 (WO2005033321 SEQ ID NO: 38), AAVrh.48 (SEQ ID NO: 115 of WO2005033321), AAVrh.49 (SEQ ID NO: 103 of WO2005033321), AAVrh.50 (SEQ ID NO: 108 of WO2005033321), AAVrh.51 (SEQ ID NO: 104 of WO2005033321), AAVrh.52 (SEQ ID NO: 96 of WO2005033321), AAVrh.53 (SEQ ID NO: 97 of WO2005033321), AAVrh.55 (WO2005033321 SEQ ID NO: 37), AAVrh.56 (SEQ ID NO: 152 of WO2005033321), AAVrh.57 (SEQ ID NO: 105 of WO2005033321), AAVrh.58 (SEQ ID NO: 106 of WO2005033321), AAVrh.59 (WO2005033321 SEQ ID NO: 42), AAVrh.60 (WO2005033321 SEQ ID NO: 31), AAVrh.61 (SEQ ID NO: 107 of WO2005033321), AAVrh.62 (SEQ ID NO: 114 of WO2005033321), AAVrh.64 (SEQ ID NO: 99 of WO2005033321), AAVrh.65 (WO2005033321 SEQ ID NO: 35), AAVrh.68 (WO2005033321 SEQ ID NO: 16), AAVrh.69 (WO2005033321 SEQ ID NO: 39), AAVrh.70 (WO2005033321 SEQ ID NO: 20), AAVrh.72 (WO2005033321 SEQ ID NO: 9), or variants thereof including, but not limited to, AAVcy.2, AAVcy.3, AAVcy.4, AAVcy.5, AAVcy.6, AAVrh.12, AAVrh.17, AAVrh.18, AAVrh.19, AAVrh.21, AAVrh.22, AAVrh.23, AAVrh.24, AAVrh.25, AAVrh.25/42 15, AAVrh.31, AAVrh.32, AAVrh.33, AAVrh.34, AAVrh.35, AAVrh.36, AAVrh.37, AAVrh14. Non limiting examples of variants include SEQ ID NO: 13, 15, 17, 19, 24, 36, 40, 45, 47, 48, 51-54, 60-62, 64-77, 79, 80, 82, 89, 90, 93-95, 98, 100, 101, 109-113, 118-120, 124, 126, 131, 139, 142, 151, 154, 158, 161, 162, 165-183, 202, 204-212, 215, 219, 224-236, of WO2005033321, the contents of which are herein incorporated by reference in their entirety.

In some embodiments, the AAV particles of the present invention may comprise or be derived from AAV serotype which may be, or have, a sequence as described in International Publication No. WO2015168666, the contents of which are herein incorporated by reference in their entirety, such as, but not limited to, AAVrh8R (SEQ ID NO: 9 of WO2015168666), AAVrh8R A586R mutant (SEQ ID NO: 10 of WO2015168666), AAVrh8R R533A mutant (SEQ ID NO: 11 of WO2015168666), or variants thereof.

In some embodiments, the AAV particles of the present invention may comprise or be derived from AAV serotype which may be, or have, a sequence as described in U.S. Pat. No. 9,233,131, the contents of which are herein incorporated by reference in their entirety, such as, but not limited to, AAVhE1.1 (SEQ ID NO:44 of U.S. Pat. No. 9,233,131), AAVhEr1.5 (SEQ ID NO:45 of U.S. Pat. No. 9,233,131), AAVhER1.14 (SEQ ID NO:46 of U.S. Pat. No. 9,233,131), AAVhEr1.8 (SEQ ID NO:47 of U.S. Pat. No. 9,233,131), AAVhEr1.16 (SEQ ID NO:48 of U.S. Pat. No. 9,233,131), AAVhEr1.18 (SEQ ID NO:49 of U.S. Pat. No. 9,233,131), AAVhEr1.35 (SEQ ID NO:50 of U.S. Pat. No. 9,233,131), AAVhEr1.7 (SEQ ID NO:51 of U.S. Pat. No. 9,233,131), AAVhEr1.36 (SEQ ID NO:52 of U.S. Pat. No. 9,233,131), AAVhEr2.29 (SEQ ID NO:53 of U.S. Pat. No. 9,233,131), AAVhEr2.4 (SEQ ID NO:54 of U.S. Pat. No. 9,233,131), AAVhEr2.16 (SEQ ID NO:55 of U.S. Pat. No. 9,233,131), AAVhEr2.30 (SEQ ID NO:56 of U.S. Pat. No. 9,233,131), AAVhEr2.31 (SEQ ID NO:58 of U.S. Pat. No. 9,233,131), AAVhEr2.36 (SEQ ID NO:57 of U.S. Pat. No. 9,233,131), AAVhER1.23 (SEQ ID NO:53 of U.S. Pat. No. 9,233,131), AAVhEr3.1 (SEQ ID NO:59 of U.S. Pat. No. 9,233,131), AAV2.5T (SEQ ID NO:42 of U.S. Pat. No. 9,233,131), or variants thereof.

In some embodiments, the AAV particles of the present invention may comprise or be derived from AAV serotype which may be, or have, a sequence as described in United States Patent Publication No. US20150376607, the contents of which are herein incorporated by reference in their entirety, such as, but not limited to, AAV-PAEC (SEQ ID NO:1 of US20150376607), AAV-LK01 (SEQ ID NO:2 of US20150376607), AAV-LK02 (SEQ ID NO:3 of US20150376607), AAV-LK03 (SEQ ID NO:4 of US20150376607), AAV-LK04 (SEQ ID NO:5 of US20150376607), AAV-LK05 (SEQ ID NO:6 of US20150376607), AAV-LK06 (SEQ ID NO:7 of US20150376607), AAV-LK07 (SEQ ID NO:8 of US20150376607), AAV-LK08 (SEQ ID NO:9 of US20150376607), AAV-LK09 (SEQ ID NO:10 of US20150376607), AAV-LK10 (SEQ ID NO:11 of US20150376607), AAV-LK11 (SEQ ID NO: 12 of US20150376607), AAV-LK12 (SEQ ID NO:13 of US20150376607), AAV-LK13 (SEQ ID NO:14 of US20150376607), AAV-LK14 (SEQ ID NO:15 of US20150376607), AAV-LK15 (SEQ ID NO:16 of US20150376607), AAV-LK16 (SEQ ID NO:17 of US20150376607), AAV-LK17 (SEQ ID NO:18 of US20150376607), AAV-LK18 (SEQ ID NO:19 of US20150376607), AAV-LK19 (SEQ ID NO:20 of US20150376607), AAV-PAEC2 (SEQ ID NO:21 of US20150376607), AAV-PAEC4 (SEQ ID NO:22 of US20150376607), AAV-PAEC6 (SEQ ID NO:23 of US20150376607), AAV-PAEC7 (SEQ ID NO:24 of US20150376607), AAV-PAEC8 (SEQ ID NO:25 of US20150376607), AAV-PAEC11 (SEQ ID NO:26 of US20150376607), AAV-PAEC12 (SEQ ID NO:27, of US20150376607), or variants thereof.

In some embodiments, the AAV particles of the present invention may comprise or be derived from AAV serotype which may be, or have, a sequence as described in U.S. Pat. No. 9,163,261, the contents of which are herein incorporated by reference in their entirety, such as, but not limited to, AAV-2-pre-miRNA-101 (SEQ ID NO: 1 U.S. Pat. No. 9,163,261), or variants thereof.

In some embodiments, the AAV particles of the present invention may comprise or be derived from AAV serotype which may be, or have, a sequence as described in United States Patent Publication No. US20150376240, the contents of which are herein incorporated by reference in their entirety, such as, but not limited to, AAV-8h (SEQ ID NO: 6 of US20150376240), AAV-8b (SEQ ID NO: 5 of US20150376240), AAV-h (SEQ ID NO: 2 of US20150376240), AAV-b (SEQ ID NO: 1 of US20150376240), or variants thereof.

In some embodiments, the AAV particles of the present invention may comprise or be derived from AAV serotype which may be, or have, a sequence as described in United States Patent Publication No. US20160017295, the contents of which are herein incorporated by reference in their entirety, such as, but not limited to, AAV SM 10-2 (SEQ ID NO: 22 of US20160017295), AAV Shuffle 100-1 (SEQ ID NO: 23 of US20160017295), AAV Shuffle 100-3 (SEQ ID NO: 24 of US20160017295), AAV Shuffle 100-7 (SEQ ID NO: 25 of US20160017295), AAV Shuffle 10-2 (SEQ ID NO: 34 of US20160017295), AAV Shuffle 10-6 (SEQ ID NO: 35 of US20160017295), AAV Shuffle 10-8 (SEQ ID NO: 36 of US20160017295), AAV Shuffle 100-2 (SEQ ID NO: 37 of US20160017295), AAV SM 10-1 (SEQ ID NO: 38 of US20160017295), AAV SM 10-8 (SEQ ID NO: 39 of US20160017295), AAV SM 100-3 (SEQ ID NO: 40 of US20160017295), AAV SM 100-10 (SEQ ID NO: 41 of US20160017295), or variants thereof.

In some embodiments, the AAV particles of the present invention may comprise or be derived from AAV serotype which may be, or have, a sequence as described in United States Patent Publication No. US20150238550, the contents of which are herein incorporated by reference in their entirety, such as, but not limited to, BNP61 AAV (SEQ ID NO: 1 of US20150238550), BNP62 AAV (SEQ ID NO: 3 of US20150238550), BNP63 AAV (SEQ ID NO: 4 of US20150238550), or variants thereof.

In some embodiments, the AAV particles of the present invention may comprise or be derived from an AAV serotype which may be or may have a sequence as described in United States Patent Publication No. US20150315612, the contents of which are herein incorporated by reference in their entirety, such as, but not limited to, AAVrh.50 (SEQ ID NO: 108 of US20150315612), AAVrh.43 (SEQ ID NO: 163 of US20150315612), AAVrh.62 (SEQ ID NO: 114 of US20150315612), AAVrh.48 (SEQ ID NO: 115 of US20150315612), AAVhu.19 (SEQ ID NO: 133 of US20150315612), AAVhu.11 (SEQ ID NO: 153 of US20150315612), AAVhu.53 (SEQ ID NO: 186 of US20150315612), AAV4-8/rh.64 (SEQ ID No: 15 of US20150315612), AAVLG-9/hu.39 (SEQ ID No: 24 of US20150315612), AAV54.5/hu.23 (SEQ ID No: 60 of US20150315612), AAV54.2/hu.22 (SEQ ID No: 67 of US20150315612), AAV54.7/hu.24 (SEQ ID No: 66 of US20150315612), AAV54.1/hu.21 (SEQ ID No: 65 of US20150315612), AAV54.4R/hu.27 (SEQ ID No: 64 of US20150315612), AAV46.2/hu.28 (SEQ ID No: 68 of US20150315612), AAV46.6/hu.29 (SEQ ID No: 69 of US20150315612), AAV128.1/hu.43 (SEQ ID No: 80 of US20150315612), or variants thereof.

In some embodiments, the AAV particles of the present invention may comprise or be derived from AAV serotype which may be, or have, a sequence as described in International Publication No. WO2015121501, the contents of which are herein incorporated by reference in their entirety, such as, but not limited to, true type AAV (ttAAV) (SEQ ID NO: 2 of WO2015121501), “UPenn AAV10” (SEQ ID NO: 8 of WO2015121501), “Japanese AAV10” (SEQ ID NO: 9 of WO2015121501), or variants thereof.

According to the present invention, the AAV particle may comprise an AAV capsid serotype which may be selected from or derived from a variety of species. In one embodiment, the AAV may be an avian AAV (AAAV). The AAAV serotype may be, or have, a sequence as described in U.S. Pat. No. 9,238,800, the contents of which are herein incorporated by reference in their entirety, such as, but not limited to, AAAV (SEQ ID NO: 1, 2, 4, 6, 8, 10, 12, and 14 of U.S. Pat. No. 9,238,800), or variants thereof.

In one embodiment, the AAV particle may comprise an AAV capsid serotype which may be or derived from a bovine AAV (BAAV). The BAAV serotype may be, or have, a sequence as described in U.S. Pat. No. 9,193,769, the contents of which are herein incorporated by reference in their entirety, such as, but not limited to, BAAV (SEQ ID NO: 1 and 6 of U.S. Pat. No. 9,193,769), or variants thereof. The BAAV serotype may be or have a sequence as described in U.S. Pat. No. 7,427,396, the contents of which are herein incorporated by reference in their entirety, such as, but not limited to, BAAV (SEQ ID NO: 5 and 6 of U.S. Pat. No. 7,427,396), or variants thereof.

In one embodiment, the AAV particle may comprise an AAV capsid serotype which may be or derived from a caprine AAV. The caprine AAV serotype may be, or have, a sequence as described in U.S. Pat. No. 7,427,396, the contents of which are herein incorporated by reference in their entirety, such as, but not limited to, caprine AAV (SEQ ID NO: 3 of US7427396), or variants thereof.

In other embodiments, the AAV particle may comprise an AAV capsid serotype which may be engineered as a hybrid AAV from two or more parental serotypes. In one embodiment, the AAV may be AAV2G9 which comprises sequences from AAV2 and AAV9. The AAV2G9 AAV serotype may be, or have, a sequence as described in United States Patent Publication No. US20160017005, the contents of which are herein incorporated by reference in its entirety.

In one embodiment, the AAV particle may comprise an AAV capsid serotype which may be generated by the AAV9 capsid library with mutations in amino acids 390-627 (VP1 numbering) as described by Pulicherla et al. (Molecular Therapy 19(6):1070-1078 (2011), the contents of which are herein incorporated by reference in their entirety. The serotype and corresponding nucleotide and amino acid substitutions may be, but is not limited to, AAV9.1 (G1594C; D532H), AAV6.2 (T1418A and T1436X; V473D and 1479K), AAV9.3 (T1238A; F413Y), AAV9.4 (T1250C and A1617T; F417S), AAV9.5 (A1235G, A1314T, A1642G, C1760T; Q412R, T548A, A587V), AAV9.6 (T1231A; F411I), AAV9.9 (G1203A, G1785T; W595C), AAV9.10 (A1500G, T1676C; M559T), AAV9.11 (A1425T, A1702C, A1769T; T568P, Q590L), AAV9.13 (A1369C, A1720T; N457H, T574S), AAV9.14 (T1340A, T1362C, T1560C, G1713A; L447H), AAV9.16 (A1775T; Q592L), AAV9.24 (T1507C, T1521G; W503R), AAV9.26 (A1337G, A1769C; Y446C, Q590P), AAV9.33 (A1667C; D556A), AAV9.34 (A1534G, C1794T; N512D), AAV9.35 (A1289T, T1450A, C1494T, A1515T, C1794A, G1816A; Q430L, Y484N, N98K, V606I), AAV9.40 (A1694T, E565V), AAV9.41 (A1348T, T1362C; T450S), AAV9.44 (A1684C, A1701T, A1737G; N562H, K567N), AAV9.45 (A1492T, C1804T; N498Y, L602F), AAV9.46 (G1441C, T1525C, T1549G; G481R, W509R, L517V), 9.47 (G1241A, G1358A, A1669G, C1745T; S414N, G453D, K557E, T582I), AAV9.48 (C1445T, A1736T; P482L, Q579L), AAV9.50 (A1638T, C1683T, T1805A; Q546H, L602H), AAV9.53 (G1301A, A1405C, C1664T, G1811T; R134Q, S469R, A555V, G604V), AAV9.54 (C1531A, T1609A; L511I, L537M), AAV9.55 (T1605A; F535L), AAV9.58 (C1475T, C1579A; T492I, H527N), AAV.59 (T1336C; Y446H), AAV9.61 (A1493T; N498I), AAV9.64 (C1531A, A1617T; L511I), AAV9.65 (C1335T, T1530C, C1568A; A523D), AAV9.68 (C1510A; P504T), AAV9.80 (G1441A; G481R), AAV9.83 (C1402A, A1500T; P468T, E500D), AAV9.87 (T1464C, T1468C; S490P), AAV9.90 (A1196T; Y399F), AAV9.91 (T1316G, A1583T, C1782G, T1806C; L439R, K528I), AAV9.93 (A1273G, A1421G, A1638C, C1712T, G1732A, A1744T, A1832T; S425G, Q474R, Q546H, P571L, G578R, T582S, D611V), AAV9.94 (A1675T; M559L) and AAV9.95 (T1605A; F535L).

In one embodiment, the AAV particle may comprise an AAV capsid serotype which may be a serotype comprising at least one AAV capsid CD8+ T-cell epitope. As a non-limiting example, the serotype may be AAV1, AAV2 or AAV8.

In one embodiment, the AAV particle may comprise an AAV capsid serotype which may be a serotype selected from any of those found in Table 1.

In one embodiment, the AAV particle may comprise an AAV capsid serotype which may comprise a sequence, fragment or variant thereof, of the sequences in Table 1.

In one embodiment, the AAV particle may comprise an AAV capsid serotype which may be encoded by a sequence, fragment or variant as described in Table 1.

TABLE 1
AAV Serotypes

| | SEQ | |
|---|---|---|
| Serotype | ID NO | Reference Information |
| AAV1 | 1 | US20150159173 SEQ ID NO: 11, |
| | | US20150315612 SEQ ID NO: 202 |
| AAV1 | 2 | US20160017295 SEQ ID NO: 1, |
| | | US20030138772 SEQ ID NO: 64, |
| | | US20150159173 SEQ ID NO: 27, |
| | | US20150315612 SEQ ID NO: 219, |
| | | U.S. Pat. No. 7,198,951 SEQ ID NO: 5 |
| AAV1 | 3 | US20030138772 SEQ ID NO: 6 |
| AAV1.3 | 4 | US20030138772 SEQ ID NO: 14 |
| AAV10 | 5 | US20030138772 SEQ ID NO: 117 |
| AAV10 | 6 | WO2015121501 SEQ ID NO: 9 |
| AAV10 | 7 | WO2015121501 SEQ ID NO: 8 |
| AAV11 | 8 | US20030138772 SEQ ID NO: 118 |
| AAV12 | 9 | US20030138772 SEQ ID NO: 119 |
| AAV2 | 10 | US20150159173 SEQ ID NO: 7, |
| | | US20150315612 SEQ ID NO: 211 |
| AAV2 | 11 | US20030138772 SEQ ID NO: 70, |
| | | US20150159173 SEQ ID NO: 23, |
| | | US20150315612 SEQ ID NO: 221, |
| | | US20160017295 SEQ ID NO: 2, |
| | | U.S. Pat. No. 6,156,303 SEQ ID NO: 4, |
| | | U.S. Pat. No. 7,198,951 SEQ ID NO: 4, |
| | | WO2015121501 SEQ ID NO: 1 |
| AAV2 | 12 | U.S. Pat. No. 6,156,303 SEQ ID NO: 8 |
| AAV2 | 13 | US20030138772 SEQ ID NO: 7 |
| AAV2 | 14 | U.S. Pat. No. 6,156,303 SEQ ID NO: 3 |
| AAV2.5T | 15 | U.S. Pat. No. 9,233,131 SEQ ID NO: 42 |
| AAV223.10 | 16 | US20030138772 SEQ ID NO: 75 |
| AAV223.2 | 17 | US20030138772 SEQ ID NO: 49 |
| AAV223.2 | 18 | US20030138772 SEQ ID NO: 76 |
| AAV223.4 | 19 | US20030138772 SEQ ID NO: 50 |
| AAV223.4 | 20 | US20030138772 SEQ ID NO: 73 |
| AAV223.5 | 21 | US20030138772 SEQ ID NO: 51 |
| AAV223.5 | 22 | US20030138772 SEQ ID NO: 74 |
| AAV223.6 | 23 | US20030138772 SEQ ID NO: 52 |
| AAV223.6 | 24 | US20030138772 SEQ ID NO: 78 |
| AAV223.7 | 25 | US20030138772 SEQ ID NO: 53 |
| AAV223.7 | 26 | US20030138772 SEQ ID NO: 77 |
| AAV29.3 | 27 | US20030138772 SEQ ID NO: 82 |
| AAV29.4 | 28 | US20030138772 SEQ ID NO: 12 |
| AAV29.5 | 29 | US20030138772 SEQ ID NO: 83 |
| AAV29.5 (AAVbb.2) | 30 | US20030138772 SEQ ID NO: 13 |
| AAV3 | 31 | US20150159173 SEQ ID NO: 12 |
| AAV3 | 32 | US20030138772 SEQ ID NO: 71, |
| | | US20150159173 SEQ ID NO: 28, |
| | | US20160017295 SEQ ID NO: 3, |
| | | U.S. Pat. No. 7,198,951 SEQ ID NO: 6 |
| AAV3 | 33 | US20030138772 SEQ ID NO: 8 |
| AAV3.3b | 34 | US20030138772 SEQ ID NO: 72 |
| AAV3-3 | 35 | US20150315612 SEQ ID NO: 200 |
| AAV3-3 | 36 | US20150315612 SEQ ID NO: 217 |
| AAV3a | 37 | U.S. Pat. No. 6,156,303 SEQ ID NO: 5 |
| AAV3a | 38 | U.S. Pat. No. 6,156,303 SEQ ID NO: 9 |
| AAV3b | 39 | U.S. Pat. No. 6,156,303 SEQ ID NO: 6 |
| AAV3b | 40 | U.S. Pat. No. 6,156,303 SEQ ID NO: 10 |
| AAV3b | 41 | U.S. Pat. No. 6,156,303 SEQ ID NO: 1 |
| AAV4 | 42 | US20140348794 SEQ ID NO: 17 |
| AAV4 | 43 | US20140348794 SEQ ID NO: 5 |
| AAV4 | 44 | US20140348794 SEQ ID NO: 3 |
| AAV4 | 45 | US20140348794 SEQ ID NO: 14 |
| AAV4 | 46 | US20140348794 SEQ ID NO: 15 |
| AAV4 | 47 | US20140348794 SEQ ID NO: 19 |
| AAV4 | 48 | US20140348794 SEQ ID NO: 12 |
| AAV4 | 49 | US20140348794 SEQ ID NO: 13 |
| AAV4 | 50 | US20140348794 SEQ ID NO: 7 |
| AAV4 | 51 | US20140348794 SEQ ID NO: 8 |
| AAV4 | 52 | US20140348794 SEQ ID NO: 9 |
| AAV4 | 53 | US20140348794 SEQ ID NO: 2 |
| AAV4 | 54 | US20140348794 SEQ ID NO: 10 |
| AAV4 | 55 | US20140348794 SEQ ID NO: 11 |
| AAV4 | 56 | US20140348794 SEQ ID NO: 18 |
| AAV4 | 57 | US20030138772 SEQ ID NO: 63, |
| | | US20160017295 SEQ ID NO: 4, |
| | | US20140348794 SEQ ID NO: 4 |
| AAV4 | 58 | US20140348794 SEQ ID NO: 16 |
| AAV4 | 59 | US20140348794 SEQ ID NO: 20 |
| AAV4 | 60 | US20140348794 SEQ ID NO: 6 |
| AAV4 | 61 | US20140348794 SEQ ID NO: 1 |
| AAV42.2 | 62 | US20030138772 SEQ ID NO: 9 |
| AAV42.2 | 63 | US20030138772 SEQ ID NO: 102 |
| AAV42.3b | 64 | US20030138772 SEQ ID NO: 36 |
| AAV42.3B | 65 | US20030138772 SEQ ID NO: 107 |
| AAV42.4 | 66 | US20030138772 SEQ ID NO: 33 |
| AAV42.4 | 67 | US20030138772 SEQ ID NO: 88 |
| AAV42.8 | 68 | US20030138772 SEQ ID NO: 27 |
| AAV42.8 | 69 | US20030138772 SEQ ID NO: 85 |
| AAV43.1 | 70 | US20030138772 SEQ ID NO: 39 |
| AAV43.1 | 71 | US20030138772 SEQ ID NO: 92 |
| AAV43.12 | 72 | US20030138772 SEQ ID NO: 41 |
| AAV43.12 | 73 | US20030138772 SEQ ID NO: 93 |
| AAV43.20 | 74 | US20030138772 SEQ ID NO: 42 |
| AAV43.20 | 75 | US20030138772 SEQ ID NO: 99 |
| AAV43.21 | 76 | US20030138772 SEQ ID NO: 43 |
| AAV43.21 | 77 | US20030138772 SEQ ID NO: 96 |
| AAV43.23 | 78 | US20030138772 SEQ ID NO: 44 |
| AAV43.23 | 79 | US20030138772 SEQ ID NO: 98 |
| AAV43.25 | 80 | US20030138772 SEQ ID NO: 45 |
| AAV43.25 | 81 | US20030138772 SEQ ID NO: 97 |
| AAV43.5 | 82 | US20030138772 SEQ ID NO: 40 |
| AAV43.5 | 83 | US20030138772 SEQ ID NO: 94 |
| AAV4-4 | 84 | US20150315612 SEQ ID NO: 201 |
| AAV4-4 | 85 | US20150315612 SEQ ID NO: 218 |
| AAV44.1 | 86 | US20030138772 SEQ ID NO: 46 |
| AAV44.1 | 87 | US20030138772 SEQ ID NO: 79 |
| AAV44.5 | 88 | US20030138772 SEQ ID NO: 47 |
| AAV44.5 | 89 | US20030138772 SEQ ID NO: 80 |
| AAV4407 | 90 | US20150315612 SEQ ID NO: 90 |
| AAV5 | 91 | U.S. Pat. No. 7,427,396 SEQ ID NO: 1 |
| AAV5 | 92 | US20030138772 SEQ ID NO: 114 |
| AAV5 | 93 | US20160017295 SEQ ID NO: 5, |
| | | U.S. Pat. No. 7,427,396 SEQ ID NO: 2, |
| | | US20150315612 SEQ ID NO: 216 |
| AAV5 | 94 | US20150315612 SEQ ID NO: 199 |
| AAV6 | 95 | US20150159173 SEQ ID NO: 13 |
| AAV6 | 96 | US20030138772 SEQ ID NO: 65, |
| | | US20150159173 SEQ ID NO: 29, |
| | | US20160017295 SEQ ID NO: 6, |
| | | U.S. Pat. No. 6,156,303 SEQ ID NO: 7 |
| AAV6 | 97 | U.S. Pat. No. 6,156,303 SEQ ID NO: 11 |
| AAV6 | 98 | U.S. Pat. No. 6,156,303 SEQ ID NO: 2 |
| AAV6 | 99 | US20150315612 SEQ ID NO: 203 |
| AAV6 | 100 | US20150315612 SEQ ID NO: 220 |
| AAV6.1 | 101 | US20150159173 |
| AAV6.12 | 102 | US20150159173 |
| AAV6.2 | 103 | US20150159173 |
| AAV7 | 104 | US20150159173 SEQ ID NO: 14 |
| AAV7 | 105 | US20150315612 SEQ ID NO: 183 |
| AAV7 | 106 | US20030138772 SEQ ID NO: 2, |
| | | US20150159173 SEQ ID NO: 30, |
| | | US20150315612 SEQ ID NO: 181, |
| | | US20160017295 SEQ ID NO: 7 |
| AAV7 | 107 | US20030138772 SEQ ID NO: 3 |
| AAV7 | 108 | US20030138772 SEQ ID NO: 1, |
| | | US20150315612 SEQ ID NO: 180 |
| AAV7 | 109 | US20150315612 SEQ ID NO: 213 |
| AAV7 | 110 | US20150315612 SEQ ID NO: 222 |
| AAV8 | 111 | US20150159173 SEQ ID NO: 15 |
| AAV8 | 112 | US20150376240 SEQ ID NO: 7 |
| AAV8 | 113 | US20030138772 SEQ ID NO: 4, |
| | | US20150315612 SEQ ID NO: 182 |
| AAV8 | 114 | US20030138772 SEQ ID NO: 95, |
| | | US20140359799 SEQ ID NO: 1, |
| | | US20150159173 SEQ ID NO: 31, |
| | | US20160017295 SEQ ID NO: 8, |
| | | U.S. Pat. No. 7,198,951 SEQ ID NO: 7, |
| | | US20150315612 SEQ ID NO: 223 |
| AAV8 | 115 | US20150376240 SEQ ID NO: 8 |
| AAV8 | 116 | US20150315612 SEQ ID NO: 214 |
| AAV-8b | 117 | US20150376240 SEQ ID NO: 5 |
| AAV-8b | 118 | US20150376240 SEQ ID NO: 3 |
| AAV-8h | 119 | US20150376240 SEQ ID NO: 6 |
| AAV-8h | 120 | US20150376240 SEQ ID NO: 4 |
| AAV9 | 121 | US20030138772 SEQ ID NO: 5 |
| AAV9 | 122 | U.S. Pat. No. 7,198,951 SEQ ID NO: 1 |
| AAV9 | 123 | US20160017295 SEQ ID NO: 9 |
| AAV9 | 124 | US20030138772 SEQ ID NO: 100, |
| | | U.S. Pat. No. 7,198,951 SEQ ID NO: 2 |
| AAV9 | 125 | U.S. Pat. No. 7,198,951 SEQ ID NO: 3 |
| AAV9 (AAVhu.14) | 126 | US20150315612 SEQ ID NO: 3 |
| AAV9 (AAVhu.14) | 127 | US20150315612 SEQ ID NO: 123 |
| AAVA3.1 | 128 | US20030138772 SEQ ID NO: 120 |
| AAVA3.3 | 129 | US20030138772 SEQ ID NO: 57 |
| AAVA3.3 | 130 | US20030138772 SEQ ID NO: 66 |
| AAVA3.4 | 131 | US20030138772 SEQ ID NO: 54 |
| AAVA3.4 | 132 | US20030138772 SEQ ID NO: 68 |
| AAVA3.5 | 133 | US20030138772 SEQ ID NO: 55 |
| AAVA3.5 | 134 | US20030138772 SEQ ID NO: 69 |
| AAVA3.7 | 135 | US20030138772 SEQ ID NO: 56 |
| AAVA3.7 | 136 | US20030138772 SEQ ID NO: 67 |
| AAV29.3 (AAVbb.1) | 137 | US20030138772 SEQ ID NO: 11 |
| AAVC2 | 138 | US20030138772 SEQ ID NO: 61 |
| AAVCh.5 | 139 | US20150159173 SEQ ID NO: 46, |
| | | US20150315612 SEQ ID NO: 234 |
| AAVcy.2 (AAV13.3) | 140 | US20030138772 SEQ ID NO: 15 |
| AAV24.1 | 141 | US20030138772 SEQ ID NO: 101 |
| AAVcy.3 (AAV24.1) | 142 | US20030138772 SEQ ID NO: 16 |
| AAV27.3 | 143 | US20030138772 SEQ ID NO: 104 |
| AAVcy.4 (AAV27.3) | 144 | US20030138772 SEQ ID NO: 17 |
| AAVcy.5 | 145 | US20150315612 SEQ ID NO: 227 |
| AAV7.2 | 146 | US20030138772 SEQ ID NO: 103 |
| AAVcy.5 (AAV7.2) | 147 | US20030138772 SEQ ID NO: 18 |
| AAV16.3 | 148 | US20030138772 SEQ ID NO: 105 |
| AAVcy.6 (AAV16.3) | 149 | US20030138772 SEQ ID NO: 10 |
| AAVcy.5 | 150 | US20150159173 SEQ ID NO: 8 |
| AAVcy.5 | 151 | US20150159173 SEQ ID NO: 24 |
| AAVCy.5R1 | 152 | US20150159173 |
| AAVCy.5R2 | 153 | US20150159173 |
| AAVCy.5R3 | 154 | US20150159173 |
| AAVCy.5R4 | 155 | US20150159173 |
| AAVDJ | 156 | US20140359799 SEQ ID NO: 3, |
| | | U.S. Pat. No. 7,588,772 SEQ ID NO: 2 |
| AAVDJ | 157 | US20140359799 SEQ ID NO: 2, |
| | | U.S. Pat. No. 7,588,772 SEQ ID NO: 1 |
| AAVDJ-8 | 158 | U.S. Pat. No. 7,588,772; Grimm |
| | | et al 2008 |
| AAVDJ-8 | 159 | U.S. Pat. No. 7,588,772; Grimm |
| | | et al 2008 |
| AAVF5 | 160 | US20030138772 SEQ ID NO: 110 |
| AAVH2 | 161 | US20030138772 SEQ ID NO: 26 |
| AAVH6 | 162 | US20030138772 SEQ ID NO: 25 |
| AAVhE1.1 | 163 | U.S. Pat. No. 9,233,131 SEQ ID NO: 44 |
| AAVhEr1.14 | 164 | U.S. Pat. No. 9,233,131 SEQ ID NO: 46 |
| AAVhEr1.16 | 165 | U.S. Pat. No. 9,233,131 SEQ ID NO: 48 |
| AAVhEr1.18 | 166 | U.S. Pat. No. 9,233,131 SEQ ID NO: 49 |
| AAVhEr1.23 | 167 | U.S. Pat. No. 9,233,131 SEQ ID NO: 53 |
| (AAVhEr2.29) | | |
| AAVhEr1.35 | 168 | U.S. Pat. No. 9,233,131 SEQ ID NO: 50 |
| AAVhEr1.36 | 169 | U.S. Pat. No. 9,233,131 SEQ ID NO: 52 |
| AAVhEr1.5 | 170 | U.S. Pat. No. 9,233,131 SEQ ID NO: 45 |
| AAVhEr1.7 | 171 | U.S. Pat. No. 9,233,131 SEQ ID NO: 51 |
| AAVhEr1.8 | 172 | U.S. Pat. No. 9,233,131 SEQ ID NO: 47 |
| AAVhEr2.16 | 173 | U.S. Pat. No. 9,233,131 SEQ ID NO: 55 |
| AAVhEr2.30 | 174 | U.S. Pat. No. 9,233,131 SEQ ID NO: 56 |
| AAVhEr2.31 | 175 | U.S. Pat. No. 9,233,131 SEQ ID NO: 58 |
| AAVhEr2.36 | 176 | U.S. Pat. No. 9,233,131 SEQ ID NO: 57 |
| AAVhEr2.4 | 177 | U.S. Pat. No. 9,233,131 SEQ ID NO: 54 |
| AAVhEr3.1 | 178 | U.S. Pat. No. 9,233,131 SEQ ID NO: 59 |
| AAVhu.1 | 179 | US20150315612 SEQ ID NO: 46 |
| AAVhu.1 | 180 | US20150315612 SEQ ID NO: 144 |
| AAVhu.10 | 181 | US20150315612 SEQ ID NO: 56 |
| (AAV16.8) | | |
| AAVhu.10 | 182 | US20150315612 SEQ ID NO: 156 |
| (AAV16.8) | | |
| AAVhu.11 | 183 | US20150315612 SEQ ID NO: 57 |
| (AAV16.12) | | |
| AAVhu.11 | 184 | US20150315612 SEQ ID NO: 153 |
| (AAV16.12) | | |
| AAVhu.12 | 185 | US20150315612 SEQ ID NO: 59 |
| AAVhu.12 | 186 | US20150315612 SEQ ID NO: 154 |
| AAVhu.13 | 187 | US20150159173 SEQ ID NO: 16, |
| | | US20150315612 SEQ ID NO: 71 |
| AAVhu.13 | 188 | US20150159173 SEQ ID NO: 32, |
| | | US20150315612 SEQ ID NO: 129 |
| AAVhu.136.1 | 189 | US20150315612 SEQ ID NO: 165 |
| AAVhu.140.1 | 190 | US20150315612 SEQ ID NO: 166 |
| AAVhu.140.2 | 191 | US20150315612 SEQ ID NO: 167 |
| AAVhu.145.6 | 192 | US20150315612 SEQ ID No: 178 |
| AAVhu.15 | 193 | US20150315612 SEQ ID NO: 147 |
| AAVhu.15 | 194 | US20150315612 SEQ ID NO: 50 |
| (AAV33.4) | | |
| AAVhu.156.1 | 195 | US20150315612 SEQ ID No: 179 |
| AAVhu.16 | 196 | US20150315612 SEQ ID NO: 148 |
| AAVhu.16 | 197 | US20150315612 SEQ ID NO: 51 |
| (AAV33.8) | | |
| AAVhu.17 | 198 | US20150315612 SEQ ID NO: 83 |
| AAVhu.17 | 199 | US20150315612 SEQ ID NO: 4 |
| (AAV33.12) | | |
| AAVhu.172.1 | 200 | US20150315612 SEQ ID NO: 171 |
| AAVhu.172.2 | 201 | US20150315612 SEQ ID NO: 172 |
| AAVhu.173.4 | 202 | US20150315612 SEQ ID NO: 173 |
| AAVhu.173.8 | 203 | US20150315612 SEQ ID NO: 175 |
| AAVhu.18 | 204 | US20150315612 SEQ ID NO: 52 |
| AAVhu.18 | 205 | US20150315612 SEQ ID NO: 149 |
| AAVhu.19 | 206 | US20150315612 SEQ ID NO: 62 |
| AAVhu.19 | 207 | US20150315612 SEQ ID NO: 133 |
| AAVhu.2 | 208 | US20150315612 SEQ ID NO: 48 |
| AAVhu.2 | 209 | US20150315612 SEQ ID NO: 143 |
| AAVhu.20 | 210 | US20150315612 SEQ ID NO: 63 |
| AAVhu.20 | 211 | US20150315612 SEQ ID NO: 134 |
| AAVhu.21 | 212 | US20150315612 SEQ ID NO: 65 |
| AAVhu.21 | 213 | US20150315612 SEQ ID NO: 135 |
| AAVhu.22 | 214 | US20150315612 SEQ ID NO: 67 |
| AAVhu.22 | 215 | US20150315612 SEQ ID NO: 138 |
| AAVhu.23 | 216 | US20150315612 SEQ ID NO: 60 |
| AAVhu.23.2 | 217 | US20150315612 SEQ ID NO: 137 |
| AAVhu.24 | 218 | US20150315612 SEQ ID NO: 66 |
| AAVhu.24 | 219 | US20150315612 SEQ ID NO: 136 |
| AAVhu.25 | 220 | US20150315612 SEQ ID NO: 49 |
| AAVhu.25 | 221 | US20150315612 SEQ ID NO: 146 |
| AAVhu.26 | 222 | US20150159173 SEQ ID NO: 17, |
| | | US20150315612 SEQ ID NO: 61 |
| AAVhu.26 | 223 | US20150159173 SEQ ID NO: 33, |
| | | US20150315612 SEQ ID NO: 139 |
| AAVhu.27 | 224 | US20150315612 SEQ ID NO: 64 |
| AAVhu.27 | 225 | US20150315612 SEQ ID NO: 140 |
| AAVhu.28 | 226 | US20150315612 SEQ ID NO: 68 |
| AAVhu.28 | 227 | US20150315612 SEQ ID NO: 130 |
| AAVhu.29 | 228 | US20150315612 SEQ ID NO: 69 |
| AAVhu.29 | 229 | US20150159173 SEQ ID NO: 42, |
| | | US20150315612 SEQ ID NO: 132 |
| AAVhu.29 | 230 | US20150315612 SEQ ID NO: 225 |
| AAVhu.29R | 231 | US20150159173 |
| AAVhu.3 | 232 | US20150315612 SEQ ID NO: 44 |
| AAVhu.3 | 233 | US20150315612 SEQ ID NO: 145 |
| AAVhu.30 | 234 | US20150315612 SEQ ID NO: 70 |
| AAVhu.30 | 235 | US20150315612 SEQ ID NO: 131 |
| AAVhu.31 | 236 | US20150315612 SEQ ID NO: 1 |
| AAVhu.31 | 237 | US20150315612 SEQ ID NO: 121 |
| AAVhu.32 | 238 | US20150315612 SEQ ID NO: 2 |
| AAVhu.32 | 239 | US20150315612 SEQ ID NO: 122 |
| AAVhu.33 | 240 | US20150315612 SEQ ID NO: 75 |
| AAVhu.33 | 241 | US20150315612 SEQ ID NO: 124 |
| AAVhu.34 | 242 | US20150315612 SEQ ID NO: 72 |
| AAVhu.34 | 243 | US20150315612 SEQ ID NO: 125 |
| AAVhu.35 | 244 | US20150315612 SEQ ID NO: 73 |
| AAVhu.35 | 245 | US20150315612 SEQ ID NO: 164 |
| AAVhu.36 | 246 | US20150315612 SEQ ID NO: 74 |
| AAVhu.36 | 247 | US20150315612 SEQ ID NO: 126 |
| AAVhu.37 | 248 | US20150159173 SEQ ID NO: 34, |
| | | US20150315612 SEQ ID NO: 88 |
| AAVhu.37 | 249 | US20150315612 SEQ ID NO: 10, |
| (AAV106.1) | | US20150159173 SEQ ID NO: 18 |
| AAVhu.38 | 250 | US20150315612 SEQ ID NO: 161 |
| AAVhu.39 | 251 | US20150315612 SEQ ID NO: 102 |
| AAVhu.39 | 252 | US20150315612 SEQ ID NO: 24 |
| (AAVLG-9) | | |
| AAVhu.4 | 253 | US20150315612 SEQ ID NO: 47 |
| AAVhu.4 | 254 | US20150315612 SEQ ID NO: 141 |
| AAVhu.40 | 255 | US20150315612 SEQ ID NO: 87 |
| AAVhu.40 | 256 | US20150315612 SEQ ID No: 11 |
| (AAV114.3) | | |
| AAVhu.41 | 257 | US20150315612 SEQ ID NO: 91 |
| AAVhu.41 | 258 | US20150315612 SEQ ID NO: 6 |
| (AAV127.2) | | |
| AAVhu.42 | 259 | US20150315612 SEQ ID NO: 85 |
| AAVhu.42 | 260 | US20150315612 SEQ ID NO: 8 |
| (AAV127.5) | | |
| AAVhu.43 | 261 | US20150315612 SEQ ID NO: 160 |
| AAVhu.43 | 262 | US20150315612 SEQ ID NO: 236 |
| AAVhu.43 | 263 | US20150315612 SEQ ID NO: 80 |
| (AAV128.1) | | |
| AAVhu.44 | 264 | US20150159173 SEQ ID NO: 45, |
| | | US20150315612 SEQ ID NO: 158 |
| AAVhu.44 | 265 | US20150315612 SEQ ID NO: 81 |
| (AAV128.3) | | |
| AAVhu.44R1 | 266 | US20150159173 |
| AAVhu.44R2 | 267 | US20150159173 |
| AAVhu.44R3 | 268 | US20150159173 |
| AAVhu.45 | 269 | US20150315612 SEQ ID NO: 76 |
| AAVhu.45 | 270 | US20150315612 SEQ ID NO: 127 |
| AAVhu.46 | 271 | US20150315612 SEQ ID NO: 82 |
| AAVhu.46 | 272 | US20150315612 SEQ ID NO: 159 |
| AAVhu.46 | 273 | US20150315612 SEQ ID NO: 224 |
| AAVhu.47 | 274 | US20150315612 SEQ ID NO: 77 |
| AAVhu.47 | 275 | US20150315612 SEQ ID NO: 128 |
| AAVhu.48 | 276 | US20150159173 SEQ ID NO: 38 |
| AAVhu.48 | 277 | US20150315612 SEQ ID NO: 157 |
| AAVhu.48 | 278 | US20150315612 SEQ ID NO: 78 |
| (AAV130.4) | | |
| AAVhu.48R1 | 279 | US20150159173 |
| AAVhu.48R2 | 280 | US20150159173 |
| AAVhu.48R3 | 281 | US20150159173 |
| AAVhu.49 | 282 | US20150315612 SEQ ID NO: 209 |
| AAVhu.49 | 283 | US20150315612 SEQ ID NO: 189 |
| AAVhu.5 | 284 | US20150315612 SEQ ID NO: 45 |
| AAVhu.5 | 285 | US20150315612 SEQ ID NO: 142 |
| AAVhu.51 | 286 | US20150315612 SEQ ID NO: 208 |
| AAVhu.51 | 287 | US20150315612 SEQ ID NO: 190 |
| AAVhu.52 | 288 | US20150315612 SEQ ID NO: 210 |
| AAVhu.52 | 289 | US20150315612 SEQ ID NO: 191 |
| AAVhu.53 | 290 | US20150159173 SEQ ID NO: 19 |
| AAVhu.53 | 291 | US20150159173 SEQ ID NO: 35 |
| AAVhu.53 | 292 | US20150315612 SEQ ID NO: 176 |
| (AAV145.1) | | |
| AAVhu.54 | 293 | US20150315612 SEQ ID NO: 188 |
| AAVhu.54 | 294 | US20150315612 SEQ ID No: 177 |
| (AAV145.5) | | |
| AAVhu.55 | 295 | US20150315612 SEQ ID NO: 187 |
| AAVhu.56 | 296 | US20150315612 SEQ ID NO: 205 |
| AAVhu.56 | 297 | US20150315612 SEQ ID NO: 168 |
| (AAV145.6) | | |
| AAVhu.56 | 298 | US20150315612 SEQ ID NO: 192 |
| (AAV145.6) | | |
| AAVhu.57 | 299 | US20150315612 SEQ ID NO: 206 |
| AAVhu.57 | 300 | US20150315612 SEQ ID NO: 169 |
| AAVhu.57 | 301 | US20150315612 SEQ ID NO: 193 |
| AAVhu.58 | 302 | US20150315612 SEQ ID NO: 207 |
| AAVhu.58 | 303 | US20150315612 SEQ ID NO: 194 |
| AAVhu.6 (AAV3.1) | 304 | US20150315612 SEQ ID NO: 5 |
| AAVhu.6 (AAV3.1) | 305 | US20150315612 SEQ ID NO: 84 |
| AAVhu.60 | 306 | US20150315612 SEQ ID NO: 184 |
| AAVhu.60 | 307 | US20150315612 SEQ ID NO: 170 |
| (AAV161.10) | | |
| AAVhu.61 | 308 | US20150315612 SEQ ID NO: 185 |
| AAVhu.61 | 309 | US20150315612 SEQ ID NO: 174 |
| (AAV161.6) | | |
| AAVhu.63 | 310 | US20150315612 SEQ ID NO: 204 |
| AAVhu.63 | 311 | US20150315612 SEQ ID NO: 195 |
| AAVhu.64 | 312 | US20150315612 SEQ ID NO: 212 |
| AAVhu.64 | 313 | US20150315612 SEQ ID NO: 196 |
| AAVhu.66 | 314 | US20150315612 SEQ ID NO: 197 |
| AAVhu.67 | 315 | US20150315612 SEQ ID NO: 215 |
| AAVhu.67 | 316 | US20150315612 SEQ ID NO: 198 |
| AAVhu.7 | 317 | US20150315612 SEQ ID NO: 226 |
| AAVhu.7 | 318 | US20150315612 SEQ ID NO: 150 |
| AAVhu.7 (AAV7.3) | 319 | US20150315612 SEQ ID NO: 55 |
| AAVhu.71 | 320 | US20150315612 SEQ ID NO: 79 |
| AAVhu.8 | 321 | US20150315612 SEQ ID NO: 53 |
| AAVhu.8 | 322 | US20150315612 SEQ ID NO: 12 |
| AAVhu.8 | 323 | US20150315612 SEQ ID NO: 151 |
| AAVhu.9 (AAV3.1) | 324 | US20150315612 SEQ ID NO: 58 |
| AAVhu.9 (AAV3.1) | 325 | US20150315612 SEQ ID NO: 155 |
| AAV-LK01 | 326 | US20150376607 SEQ ID NO: 2 |
| AAV-LK01 | 327 | US20150376607 SEQ ID NO: 29 |
| AAV-LK02 | 328 | US20150376607 SEQ ID NO: 3 |
| AAV-LK02 | 329 | US20150376607 SEQ ID NO: 30 |
| AAV-LK03 | 330 | US20150376607 SEQ ID NO: 4 |
| AAV-LK03 | 331 | WO2015121501 SEQ ID NO: 12, |
| | | US20150376607 SEQ ID NO: 31 |
| AAV-LK04 | 332 | US20150376607 SEQ ID NO: 5 |
| AAV-LK04 | 333 | US20150376607 SEQ ID NO: 32 |
| AAV-LK05 | 334 | US20150376607 SEQ ID NO: 6 |
| AAV-LK05 | 335 | US20150376607 SEQ ID NO: 33 |
| AAV-LK06 | 336 | US20150376607 SEQ ID NO: 7 |
| AAV-LK06 | 337 | US20150376607 SEQ ID NO: 34 |
| AAV-LK07 | 338 | US20150376607 SEQ ID NO: 8 |
| AAV-LK07 | 339 | US20150376607 SEQ ID NO: 35 |
| AAV-LK08 | 340 | US20150376607 SEQ ID NO: 9 |
| AAV-LK08 | 341 | US20150376607 SEQ ID NO: 36 |
| AAV-LK09 | 342 | US20150376607 SEQ ID NO: 10 |
| AAV-LK09 | 343 | US20150376607 SEQ ID NO: 37 |
| AAV-LK10 | 344 | US20150376607 SEQ ID NO: 11 |
| AAV-LK10 | 345 | US20150376607 SEQ ID NO: 38 |
| AAV-LK11 | 346 | US20150376607 SEQ ID NO: 12 |
| AAV-LK11 | 347 | US20150376607 SEQ ID NO: 39 |
| AAV-LK12 | 348 | US20150376607 SEQ ID NO: 13 |
| AAV-LK12 | 349 | US20150376607 SEQ ID NO: 40 |
| AAV-LK13 | 350 | US20150376607 SEQ ID NO: 14 |
| AAV-LK13 | 351 | US20150376607 SEQ ID NO: 41 |
| AAV-LK14 | 352 | US20150376607 SEQ ID NO: 15 |
| AAV-LK14 | 353 | US20150376607 SEQ ID NO: 42 |
| AAV-LK15 | 354 | US20150376607 SEQ ID NO: 16 |
| AAV-LK15 | 355 | US20150376607 SEQ ID NO: 43 |
| AAV-LK16 | 356 | US20150376607 SEQ ID NO: 17 |
| AAV-LK16 | 357 | US20150376607 SEQ ID NO: 44 |
| AAV-LK17 | 358 | US20150376607 SEQ ID NO: 18 |
| AAV-LK17 | 359 | US20150376607 SEQ ID NO: 45 |
| AAV-LK18 | 360 | US20150376607 SEQ ID NO: 19 |
| AAV-LK18 | 361 | US20150376607 SEQ ID NO: 46 |
| AAV-LK19 | 362 | US20150376607 SEQ ID NO: 20 |
| AAV-LK19 | 363 | US20150376607 SEQ ID NO: 47 |
| AAV-PAEC | 364 | US20150376607 SEQ ID NO: 1 |
| AAV-PAEC | 365 | US20150376607 SEQ ID NO: 48 |
| AAV-PAEC11 | 366 | US20150376607 SEQ ID NO: 26 |
| AAV-PAEC11 | 367 | US20150376607 SEQ ID NO: 54 |
| AAV-PAEC12 | 368 | US20150376607 SEQ ID NO: 27 |
| AAV-PAEC12 | 369 | US20150376607 SEQ ID NO: 51 |
| AAV-PAEC13 | 370 | US20150376607 SEQ ID NO: 28 |
| AAV-PAEC13 | 371 | US20150376607 SEQ ID NO: 49 |
| AAV-PAEC2 | 372 | US20150376607 SEQ ID NO: 21 |
| AAV-PAEC2 | 373 | US20150376607 SEQ ID NO: 56 |
| AAV-PAEC4 | 374 | US20150376607 SEQ ID NO: 22 |
| AAV-PAEC4 | 375 | US20150376607 SEQ ID NO: 55 |
| AAV-PAEC6 | 376 | US20150376607 SEQ ID NO: 23 |
| AAV-PAEC6 | 377 | US20150376607 SEQ ID NO: 52 |
| AAV-PAEC7 | 378 | US20150376607 SEQ ID NO: 24 |
| AAV-PAEC7 | 379 | US20150376607 SEQ ID NO: 53 |
| AAV-PAEC8 | 380 | US20150376607 SEQ ID NO: 25 |
| AAV-PAEC8 | 381 | US20150376607 SEQ ID NO: 50 |
| AAVpi.1 | 382 | US20150315612 SEQ ID NO: 28 |
| AAVpi.1 | 383 | US20150315612 SEQ ID NO: 93 |
| AAVpi.2 | 384 | US20150315612 SEQ ID NO: 30 |
| AAVpi.2 | 385 | US20150315612 SEQ ID NO: 95 |
| AAVpi.3 | 386 | US20150315612 SEQ ID NO: 29 |
| AAVpi.3 | 387 | US20150315612 SEQ ID NO: 94 |
| AAVrh.10 | 388 | US20150159173 SEQ ID NO: 9 |
| AAVrh.10 | 389 | US20150159173 SEQ ID NO: 25 |
| AAV44.2 | 390 | US20030138772 SEQ ID NO: 59 |
| AAVrh.10 | 391 | US20030138772 SEQ ID NO: 81 |
| (AAV44.2) | | |
| AAV42.1B | 392 | US20030138772 SEQ ID NO: 90 |
| AAVrh.12 | 393 | US20030138772 SEQ ID NO: 30 |
| (AAV42.1b) | | |
| AAVrh.13 | 394 | US20150159173 SEQ ID NO: 10 |
| AAVrh.13 | 395 | US20150159173 SEQ ID NO: 26 |
| AAVrh.13 | 396 | US20150315612 SEQ ID NO: 228 |
| AAVrh.13R | 397 | US20150159173 |
| AAV42.3A | 398 | US20030138772 SEQ ID NO: 87 |
| AAVrh.14 | 399 | US20030138772 SEQ ID NO: 32 |
| (AAV42.3a) | | |
| AAV42.5A | 400 | US20030138772 SEQ ID NO: 89 |
| AAVrh.17 | 401 | US20030138772 SEQ ID NO: 34 |
| (AAV42.5a) | | |
| AAV42.5B | 402 | US20030138772 SEQ ID NO: 91 |
| AAVrh.18 | 403 | US20030138772 SEQ ID NO: 29 |
| (AAV42.5b) | | |
| AAV42.6B | 404 | US20030138772 SEQ ID NO: 112 |
| AAVrh.19 | 405 | US20030138772 SEQ ID NO: 38 |
| (AAV42.6b) | | |
| AAVrh.2 | 406 | US20150159173 SEQ ID NO: 39 |
| AAVrh.2 | 407 | US20150315612 SEQ ID NO: 231 |
| AAVrh.20 | 408 | US20150159173 SEQ ID NO: 1 |
| AAV42.10 | 409 | US20030138772 SEQ ID NO: 106 |
| AAVrh.21 | 410 | US20030138772 SEQ ID NO: 35 |
| (AAV42.10) | | |
| AAV42.11 | 411 | US20030138772 SEQ ID NO: 108 |
| AAVrh.22 | 412 | US20030138772 SEQ ID NO: 37 |
| (AAV42.11) | | |
| AAV42.12 | 413 | US20030138772 SEQ ID NO: 113 |
| AAVrh.23 | 414 | US20030138772 SEQ ID NO: 58 |
| (AAV42.12) | | |
| AAV42.13 | 415 | US20030138772 SEQ ID NO: 86 |
| AAVrh.24 | 416 | US20030138772 SEQ ID NO: 31 |
| (AAV42.13) | | |
| AAV42.15 | 417 | US20030138772 SEQ ID NO: 84 |
| AAVrh.25 | 418 | US20030138772 SEQ ID NO: 28 |
| (AAV42.15) | | |
| AAVrh.2R | 419 | US20150159173 |
| AAVrh.31 | 420 | US20030138772 SEQ ID NO: 48 |
| (AAV223.1) | | |
| AAVC1 | 421 | US20030138772 SEQ ID NO: 60 |
| AAVrh.32 (AAVC1) | 422 | US20030138772 SEQ ID NO: 19 |
| AAVrh.32/33 | 423 | US20150159173 SEQ ID NO: 2 |
| AAVrh.33 (AAVC3) | 424 | US20030138772 SEQ ID NO: 20 |
| AAVC5 | 425 | US20030138772 SEQ ID NO: 62 |
| AAVrh.34 (AAVC5) | 426 | US20030138772 SEQ ID NO: 21 |
| AAVF1 | 427 | US20030138772 SEQ ID NO: 109 |
| AAVrh.35 (AAVF1) | 428 | US20030138772 SEQ ID NO: 22 |
| AAVF3 | 429 | US20030138772 SEQ ID NO: 111 |
| AAVrh.36 (AAVF3) | 430 | US20030138772 SEQ ID NO: 23 |
| AAVrh.37 | 431 | US20030138772 SEQ ID NO: 24 |
| AAVrh.37 | 432 | US20150159173 SEQ ID NO: 40 |
| AAVrh.37 | 433 | US20150315612 SEQ ID NO: 229 |
| AAVrh.37R2 | 434 | US20150159173 |
| AAVrh.38 | 435 | US20150315612 SEQ ID NO: 7 |
| (AAVLG-4) | | |
| AAVrh.38 | 436 | US20150315612 SEQ ID NO: 86 |
| (AAVLG-4) | | |
| AAVrh.39 | 437 | US20150159173 SEQ ID NO: 20, |
| | | US20150315612 SEQ ID NO: 13 |
| AAVrh.39 | 438 | US20150159173 SEQ ID NO: 3, |
| | | US20150159173 SEQ ID NO: 36, |
| | | US20150315612 SEQ ID NO: 89 |
| AAVrh.40 | 439 | US20150315612 SEQ ID NO: 92 |
| AAVrh.40 | 440 | US20150315612 SEQ ID No: 14 |
| (AAVLG-10) | | |
| AAVrh.43 | 441 | US20150315612 SEQ ID NO: 43, |
| (AAVN721-8) | | US20150159173 SEQ ID NO: 21 |
| AAVrh.43 | 442 | US20150315612 SEQ ID NO: 163, |
| (AAVN721-8) | | US20150159173 SEQ ID NO: 37 |
| AAVrh.44 | 443 | US20150315612 SEQ ID NO: 34 |
| AAVrh.44 | 444 | US20150315612 SEQ ID NO: 111 |
| AAVrh.45 | 445 | US20150315612 SEQ ID NO: 41 |
| AAVrh.45 | 446 | US20150315612 SEQ ID NO: 109 |
| AAVrh.46 | 447 | US20150159173 SEQ ID NO: 22, |
| | | US20150315612 SEQ ID NO: 19 |
| AAVrh.46 | 448 | US20150159173 SEQ ID NO: 4, |
| | | US20150315612 SEQ ID NO: 101 |
| AAVrh.47 | 449 | US20150315612 SEQ ID NO: 38 |
| AAVrh.47 | 450 | US20150315612 SEQ ID NO: 118 |
| AAVrh.48 | 451 | US20150159173 SEQ ID NO: 44, |
| | | US20150315612 SEQ ID NO: 115 |
| AAVrh.48.1 | 452 | US20150159173 |
| AAVrh.48.1.2 | 453 | US20150159173 |
| AAVrh.48.2 | 454 | US20150159173 |
| AAVrh.48 (AAV1-7) | 455 | US20150315612 SEQ ID NO: 32 |
| AAVrh.49 (AAV1-8) | 456 | US20150315612 SEQ ID NO: 25 |
| AAVrh.49 (AAV1-8) | 457 | US20150315612 SEQ ID NO: 103 |
| AAVrh.50 (AAV2-4) | 458 | US20150315612 SEQ ID NO: 23 |
| AAVrh.50 (AAV2-4) | 459 | US20150315612 SEQ ID NO: 108 |
| AAVrh.51 (AAV2-5) | 460 | US20150315612 SEQ ID No: 22 |
| AAVrh.51 (AAV2-5) | 461 | US20150315612 SEQ ID NO: 104 |
| AAVrh.52 (AAV3-9) | 462 | US20150315612 SEQ ID NO: 18 |
| AAVrh.52 (AAV3-9) | 463 | US20150315612 SEQ ID NO: 96 |
| AAVrh.53 | 464 | US20150315612 SEQ ID NO: 97 |
| AAVrh.53 | 465 | US20150315612 SEQ ID NO: 17 |
| (AAV3-11) | | |
| AAVrh.53 | 466 | US20150315612 SEQ ID NO: 186 |
| (AAV3-11) | | |
| AAVrh.54 | 467 | US20150315612 SEQ ID NO: 40 |
| AAVrh.54 | 468 | US20150159173 SEQ ID NO: 49, |
| | | US20150315612 SEQ ID NO: 116 |
| AAVrh.55 | 469 | US20150315612 SEQ ID NO: 37 |
| AAVrh.55 | 470 | US20150315612 SEQ ID NO: 117 |
| (AAV4-19) | | |
| AAVrh.56 | 471 | US20150315612 SEQ ID NO: 54 |
| AAVrh.56 | 472 | US20150315612 SEQ ID NO: 152 |
| AAVrh.57 | 473 | US20150315612 SEQ ID NO: 26 |
| AAVrh.57 | 474 | US20150315612 SEQ ID NO: 105 |
| AAVrh.58 | 475 | US20150315612 SEQ ID NO: 27 |
| AAVrh.58 | 476 | US20150159173 SEQ ID NO: 48, |
| | | US20150315612 SEQ ID NO: 106 |
| AAVrh.58 | 477 | US20150315612 SEQ ID NO: 232 |
| AAVrh.59 | 478 | US20150315612 SEQ ID NO: 42 |
| AAVrh.59 | 479 | US20150315612 SEQ ID NO: 110 |
| AAVrh.60 | 480 | US20150315612 SEQ ID NO: 31 |
| AAVrh.60 | 481 | US20150315612 SEQ ID NO: 120 |
| AAVrh.61 | 482 | US20150315612 SEQ ID NO: 107 |
| AAVrh.61 | 483 | US20150315612 SEQ ID NO: 21 |
| (AAV2-3) | | |
| AAVrh.62 | 484 | US20150315612 SEQ ID No: 33 |
| (AAV2-15) | | |
| AAVrh.62 | 485 | US20150315612 SEQ ID NO: 114 |
| (AAV2-15) | | |
| AAVrh.64 | 486 | US20150315612 SEQ ID No: 15 |
| AAVrh.64 | 487 | US20150159173 SEQ ID NO: 43, |
| | | US20150315612 SEQ ID NO: 99 |
| AAVrh.64 | 488 | US20150315612 SEQ ID NO: 233 |
| AAVRh.64R1 | 489 | US20150159173 |
| AAVRh.64R2 | 490 | US20150159173 |
| AAVrh.65 | 491 | US20150315612 SEQ ID NO: 35 |
| AAVrh.65 | 492 | US20150315612 SEQ ID NO: 112 |
| AAVrh.67 | 493 | US20150315612 SEQ ID NO: 36 |
| AAVrh.67 | 494 | US20150315612 SEQ ID NO: 230 |
| AAVrh.67 | 495 | US20150159173 SEQ ID NO: 47, |
| | | US20150315612 SEQ ID NO: 113 |
| AAVrh.68 | 496 | US20150315612 SEQ ID NO: 16 |
| AAVrh.68 | 497 | US20150315612 SEQ ID NO: 100 |
| AAVrh.69 | 498 | US20150315612 SEQ ID NO: 39 |
| AAVrh.69 | 499 | US20150315612 SEQ ID NO: 119 |
| AAVrh.70 | 500 | US20150315612 SEQ ID NO: 20 |
| AAVrh.70 | 501 | US20150315612 SEQ ID NO: 98 |
| AAVrh.71 | 502 | US20150315612 SEQ ID NO: 162 |
| AAVrh.72 | 503 | US20150315612 SEQ ID NO: 9 |
| AAVrh.73 | 504 | US20150159173 SEQ ID NO: 5 |
| AAVrh.74 | 505 | US20150159173 SEQ ID NO: 6 |
| AAVrh.8 | 506 | US20150159173 SEQ ID NO: 41 |
| AAVrh.8 | 507 | US20150315612 SEQ ID NO: 235 |
| AAVrh.8R | 508 | US20150159173, |
| | | WO2015168666 SEQ ID NO: 9 |
| AAVrh.8R A586R | 509 | WO2015168666 SEQ ID NO: 10 |
| mutant | | |
| AAVrh.8R R533A | 510 | WO2015168666 SEQ ID NO: 11 |
| mutant | | |
| BAAV (bovine | 511 | U.S. Pat. No. 9,193,769 SEQ ID NO: 8 |
| AAV) | | |
| BAAV (bovine | 512 | U.S. Pat. No. 9,193,769 SEQ ID NO: 10 |
| AAV) | | |
| BAAV (bovine | 513 | U.S. Pat. No. 9,193,769 SEQ ID NO: 4 |
| AAV) | | |
| BAAV (bovine | 514 | U.S. Pat. No. 9,193,769 SEQ ID NO: 2 |
| AAV) | | |
| BAAV (bovine | 515 | U.S. Pat. No. 9,193,769 SEQ ID NO: 6 |
| AAV) | | |
| BAAV (bovine | 516 | U.S. Pat. No. 9,193,769 SEQ ID NO: 1 |
| AAV) | | |
| BAAV (bovine | 517 | U.S. Pat. No. 9,193,769 SEQ ID NO: 5 |
| AAV) | | |
| BAAV (bovine | 518 | U.S. Pat. No. 9,193,769 SEQ ID NO: 3 |
| AAV) | | |
| BAAV (bovine | 519 | U.S. Pat. No. 9,193,769 SEQ ID NO: 11 |
| AAV) | | |
| BAAV (bovine | 520 | U.S. Pat. No. 7,427,396 SEQ ID NO: 5 |
| AAV) | | |
| BAAV (bovine | 521 | U.S. Pat. No. 7,427,396 SEQ ID NO: 6 |
| AAV) | | |
| BAAV (bovine | 522 | U.S. Pat. No. 9,193,769 SEQ ID NO: 7 |
| AAV) | | |
| BAAV (bovine | 523 | U.S. Pat. No. 9,193,769 SEQ ID NO: 9 |
| AAV) | | |
| BNP61 AAV | 524 | US20150238550 SEQ ID NO: 1 |
| BNP61 AAV | 525 | US20150238550 SEQ ID NO: 2 |
| BNP62 AAV | 526 | US20150238550 SEQ ID NO: 3 |
| BNP63 AAV | 527 | US20150238550 SEQ ID NO: 4 |
| caprine AAV | 528 | U.S. Pat. No. 7,427,396 SEQ ID NO: 3 |
| caprine AAV | 529 | U.S. Pat. No. 7,427,396 SEQ ID NO: 4 |
| true type AAV | 530 | WO2015121501 SEQ ID NO: 2 |
| (ttAAV) | | |
| AAAV (Avian AAV) | 531 | U.S. Pat. No. 9,238,800 SEQ ID NO: 12 |
| AAAV (Avian AAV) | 532 | U.S. Pat. No. 9,238,800 SEQ ID NO: 2 |
| AAAV (Avian AAV) | 533 | U.S. Pat. No. 9,238,800 SEQ ID NO: 6 |
| AAAV (Avian AAV) | 534 | U.S. Pat. No. 9,238,800 SEQ ID NO: 4 |
| AAAV (Avian AAV) | 535 | U.S. Pat. No. 9,238,800 SEQ ID NO: 8 |
| AAAV (Avian AAV) | 536 | U.S. Pat. No. 9,238,800 SEQ ID NO: 14 |
| AAAV (Avian AAV) | 537 | U.S. Pat. No. 9,238,800 SEQ ID NO: 10 |
| AAAV (Avian AAV) | 538 | U.S. Pat. No. 9,238,800 SEQ ID NO: 15 |
| AAAV (Avian AAV) | 539 | U.S. Pat. No. 9,238,800 SEQ ID NO: 5 |
| AAAV (Avian AAV) | 540 | U.S. Pat. No. 9,238,800 SEQ ID NO: 9 |
| AAAV (Avian AAV) | 541 | U.S. Pat. No. 9,238,800 SEQ ID NO: 3 |
| AAAV (Avian AAV) | 542 | U.S. Pat. No. 9,238,800 SEQ ID NO: 7 |
| AAAV (Avian AAV) | 543 | U.S. Pat. No. 9,238,800 SEQ ID NO: 11 |
| AAAV (Avian AAV) | 544 | U.S. Pat. No. 9,238,800 SEQ ID NO: 13 |
| AAAV (Avian AAV) | 545 | U.S. Pat. No. 9,238,800 SEQ ID NO: 1 |
| AAV Shuffle 100-1 | 546 | US20160017295 SEQ ID NO: 23 |
| AAV Shuffle 100-1 | 547 | US20160017295 SEQ ID NO: 11 |
| AAV Shuffle 100-2 | 548 | US20160017295 SEQ ID NO: 37 |
| AAV Shuffle 100-2 | 549 | US20160017295 SEQ ID NO: 29 |
| AAV Shuffle 100-3 | 550 | US20160017295 SEQ ID NO: 24 |
| AAV Shuffle 100-3 | 551 | US20160017295 SEQ ID NO: 12 |
| AAV Shuffle 100-7 | 552 | US20160017295 SEQ ID NO: 25 |
| AAV Shuffle 100-7 | 553 | US20160017295 SEQ ID NO: 13 |
| AAV Shuffle 10-2 | 554 | US20160017295 SEQ ID NO: 34 |
| AAV Shuffle 10-2 | 555 | US20160017295 SEQ ID NO: 26 |
| AAV Shuffle 10-6 | 556 | US20160017295 SEQ ID NO: 35 |
| AAV Shuffle 10-6 | 557 | US20160017295 SEQ ID NO: 27 |
| AAV Shuffle 10-8 | 558 | US20160017295 SEQ ID NO: 36 |
| AAV Shuffle 10-8 | 559 | US20160017295 SEQ ID NO: 28 |
| AAV SM 100-10 | 560 | US20160017295 SEQ ID NO: 41 |
| AAV SM 100-10 | 561 | US20160017295 SEQ ID NO: 33 |
| AAV SM 100-3 | 562 | US20160017295 SEQ ID NO: 40 |
| AAV SM 100-3 | 563 | US20160017295 SEQ ID NO: 32 |
| AAV SM 10-1 | 564 | US20160017295 SEQ ID NO: 38 |
| AAV SM 10-1 | 565 | US20160017295 SEQ ID NO: 30 |
| AAV SM 10-2 | 566 | US20160017295 SEQ ID NO: 10 |
| AAV SM 10-2 | 567 | US20160017295 SEQ ID NO: 22 |
| AAV SM 10-8 | 568 | US20160017295 SEQ ID NO: 39 |
| AAV SM 10-8 | 569 | US20160017295 SEQ ID NO: 31 |

Each of the patents, applications and/or publications listed in Table 1 are hereby incorporated by reference in their entirety.

In one embodiment, the AAV serotype may be engineered to comprise at least one AAV capsid CD8+ T-cell epitope. Hui et al. (Molecular Therapy—Methods & Clinical Development (2015) 2, 15029 doi:10.1038/mtm.2015.29; the contents of which are herein incorporated by reference in its entirety) identified AAV capsid-specific CD8+ T-cell epitopes for AAV1 and AAV2 (see e.g., Table 2 in the publication). As a non-limiting example, the capsid-specific CD8+ T-cell epitope may be for an AAV2 serotype. As a non-limiting example, the capsid-specific CD8+ T-cell epitope may be for an AAV1 serotype.

In one embodiment, peptides for inclusion in an AAV serotype may be identified using the methods described by Hui et al. (Molecular Therapy—Methods & Clinical Development (2015) 2, 15029 doi:10.1038/mtm.2015.29; the contents of which are herein incorporated by reference in its entirety). As a non-limiting example, the procedure includes isolating human splenocytes, restimulating the splenocytes in vitro using individual peptides spanning the amino acid sequence of the AAV capsid protein, IFN-gamma ELISpot with the individual peptides used for the in vitro restimulation, bioinformatics analysis to determine the HLA restriction of 15-mers identified by IFN-gamma ELISpot, identification of candidate reactive 9-mer epitopes for a given HLA allele, synthesis candidate 9-mers, second IFN-gamma ELISpot screening of splenocytes from subjects carrying the HLA alleles to which identified AAV epitopes are predicted to bind, determine the AAV capsid-reactive CD8+ T cell epitopes and determine the frequency of subjects reacting to a given AAV epitope.

In one embodiment, peptides for inclusion in an AAV serotype may be identified by isolating human splenocytes, restimulating the splenocytes in vitro using individual peptides spanning the amino acid sequence of the AAV capsid protein, IFN-gamma ELISpot with the individual peptides used for the in vitro restimulation, bioinformatics analysis to determine the given allele restriction of 15-mers identified by IFN-gamma ELISpot, identification of candidate reactive 9-mer epitopes for a given allele, synthesis candidate 9-mers, second IFN-gamma ELISpot screening of splenocytes from subjects carrying the specific alleles to which identified AAV epitopes are predicted to bind, determine the AAV capsid-reactive CD8+ T cell epitopes and determine the frequency of subjects reacting to a given AAV epitope.

AAV vectors comprising the nucleic acid sequence for the siRNA molecules may be prepared or derived from various serotypes of AAVs, including, but not limited to, AAV1, AAV2, AAV3, AAV4, AAV5, AAV6, AAV7, AAV8, AAV9, AAV9.47, AAV9(hu14), AAV10, AAV11, AAV12, AAVrh8, AAVrh10, AAV-DJ8 and AAV-DJ. In some cases, different serotypes of AAVs may be mixed together or with other types of viruses to produce chimeric AAV vectors. As a non-limiting example, the AAV vector is derived from the AAV9 serotype.

In one embodiment, AAV particles of the present invention may comprise capsid proteins having sequences of SEQ ID NOs: 1 and 3, which have increased tropism to the brain, of International Publication No. WO2014160092, the content of which is incorporated herein by reference in its entirety.

In one embodiment, AAV particles of the present invention may comprise capsid proteins which may target to oligodendrocytes in the central nervous system. The capsid proteins may comprise AAV capsid coding sequence of SEQ ID NO: 1 or AAV capsid proteins comprising amino acid sequences of SEQ ID NOs: 2 to 4 of International Publication No. WO2014052789, the content of which is herein incorporated by reference in its entirety.

In one embodiment, AAV particles of the present invention may comprise capsid proteins having increased capacity to cross the blood-brain barrier in CNS as disclosed in U.S. Pat. No. 8,927,514, the content of which is incorporated herein by reference in its entirety. The amino acid sequences and nucleic acid sequences of such capsid proteins may include, but are not limited to, SEQ ID NOs: 2-17 and SEQ ID NOs: 25-33, respectively, of U.S. Pat. No. 8,927,514.

In some embodiments, AAV particles of the present invention may comprise AAV2 capsid proteins or variants thereof. AAV particles with AAV2 capsid proteins have been shown to deliver genes to neurons effectively in the brain, retina and spinal cord. In one embodiment, AAV2 capsid proteins may be further modified such as addition of a targeting peptide to the capsid proteins that targets an AAV particle to brain vascular endothelium as disclosed in U.S. Pat. Nos. 6,691,948 and 8,299,215, the contents of each of which are herein incorporated by reference in their entirety. Such AAV particles may be used to deliver a functional payload of interest to treat a brain disease such as mucopolysaccharide (MPS).

In some embodiments, AAV particles of the present invention may comprise AAV5 capsid proteins or variants thereof. AAV particles with AAV5 capsid proteins can transduce neurons in various regions of the CNS, including the cortex, the hippocampus (HPC), cerebellum, substantia nigra (SN), striatum, globus pallidus, and spinal cord (Burger C et al., Mol Ther., 2004, 10(2): 302-317; Liu G et al., Mol Ther. 2007, 15(2): 242-247; and Colle M et al., Hum, Mol. Genet. 2010, 19(1): 147-158). In one embodiment, AAV particles having AAV5 capsid proteins with increased transduction to cells in CNS may be those particles from U.S. Pat. No. 7,056,502, the content of which is incorporated herein by reference in its entirety.

In some embodiments, AAV particles of the present invention may comprise AAV6 capsid proteins or variants thereof. Recombinant AAV6 serotype can target motor neurons in the spinal cord by Intracerebroventricular (ICV) injection (Dirren E et al., Hum Gene Ther., 2014, 25(2): 109-120). In addition, a study from San Sebastian et al indicated that AAV6 serotype can be retrogradely transported from terminals to neuronal cell bodies in the rat brain (San Sebastian et al, Gen Ther., 2014, 20(12): 1178-1183).

In some embodiments, AAV particles of the present invention may comprise AAV8 capsid proteins or variants thereof. AAV particles with AAV8 capsid proteins can transduce neurons, for example in hippocampus (Klein R L et al., Mol Ther., 2006, 13(3): 517-527). In one embodiment, AAV8 capsid proteins may comprise the amino acid sequence of SEQ ID NO: 2 of U.S. Pat. No. 8,318,480, the content of which is herein incorporated by reference in its entirety.

In some embodiments, AAV particles of the present invention may comprise AAV9 capsid proteins or variants thereof. AAV9 capsid serotype mediated gene delivery has been observed in the brain with efficient and long-term expression of transgene after intraparenchymal injections to the CNS (Klein R L et al., Eur J Neurosci., 2008, 27: 1615-1625). AAV9 serotype can produce robust and wide-scale neuronal transduction throughout the CNS after a peripheral, systemic (e.g., intravenous) administration in neonatal subjects (Foust K D et al., Nat. Biotechnol., 2009, 27: 59-65; and Duque S et al, Mol Ther., 2009, 17: 1187-1196). Intrathecal (intra-cisterna magna routes) administration of AAV9 serotypes can also produce widespread spinal expression. In one embodiment, AAV9 serotype may comprise an AAV capsid protein having the amino acid sequence of SEQ ID NO: 2 of U.S. Pat. No. 7,198,951, the content of which is incorporated herein by reference in its entirety. In another aspect, AAV9 serotype may comprise VP1 capsid proteins of SEQ ID NOs: 2, 4 or 6 in which at least one of surface-exposed tyrosine residues in the amino acid sequence is substituted with another amino acid residue, as disclosed in US patent publication No. US20130224836, the content of which is incorporated herein by reference in its entirety.

In some embodiments, AAV particles of the present invention may comprise AAVrh10 capsid proteins or variants thereof. AAV particles comprising AAVrh10 capsid proteins can target neurons, other cells as well, in the spinal cord after intrathecal (IT) administration. In one embodiment, AAVrh10 capsid proteins may comprise the amino acid sequence of SEQ ID NO: 81 of EP patent NO: 2341068.

In some embodiments, AAV of the present invention may comprise AAVDJ capsid proteins, AAVDJ/8 capsid proteins, or variants thereof. Holehonnur et al showed that AAVDJ/8 serotype can target neurons within the Basal and Lateral Amygdala (BLA) (Holennur R et al., BMC Neurosci, 2014, Feb. 18:15:28). In one embodiment, AAVDJ capsid proteins and/or AAVDJ/8 capsid proteins may comprise an amino acid sequence comprising a first region that is derived from a first AAV serotype (e.g., AAV2), a second region that is derived from a second AAV serotype (e.g., AAV8), and a third region that is derived from a third AAV serotype (e.g., AAV9), wherein the first, second and third region may include any amino acid sequences that are disclosed in this description.

In some embodiment, AAV particles produced according to the present invention may comprise single stranded DNA viral genomes (ssAAVs) or self-complementary AAV genomes (scAAVs). scAAV genomes contain both DNA strands which anneal together to form double stranded DNA. By skipping second strand synthesis, scAAVs allow for rapid expression in the cell.

In one embodiment, AAV particles of the present invention may comprise capsid proteins that have been shown to or are known to transduce dorsal root ganglions (DRGs).

In one embodiment, AAV particles of the present invention may comprise capsid proteins that have been shown or are known to transduce motor neurons.

In one embodiment, the AAV particles comprise a self-complementary (SC) vector genome.

In one embodiment, the AAV particles comprise a single stranded (SS) genome.

In one embodiment, an AAV particle comprising a self-complementary (sc) vector may be used to yield higher expression than an AAV particle comprising a corresponding single stranded vector genome.

In one embodiment, the serotype of the AAV particles described herein may depend on the desired distribution, transduction efficiency and cellular targeting required. As described by Sorrentino et al. (comprehensive map of CNS transduction by eight adeno-associated virus serotypes upon cerebrospinal fluid administration in pigs, Molecular Therapy accepted article preview online 7 Dec. 2015; doi:10.1038/mt.2015.212; the contents of which are herein incorporated by reference in its entirety), AAV serotypes provided different distributions, transduction efficiencies and cellular targeting. In order to provide the desired efficacy, the AAV serotype needs to be selected that best matches not only the cells to be targeted but also the desired transduction efficiency and distribution.

### Formulation and Delivery

### Formulation

Formulations of the present invention can include, without limitation, saline, liposomes, lipid nanoparticles, polymers, peptides, proteins, cells transfected with viral vectors (e.g., for transplantation into a subject) and combinations thereof.

Formulations of the pharmaceutical compositions described herein may be prepared by any method known or hereafter developed in the art of pharmacology. In general, such preparatory methods include the step of bringing the active ingredient into association with an excipient and/or one or more other accessory ingredients, and then, if necessary and/or desirable, dividing, shaping and/or packaging the product into a desired single- or multi-dose unit.

A pharmaceutical composition in accordance with the present disclosure may be prepared, packaged, and/or sold in bulk, as a single unit dose, and/or as a plurality of single unit doses. As used herein, a “unit dose” refers to a discrete amount of the pharmaceutical composition comprising a predetermined amount of the active ingredient. As used herein, a “single unit dose” is a dose of any therapeutic administered in one dose/at one time/single route/single point of contact, i.e., single administration event. As used herein, a “total daily dose” is an amount given or prescribed in 24 hour period. It may be administered as a single unit dose. The amount of the active ingredient is generally equal to the dosage of the active ingredient which would be administered to a subject and/or a convenient fraction of such a dosage such as, for example, one-half or one-third of such a dosage.

Relative amounts of the active ingredient (e.g. AAV particle), the pharmaceutically acceptable excipient, and/or any additional ingredients in a pharmaceutical composition in accordance with the present disclosure may vary, depending upon the identity, size, and/or condition of the subject being treated and further depending upon the route by which the composition is to be administered. For example, the composition may comprise between 0.1% and 99% (w/w) of the active ingredient. By way of example, the composition may comprise between 0.1% and 100%, e.g., between 0.5 and 50%, between 1-30%, between 5-80%, at least 80% (w/w) active ingredient.

In one embodiment, the viral particles (e.g., AAV particles) of the invention may be formulated in buffer only or in a formulation described herein.

In one embodiment, the AAV particles of the invention may be formulated in PBS with 0.001% of pluronic acid (F-68) at a pH of about 7.0.

In some embodiments, the AAV particle formulations described herein may contain a nucleic acid encoding at least one payload. As a non-limiting example, the formulations may contain a nucleic acid encoding 1, 2, 3, 4 or 5 payloads.

In one embodiment, factors which may influence drug distribution such as, but not limited to, catheter location (e.g., cervical or lumbar, and one or multi-site delivery), dosing regimen (e.g., continuous or bolus, and dose including rate, volume, and duration) formulation (e.g., baricity, temperature, etc.), spinal anatomy and pathology of a subject (e.g., scoliosis) and spatial orientation of a subject (e.g., horizontal or vertical) is evaluated prior to delivery of the AAV particles described herein.

The formulations of the invention can include one or more excipients, each in an amount that together increases the stability of the AAV particle, increases cell transfection or transduction by the viral particle, increases the expression of viral particle encoded protein, and/or alters the release profile of AAV particle encoded proteins. In some embodiments, a pharmaceutically acceptable excipient may be at least 95%, at least 96%, at least 97%, at least 98%, at least 99%, or 100% pure. In some embodiments, an excipient is approved for use for humans and for veterinary use. In some embodiments, an excipient may be approved by United States Food and Drug Administration. In some embodiments, an excipient may be of pharmaceutical grade. In some embodiments, an excipient may meet the standards of the United States Pharmacopoeia (USP), the European Pharmacopoeia (EP), the British Pharmacopoeia, and/or the International Pharmacopoeia.

Excipients, which, as used herein, includes, but is not limited to, any and all solvents, dispersion media, diluents, or other liquid vehicles, dispersion or suspension aids, surface active agents, isotonic agents, thickening or emulsifying agents, preservatives, and the like, as suited to the particular dosage form desired. Various excipients for formulating pharmaceutical compositions and techniques for preparing the composition are known in the art (see Remington: The Science and Practice of Pharmacy, 21^{st} Edition, A. R. Gennaro, Lippincott, Williams & Wilkins, Baltimore, Md., 2006; incorporated herein by reference in its entirety). The use of a conventional excipient medium may be contemplated within the scope of the present disclosure, except insofar as any conventional excipient medium may be incompatible with a substance or its derivatives, such as by producing any undesirable biological effect or otherwise interacting in a deleterious manner with any other component(s) of the pharmaceutical composition.

In one embodiment, the AAV particles may be formulated in a formulation which has been optimized to ensure optimal drug distribution in the central nervous system or a region or component of the central nervous system. As a non-limiting example, the baricity and/or osmolarity may be adjusted to ensure optimal drug distribution.

In one embodiment, the AAV particle formulation may include at least one inactive ingredient.

Although the descriptions of pharmaceutical compositions, e.g., AAV comprising a payload to be delivered, provided herein are principally directed to pharmaceutical compositions which are suitable for administration to humans, it will be understood by the skilled artisan that such compositions are generally suitable for administration to any other animal, e.g., to non-human animals, e.g. non-human mammals. Modification of pharmaceutical compositions suitable for administration to humans in order to render the compositions suitable for administration to various animals is well understood, and the ordinarily skilled veterinary pharmacologist can design and/or perform such modification with merely ordinary, if any, experimentation. Subjects to which administration of the pharmaceutical compositions is contemplated include, but are not limited to, humans and/or other primates; mammals, including commercially relevant mammals such as cattle, pigs, horses, sheep, cats, dogs, mice, and/or rats; and/or birds, including commercially relevant birds such as poultry, chickens, ducks, geese, and/or turkeys.

In some embodiments, compositions are administered to humans, human patients or subjects. For the purposes of the present disclosure, the phrase “active ingredient” generally refers either to the viral particle carrying the payload or to the payload delivered by the viral particle as described herein.

### Inactive Ingredients

In some embodiments, AAV formulations may comprise at least one excipient which is an inactive ingredient. As used herein, the term “inactive ingredient” refers to one or more agents that do not contribute to the activity of the pharmaceutical composition included in formulations. In some embodiments, all, none or some of the inactive ingredients which may be used in the formulations of the present invention may be approved by the US Food and Drug Administration (FDA).

Formulations of AAV particles disclosed herein may include cations or anions. In one embodiment, the formulations include metal cations such as, but not limited to, Zn2+, Ca2+, Cu2+, Mg+, MgSO₄, and combinations thereof. As a non-limiting example, MgSO₄ may be used to increase the ionic strength of a formulation.

### Composition pH

In one embodiment, formulations of AAV particles comprises a buffered composition of between pH 4.5 and 8.0. As a non-limiting example, the AAV particles may be delivered to the cells of the central nervous system (e.g., parenchyma).

In some embodiments, the formulation of AAV particles may comprise a buffered composition of about pH 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, or 8.0.

In one embodiment, the formulation of AAV particles comprises a buffered composition of pH 7.4, which is considered physiological pH.

In one embodiment, the formulation of AAV particles comprises a buffered composition of pH 7.0.

In one embodiment, the formulation has a relatively very low buffer strength, or ability to hold pH, which may allow the infused composition of AAV particles to quickly adjust to the prevailing physiological pH of the CSF (˜pH 7.4).

### Composition Baricity

It is known in the art that CSF comprises a baricity, or density of solution, of approximately 1 g/mL at 37° C. In one embodiment, delivery of AAV particles to cells of the central nervous system (e.g., parenchyma) comprises an isobaric composition wherein the baricity of the composition at 37° C. is approximately 1 g/mL. In one embodiment, delivery comprises a hypobaric composition wherein the baricity of the composition at 37° C. is less than 1 g/mL. In one embodiment, delivery comprises a hyperbaric composition wherein the baricity of the composition at 37° C. is greater than 1 g/mL (e.g., greater than 1.001 g/mL).

In one embodiment, the composition is a hyperbaric composition comprising AAV particles and a sugar such as, but not limited to, a sugar approved by the FDA (US Food and Drug Administration) for delivery. In one embodiment, delivery comprises a hyperbaric composition wherein the baricity of the composition at 37° C. is increased by addition of 5.0%, 5.1%, 5.2%, 5.3%, 5.4%, 5.5%, 5.6%, 5.7%, 5.8%, 5.9%, 6.0%, 6.1%, 6.2%, 6.3%, 6.4%, 6.5%, 6.6%, 6.7%, 6.8%, 6.9%, 7.0%, 7.1%, 7.2%, 7.3%, 7.4%, 7.5%, 7.6%, 7.7%, 7.8%, 7.9%, or 8.0% sugar. As a non-limiting example, the sugar may be dextrose, mannitol or sorbitol.

In one embodiment, the composition is a hyperbaric composition wherein the baricity of the composition at 37° C. is increased by addition of approximately 5% to 8% dextrose. In one embodiment, delivery comprises a hyperbaric composition wherein the baricity of the composition at 37° C. is increased by addition of 5.0%, 5.1%, 5.2%, 5.3%, 5.4%, 5.5%, 5.6%, 5.7%, 5.8%, 5.9%, 6.0%, 6.1%, 6.2%, 6.3%, 6.4%, 6.5%, 6.6%, 6.7%, 6.8%, 6.9%, 7.0%, 7.1%, 7.2%, 7.3%, 7.4%, 7.5%, 7.6%, 7.7%, 7.8%, 7.9%, or 8.0% dextrose.

In one embodiment, the composition is a hyperbaric composition wherein the baricity of the composition at 37° C. is increased by addition of approximately 4% to 8% mannitol. In one embodiment, delivery comprises a hyperbaric composition wherein the baricity of the composition at 37° C. is increased by addition of 4.0%, 4.1%, 4.2%, 4.3%, 4.4%, 4.5%, 4.6%, 4.7%, 4.8%, 4.9%, 5.0%5.1%5.2%5, 5.1%, 5.2%, 5.3%, 5.4%, 5.5%, 5.6%, 5.7%, 5.8%, 5.9%, 6.0%, 6.1%, 6.2%, 6.3%, 6.4%, 6.5%, 6.6%, 6.7%, 6.8%, 6.9%, 7.0%, 7.1%, 7.2%, 7.3%, 7.4%, 7.5%, 7.6%, 7.7%, 7.8%, 7.9%, or 8.0% mannitol.

In one embodiment, the composition is a hyperbaric composition wherein the baricity of the composition at 37° C. is increased by addition of approximately 4% to 8% sorbitol. In one embodiment, delivery comprises a hyperbaric composition wherein the baricity of the composition at 37° C. is increased by addition of 4.0%, 4.1%, 4.2%, 4.3%, 4.4%, 4.5%, 4.6%, 4.7%, 4.8%, 4.9%, 5.0%, 5.1%, 5.2%, 5.3%, 5.4%, 5.5%, 5.6%, 5.7%, 5.8%, 5.9%, 6.0%, 6.1%, 6.2%, 6.3%, 6.4%, 6.5%, 6.6%, 6.7%, 6.8%, 6.9%, 7.0%, 7.1%, 7.2%, 7.3%, 7.4%, 7.5%, 7.6%, 7.7%, 7.8%, 7.9%, or 8.0% sorbitol.

### Composition Osmolarity

In one embodiment, delivery of AAV particles to cells of the central nervous system (e.g., parenchyma) comprises co-administration of agents that increase serum osmolarity. As used herein, “co-administered” means the administration of two or more components. Co-administration refers to the administration of two or more components simultaneously or with a time lapse between administration such as 1 second, 5 seconds, 10 seconds, 15 seconds, 30 seconds, 45 seconds, 1 minute, 2 minutes, 3 minutes, 4 minutes, 5 minutes, 6 minutes, 7 minutes, 8 minutes, 9 minutes, 10 minutes, 11 minutes, 12 minutes, 13 minutes, 14 minutes, 15 minutes, 16 minutes, 17 minutes, 18 minutes, 19 minutes, 20 minutes, 21 minutes, 22 minutes, 23 minutes, 24 minutes, 25 minutes, 26 minutes, 27 minutes, 28 minutes, 29 minutes, 30 minutes, 31 minutes, 32 minutes, 33 minutes, 34 minutes, 35 minutes, 36 minutes, 37 minutes, 38 minutes, 39 minutes, 40 minutes, 41 minutes, 42 minutes, 43 minutes, 44 minutes, 45 minutes, 46 minutes, 47 minutes, 48 minutes, 49 minutes, 50 minutes, 51 minutes, 52 minutes, 53 minutes, 54 minutes, 55 minutes, 56 minutes, 57 minutes, 58 minutes, 59 minutes, 1 hour, 1.5 hours, 2 hours, 2.5 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 1 day, 1.5 days, 2 days, or more than 3 days.

In one embodiment, delivery comprises co-administration of mannitol. In one embodiment, delivery comprises co-administration of approximately 0.25 to 1.0 g/kg intravenous mannitol. In one embodiment, delivery comprises co-administration of 0.25, 0.26, 0.27, 0.28, 0.29, 0.30, 0.31, 0.32, 0.33, 0.34, 0.35, 0.36, 0.37, 0.38, 0.39, 0.40, 0.41, 0.42, 0.43, 0.44, 0.45, 0.46, 0.47, 0.48, 0.49, 0.50, 0.51, 0.52, 0.53, 0.54, 0.55, 0.56, 0.57, 0.58, 0.59, 0.60, 0.61, 0.62, 0.63, 0.64, 0.65, 0.66, 0.67, 0.68, 0.69, 0.70, 0.71, 0.72, 0.73, 0.74, 0.75, 0.76, 0.77, 0.78, 0.79, 0.80, 0.81, 0.82, 0.83, 0.84, 0.85, 0.86, 0.87, 0.88, 0.89, 0.90, 0.91, 0.92, 0.93, 0.94, 0.95, 0.96, 0.97, 0.98, 0.99, 1.00 g/kg intravenous mannitol.

### Composition Temperature

In one embodiment, delivery of AAV particles to cells of the central nervous system (e.g., parenchyma) comprises a composition wherein the temperature of the composition is 37° C. In one embodiment, delivery comprises a composition wherein the temperature of the composition is between approximately 20° C. and 26° C. In one embodiment, delivery comprises a composition wherein the temperature of the composition is approximately 20.0° C., 20.1° C., 20.2° C., 20.3° C., 20.4° C., 20.5° C., 20.6° C., 20.7° C., 20.8° C., 20.9° C., 21.0° C., 21.1° C., 21.2° C., 21.3° C., 21.4° C., 21.5° C., 21.6° C., 21.7° C., 21.8° C., 21.9° C., 22.0° C., 22.1° C., 22.2° C., 22.3° C., 22.4° C., 22.5° C., 22.6° C., 22.7° C., 22.8° C., 22.9° C., 23.0° C., 23.1° C., 23.2° C., 23.3° C., 23.4° C., 23.5° C., 23.6° C., 23.7° C., 23.8° C., 23.9° C., 24.0° C., 24.1° C., 24.2° C., 24.3° C., 24.4° C., 24.5° C., 24.6° C., 24.7° C., 24.8° C., 24.9° C., 25.0° C., 25.1° C., 25.2° C., 25.3° C., 25.4° C., 25.5° C., 25.6° C., 25.7° C., 25.8° C., 25.9° C., or 26.0° C.

### Drug Physiochemical & Biochemical Properties

In one embodiment, delivery of AAV particles to cells of the central nervous system (e.g., parenchyma) comprises a composition wherein the AAV capsid is hydrophilic. In one embodiment, delivery comprises a composition wherein the AAV capsid is lipophilic.

In one embodiment, delivery of AAV particles to cells of the central nervous system (e.g., parenchyma) comprises a composition wherein the AAV capsid targets a specific receptor. In one embodiment, delivery of AAV particles to cells of the central nervous system (e.g., parenchyma) comprises a composition wherein the AAV capsid further comprises a specific ligand.

In one embodiment, delivery of AAV particles to cells of the central nervous system (e.g., parenchyma) comprises a composition wherein the AAV genome further comprises a cell specific promoter region. In one embodiment, delivery comprises a composition wherein the AAV genome further comprises a ubiquitous promoter region.

### Administration and Delivery

The AAV particles of the present invention may be administered by any route which results in a therapeutically effective outcome. These include, but are not limited to epidural, peridural, subdural (in particular delivery of AAV over one or more targeted regions of the neocortex), intracerebral (into the cerebrum), intracerebroventricular (into the cerebral ventricles), intrathecal (into the spinal canal or within the cerebrospinal fluid at any level of the cerebrospinal axis), intradiscal (within a disc), intradural (within or beneath the dura), intraspinal (within the vertebral column), caudal block, diagnostic, nerve block, or spinal. In specific embodiments, compositions may be administered in a way which allows them cross the blood-brain barrier, vascular barrier, or other epithelial barrier.

In one embodiment, the AAV particles may be delivered by systemic delivery.

In one embodiment, the AAV particles may be delivered by direct injection into the brain. As a non-limiting example, the brain delivery may be by intrastriatal administration.

In one embodiment, the AAV particles may be delivered by a route to bypass the liver metabolism.

In one embodiment, the AAV particles may be delivered to reduce degradation of the AAV particles and/or degradation of the formulation in the blood.

In one embodiment, the AAV particles may be delivered to bypass anatomical blockages such as, but not limited to the blood brain barrier.

In one embodiment, the AAV particles may be formulated and delivered to a subject by a route which increases the speed of drug effect as compared to oral delivery.

In one embodiment, the AAV particles may be delivered to a subject via a single site of administration.

In one embodiment, the AAV particles may be delivered to a subject via a multi-site route of administration. For example, a subject may be administered the AAV particles at 2, 3, 4, 5 or more than 5 sites.

In one embodiment, a subject may be delivered the AAV particles herein using two or more delivery routes.

In one embodiment, the AAV particles may be delivered using convection-enhanced delivery (CED) which is a parenchymal infusion that uses a pressure gradient at a cannula tip within a target structure to deliver a large flow of AAV particles within the interstitial fluid space.

In one embodiment, the AAV particles may be delivered using CED in combination with a tracer visible with magnetic resonance (MR) such as, but not limited to, Gadoteridol. As shown by Bankiewicz et al. (J Control Release 2016 Feb. 27 Epub), the combination of CED and Gadoteridol enhances the accuracy and effectiveness of AAV delivery as it provides a visualization of the infusion in real-time.

In one embodiment, the AAV particles may be delivered to a subject who is using or who has used a treatment stimulator for brain diseases. Non-limiting examples include treatment stimulators from THERATAXIS™ and the treatment stimulators described in International Patent Publication No. WO2008144232, the contents of which are herein incorporated by reference in its entirety.

In one embodiment, the delivery of the AAV particles in a subject may be determined and/or predicted using the prediction methods described in International Patent Publication No. WO2001085230, the contents of which are herein incorporated by reference in its entirety.

In one embodiment, a subject may be imaged prior to, during and/or after administration of the AAV particles. The imaging method may be a method known in the art and/or described herein. As a non-limiting example, the imaging method which may be used to classify brain tissue includes the medical image processing method described in U.S. Pat. Nos. 7,848,543, 9,101,282 and EP Application No. EP1768041, the contents of each of which are herein incorporated by reference in their entireties. As yet another non-limiting example, the physiological states and the effects of treatment of a neurological disease in a subject may be tracked using the methods described in US Patent Publication No. US20090024181, the contents of which are herein incorporated by reference in its entirety.

In one embodiment, the flow of a composition comprising the AAV particles may be controlled using acoustic waveform outside the target area. Non-limiting examples of devices, methods and controls for using sonic guidance to control molecules is described in US Patent Application No. US20120215157, U.S. Pat. No. 8,545,405, International Patent Publication Nos. WO2010096495 and WO2010080701, the contents of each of which are herein incorporated by reference in their entireties.

In one embodiment, the flow of a composition comprising the AAV particles may be modeled prior to administration using the methods and apparatus described in U.S. Pat. Nos. 6,549,803 and 8,406,850 and US Patent Application No. US20080292160, the content of each of which is incorporated by reference in their entireties. As a non-limiting example, the physiological parameters defining edema induced upon infusion of fluid from an intraparenchymally placed catheter may be estimated using the methods described in U.S. Pat. No. 8,406,850 and US Patent Application No. US20080292160, the contents of which is herein incorporated by reference in its entirety.

In one embodiment, the distribution of the AAV particles described herein may be evaluated using imaging technology from Therataxis and/or Brain Lab.

### Delivery to the CNS

In one embodiment, the AAV particles may be delivered to the central nervous system using any of the methods described herein.

Factors affecting delivery of payloads by parvovirus, e.g., AAV particles to cells of the central nervous system (e.g., parenchyma) as provided by the invention may include, but are not limited to, infusion parameters and devices, spatial orientation of the subject, composition physiochemical properties, and viral physiochemical and biochemical properties.

In one embodiment, the delivery method and duration is chosen to provide broad transduction in the spinal cord. As a non-limiting example, intrathecal delivery is used to provide broad transduction along the rostral-caudal length of the spinal cord. As another non-limiting example, multi-site infusions provide a more uniform transduction along the rostral-caudal length of the spinal cord. As yet another non-limiting example, prolonged infusions provide a more uniform transduction along the rostral-caudal length of the spinal cord.

In one embodiment, delivery of payloads by adeno-associated virus (AAV) particles to the central nervous system (e.g., parenchyma) may be by prolonged delivery to the cerebrospinal fluid (CSF). CSF is produced by specialized ependymal cells that comprise the choroid plexus located in the ventricles of the brain. CSF produced within the brain then circulates and surrounds the central nervous system including the brain and spinal cord.

In one embodiment, the AAV particles described herein may be delivered by a method which allows even distribution of the AAV particles along the CNS taking into account cerebrospinal fluid (CSF) dynamics. CSF continually circulates around the central nervous system, including the ventricles of the brain and subarachnoid space that surrounds both the brain and spinal cord, while maintaining a homeostatic balance of production and reabsorption into the vascular system. The entire volume of CSF is replaced (CSF turnover (TO)) approximately four to six times per day or approximately once every four hours, though values for individuals may vary. Non-limiting examples of delivery to the CSF pathway include intrathecal (IT) and intracerebroventricular (ICV) administration.

In one embodiment, a subject may be delivered the AAV particles described herein to a region of the spinal cord which has been determined to have a higher CSF flow along the anterior aspect of the cord as compared to the flow along the entire cord.

In one embodiment, a subject may be delivered the AAV particles described herein to a region of the spinal cord which has been determined to have a higher CSF flow along the ventral aspect of the cord as compared to the flow along the entire cord.

In one embodiment, AAV particles are delivered taking into account the oscillating movement and vortexes of the CSF around the spinal cord. Vortexes are formed by the oscillating movement of the CSF around the cord and these individual vortices combine to form vortex arrays. The arrays combine to form fluid paths for movement of the AAV particles along the spinal cord.

In one embodiment, the CSF flow dynamics of a subject are evaluated prior to administration of the AAV particles described herein. As a non-limiting example, a subject is evaluated pre and post-catheter implant to determine the flow dynamics of the CSF and an imaging enhancer such as, but not limited to, gadoluminate may be used during the evaluation.

In one embodiment, the macrodistribution of the AAV particles described herein across the spinal cord and brain may be governed by CSF flow and/or dosing parameters such as, but not limited to, infusion rate.

In one embodiment, the microdistribution of the AAV particles described herein into tissue may be dependent on CSF flow, exposure time and amount of AAV with the tissue and the properties of the AAV particles.

In one embodiment, the fine distribution of the AAV particles described herein into cells may be a function of the biology of the AAV particle such as, but not limited to, receptor binding, retrograde transport and/or anterograde transport of the AAV particles.

### Intraparenchymal (IPa) Administration

In one embodiment, delivery of AAV particles to cells of the central nervous system is performed by intraparenchymal (IPa) administration. IPa administration delivers the AAV particles directly into the brain parenchyma.

In one embodiment, AAV particles may be delivered to a subject using IPa delivery in at least one location in the brain parenchyma. The location or locations may be located in the right brain, the left brain or both the right and left brain. As a non-limiting example, the location of the IPa delivery is in the right brain in the caudate and the putamen. As a non-limiting example, the location of the IPa delivery is in the left brain in the caudate and the putamen. As a non-limiting example, the location of the IPa delivery is in the right brain in the caudate and the putamen and in the left brain in the caudate and the putamen.

In one embodiment, AAV particles may be delivered to a subject using IPa delivery in the brain parenchyma in at least 1, 2, 3, 4, 5, 6, 7, 8, 9, 10 or more than 10 locations in the brain parenchyma. As a non-limiting example, the AAV particles may be delivered in the right brain in 3 sites. As a non-limiting example, the AAV particles may be delivered in the left brain in 3 sites. As a non-limiting example, the AAV particles may be delivered in the right brain in 3 sites and in the left brain in 3 sites.

In one embodiment, the AAV particles may be delivered to a subject using IPa delivery in 3 sites of the caudate and putamen in the right brain and 3 sites of the caudate and putamen in the left brain.

In one embodiment, the AAV particles may be delivered to a subject using IPa delivery in the caudate of the left brain.

In one embodiment, the AAV particles may be delivered to a subject using IPa delivery in the caudate of the right brain.

In one embodiment, the AAV particles may be delivered to a subject using IPa delivery in the putamen of the left brain.

In one embodiment, the AAV particles may be delivered to a subject using IPa delivery in the putamen of the right brain.

In one embodiment, the AAV particles may be delivered to a subject using IPa delivery to the caudate of the left brain and the caudate of the right brain.

In one embodiment, the AAV particles may be delivered to a subject using IPa delivery to the putamen of the left brain and the putamen of the right brain.

In one embodiment, the AAV particles may be delivered to a subject using IPa delivery to the caudate of the left brain and the putamen of the right brain.

In one embodiment, the AAV particles may be delivered to a subject using IPa delivery to the caudate of the right brain and the putamen of the left brain.

In one embodiment, intraparenchymal delivery of the AAV particles described herein may use convection enhanced delivery. While not wishing to be bound by theory, convection enhanced delivery uses sustained pressure (or convection) to push a drug solution through brain tissue causing the drug to infuse at a higher rate than it can diffuse away from the injection site.

In one embodiment, the volume of delivery of the AAV particles per site may be 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60 or more than 60 ul per site of administration. As a non-limiting example, the volume of delivery may be 30 ul per site of administration.

In one embodiment, the administration of the AAV particles to a subject provides coverage of the putamen of a subject (e.g., the left and/or right putamen). The administration of the AAV particles may provide at least 8%, 9%, 10%, 13%, 14%, 15%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 60%, 65%, 70%, 75%, 80%, 85%, 90%, 95% or more than 95% to the left and/or right putamen of a subject. As a non-limiting example, the coverage is at least 20%. As another non-limiting example, the coverage is at least 30%. As a non-limiting example, the coverage is at least 40%. The administration of the AAV particles may provide at least 8%, 9%, 10%, 13%, 14%, 15%, 19%, 20%, 21%, 22%, 23%, 24%, 25%, 26%, 27%, 28%, 29%, 30%, 31%, 32%, 33^{%}, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 60%, 65%, 70%, 75%, 80%, 85%, 90%, 95% or more than 95% coverage of the surface area of the left and/or right putamen of a subject. As a non-limiting example, the total coverage is at least 20%. As another non-limiting example, the total coverage is at least 30%. As a non-limiting example, the total coverage is at least 40%. The administration of the AAV particles may provide 10-40%, 19-25%, 20-40%, 20-30%, 20-35%, 20-50%, 25-38%, 30-40%, 35-40%, 30-60%, 40-70%, 50-80% or 60-90% coverage to the left and/or right putamen of a subject or to the total surface area of the left and/or right putamen of a subject.

In one embodiment, the total dose of AAV particles delivered via IPa administration may be between about 1×10⁶ VG and about 1×10¹⁶ VG. In some embodiments, delivery may comprise a total dose of about 1×10⁶, 2×10⁶, 3×10⁶, 4×10⁶, 5×10⁶, 6×10⁶, 7×10⁶, 8×10⁶, 9×10⁶, 1×10⁷, 2×10⁷, 3×10⁷, 4×10⁷, 5×10⁷, 6×10⁷, 7×10⁷, 8×10⁷, 9×10⁷, 1×10⁸, 2×10⁸, 3×10⁸, 4×10⁸, 5×10⁸, 6×10⁸, 7×10⁸, 8×10⁸, 9×10⁸, 1×10⁹, 1.9×10⁹, 2×10⁹, 3×10⁹, 4×10⁹, 5×10⁹, 6×10⁹, 7×10⁹, 8×10⁹, 9×10⁹, 1×10¹⁰, 2×10¹⁰, 3×10¹⁰, 4×10¹⁰, 5×10¹⁰, 5.7×10¹⁰, 6×10¹⁰, 7×10¹⁰, 8×10¹⁰, 9×10¹⁰, 1×10¹¹, 1.1×10¹¹, 2×10¹¹, 2.5×10¹¹, 3×10¹¹, 3.4×10¹¹, 4×10¹¹, 5×10¹¹, 6×10¹¹, 7×10¹¹, 8×10¹¹, 9×10¹¹, 1×10¹², 2×10¹², 3×10¹², 4×10¹², 5×10¹², 6×10¹², 7×10¹², 8×10¹², 9×10¹², 1×10¹³, 2×10¹³ 3×10¹³, 4×10¹³, 5×10¹³, 6×10¹³, 7×10¹³, 8×10¹³, 9×10¹³, 1×10¹⁴, 2×10¹⁴, 3×10¹⁴, 4×10¹⁴, 5×10¹⁴ 6×10¹⁴, 7×10¹⁴, 8×10¹⁴, 9×10¹⁴, 1×10¹⁵, 2×10¹⁵, 3×10¹⁵, 4×10¹⁵, 5×10¹⁵, 6×10¹⁵, 7×10¹⁵, 8×10¹⁵, 9×10¹⁵, or 1×10¹⁶ VG.

In one embodiment, delivery of AAV particles via IPa delivery may comprise a composition concentration between about 1×10⁶ VG/mL and about 1×10¹⁶ VG/mL. In some embodiments, delivery may comprise a composition concentration of about 1×10⁶, 2×10⁶, 3×10⁶, 4×10⁶, 5×10⁶, 6×10⁶, 7×10⁶, 8×10⁶, 9×10⁶, 1×10⁷, 2×10⁷, 3×10⁷, 4×10⁷, 5×10⁷, 6×10⁷, 7×10⁷, 8×10⁷, 9×10⁷, 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 2×10¹⁰, 3×10¹⁰, 4×10¹⁰, 5×10¹⁰, 6×10¹⁰, 7×10¹⁰, 8×10¹⁰, 9×10¹⁰, 1×10¹¹, 2×10¹¹, 3×10¹¹, 4×10¹¹, 5×10¹¹, 6×10¹¹, 7×10¹¹, 8×10¹¹, 9×10¹¹, 1×10¹², 1.9×10¹², 2×10¹², 3×10¹², 4×10¹², 5×10¹², 6×10¹², 7×10¹², 8×10¹², 9×10¹², 1×10¹³, 2×10¹³, 3×10¹³, 4×10¹³, 5×10¹³, 6×10¹³, 7×10¹³, 8×10¹³, 9×10¹³, 1×10¹⁴, 2×10¹⁴, 3×10¹⁴, 4×10¹⁴, 5×10¹⁴ 6×10¹⁴, 7×10¹⁴, 8×10¹⁴, 9×10¹⁴, 1×10¹⁵, 2×10¹⁵, 3×10¹⁵, 4×10¹⁵, 5×10¹⁵, 6×10¹⁵, 7×10¹⁵, 8×10¹⁵, 9×10¹⁵, or 1×10¹⁶ VG/mL. In one embodiment, delivery comprises a composition concentration of 1.9×10¹² VG/mL.

In one embodiment, delivery of AAV particles via IPa delivery may comprise a dose per site of between about 1×10⁶ VG/site and about 1×10¹⁶ VG/site. In some embodiments, delivery may comprise a composition concentration of about 1×10⁶, 2×10⁶, 3×10⁶, 4×10⁶, 5×10⁶, 6×10⁶, 7×10⁶, 8×10⁶, 9×10⁶, 1×10⁷, 2×10⁷, 3×10⁷, 4×10⁷, 5×10⁷, 6×10⁷, 7×10⁷, 8×10⁷, 9×10⁷, 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 2×10¹⁰, 3×10¹⁰, 4×10¹⁰, 5×10¹⁰, 5.7×10¹⁰, 6×10¹⁰, 7×10¹⁰, 8×10¹⁰, 9×10¹⁰, 1×10¹¹, 2×10¹¹, 3×10¹¹, 4×10¹¹, 5×10¹¹, 6×10¹¹, 7×10¹¹, 8×10¹¹, 9×10¹¹, 1×10¹², 1.9×10¹², 2×10¹², 3×10¹², 4×10¹², 5×10¹², 6×10¹², 7×10¹², 8×10¹², 9×10¹², 1×10¹³, 2×10¹³, 3×10¹³, 4×10¹³, 5×10¹³, 6×10¹³, 7×10¹³, 8×10¹³, 9×10¹³, 1×10¹⁴, 2×10¹⁴, 3×10¹⁴, 4×10¹⁴, 5×10¹⁴ 6×10¹⁴, 7×10¹⁴, 8×10¹⁴, 9×10¹⁴, 1×10¹⁵, 2×10¹⁵, 3×10¹⁵, 4×10¹⁵, 5×10¹⁵, 6×10¹⁵, 7×10¹⁵, 8×10¹⁵, 9×10¹⁵, or 1×10¹⁶ VG/site. In one embodiment, the dose per site is 5.7×10¹⁰ VG/site.

In one embodiment, the maximum flowrate of a formulation comprising the AAV particles described herein is 0.1 uL/min, 0.2 uL/min, 0.3 uL/min, 0.4 uL/min, 0.5 uL/min, 0.6 uL/min, 0.7 uL/min, 0.8 uL/min, 0.9 uL/min, 1 uL/min, 2 uL/min, 3 uL/min, 4 uL/min, 5 uL/min, 6 uL/min, 7 uL/min, 8 uL/min, 9 uL/min, 10 uL/min, 11 uL/min, 12 uL/min, 13 uL/min, 14 uL/min, 15 uL/min, 16 uL/min, 17 uL/min, 18 uL/min, 19 uL/min, 20 uL/min, 21 uL/min, 22 uL/min, 23 uL/min, 24 uL/min, 25 uL/min, 26 uL/min, 27 uL/min, 28 uL/min, 29 uL/min, 30 uL/min, 31 uL/min, 32 uL/min, 33 uL/min, 34 uL/min, 35 uL/min, 36 uL/min, 37 uL/min, 38 uL/min, 39 uL/min, 40 uL/min, 41 uL/min, 42 uL/min, 43 uL/min, 44 uL/min, 45 uL/min, 46 uL/min, 47 uL/min, 48 uL/min, 49 uL/min, 50 uL/min, or more than 50 uL/min. The maximum flowrate may depend on various factors including, but not limited to, the tissue for delivery, the progression of the disease, formulation, and temperature of formulation. As a non-limiting example, the maximum flowrate for white matter tissue may be 40 uL/min. As another non-limiting example, the maximum flowrate for thalamus tissue is 20 uL/min. As yet another non-limiting example, the maximum flowrate for putamen tissue is 15 uL/min.

In one embodiment, delivery of AAV particles to cells of the central nervous system is performed by intraparenchymal (IPa) administration in a subject who has been diagnosed with or used for treatment of a subject who may have Parkinson's Disease (PD), Huntington's Disease (HD), and/or Alzheimer's Disease (AD).

In one embodiment, delivery of AAV particles to brain tissue is performed by intraparenchymal (IPa) administration in a subject who has been diagnosed with or used for treatment of a subject who may have Parkinson's Disease (PD), Huntington's Disease (HD), and/or Alzheimer's Disease (AD).

In one embodiment, a catheter used for IPa administration of the AAV particles is compatible with stereotactic fixtures, is MRI-safe (up to 3T), has a CED flow rate of greater than 15 ul/min, reflux-resistant and/or is repositionable. The catheter may also include a pressure sensor and may have individual flow channels to provide multiple infusion levels.

In one embodiment, a catheter used for IPa administration of the AAV particles may include, but is not limited to, the SmartFlow catheter (MRI Interventions), SmartFlow Adjustable Tip Catheter (MRI Interventions), Cleveland Multiport Catheter (Infuseon Therapeutics, Inc.), MEMS catheter (Alcyone Lifesciences, Inc.), Carbothane CED cannula (Renishaw) Smartflow Flex (BrainLab) and/or Intracerebral Microinj ection Instrument (IMI) (Atanse).

In one embodiment, the device used to deliver the AAV particles of the invention by IPa administration may be, but is not limited to, a device from MRI Intervention, Alcyone, Atanse and/or Medgenesis.

In one embodiment, the AAV particles are delivered by intraparenchymal administration to a subject using at least one site. As a non-limiting example, the dose of AAV particles may be 3.4×10¹¹ vg administered bilaterally to the caudate and putamen at a dose a volume of 30 ul/site. As a non-limiting example, the dose of AAV particles may be 3.4×10¹¹ vg administered bilaterally to the left caudate and right putamen at a dose a volume of 30 ul/site. As a non-limiting example, the dose of AAV particles may be 3.4×10¹¹ vg administered bilaterally to the right caudate and left putamen at a dose a volume of 30 ul/site. As a non-limiting example, the dose of AAV particles may be 3.4×10¹¹ vg administered bilaterally to the left caudate and left putamen at a dose a volume of 30 ul/site. As a non-limiting example, the dose of AAV particles may be 3.4×10¹¹ vg administered bilaterally to the right caudate and right putamen at a dose a volume of 30 ul/site. As a non-limiting example, the dose of AAV particles may be 1.1×10¹¹ vg administered bilaterally to the caudate and putamen at a dose a volume of 30 ul/site. As a non-limiting example, the dose of AAV particles may be 1.1×10¹¹ vg administered bilaterally to the left caudate and right putamen at a dose a volume of 30 ul/site. As a non-limiting example, the dose of AAV particles may be 1.1×10¹¹ vg administered bilaterally to the right caudate and left putamen at a dose a volume of 30 ul/site. As a non-limiting example, the dose of AAV particles may be 1.1×10¹¹ vg administered bilaterally to the left caudate and left putamen at a dose a volume of 30 ul/site. As a non-limiting example, the dose of AAV particles may be 1.1×10¹¹ vg administered bilaterally to the right caudate and right putamen at a dose a volume of 30 ul/site. As a non-limiting example, the dose of AAV particles may be 5.7×10¹⁰ vg administered to either the left or right caudate at a dose a volume of 30 ul/site. As a non-limiting example, the dose of AAV particles may be 5.7×10¹⁰ vg administered to both the left and right caudate at a dose a volume of 30 ul/site. As a non-limiting example, the dose of AAV particles may be 5.7×10¹⁰ vg administered to either the left or right putamen at a dose a volume of 30 ul/site. As a non-limiting example, the dose of AAV particles may be 5.7×10¹⁰ vg administered to both the left and right putamen at a dose a volume of 30 ul/site.

In one embodiment, the AAV particles comprise an AAV1 capsid are delivered by intraparenchymal administration to a subject using at least one site. As a non-limiting example, the dose of AAV particles may be 3.4×10¹¹ vg administered bilaterally to the caudate and putamen at a dose a volume of 30 ul/site. As a non-limiting example, the dose of AAV particles may be 3.4×10¹¹ vg administered bilaterally to the left caudate and right putamen at a dose a volume of 30 ul/site. As a non-limiting example, the dose of AAV particles may be 3.4×10¹¹ vg administered bilaterally to the right caudate and left putamen at a dose a volume of 30 ul/site. As a non-limiting example, the dose of AAV particles may be 3.4×10¹¹ vg administered bilaterally to the left caudate and left putamen at a dose a volume of 30 ul/site. As a non-limiting example, the dose of AAV particles may be 3.4×10¹¹ vg administered bilaterally to the right caudate and right putamen at a dose a volume of 30 ul/site. As a non-limiting example, the dose of AAV particles may be 1.1×10¹¹ vg administered bilaterally to the caudate and putamen at a dose a volume of 30 ul/site. As a non-limiting example, the dose of AAV particles may be 1.1×10¹¹ vg administered bilaterally to the left caudate and right putamen at a dose a volume of 30 ul/site. As a non-limiting example, the dose of AAV particles may be 1.1×10¹¹ vg administered bilaterally to the right caudate and left putamen at a dose a volume of 30 ul/site. As a non-limiting example, the dose of AAV particles may be 1.1×10¹¹ vg administered bilaterally to the left caudate and left putamen at a dose a volume of 30 ul/site. As a non-limiting example, the dose of AAV particles may be 1.1×10¹¹ vg administered bilaterally to the right caudate and right putamen at a dose a volume of 30 ul/site. As a non-limiting example, the dose of AAV particles may be 5.7×10¹⁰ vg administered to either the left or right caudate at a dose a volume of 30 ul/site. As a non-limiting example, the dose of AAV particles may be 5.7×10¹⁰ vg administered to both the left and right caudate at a dose a volume of 30 ul/site. As a non-limiting example, the dose of AAV particles may be 5.7×10¹⁰ vg administered to either the left or right putamen at a dose a volume of 30 ul/site. As a non-limiting example, the dose of AAV particles may be 5.7×10¹⁰ vg administered to both the left and right putamen at a dose a volume of 30 ul/site.

### Intracerebroventricular (ICV) Administration

In one embodiment, delivery of AAV particles to cells of the central nervous system (e.g., parenchyma) is performed by intracerebroventricular (ICV) administration. ICV administration comprises delivery by injection into the ventricular system of the brain usually by prolonged infusion. ICV prolonged infusion may comprise delivery to any of the ventricles of the brain, including, but not limited to, either of the two lateral ventricles left and right, third ventricle, and/or fourth ventricle. ICV prolonged infusion may comprise delivery to any of the foramina, or channels that connect the ventricles, including, but not limited to, interventricular foramina, also called the foramina of Monroe, cerebral aqueduct, cistema magna, and/or central canal. ICV prolonged infusion may comprise delivery to any of the apertures of the ventricular system including, but not limited to, the median aperture (aka foramen of Magendie), right lateral aperture, and/or left lateral aperture (aka foramina of Lushka). In one embodiment, ICV prolonged infusion comprises delivery to the perivascular space in the brain.

In one embodiment, a catheter used for ICV administration of the AAV particles may include, but is not limited to, the SmartFlow catheter (MRI Interventions), SmartFlow Adjustable Tip Catheter (MRI Interventions), Cleveland Multiport Catheter (Infuseon Therapeutics, Inc.), MEMS catheter (Alcyone Lifesciences, Inc.), Carbothane CED cannula (Renishaw) Smartflow Flex (BrainLab) and/or Intracerebral Microinj ection Instrument (IMI) (Atanse).

In one embodiment, subjects such as mammals (e.g., non-human primates (NHPs)) are administered by intracerebroventricular (ICV) infusion the AAV particles described herein. The AAV particles may comprise scAAV or ssAAV, of any of the serotypes described herein, comprising a payload (e.g., a transgene). The dose may be 1×10¹³ to 3×10¹³ vg per subject. The subject may be administered a dose of the AAV particles over an extended period of time such as, but not limited to, 10 ml over 10 hours. The subjects may be evaluated 14-30 days (e.g., 14, 21, 28, or 30 days) after administration to determine the expression of the payload in the subject. Further, the subject may be evaluated prior to administration and after administration to determine changes in behavior and activity such as, but not limited to, tremors, lethargic behavior, motor deficits in limbs, strength, spinal reflex deficits, food consumption. (For AAV9 in Non Human Primates (Cyno) see: Samaranch et al. Human Gene Therapy 23:382-389 April 2012, Samaranch et al. Human Gene Therapy 24: 526-532 May 2013, Samaranch et al. Molecular Therapy 22(2) 329-337 February 2014, Gray et al. Gene Ther. 20(4) 450-459 April 2013; the contents of each of which are herein incorporated by reference in their entireties).

In one embodiment, the AAV particles are delivered by intracerebroventricular infusion. As a non-limiting example, the dose of AAV particles may be 1.0×10¹³ vg administered for 10 hours.

In one embodiment, the AAV particles are ssAAV particles and they are delivered by intracerebroventricular infusion. As a non-limiting example, the dose of ssAAV particles may be 1.0×10¹³ vg administered for 10 hours.

In one embodiment, the AAV particles are scAAV particles and they are delivered by intracerebroventricular infusion. As a non-limiting example, the dose of scAAV particles may be 1.0×10¹³ vg administered for 10 hours.

In one embodiment, the AAV particles comprise an AAV1 capsid and are delivered by intracerebroventricular infusion. As a non-limiting example, the dose of AAV particles may be 1.0×10¹³ vg administered for 10 hours.

In one embodiment, the AAV particles comprise an AAV1 capsid and are ssAAV particles and they are delivered by intracerebroventricular infusion. As a non-limiting example, the dose of ssAAV particles may be 1.0×10¹³ vg administered for 10 hours.

In one embodiment, the AAV particles comprise an AAV1 capsid and are scAAV particles and they are delivered by intracerebroventricular infusion. As a non-limiting example, the dose of scAAV particles may be 1.0×10¹³ vg administered for 10 hours.

In one embodiment, the AAV particles comprise an AAV-DJ8 capsid and are delivered by intracerebroventricular infusion. As a non-limiting example, the dose of AAV particles may be 1.0×10¹³ vg administered for 10 hours.

In one embodiment, the AAV particles comprise an AAV-DJ8 capsid and are ssAAV particles and they are delivered by intracerebroventricular infusion. As a non-limiting example, the dose of ssAAV particles may be 1.0×10¹³ vg administered for 10 hours.

In one embodiment, the AAV particles comprise an AAV-DJ8 capsid and are scAAV particles and they are delivered by intracerebroventricular infusion. As a non-limiting example, the dose of scAAV particles may be 1.0×10¹³ vg administered for 10 hours.

### Intrathecal (IT) Administration

In one embodiment, the AAV particles described herein may be administered to a subject by intrathecal (IT) administration such as by infusion.

In one embodiment, intrathecal administration delivers AAV particles to targeted regions of the CNS. Non-limiting examples of regions of the CNS to deliver AAV particles include dorsal root ganglion, dentate nucleus-cerebellum and the auditory pathway.

In one embodiment, intrathecal administration of AAV particles provides peripheral exposure which is as low as possible or a moderate level that is beneficial. As a non-limiting example, intrathecal administration of AAV particles shows almost no peripheral exposure to the liver.

For intrathecal infusion, while not wishing to be bound by theory, AAV macrodistribution across the spinal cord and brain can be governed by CSF flow and dosing parameters such as infusion rate. AAV microdistribution into tissue can be controlled by a variety of properties including CSF flow, AAV tissue exposure to enhance interstitial movement (time and concentration) and the biological properties of the AAV. AAV fine distribution into cells may be a function of the biology of the AAV such as, but not limited to, receptor binding, retrograde transport and anterograde transport.

In some embodiments IT infusion comprises delivery to the cervical, thoracic, and or lumbar regions of the spine.

In one embodiment, the catheter used to deliver the AAV particles via intrathecal administration is located in the lumbar region of the spinal cord. The catheter may be located in one or more than one location in the lumbar region.

In one embodiment, the catheter used to deliver the AAV particles via intrathecal administration is located in the cervical region of the spinal cord. The catheter may be located in one or more than one location in the cervical region.

In one embodiment, the catheter used to deliver the AAV particles via intrathecal administration is located in the lumbar region and the cervical region of the spinal cord. As a non-limiting example, a catheter may be located in the cervical and the lumbar region. As another non-limiting example, a catheter may be located in the cervical region and two catheters may be located in the lumbar region.

As used herein, IT infusion into the spine is defined by the vertebral level at the site of prolonged infusion. In some embodiments IT infusion comprises delivery to the cervical region of the spine at any location including, but not limited to C1, C2, C3, C4, C5, C6, C7, and/or C8. In some embodiments IT infusion comprises delivery to the thoracic region of the spine at any location including, but not limited to T1, T2, T3, T3, T4, T5, T6, T7, T8, T9, T10, T11, and/or T12. In some embodiments IT infusion comprises delivery to the lumbar region of the spine at any location including, but not limited to L1, L2, L3, L3, L4, L5, and/or L6. In some embodiments IT infusion comprises delivery to the sacral region of the spine at any location including, but not limited to S1, S2, S3, S4, or S5. In some embodiments, delivery by IT infusion comprises one or more than one site of prolonged infusion.

In some embodiments, delivery by IT infusion may comprise 1, 2, 3, 4, 5, 6, 7, 8, 9, 10, 11, 12, 13, 14, 15, 16, 17, 18, 19, 20, 21, 22, 23, 24, or 25 sites infusion. In one embodiment, delivery by IT infusion comprises at least three sites of infusion. In one embodiment, delivery by IT infusion consists of three sites of infusion. In one embodiment, delivery by IT infusion comprises three sites of infusion at C1, T1, and L1.

In one embodiment, delivery by IT infusion includes administration using a cervical catheter located at C5.

In one embodiment, delivery by IT infusion includes administration using a cervical catheter located at C1.

In one embodiment, delivery by IT infusion may be via a cervical catheter placed between C1 and C2.

In one embodiment, delivery by IT infusion may be via a thoracolumbar catheter placed between T10 and L1.

In one embodiment, the catheter for IT infusion may be placed in the cervical region such as, but not limited to, C1-C2.

In one embodiment, the catheter for IT infusion may be placed thoracolumbar such as, but not limited to, T10/L1.

In one embodiment, IT administration may be used to deliver AAV particles to motor neurons. As a non-limiting example, the motor neurons are located in the ventral horn of the spinal cord.

In one embodiment, IT administration may be used to deliver AAV particles to motor neurons to treat ALS and/or the symptoms or ALS. As a non-limiting example, the motor neurons are located in the ventral horn of the spinal cord.

In one embodiment, IT administration may be used to deliver AAV particles to motor neurons to treat SMA and/or the symptoms or SMA. As a non-limiting example, the motor neurons are located in the ventral horn of the spinal cord.

In one embodiment, IT administration may be used to deliver AAV particles to sensory neurons and/or dorsal root ganglion.

In one embodiment, IT administration may be used to deliver AAV particles to sensory neurons and/or dorsal root ganglion to treat FA and/or the symptoms of FA.

In one embodiment, IT administration may be used to deliver AAV particles to sensory neurons and/or dorsal root ganglion to treat Neuropathic Pain and/or the symptoms of Neuropathic Pain.

In one embodiment, subjects such as mammals (e.g., non-human primates (NHPs)) are administered by intrathecal (IT) infusion the AAV particles described herein. The AAV particles may comprise scAAV or ssAAV, of any of the serotypes described herein, comprising a payload (e.g., atransgene). The dose may be 1×10¹³ to 3×10¹³ vg per subject. The subject may be administered a dose of the AAV particles over an extended period of time such as, but not limited to, 10 ml over 10 hours. The subjects may be evaluated 14-30 days (e.g., 14, 21, 28, or 30 days) after administration to determine the expression of the payload in the subject. Further, the subject may be evaluated prior to administration and after administration to determine changes in behavior and activity such as, but not limited to, tremors, lethargic behavior, motor deficits in limbs, strength, spinal reflex deficits, food consumption. (For AAV9 in Non Human Primates (Cyno) see: Samaranch et al. Human Gene Therapy 23:382-389 April 2012, Samaranch et al. Human Gene Therapy 24: 526-532 May 2013, Samaranch et al. Molecular Therapy 22(2) 329-337 February 2014, Gray et al. Gene Ther. 20(4) 450-459 April 2013; the contents of each of which are herein incorporated by reference in their entireties).

In one embodiment, administration of the AAV particles may be by IT administration and the AAV particles comprise an AAVrh10 capsid. As a non-limiting example, the AAV particle is single stranded (ss). As another non-limiting example, the AAV particle is self-complementary (sc).

In one embodiment, administration of the AAV particles may be by IT administration and the AAV particles comprise an AAV6 capsid. As a non-limiting example, the AAV particle is single stranded (ss). As another non-limiting example, the AAV particle is self-complementary (sc).

In one embodiment, administration of the AAV particles may be by IT administration and the AAV particles comprise an AAV5 capsid. As a non-limiting example, the AAV particle is single stranded (ss). As another non-limiting example, the AAV particle is self-complementary (sc).

In one embodiment, administration of the AAV particles targets the motor neurons via IT administration of the AAV particles described herein. As a non-limiting example, the AAV particle comprises an AAVrh10 capsid and is single stranded (ss). As a non-limiting example, the AAV particle comprises an AAVrh10 capsid and is self-complementary (sc). As a non-limiting example, the AAV particle comprises an AAV6 capsid and is single stranded (ss). As a non-limiting example, the AAV particle comprises an AAV6 capsid and is self-complementary (sc).

In one embodiment, administration of the AAV particles targets the proprioceptive sensory neurons via IT administration of the AAV particles described herein. As a non-limiting example, the AAV particle comprises an AAVrh10 capsid and is single stranded (ss). As a non-limiting example, the AAV particle comprises an AAVrh10 capsid and is self-complementary (sc). As a non-limiting example, the AAV particle comprises an AAV6 capsid and is single stranded (ss). As a non-limiting example, the AAV particle comprises an AAV6 capsid and is self-complementary (sc).

In one embodiment, administration of the AAV particles targets the motor neurons via IT administration of the AAV particles described herein to treat and/or mitigate the symptoms of amyotrophic lateral sclerosis (ALS). As a non-limiting example, the AAV particle comprises an AAVrh10 capsid and is single stranded (ss). As a non-limiting example, the AAV particle comprises an AAVrh10 capsid and is self-complementary (sc). As a non-limiting example, the AAV particle comprises an AAV6 capsid and is single stranded (ss). As a non-limiting example, the AAV particle comprises an AAV6 capsid and is self-complementary (sc).

In one embodiment, administration of the AAV particles targets the proprioceptive sensory neurons via IT administration of the AAV particles described herein to treat and/or mitigate the symptoms of Friedreich's Ataxia (FA). As a non-limiting example, the AAV particle comprises an AAVrh10 capsid and is single stranded (ss). As a non-limiting example, the AAV particle comprises an AAVrh10 capsid and is self-complementary (sc). As a non-limiting example, the AAV particle comprises an AAV6 capsid and is single stranded (ss). As a non-limiting example, the AAV particle comprises an AAV6 capsid and is self-complementary (sc).

In one embodiment, a catheter used for IT administration of the AAV particles may include, but is not limited to, the SmartFlow catheter (MRI Interventions), SmartFlow Adjustable Tip Catheter (MRI Interventions), Cleveland Multiport Catheter (Infuseon Therapeutics, Inc.), MEMS catheter (Alcyone Lifesciences, Inc.), Carbothane CED cannula (Renishaw) Smartflow Flex (BrainLab) and/or Intracerebral Microinj ection Instrument (IMI) (Atanse).

In one embodiment, the device used to deliver the AAV particles of the invention by IT infusion may be, but is not limited to, a device from Medtronic Neuromodulation, Codman Neuro and/or Alcyone.

In one embodiment, an intrathecal delivery method from Alcyone may be used to administer the AAV particles described herein. As a non-limiting example, the method leverages the natural pulsatility of CSF to ensure effective delivery of. Additionally, a sensor and camera enabled steerable catheter may be used in the intrathecal delivery of the AAV particles described herein.

In one embodiment, the AAV particles are delivered by intrathecal administration to a subject using at least one site. As a non-limiting example, the dose of AAV particles may be 3×10¹³ vg administered at 3 sites at a volume/rate of 3 ml/3 hours. As another non-limiting example, the dose of AAV particles may be 1×10¹³ vg or 3×10¹³ vg administered at one site in the L (e.g., L1) or C region at a volume/rate of 10 ml/10 hours. As another non-limiting example, the dose of AAV particles may be 3×10¹³ vg administered at 3 sites as a bolus infusion of 1 ml or 3 ml. As another non-limiting example, the dose of AAV particles may be 1×10¹³ vg administered at 1 site (e.g., L or C region) as a bolus infusion of 1 ml. As another non-limiting example, the dose of AAV particles may be 3×10¹³ vg administered at 1 site (e.g., L or C region) as a bolus infusion of 3 ml. As another non-limiting example, the dose of AAV particles may be 2×10¹³ vg, 2×10¹² vg, 2×10¹¹ vg, or 2×10¹⁰ vg administered at 1 site (e.g., L or C region) as 2 bolus infusions.

In one embodiment, the AAV particles are scAAV particles and are delivered by intrathecal administration to a subject using at least one site. As a non-limiting example, the dose of scAAV particles may be 3×10¹³ vg administered at 3 sites at a volume/rate of 3 ml/3 hours. As another non-limiting example, the dose of scAAV particles may be 1×10¹³ vg or 3×10¹³ vg administered at one site in the L (e.g., L1) or C region at a volume/rate of 10 ml/10 hours. As another non-limiting example, the dose of scAAV particles may be 3×10¹³ vg administered at 3 sites as a bolus infusion. As another non-limiting example, the dose of scAAV particles may be 1×10¹³ vg administered at 1 site as a bolus infusion of 1 ml. As another non-limiting example, the dose of scAAV particles may be 3×10¹³ vg administered at 1 site (e.g., L or C region) as a bolus infusion of 3 ml. As another non-limiting example, the dose of scAAV particles may be 2×10¹³ vg, 2×10¹² vg, 2×10¹¹ vg, or 2×10¹⁰ vg administered at 1 site (e.g., L or C region) as 2 bolus infusions.

In one embodiment, the AAV particles are ssAAV particles and are delivered by intrathecal administration to a subject using at least one site. As a non-limiting example, the dose of ssAAV particles may be 3×10¹³ vg administered at 3 sites at a volume/rate of 3 ml/3 hours. As another non-limiting example, the dose of ssAAV particles may be 1×10¹³ vg or 3×10¹³ vg administered at one site in the L (e.g., L1) or C region at a volume/rate of 10 ml/10 hours. As another non-limiting example, the dose of ssAAV particles may be 3×10¹³ vg administered at 3 sites as a bolus infusion. As another non-limiting example, the dose of ssAAV particles may be 1×10¹³ vg administered at 1 site as a bolus infusion of 1 ml. As another non-limiting example, the dose of ssAAV particles may be 3×10¹³ vg administered at 1 site (e.g., L or C region) as a bolus infusion of 3 ml. As another non-limiting example, the dose of ssAAV particles may be 2×10¹³ vg, 2×10¹² vg, 2×10¹⁰ vg, or 2×10¹⁰ vg administered at 1 site (e.g., L or C region) as 2 bolus infusions.

In one embodiment, the AAV particles comprise an rh10 capsid and are delivered to a subject by intrathecal administration using at least one site. As a non-limiting example, the dose of AAV particles may be 3×10¹³ vg administered at 3 sites at a volume/rate of 3 ml/3 hours. As another non-limiting example, the dose of AAV particles may be 1×10¹³ vg or 3×10¹³ vg administered at one site in the L (e.g., L1) or C region at a volume/rate of 10 ml/10 hours. As another non-limiting example, the dose of AAV particles may be 3×10¹³ vg administered at 3 sites as a bolus infusion. As another non-limiting example, the dose of AAV particles may be 1×10¹³ vg administered at 1 site as a bolus infusion of 1 ml. As another non-limiting example, the dose of AAV particles may be 3×10¹³ vg administered at 1 site (e.g., L or C region) as a bolus infusion of 3 ml. As another non-limiting example, the dose of AAV particles may be 2×10¹³ vg, 2×10¹² vg, 2×10¹¹ vg, or 2×10¹⁰ vg administered at 1 site (e.g., L or C region) as 2 bolus infusions.

In one embodiment, the AAV particles are ssAAV particles and comprise an rh10 capsid and are delivered to a subject by intrathecal administration using at least one site. As a non-limiting example, the dose of ssAAV particles may be 3×10¹³ vg administered at 3 sites at a volume/rate of 3 ml/3 hours. As another non-limiting example, the dose of ssAAV particles may be 1×10¹³ vg or 3×10¹³ vg administered at one site in the L (e.g., L1) or C region at a volume/rate of 10 ml/10 hours. As another non-limiting example, the dose of ssAAV particles may be 3×10¹³ vg administered at 3 sites as a bolus infusion. As another non-limiting example, the dose of ssAAV particles may be 1×10¹³ vg administered at 1 site as a bolus infusion of 1 ml. As another non-limiting example, the dose of ssAAV particles may be 3×10¹³ vg administered at 1 site (e.g., L or C region) as a bolus infusion of 3 ml. As another non-limiting example, the dose of ssAAV particles may be 2×10¹³ vg, 2×10¹² vg, 2×10¹¹ vg, or 2×10¹⁰ vg administered at 1 site (e.g., L or C region) as 2 bolus infusions.

In one embodiment, the AAV particles are scAAV particles and comprise an rh10 capsid and are delivered to a subject by intrathecal administration using at least one site. As a non-limiting example, the dose of scAAV particles may be 3×10¹³ vg administered at 3 sites at a volume/rate of 3 ml/3 hours. As another non-limiting example, the dose of scAAV particles may be 1×10¹³ vg or 3×10¹³ vg administered at one site in the L (e.g., L1) or C region at a volume/rate of 10 ml/10 hours. As another non-limiting example, the dose of scAAV particles may be 3×10¹³ vg administered at 3 sites as a bolus infusion. As another non-limiting example, the dose of scAAV particles may be 1×10¹³ vg administered at 1 site as a bolus infusion of 1 ml. As another non-limiting example, the dose of scAAV particles may be 3×10¹³ vg administered at 1 site (e.g., L or C region) as a bolus infusion of 3 ml. As another non-limiting example, the dose of scAAV particles may be 2×10¹³ vg, 2×10¹² vg, 2×10¹¹ vg, or 2×10¹⁰ vg administered at 1 site (e.g., L or C region) as 2 bolus infusions.

In one embodiment, the AAV particles comprise an AAV1 capsid and are delivered to a subject by intrathecal administration using at least one site. As a non-limiting example, the dose of AAV particles may be 3×10¹³ vg administered at 3 sites at a volume/rate of 3 ml/3 hours. As another non-limiting example, the dose of AAV particles may be 1×10¹³ vg or 3×10¹³ vg administered at one site in the L (e.g., L1) or C region at a volume/rate of 10 ml/10 hours. As another non-limiting example, the dose of AAV particles may be 3×10¹³ vg administered at 3 sites as a bolus infusion. As another non-limiting example, the dose of AAV particles may be 1×10¹³ vg administered at 1 site as a bolus infusion of 1 ml. As another non-limiting example, the dose of AAV particles may be 3×10¹³ vg administered at 1 site (e.g., L or C region) as a bolus infusion of 3 ml. As another non-limiting example, the dose of AAV particles may be 2×10¹³ vg, 2×10¹² vg, 2×10¹¹ vg, or 2×10¹⁰ vg administered at 1 site (e.g., L or C region) as 2 bolus infusions.

In one embodiment, the AAV particles are ssAAV particles and comprise an AAV1 capsid and are delivered to a subject by intrathecal administration using at least one site. As a non-limiting example, the dose of ssAAV particles may be 3×10¹³ vg administered at 3 sites at a volume/rate of 3 ml/3 hours. As another non-limiting example, the dose of ssAAV particles may be 1×10¹³ vg or 3×10¹³ vg administered at one site in the L (e.g., L1) or C region at a volume/rate of 10 ml/10 hours. As another non-limiting example, the dose of ssAAV particles may be 3×10¹³ vg administered at 3 sites as a bolus infusion. As another non-limiting example, the dose of ssAAV particles may be 1×10¹³ vg administered at 1 site as a bolus infusion of 1 ml. As another non-limiting example, the dose of ssAAV particles may be 3×10¹³ vg administered at 1 site (e.g., L or C region) as a bolus infusion of 3 ml. As another non-limiting example, the dose of ssAAV particles may be 2×10¹³ vg, 2×10¹² vg, 2×10¹¹ vg, or 2×10¹⁰ vg administered at 1 site (e.g., L or C region) as 2 bolus infusions.

In one embodiment, the AAV particles are scAAV particles and comprise an AAV1 capsid and are delivered to a subject by intrathecal administration using at least one site. As a non-limiting example, the dose of scAAV particles may be 3×10¹³ vg administered at 3 sites at a volume/rate of 3 ml/3 hours. As another non-limiting example, the dose of scAAV particles may be 1×10¹³ vg or 3×10¹³ vg administered at one site in the L (e.g., L1) or C region at a volume/rate of 10 ml/10 hours. As another non-limiting example, the dose of scAAV particles may be 3×10¹³ vg administered at 3 sites as a bolus infusion. As another non-limiting example, the dose of scAAV particles may be 1×10¹³ vg administered at 1 site as a bolus infusion of 1 ml. As another non-limiting example, the dose of scAAV particles may be 3×10¹³ vg administered at 1 site (e.g., L or C region) as a bolus infusion of 3 ml. As another non-limiting example, the dose of scAAV particles may be 2×10¹³ vg, 2×10¹² vg, 2×10¹¹ vg, or 2×10¹⁰ vg administered at 1 site (e.g., L or C region) as 2 bolus infusions.

In one embodiment, the AAV particles comprise an AAV2 capsid and are delivered to a subject by intrathecal administration using at least one site. As a non-limiting example, the dose of AAV particles may be 3×10¹³ vg administered at 3 sites at a volume/rate of 3 ml/3 hours. As another non-limiting example, the dose of AAV particles may be 1×10¹³ vg or 3×10¹³ vg administered at one site in the L (e.g., L1) or C region at a volume/rate of 10 ml/10 hours. As another non-limiting example, the dose of AAV particles may be 3×10¹³ vg administered at 3 sites as a bolus infusion. As another non-limiting example, the dose of AAV particles may be 1×10¹³ vg administered at 1 site as a bolus infusion of 1 ml. As another non-limiting example, the dose of AAV particles may be 3×10¹³ vg administered at 1 site (e.g., L or C region) as a bolus infusion of 3 ml. As another non-limiting example, the dose of AAV particles may be 2×10¹³ vg, 2×10¹² vg, 2×10¹¹ vg, or 2×10¹⁰ vg administered at 1 site (e.g., L or C region) as 2 bolus infusions.

In one embodiment, the AAV particles are ssAAV particles and comprise an AAV2 capsid and are delivered to a subject by intrathecal administration using at least one site. As a non-limiting example, the dose of ssAAV particles may be 3×10¹³ vg administered at 3 sites at a volume/rate of 3 ml/3 hours. As another non-limiting example, the dose of ssAAV particles may be 1×10¹³ vg or 3×10¹³ vg administered at one site in the L (e.g., L1) or C region at a volume/rate of 10 ml/10 hours. As another non-limiting example, the dose of ssAAV particles may be 3×10¹³ vg administered at 3 sites as a bolus infusion. As another non-limiting example, the dose of ssAAV particles may be 1×10¹³ vg administered at 1 site as a bolus infusion of 1 ml. As another non-limiting example, the dose of ssAAV particles may be 3×10¹³ vg administered at 1 site (e.g., L or C region) as a bolus infusion of 3 ml. As another non-limiting example, the dose of ssAAV particles may be 2×10¹³ vg, 2×10¹² vg, 2×10¹¹ vg, or 2×10¹⁰ vg administered at 1 site (e.g., L or C region) as 2 bolus infusions.

In one embodiment, the AAV particles are scAAV particles and comprise an AAV2 capsid and are delivered to a subject by intrathecal administration using at least one site. As a non-limiting example, the dose of scAAV particles may be 3×10¹³ vg administered at 3 sites at a volume/rate of 3 ml/3 hours. As another non-limiting example, the dose of scAAV particles may be 1×10¹³ vg or 3×10¹³ vg administered at one site in the L (e.g., L1) or C region at a volume/rate of 10 ml/10 hours. As another non-limiting example, the dose of scAAV particles may be 3×10¹³ vg administered at 3 sites as a bolus infusion. As another non-limiting example, the dose of scAAV particles may be 1×10¹³ vg administered at 1 site as a bolus infusion of 1 ml. As another non-limiting example, the dose of scAAV particles may be 3×10¹³ vg administered at 1 site (e.g., L or C region) as a bolus infusion of 3 ml. As another non-limiting example, the dose of scAAV particles may be 2×10¹³ vg, 2×10¹² vg, 2×10¹¹ vg, or 2×10¹⁰ vg administered at 1 site (e.g., L or C region) as 2 bolus infusions.

In one embodiment, the AAV particles comprise an AAV5 capsid and are delivered to a subject by intrathecal administration using at least one site. As a non-limiting example, the dose of AAV particles may be 3×10¹³ vg administered at 3 sites at a volume/rate of 3 ml/3 hours. As another non-limiting example, the dose of AAV particles may be 1×10¹³ vg or 3×10¹³ vg administered at one site in the L (e.g., L1) or C region at a volume/rate of 10 ml/10 hours. As another non-limiting example, the dose of AAV particles may be 3×10¹³ vg administered at 3 sites as a bolus infusion. As another non-limiting example, the dose of AAV particles may be 1×10¹³ vg administered at 1 site as a bolus infusion of 1 ml. As another non-limiting example, the dose of AAV particles may be 3×10¹³ vg administered at 1 site (e.g., L or C region) as a bolus infusion of 3 ml. As another non-limiting example, the dose of AAV particles may be 2×10¹³ vg, 2×10¹² vg, 2×10¹¹ vg, or 2×10¹⁰ vg administered at 1 site (e.g., L or C region) as 2 bolus infusions.

In one embodiment, the AAV particles are ssAAV particles and comprise an AAV5 capsid and are delivered to a subject by intrathecal administration using at least one site. As a non-limiting example, the dose of ssAAV particles may be 3×10¹³ vg administered at 3 sites at a volume/rate of 3 ml/3 hours. As another non-limiting example, the dose of ssAAV particles may be 1×10¹³ vg or 3×10¹³ vg administered at one site in the L (e.g., L1) or C region at a volume/rate of 10 ml/10 hours. As another non-limiting example, the dose of ssAAV particles may be 3×10¹³ vg administered at 3 sites as a bolus infusion. As another non-limiting example, the dose of ssAAV particles may be 1×10¹³ vg administered at 1 site as a bolus infusion of 1 ml. As another non-limiting example, the dose of ssAAV particles may be 3×10¹³ vg administered at 1 site (e.g., L or C region) as a bolus infusion of 3 ml. As another non-limiting example, the dose of ssAAV particles may be 2×10¹³ vg, 2×10¹² vg, 2×10¹¹ vg, or 2×10¹⁰ vg administered at 1 site (e.g., L or C region) as 2 bolus infusions.

In one embodiment, the AAV particles are scAAV particles and comprise an AAV5 capsid and are delivered to a subject by intrathecal administration using at least one site. As a non-limiting example, the dose of scAAV particles may be 3×10¹³ vg administered at 3 sites at a volume/rate of 3 ml/3 hours. As another non-limiting example, the dose of scAAV particles may be 1×10¹³ vg or 3×10¹³ vg administered at one site in the L (e.g., L1) or C region at a volume/rate of 10 ml/10 hours. As another non-limiting example, the dose of scAAV particles may be 3×10¹³ vg administered at 3 sites as a bolus infusion. As another non-limiting example, the dose of scAAV particles may be 1×10¹³ vg administered at 1 site as a bolus infusion of 1 ml. As another non-limiting example, the dose of scAAV particles may be 3×10¹³ vg administered at 1 site (e.g., L or C region) as a bolus infusion of 3 ml. As another non-limiting example, the dose of scAAV particles may be 2×10¹³ vg, 2×10¹² vg, 2×10¹¹ vg, or 2×10¹⁰ vg administered at 1 site (e.g., L or C region) as 2 bolus infusions.

In one embodiment, the AAV particles comprise an AAV6 capsid and are delivered to a subject by intrathecal administration using at least one site. As a non-limiting example, the dose of AAV particles may be 3×10¹³ vg administered at 3 sites at a volume/rate of 3 ml/3 hours. As another non-limiting example, the dose of AAV particles may be 1×10¹³ vg or 3×10¹³ vg administered at one site in the L (e.g., L1) or C region at a volume/rate of 10 ml/10 hours. As another non-limiting example, the dose of AAV particles may be 3×10¹³ vg administered at 3 sites as a bolus infusion. As another non-limiting example, the dose of AAV particles may be 1×10¹³ vg administered at 1 site as a bolus infusion of 1 ml. As another non-limiting example, the dose of AAV particles may be 3×10¹³ vg administered at 1 site (e.g., L or C region) as a bolus infusion of 3 ml. As another non-limiting example, the dose of AAV particles may be 2×10¹³ vg, 2×10¹² vg, 2×10¹¹ vg, or 2×10¹⁰ vg administered at 1 site (e.g., L or C region) as 2 bolus infusions.

In one embodiment, the AAV particles are ssAAV particles and comprise an AAV6 capsid and are delivered to a subject by intrathecal administration using at least one site. As a non-limiting example, the dose of ssAAV particles may be 3×10¹³ vg administered at 3 sites at a volume/rate of 3 ml/3 hours. As another non-limiting example, the dose of ssAAV particles may be 1×10¹³ vg or 3×10¹³ vg administered at one site in the L (e.g., L1) or C region at a volume/rate of 10 ml/10 hours. As another non-limiting example, the dose of ssAAV particles may be 3×10¹³ vg administered at 3 sites as a bolus infusion. As another non-limiting example, the dose of ssAAV particles may be 1×10¹³ vg administered at 1 site as a bolus infusion of 1 ml. As another non-limiting example, the dose of ssAAV particles may be 3×10¹³ vg administered at 1 site (e.g., L or C region) as a bolus infusion of 3 ml. As another non-limiting example, the dose of ssAAV particles may be 2×10¹³ vg, 2×10¹² vg, 2×10¹¹ vg, or 2×10¹⁰ vg administered at 1 site (e.g., L or C region) as 2 bolus infusions.

In one embodiment, the AAV particles are scAAV particles and comprise an AAV6 capsid and are delivered to a subject by intrathecal administration using at least one site. As a non-limiting example, the dose of scAAV particles may be 3×10¹³ vg administered at 3 sites at a volume/rate of 3 ml/3 hours. As another non-limiting example, the dose of scAAV particles may be 1×10¹³ vg or 3×10¹³ vg administered at one site in the L (e.g., L1) or C region at a volume/rate of 10 ml/10 hours. As another non-limiting example, the dose of scAAV particles may be 3×10¹³ vg administered at 3 sites as a bolus infusion. As another non-limiting example, the dose of scAAV particles may be 1×10¹³ vg administered at 1 site as a bolus infusion of 1 ml. As another non-limiting example, the dose of scAAV particles may be 3×10¹³ vg administered at 1 site (e.g., L or C region) as a bolus infusion of 3 ml. As another non-limiting example, the dose of scAAV particles may be 2×10¹³ vg, 2×10¹² vg, 2×10¹¹ vg, or 2×10¹⁰ vg administered at 1 site (e.g., L or C region) as 2 bolus infusions.

In one embodiment, the AAV particles comprise an AAV9 capsid and are delivered to a subject by intrathecal administration using at least one site. As a non-limiting example, the dose of AAV particles may be 3×10¹³ vg administered at 3 sites at a volume/rate of 3 ml/3 hours. As another non-limiting example, the dose of AAV particles may be 1×10¹³ vg or 3×10¹³ vg administered at one site in the L (e.g., L1) or C region at a volume/rate of 10 ml/10 hours. As another non-limiting example, the dose of AAV particles may be 3×10¹³ vg administered at 3 sites as a bolus infusion. As another non-limiting example, the dose of AAV particles may be 1×10¹³ vg administered at 1 site as a bolus infusion of 1 ml. As another non-limiting example, the dose of AAV particles may be 3×10¹³ vg administered at 1 site (e.g., L or C region) as a bolus infusion of 3 ml. As another non-limiting example, the dose of AAV particles may be 2×10¹³ vg, 2×10¹² vg, 2×10¹¹ vg, or 2×10¹⁰ vg administered at 1 site (e.g., L or C region) as 2 bolus infusions.

In one embodiment, the AAV particles are ssAAV particles and comprise an AAV9 capsid and are delivered to a subject by intrathecal administration using at least one site. As a non-limiting example, the dose of ssAAV particles may be 3×10¹³ vg administered at 3 sites at a volume/rate of 3 ml/3 hours. As another non-limiting example, the dose of ssAAV particles may be 1×10¹³ vg or 3×10¹³ vg administered at one site in the L (e.g., L1) or C region at a volume/rate of 10 ml/10 hours. As another non-limiting example, the dose of ssAAV particles may be 3×10¹³ vg administered at 3 sites as a bolus infusion. As another non-limiting example, the dose of ssAAV particles may be 1×10¹³ vg administered at 1 site as a bolus infusion of 1 ml. As another non-limiting example, the dose of ssAAV particles may be 3×10¹³ vg administered at 1 site (e.g., L or C region) as a bolus infusion of 3 ml. As another non-limiting example, the dose of ssAAV particles may be 2×10¹³ vg, 2×10¹² vg, 2×10¹⁰ vg, or 2×10¹⁰ vg administered at 1 site (e.g., L or C region) as 2 bolus infusions.

In one embodiment, the AAV particles are scAAV particles and comprise an AAV9 capsid and are delivered to a subject by intrathecal administration using at least one site. As a non-limiting example, the dose of scAAV particles may be 3×10¹³ vg administered at 3 sites at a volume/rate of 3 ml/3 hours. As another non-limiting example, the dose of scAAV particles may be 1×10¹³ vg or 3×10¹³ vg administered at one site in the L (e.g., L1) or C region at a volume/rate of 10 ml/10 hours. As another non-limiting example, the dose of scAAV particles may be 3×10¹³ vg administered at 3 sites as a bolus infusion. As another non-limiting example, the dose of scAAV particles may be 1×10¹³ vg administered at 1 site as a bolus infusion of 1 ml. As another non-limiting example, the dose of scAAV particles may be 3×10¹³ vg administered at 1 site (e.g., L or C region) as a bolus infusion of 3 ml. As another non-limiting example, the dose of scAAV particles may be 2×10¹³ vg, 2×10¹² vg, 2×10¹¹ vg, or 2×10¹⁰ vg administered at 1 site (e.g., L or C region) as 2 bolus infusions.

In one embodiment, the AAV particles comprise an AAV-DJ capsid and are delivered to a subject by intrathecal administration using at least one site. As a non-limiting example, the dose of AAV particles may be 3×10¹³ vg administered at 3 sites at a volume/rate of 3 ml/3 hours. As another non-limiting example, the dose of AAV particles may be 1×10¹³ vg or 3×10¹³ vg administered at one site in the L (e.g., L1) or C region at a volume/rate of 10 ml/10 hours. As another non-limiting example, the dose of AAV particles may be 3×10¹³ vg administered at 3 sites as a bolus infusion. As another non-limiting example, the dose of AAV particles may be 1×10¹³ vg administered at 1 site as a bolus infusion of 1 ml. As another non-limiting example, the dose of AAV particles may be 3×10¹³ vg administered at 1 site (e.g., L or C region) as a bolus infusion of 3 ml. As another non-limiting example, the dose of AAV particles may be 2×10¹³ vg, 2×10¹² vg, 2×10¹¹ vg, or 2×10¹⁰ vg administered at 1 site (e.g., L or C region) as 2 bolus infusions.

In one embodiment, the AAV particles are ssAAV particles and comprise an AAV-DJ capsid and are delivered to a subject by intrathecal administration using at least one site. As a non-limiting example, the dose of ssAAV particles may be 3×10¹³ vg administered at 3 sites at a volume/rate of 3 ml/3 hours. As another non-limiting example, the dose of ssAAV particles may be 1×10¹³ vg or 3×10¹³ vg administered at one site in the L (e.g., L1) or C region at a volume/rate of 10 ml/10 hours. As another non-limiting example, the dose of ssAAV particles may be 3×10¹³ vg administered at 3 sites as a bolus infusion. As another non-limiting example, the dose of ssAAV particles may be 1×10¹³ vg administered at 1 site as a bolus infusion of 1 ml. As another non-limiting example, the dose of ssAAV particles may be 3×10¹³ vg administered at 1 site (e.g., L or C region) as a bolus infusion of 3 ml. As another non-limiting example, the dose of ssAAV particles may be 2×10¹³ vg, 2×10¹² vg, 2×10¹¹ vg, or 2×10¹⁰ vg administered at 1 site (e.g., L or C region) as 2 bolus infusions.

In one embodiment, the AAV particles are scAAV particles and comprise an AAV-DJ capsid and are delivered to a subject by intrathecal administration using at least one site. As a non-limiting example, the dose of scAAV particles may be 3×10¹³ vg administered at 3 sites at a volume/rate of 3 ml/3 hours. As another non-limiting example, the dose of scAAV particles may be 1×10¹³ vg or 3×10¹³ vg administered at one site in the L (e.g., L1) or C region at a volume/rate of 10 ml/10 hours. As another non-limiting example, the dose of scAAV particles may be 3×10¹³ vg administered at 3 sites as a bolus infusion. As another non-limiting example, the dose of scAAV particles may be 1×10¹³ vg administered at 1 site as a bolus infusion of 1 ml. As another non-limiting example, the dose of scAAV particles may be 3×10¹³ vg administered at 1 site (e.g., L or C region) as a bolus infusion of 3 ml. As another non-limiting example, the dose of scAAV particles may be 2×10¹³ vg, 2×10¹² vg, 2×10¹¹ vg, or 2×10¹⁰ vg administered at 1 site (e.g., L or C region) as 2 bolus infusions.

In one embodiment, the AAV particles comprise an AAV-DJ8 capsid and are delivered to a subject by intrathecal administration using at least one site. As a non-limiting example, the dose of AAV particles may be 3×10¹³ vg administered at 3 sites at a volume/rate of 3 ml/3 hours. As another non-limiting example, the dose of AAV particles may be 1×10¹³ vg or 3×10¹³ vg administered at one site in the L (e.g., L1) or C region at a volume/rate of 10 ml/10 hours. As another non-limiting example, the dose of AAV particles may be 3×10¹³ vg administered at 3 sites as a bolus infusion. As another non-limiting example, the dose of AAV particles may be 1×10¹³ vg administered at 1 site as a bolus infusion of 1 ml. As another non-limiting example, the dose of AAV particles may be 3×10¹³ vg administered at 1 site (e.g., L or C region) as a bolus infusion of 3 ml. As another non-limiting example, the dose of AAV particles may be 2×10¹³ vg, 2×10¹² vg, 2×10¹¹ vg, or 2×10¹⁰ vg administered at 1 site (e.g., L or C region) as 2 bolus infusions.

In one embodiment, the AAV particles are ssAAV particles and comprise an AAV-DJ8 capsid and are delivered to a subject by intrathecal administration using at least one site. As a non-limiting example, the dose of ssAAV particles may be 3×10¹³ vg administered at 3 sites at a volume/rate of 3 ml/3 hours. As another non-limiting example, the dose of ssAAV particles may be 1×10¹³ vg or 3×10¹³ vg administered at one site in the L (e.g., L1) or C region at a volume/rate of 10 ml/10 hours. As another non-limiting example, the dose of ssAAV particles may be 3×10¹³ vg administered at 3 sites as a bolus infusion. As another non-limiting example, the dose of ssAAV particles may be 1×10¹³ vg administered at 1 site as a bolus infusion of 1 ml. As another non-limiting example, the dose of ssAAV particles may be 3×10¹³ vg administered at 1 site (e.g., L or C region) as a bolus infusion of 3 ml. As another non-limiting example, the dose of ssAAV particles may be 2×10¹³ vg, 2×10¹² vg, 2×10¹¹ vg, or 2×10¹⁰ vg administered at 1 site (e.g., L or C region) as 2 bolus infusions.

In one embodiment, the AAV particles are scAAV particles and comprise an AAV-DJ8 capsid and are delivered to a subject by intrathecal administration using at least one site. As a non-limiting example, the dose of scAAV particles may be 3×10¹³ vg administered at 3 sites at a volume/rate of 3 ml/3 hours. As another non-limiting example, the dose of scAAV particles may be 1×10¹³ vg or 3×10¹³ vg administered at one site in the L (e.g., L1) or C region at a volume/rate of 10 ml/10 hours. As another non-limiting example, the dose of scAAV particles may be 3×10¹³ vg administered at 3 sites as a bolus infusion. As another non-limiting example, the dose of scAAV particles may be 1×10¹³ vg administered at 1 site as a bolus infusion of 1 ml. As another non-limiting example, the dose of scAAV particles may be 3×10¹³ vg administered at 1 site (e.g., L or C region) as a bolus infusion of 3 ml. As another non-limiting example, the dose of scAAV particles may be 2×10¹³ vg, 2×10¹² vg, 2×10¹⁰ vg, or 2×10¹⁰ vg administered at 1 site (e.g., L or C region) as 2 bolus infusions.

In one embodiment, the AAV particles are scAAV particles and are delivered by intrathecal administration to a subject using at least one site. As a non-limiting example, the dose of scAAV particles may be 3.4×10¹¹ vg administered bilaterally to the caudate and putamen at a dose a volume of 30 ul/site. As a non-limiting example, the dose of scAAV particles may be 3.4×10¹¹ vg administered bilaterally to the left caudate and right putamen at a dose a volume of 30 ul/site. As a non-limiting example, the dose of scAAV particles may be 3.4×10¹¹ vg administered bilaterally to the right caudate and left putamen at a dose a volume of 30 ul/site. As a non-limiting example, the dose of scAAV particles may be 3.4×10¹¹ vg administered bilaterally to the left caudate and left putamen at a dose a volume of 30 ul/site. As a non-limiting example, the dose of scAAV particles may be 3.4×10¹¹ vg administered bilaterally to the right caudate and right putamen at a dose a volume of 30 ul/site. As a non-limiting example, the dose of scAAV particles may be 1.1×10¹¹ vg administered bilaterally to the caudate and putamen at a dose a volume of 30 ul/site. As a non-limiting example, the dose of scAAV particles may be 1.1×10¹¹ vg administered bilaterally to the left caudate and right putamen at a dose a volume of 30 ul/site. As a non-limiting example, the dose of scAAV particles may be 1.1×10¹¹ vg administered bilaterally to the right caudate and left putamen at a dose a volume of 30 ul/site. As a non-limiting example, the dose of scAAV particles may be 1.1×10¹¹ vg administered bilaterally to the left caudate and left putamen at a dose a volume of 30 ul/site. As a non-limiting example, the dose of scAAV particles may be 1.1×10¹¹ vg administered bilaterally to the right caudate and right putamen at a dose a volume of 30 ul/site. As a non-limiting example, the dose of scAAV particles may be 5.7×10¹⁰ vg administered to either the left or right caudate at a dose a volume of 30 ul/site. As a non-limiting example, the dose of scAAV particles may be 5.7×10¹⁰ vg administered to both the left and right caudate at a dose a volume of 30 ul/site. As a non-limiting example, the dose of scAAV particles may be 5.7×10¹⁰ vg administered to either the left or right putamen at a dose a volume of 30 ul/site. As a non-limiting example, the dose of scAAV particles may be 5.7×10¹⁰ vg administered to both the left and right putamen at a dose a volume of 30 ul/site.

In one embodiment, the AAV particles are ssAAV particles and are delivered by intrathecal administration to a subject using at least one site. As a non-limiting example, the dose of ssAAV particles may be 3.4×10¹¹ vg administered bilaterally to the caudate and putamen at a dose a volume of 30 ul/site. As a non-limiting example, the dose of ssAAV particles may be 3.4×10¹¹ vg administered bilaterally to the left caudate and right putamen at a dose a volume of 30 ul/site. As a non-limiting example, the dose of ssAAV particles may be 3.4×10¹¹ vg administered bilaterally to the right caudate and left putamen at a dose a volume of 30 ul/site. As a non-limiting example, the dose of ssAAV particles may be 3.4×10¹¹ vg administered bilaterally to the left caudate and left putamen at a dose a volume of 30 ul/site. As a non-limiting example, the dose of ssAAV particles may be 3.4×10¹¹ vg administered bilaterally to the right caudate and right putamen at a dose a volume of 30 ul/site. As a non-limiting example, the dose of ssAAV particles may be 1.1×10¹¹ vg administered bilaterally to the caudate and putamen at a dose a volume of 30 ul/site. As a non-limiting example, the dose of ssAAV particles may be 1.1×10¹¹ vg administered bilaterally to the left caudate and right putamen at a dose a volume of 30 ul/site. As a non-limiting example, the dose of ssAAV particles may be 1.1×10¹¹ vg administered bilaterally to the right caudate and left putamen at a dose a volume of 30 ul/site. As a non-limiting example, the dose of ssAAV particles may be 1.1×10¹¹ vg administered bilaterally to the left caudate and left putamen at a dose a volume of 30 ul/site. As a non-limiting example, the dose of ssAAV particles may be 1.1×10¹¹ vg administered bilaterally to the right caudate and right putamen at a dose a volume of 30 ul/site. As a non-limiting example, the dose of ssAAV particles may be 5.7×10¹⁰ vg administered to either the left or right caudate at a dose a volume of 30 ul/site. As a non-limiting example, the dose of ssAAV particles may be 5.7×10¹⁰ vg administered to both the left and right caudate at a dose a volume of 30 ul/site. As a non-limiting example, the dose of ssAAV particles may be 5.7×10¹⁰ vg administered to either the left or right putamen at a dose a volume of 30 ul/site. As a non-limiting example, the dose of ssAAV particles may be 5.7×10¹⁰ vg administered to both the left and right putamen at a dose a volume of 30 ul/site.

In one embodiment, the AAV particles comprise an AAV1 capsid are scAAV particles and are delivered by intrathecal administration to a subject using at least one site. As a non-limiting example, the dose of scAAV particles may be 3.4×10¹¹ vg administered bilaterally to the caudate and putamen at a dose a volume of 30 ul/site. As a non-limiting example, the dose of scAAV particles may be 3.4×10¹¹ vg administered bilaterally to the left caudate and right putamen at a dose a volume of 30 ul/site. As a non-limiting example, the dose of scAAV particles may be 3.4×10¹¹ vg administered bilaterally to the right caudate and left putamen at a dose a volume of 30 ul/site. As a non-limiting example, the dose of scAAV particles may be 3.4×10¹¹ vg administered bilaterally to the left caudate and left putamen at a dose a volume of 30 ul/site. As a non-limiting example, the dose of scAAV particles may be 3.4×10¹¹ vg administered bilaterally to the right caudate and right putamen at a dose a volume of 30 ul/site. As a non-limiting example, the dose of scAAV particles may be 1.1×10¹¹ vg administered bilaterally to the caudate and putamen at a dose a volume of 30 ul/site. As a non-limiting example, the dose of scAAV particles may be 1.1×10¹¹ vg administered bilaterally to the left caudate and right putamen at a dose a volume of 30 ul/site. As a non-limiting example, the dose of scAAV particles may be 1.1×10¹¹ vg administered bilaterally to the right caudate and left putamen at a dose a volume of 30 ul/site. As a non-limiting example, the dose of scAAV particles may be 1.1×10¹¹ vg administered bilaterally to the left caudate and left putamen at a dose a volume of 30 ul/site. As a non-limiting example, the dose of scAAV particles may be 1.1×10¹¹ vg administered bilaterally to the right caudate and right putamen at a dose a volume of 30 ul/site. As a non-limiting example, the dose of scAAV particles may be 5.7×10¹⁰ vg administered to either the left or right caudate at a dose a volume of 30 ul/site. As a non-limiting example, the dose of scAAV particles may be 5.7×10¹⁰ vg administered to both the left and right caudate at a dose a volume of 30 ul/site. As a non-limiting example, the dose of scAAV particles may be 5.7×10¹⁰ vg administered to either the left or right putamen at a dose a volume of 30 ul/site. As a non-limiting example, the dose of scAAV particles may be 5.7×10¹⁰ vg administered to both the left and right putamen at a dose a volume of 30 ul/site.

In one embodiment, the AAV particles comprise an AAV1 capsid are ssAAV particles and are delivered by intrathecal administration to a subject using at least one site. As a non-limiting example, the dose of ssAAV particles may be 3.4×10¹¹ vg administered bilaterally to the caudate and putamen at a dose a volume of 30 ul/site. As a non-limiting example, the dose of ssAAV particles may be 3.4×10¹¹ vg administered bilaterally to the left caudate and right putamen at a dose a volume of 30 ul/site. As a non-limiting example, the dose of ssAAV particles may be 3.4×10¹¹ vg administered bilaterally to the right caudate and left putamen at a dose a volume of 30 ul/site. As a non-limiting example, the dose of ssAAV particles may be 3.4×10¹¹ vg administered bilaterally to the left caudate and left putamen at a dose a volume of 30 ul/site. As a non-limiting example, the dose of ssAAV particles may be 3.4×10¹¹ vg administered bilaterally to the right caudate and right putamen at a dose a volume of 30 ul/site. As a non-limiting example, the dose of ssAAV particles may be 1.1×10¹¹ vg administered bilaterally to the caudate and putamen at a dose a volume of 30 ul/site. As a non-limiting example, the dose of ssAAV particles may be 1.1×10¹¹ vg administered bilaterally to the left caudate and right putamen at a dose a volume of 30 ul/site. As a non-limiting example, the dose of ssAAV particles may be 1.1×10¹¹ vg administered bilaterally to the right caudate and left putamen at a dose a volume of 30 ul/site. As a non-limiting example, the dose of ssAAV particles may be 1.1×10¹¹ vg administered bilaterally to the left caudate and left putamen at a dose a volume of 30 ul/site. As a non-limiting example, the dose of ssAAV particles may be 1.1×10¹¹ vg administered bilaterally to the right caudate and right putamen at a dose a volume of 30 ul/site. As a non-limiting example, the dose of ssAAV particles may be 5.7×10¹⁰ vg administered to either the left or right caudate at a dose a volume of 30 ul/site. As a non-limiting example, the dose of ssAAV particles may be 5.7×10¹⁰ vg administered to both the left and right caudate at a dose a volume of 30 ul/site. As a non-limiting example, the dose of ssAAV particles may be 5.7×10¹⁰ vg administered to either the left or right putamen at a dose a volume of 30 ul/site. As a non-limiting example, the dose of ssAAV particles may be 5.7×10¹⁰ vg administered to both the left and right putamen at a dose a volume of 30 ul/site.

### Continuous/Prolonged Infusion

In one embodiment, delivery of AAV particles to cells of the central nervous system (e.g., parenchyma) is performed by prolonged intrathecal (IT) infusion (also referred to herein as continuous IT infusion). It has been discovered that intrathecal (IT) administration by prolonged IT infusion (also referred to as IT continuous infusion) leads to stable AAV particle levels within the cerebral spinal fluid (CSF) circulating around the brain and spinal cord by maintaining favorable concentration gradients for AAV particle movement into the parenchyma and increases the total area of spinal cord exposed to efficacious AAV particle concentrations. Consequently, prolonged exposure to the spinal cord will allow for a single site of delivery for widespread neuraxial transfection. Prolonged IT infusion provides increased exposure that favors tissue interactions with AAV by extracellular and intraneuronal processes. As a non-limiting example, the even distribution across targeted neuraxis may avoid hot spots of transduction.

In one embodiment, IT prolonged infusion comprises delivery by injection into the subarachnoid space, between the arachnoid membrane and pia mater, which comprises the channels through which CSF circulates. IT prolonged infusion comprises delivery to any area of the subarachnoid space including, but not limited to, perivascular space and the subarachnoid space along the entire length of the spinal cord and surrounding the brain. As a non-limiting example, the AAV particles may be used to treat Friedreich's Ataxia (FA), amyotrophic lateral sclerosis (ALS), spinal muscular atrophy (SMA) and/or neuropathic pain.

In one embodiment, AAV may move along the outside of neural axons including, but not limited to, nerves such as the dorsal and ventral roots that transect the IT space and are bathed by CSF. Intraneuronal exposure comprises uptake and transport within and along the interior of axons towards (retrograde) or away from (anterograde) the neuronal cell body; AAV has been shown to move in both directions dependent on the serotype.

In one embodiment extracellular ‘paravascular capture’ comprises the inward movement of AAV along blood vessels.

In some embodiments IT prolonged infusion comprises delivery to the cervical, thoracic, and or lumbar regions of the spine. As used herein, IT prolonged infusion into the spine is defined by the vertebral level at the site of prolonged infusion. In some embodiments IT prolonged infusion comprises delivery to the cervical region of the spine at any location including, but not limited to C1, C2, C3, C4, C5, C6, C7, and/or C8. In some embodiments IT prolonged infusion comprises delivery to the thoracic region of the spine at any location including, but not limited to T1, T2, T3, T3, T4, T5, T6, T7, T8, T9, T10, T11, and/or T12. In some embodiments IT prolonged infusion comprises delivery to the lumbar region of the spine at any location including, but not limited to L1, L2, L3, L3, L4, L5, and/or L6. In some embodiments IT prolonged infusion comprises delivery to the sacral region of the spine at any location including, but not limited to S1, S2, S3, S4, or S5. In some embodiments, delivery by IT prolonged infusion comprises one or more than one site of prolonged infusion.

In some embodiments, delivery by IT prolonged infusion may comprise 1, 2, 3, 4, 5, 6, 7, 8, 9, 10, 11, 12, 13, 14, 15, 16, 17, 18, 19, 20, 21, 22, 23, 24, or 25 sites of prolonged infusion. In one embodiment, delivery by IT prolonged infusion comprises at least three sites of prolonged infusion. In one embodiment, delivery by IT prolonged infusion consists of three sites of prolonged infusion. In one embodiment, delivery by IT prolonged infusion comprises three sites of prolonged infusion at C1, T1, and L1.

In one embodiment, delivery by prolonged IT infusion includes administration using a cervical catheter located at C5.

In one embodiment, delivery by prolonged IT infusion includes administration using a cervical catheter located at C1.

In one embodiment, delivery by prolonged IT infusion may be via a cervical catheter placed between C1 and C2.

In one embodiment, delivery by prolonged IT infusion may be via a thoracolumbar catheter placed between T10 and L1.

In one embodiment, the catheter for prolonged IT infusion may be placed in the cervical region such as, but not limited to, C1-C2.

In one embodiment, the catheter for prolonged IT infusion may be placed thoracolumbar such as, but not limited to, T10/L1.

In one embodiment, the catheter for intrathecal delivery may be located in the cervical region. The AAV particles may be delivered in a continuous infusion.

In one embodiment, the catheter for intrathecal delivery may be located in the lumbar region. The AAV particles may be delivered in a continuous infusion.

The large size of AAV particles, about 25 nm diameter, leads to steric hindrance wherein there is a limit to the number of AAV particles that may access tissue binding sites and achieve subsequent uptake into cells at any given point in time. Bolus delivery of high numbers of AAV particles over a short period of infusion makes it nearly impossible for much of the delivered AAV dose to reach binding sites for uptake into neurons, astrocytes, oligodendrocytes, microglia and other CNS cells. In contrast, prolonged continuous IT infusion may provide more even and complete distribution of AAV along the neuraxis as AAV concentration reaches equilibrium, thereby reducing the risk of steric hindrance due to the large size of AAV as well as providing a longer timeframe for uptake of AAV into neural cells, tissues, and structures.

In one embodiment, prolonged IT infusion allows for slower, more controlled infusion that yields more reproducible results as compared to bolus IT delivery which can lead to wastage of AAV drug product and ‘hot spots’ comprising uneven, high levels of transduction along the spinal cord or adjacent dorsal root ganglion.

In one embodiment, prolonged IT infusion of the AAV particles allows for AAV levels in the spinal cord to approach steady state, i.e., the maximum possible level of particles in the CSF for a given infusion rate and concentration. Steady state for AAV levels is reached when the amount of AAV infused into the CSF is equal to AAV clearance rate. The longer that AAV is delivered at or near steady state, the longer there is maintained a favorable diffusion gradient from CSF into parenchyma, which maximizes the opportunity for particles to be transported via extra- and intra-neuronal routes.

### Bolus Infusion

In one embodiment, the AAV particles may be delivered to a subject using bolus IT infusion.

In one embodiment, a subject may be delivered the AAV particles herein by bolus IT infusion at more than one site such as, but not limited to, 2, 3, 4, 5, 6, 7, 8 or more than 8 sites.

In one embodiment, a subject may be delivered the AAV particles described herein by intrathecal delivery in the lumbar region via a 10 hour bolus injection.

In one embodiment, the catheter for intrathecal delivery may be located in the cervical region via a bolus infusion.

In one embodiment, the catheter for intrathecal delivery may be located in the lumbar region via a bolus infusion.

### The Spinal Cord

The human spinal cord was first mapped by Bruce, 1901 (Bruce, A., 1901. A Topographical Atlas of the Spinal Cord. Williams and Norgate, London, Available from: www.archive.org/details/cu31924024791406 [24.07.14]) and later by others including Sengul et al., 2013 (Sengul, G., Watson, C., Tanaka, I., Paxinos, G., 2013. Atlas of the Spinal Cord of the Rat, Mouse, Marmoset, Rhesus, and Human. Elsevier Academic Press, San Diego), the contents of each of which is herein incorporated by reference in its entirety. The spinal cord can be divided into 5 regions, into an organization which is derived from the adjacent vertebrae: cervical, thoracic, lumbar, sacral, and coccygeal (caudal) as described in Watson et al., 2015 (Neuroscience Research 93 (2015) 164-175 The spinal cord of the common marmoset (Callithrix jacchus) Charles Watson, Gulgun Senguld, Ikuko Tanakae, Zoltan Rusznakb, and Hironobu Tokunoe), and Pardo et al., 2012 (Toxicologic Pathology, 40: 624-636, 2012 “Technical Guide for Nervous System Sampling of the Cynomolgus Monkey for General Toxicity Studies” Ingrid D. Pardo, Robert H. Garman, Klaus Weber, Walter F. Bobrowski, Jerry F. Hardisty, And Daniel Morton), the contents of each of which is herein incorporated by reference in its entirety. The segments in each region and their numbering are shown in Table 2.

In some instances, rhesus and cynomolgus monkey each have the same number of segments in each region. Rhesus monkey and Cynomolgus monkeys have 7 or 8 segments in the cervical region. Humans have 7 or 8 segments in the cervical region. Humans, Rhesus monkeys and Cynomolgus monkeys have 12 thoracic segments. Humans have 5 lumbar segments while Rhesus and Cynomolgus monkeys have 7 lumbar segments. The sacral region includes 5 segments in humans, but three segments in Cynomolgus monkey and Rhesus monkey. The coccygeal region has 3 segments in rhesus monkey and cynomolgus monkey, and one segment in human.

TABLE 2
Spinal cord segments in human, cynomolgus and rhesus monkeys

| Spinal Cord | | Cynomolgus | Rhesus |
|---|---|---|---|
| Region | Human | Monkey | Monkey |
| Cervical | C1-C7 | C1-C7 | C1-C7 |
| Thoracic | T1-T12 | T1-T12 | T1-T12 |
| Lumbar | L1-L5 | L1-L7 | L1-L7 |
| Sacral | S1-S5 | S1-S3 | S1-S3 |
| Coccygeal (caudal) | Co1 | Co1-3 | Co1-3 |

Additionally, the spinal cord can also be divided into six regions anatomically and functionally (Sengul et al., 2013 (Sengul, G., Watson, C., Tanaka, I., Paxinos, G., 2013. Atlas of the Spinal Cord of the Rat, Mouse, Marmoset, Rhesus, and Human. Elsevier Academic Press, San Diego), and also Watson et al., Neuroscience Research 93:164-175 (2015)). These regions are the neck muscle region, the upper limb muscle region, the sympathetic outflow region, the lower limb muscle region, the parasympathetic outflow region, and the tail muscle region. These six regions also correlate with territories defined by gene expression during development (see, e.g., Watson et al., supra). The six regions can be defined histologically by the presence or absence of 2 features, the lateral motor column (LMC) and the preganglionic (intermediolateral) column (PGC) (Watson et al., 2015, incorporated herein by reference in its entirety). The limb enlargements are characterized by the presence of a lateral motor column (LMC) and the autonomic regions containing a preganglionic column (PGC). The neck (parabrachial) and tail (caudal) regions have neither an LMC nor a PGC. The limb enlargements and the sympathetic outflow region are marked by particular patterns of hox gene expression in the mouse and chicken, further supporting the division of the spinal cord into these functional regions. Table 3 maps the C, T, L, S and Co designations described in Table 2 to the functional regions according to Sengul et al. and Watson et al. and maps the functional equivalents for Human, Rhesus Monkey, and Japanese Monkey (another macaque). Note: S1 in Rhesus Monkey and L7 in Japanese monkey is located in both crural and postcrural regions.

TABLE 3
Spinal cord regions and sections by function

| Spinal Cord Region | Human | Rhesus Monkey | Japanese Monkey |
|---|---|---|---|
| Neck Muscle Region | C1-C4 (according to | C1-C4 (according to | C1-C3 (as described in |
| (parabrachial region) | Bruce) | Sengul et al.) | Watson et al.) |
| | C1-C3 (according to | | |
| | Sengul et al.) | | |
| Upper limb Region | C5-T1 (according to | C5-T1 (according to | C4-C8 (as described in |
| (brachial region) | Bruce) | Sengul et al.) | Watson et al.) |
| | C4-T1 (according to | | |
| | Sengul et al.) | | |
| Sympathetic outflow | T2-L1 (according to | T2-L3 (according to | T1-L2 (as described in |
| region (postbrachial | Bruce) | Sengul et al.) | Watson et al.) |
| region) | T2-L1 (according to | | |
| | Sengul et al.) | | |
| Lower limb muscle | L2-S2 (according to | L4-S1 (according to | L3-L7 (as described in |
| region (crural region) | Bruce) | Sengul et al.) | Watson et al.) |
| | L3-S2 (according to | | |
| | Sengul et al.) | | |
| Parasympathetic outflow | S3-S4 (according to | S1-S3 (according to | L7-S3 (as described in |
| region (postcrural | Bruce) | Sengul et al.) | Watson et al.) |
| region) | S3-S5 (according to | | |
| | Sengul et al.) | | |
| Tail muscle region | S5-Co1 (according to | Co1-Co3 (according to | Co1-Co3 (as described in |
| (caudal region) | Bruce) | Sengul et al.) | Watson et al.) |
| | Co1 (according to Sengul | | |
| | et al.) | | |

In one embodiment, a subject may be analyzed for spinal anatomy and pathology prior to delivery of the AAV particles described herein. As a non-limiting example, a subject with scoliosis may have a different dosing regimen and/or catheter location compared to a subject without scoliosis.

Cross-sections may be labeled according to vertebral segmentation numbering and/or spinal segment numbering. From the mid-thoracic region through the sacral region, the spinal cord is compressed relative to the vertebrae, resulting in a difference of vertebral and spinal levels.

In one embodiment, a subject may be delivered the AAV particles described herein along the anterior aspect of the spinal cord.

In one embodiment, a subject may be delivered the AAV particles described herein along the ventral aspect of the spinal cord.

In one embodiment, the spinal anatomy and pathology of a subject is evaluated prior to delivery of the AAV particles described herein. As shown by Pahlavian et al., the anatomy of the spinal cord can affect the flow of the CSF (see e.g., Pahlavian et al., Plos One 2014; herein incorporated by reference in its entirety). As a non-limiting example, a subject who has scoliosis or scoliosis related symptoms may affect the delivery route, location, regimen, formulation and orientation of the subject in order to ensure a desired AAV distribution.

### IT and ICV Infusion

In one embodiment, a subject may be delivered the AAV particles herein using intrathecal administration and intracerebroventricular administration.

In one embodiment, delivery of AAV particles to cells of the central nervous system (e.g., parenchyma) is performed by intracerebroventricular (ICV) prolonged infusion and intrathecal (IT) infusion described herein.

In one embodiment, the distribution of AAV particles to cells of the central nervous system may be increased by delivery of AAV particles using intrathecal (IT) administration and intracerebroventricular administration as compared to delivery with a single route of administration. The increase may be 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 60%, 65%, 70%, 75%, 80%, 85%, 90%, 95% or more than 95%, 1-5%, 1-10%, 1-15%, 1-20%, 5-10%, 5-15%, 5-20%, 5-25%, 10-20%, 10-30%, 15-35%, 20-40%, 20-50%, 30-50%, 30-60%, 40-60%, 40-70%, 50-60%, 50-70%, 60-80%, 60-90%, 70-80%, 70-90%, 80-90%, 80-99% or 90-100%.

In one embodiment, delivery of AAV particles to cells of the central nervous system (e.g., parenchyma) is performed by delivery to the cerebrospinal fluid (CSF) via intracerebroventricular (ICV) prolonged infusion and intrathecal (IT) infusion described herein.

In one embodiment, the distribution of AAV particles to spinal column and brain may be increased by delivery of AAV particles using intrathecal (IT) administration and intracerebroventricular administration as compared to delivery with a single route of administration. The increase may be 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 60%, 65%, 70%, 75%, 80%, 85%, 90%, 95% or more than 95%, 1-5%, 1-10%, 1-15%, 1-20%, 5-10%, 5-15%, 5-20%, 5-25%, 10-20%, 10-30%, 15-35%, 20-40%, 20-50%, 30-50%, 30-60%, 40-60%, 40-70%, 50-60%, 50-70%, 60-80%, 60-90%, 70-80%, 70-90%, 80-90%, 80-99% or 90-100%.

In one embodiment, the AAV particles may be delivered to a subject using intracerebroventricular (ICV) and intrathecal (IT) administration to treat a disease or disorder such as, but not limited to, Friedreich's Ataxia (FA), Amyotrophic Lateral Sclerosis (ALS), Spinal Muscular Atrophy (SMA) and/or Neuropathic Pain.

In one embodiment, a catheter used for ICV and/or IT administration of the AAV particles may include, but is not limited to, the SmartFlow catheter (MRI Interventions), SmartFlow Adjustable Tip Catheter (MRI Interventions), Cleveland Multiport Catheter (Infuseon Therapeutics, Inc.), MEMS catheter (Alcyone Lifesciences, Inc.), Carbothane CED cannula (Renishaw) Smartflow Flex (BrainLab) and/or Intracerebral Microinjection Instrument (IMI) (Atanse).

In one embodiment, the AAV particles may be delivered via intracerebroventricular (ICV) and/or intrathecal (IT) infusion and therapeutic agent may also be delivered to a subject via intravascular limb infusion in order to deliver the therapeutic agent to the skeletal muscle. Delivery of adeno-associated virus by intravascular limb infusion is described by Gruntman and Flotte (Human Gene Therapy Clinical Development, Vol. 26(3), 2015 159-164; the contents of which is herein incorporated by reference in its entirety).

### Delivery to Cells and Tissues

The present disclosure provides a method of delivering to a cell or tissue any of the above-described AAV particles, comprising contacting the cell or tissue with said AAV particle or contacting the cell or tissue with a particle comprising said AAV particle, or contacting the cell or tissue with any of the described compositions, including pharmaceutical compositions. The method of delivering the AAV particle to a cell or tissue can be accomplished in vitro, ex vivo, or in vivo.

In one embodiment, the AAV particles described herein are delivered to the DRG neurons in a volume required for clinical benefit. The AAV particles may be delivered to at least 10%, 15%, 20%, 25%, 30%, 35%, 40%, 45%, 50%, 55%, 60%, 65%, 70%, 75%, 80%, 85%, 90%, 95% or more than 95% of DRG neurons. As a non-limiting example, the AAV particles are delivered to at least 30% of the DRG neurons.

In one embodiment, the AAV particles may be delivered by a method to provide uniform transduction of the spinal cord and dorsal root ganglion (DRG). As a non-limiting example, the AAV particles may be delivered using intrathecal infusion. The intrathecal infusion may be a bolus infusion or it may be a continuous infusion. As another non-limiting example, the AAV particles are delivered using continuous intrathecal infusion over a period of about 10 hours.

In one embodiment, delivery of AAV particles comprising a viral genome encoding a payload described herein to sensory neurons in the dorsal root ganglion (DRG), ascending spinal cord sensory tracts, and cerebellum will lead to an increased expression of the encoded payload. The increased expression may lead to improved survival and function of various cell types.

In one embodiment, delivery of AAV particles comprising a nucleic acid sequence encoding frataxin to sensory neurons in the dorsal root ganglion (DRG), ascending spinal cord sensory tracts, and cerebellum leads to an increased expression of frataxin. The increased expression of frataxin then leads to improved survival, ataxia (balance) and gait, sensory capability, coordination of movement and strength, functional capacity and quality of life and/or improved function of various cell types.

In one embodiment, DRG and/or cortical brain expression may be higher with shorter, high concentration infusions.

In one embodiment, the AAV particles comprise a capsid from an AAV serotype which can infiltrate ganglion, there is microgliosis in the spinal cord gray matter and neuronal necrosis and generation in the spinal cord and DRG. As a non-limiting example, the viral genome is self-complementary and the capsid is from the AAVrh10 serotype. As another non-limiting example, the viral genome is single stranded and the capsid is from the AAVrh10 serotype.

In one embodiment, the AAV particles comprise a capsid from an AAV serotype which can infiltrate ganglion, there is microgliosis in the spinal cord gray matter and neuronal necrosis and generation in the spinal cord and DRG. As a non-limiting example, the viral genome is single stranded and the capsid is from the AAV6 serotype. As a non-limiting example, the viral genome is self-complementary and the capsid is from the AAV6 serotype.

In one embodiment, the AAV particles comprise a capsid from an AAV serotype which can infiltrate ganglion, there is microgliosis in the spinal cord gray matter and neuronal necrosis and generation in the DRG. As a non-limiting example, the viral genome is single stranded and the capsid is from the AAV9 serotype. As a non-limiting example, the viral genome is self-complementary and the capsid is from the AAV9 serotype. As a non-limiting example, the viral genome is single stranded and the capsid is from the AAV5 serotype. As a non-limiting example, the viral genome is self-complementary and the capsid is from the AAV5 serotype.

In one embodiment, the AAV particles comprise a capsid from an AAV serotype which can mildly infiltrate ganglion. As a non-limiting example, the viral genome is single stranded and the capsid is from the AAVDJ serotype. As a non-limiting example, the viral genome is self-complementary and the capsid is from the AAVDJ serotype. As a non-limiting example, the viral genome is single stranded and the capsid is from the AAVDJ8 serotype. As a non-limiting example, the viral genome is self-complementary and the capsid is from the AAVDJ8 serotype.

### Delivery Devices

Devices for administration may be employed for delivery of AAV particles to cells of the central nervous system (e.g., parenchyma) according to the present invention according to single, multi- or split-dosing regimens taught herein.

Method and devices known in the art for multi-administration to cells, organs and tissues are contemplated for use in conjunction with the methods and compositions disclosed herein as embodiments of the present invention. These include, for example, those methods and devices having multiple needles, hybrid devices employing for example lumens or catheters as well as devices utilizing heat, electric current or radiation driven mechanisms.

In one embodiment, the AAV particles may administered to a subject using a device to deliver the AAV particles and a head fixation assembly. The head fixation assembly may be, but is not limited to, Leksell, CRW and/or Medtech ROSA, or any of the head fixation assemblies sold by MRI interventions (e.g., SmartFrame), BrainLab (e.g., Kick or Varioguide), Medtronic (e.g., StealthStation). As a non-limiting example, the head fixation assembly may be any of the assemblies described in U.S. Pat. Nos. 8,099,150, 8,548,569 and 9,031,636 and International Patent Publication Nos. WO201108495 and WO2014014585, the contents of each of which are incorporated by reference in their entireties. A head fixation assembly may be used in combination with an MRI compatible drill such as, but not limited to, the MRI compatible drills described in International Patent Publication No. WO2013181008 and US Patent Publication No. US20130325012, the contents of which are herein incorporated by reference in its entirety.

In one embodiment, the AAV particles may be delivered to a subject using the Clearpoint system from MRI Intervention. The Clearpoint system provides assistance with cannula placement and infusion monitoring, and uses a frame/trajectory device (e.g., SmartFlow trajectory device), and a neuronavigational system that allows for real time adjustment of infusion. The Clearpoint system may be used in combination with a cannula such as, but not limited to, a SmartFlow cannula.

In one embodiment, the AAV particles may be delivered to a subject using a system which may be used in combination with an MRI and/or in an operating room and provides for MRI monitoring of the infusion and can use neuronavigational software. As a non-limiting example, the delivery systems may allow for surgical times of less than 8 hours. As another non-limiting example, the delivery system can maintain real-time MRI-guided navigation and adjustment and also provides for maximum coverage of the therapeutic area of a subject. As yet another non-limiting example, the delivery system may be used in combination with existing navigation software which is currently commonly used by neurosurgeons.

In one embodiment, the AAV particles may be delivered to a subject while the subject is wearing a skull frame connected to the skull using burr holes.

In one embodiment, the AAV particles may be delivered to a subject while the subject is wearing a scalp mounted frame connected to the scalp using key holes. The scalp mounted frame may allow the frame to be reposition if more than one entry site is required for administration (e.g., for an additional infusion).

In one embodiment, the AAV particles may be delivered to a subject using a trajectory frame as described in US Patent Publication Nos. US20150031982 and US20140066750 and International Patent Publication Nos. WO2015057807 and WO2014039481, the contents of each of which are herein incorporated by reference in their entireties.

In one embodiment, the AAV particles may be delivered to a subject using a trajectory guide or frame such as, but not limited to, SmartFrame by MRI Interventions, SmartFlow catheter with a bone anchor (BrainLab and MRI Interventions), neuro Convect (Renishaw) Navigus or bone anchor from Medtronic, KB ball guide, Monteris AXiiiS or mini-bolt, and FHC STarFix.

In one embodiment, the AAV particles may be delivered to a subject using a trajectory guide or frame designed and/or developed by C2C Development, LLC.

In one embodiment, the AAV particles may be delivered using a method, system and/or computer program for positioning apparatus to a target point on a subject to deliver the AAV particles. As a non-limiting example, the method, system and/or computer program may be the methods, systems and/or computer programs described in U.S. Pat. No. 8,340,743, the contents of which are herein incorporated by reference in its entirety. The method may include: determining a target point in the body and a reference point, wherein the target point and the reference point define a planned trajectory line (PTL) extending through each; determining a visualization plane, wherein the PTL intersects the visualization plane at a sighting point; mounting the guide device relative to the body to move with respect to the PTL, wherein the guide device does not intersect the visualization plane; determining a point of intersection (GPP) between the guide axis and the visualization plane; and aligning the GPP with the sighting point in the visualization plane.

In one embodiment, a surgical alignment device may be used to deliver the AAV particles to a subject. The surgical alignment device may be a device described herein and/or is known in the art. As a non-limiting example, the surgical alignment device may be controlled remotely (i.e., robotic) such as the alignment devices described in U.S. Pat. Nos. 7,366,561 and 8,083,753, the contents of each of which is incorporated by reference in their entireties.

In one embodiment, a trajectory guide device may be used in preparation and delivery of the AAV particles described herein. Non-limiting examples of trajectory guide devices include Navigus from Medtronic, Varioguide skull adapter from BrainLab, Neuromate robot from Renishaw, and a ball joint fixture from MRI Interventions.

In one embodiment, prior to intraparenchymal administration of the AAV particles described herein, neuronavigational software is used to determine the administration site. Non-limiting examples of neuronavigational software includes StealthViz from Medtronic, iPlan from BrainLab and neuro Inspire from Renishaw. As a non-limiting example, the neuronavigational software includes pre-planning and intraoperative modules which may be separate and customizable depending on the procedure being conducted.

In one embodiment, neuronavigational software, a trajectory guide device, a catheter and imaging analysis is used prior to, during and/or after administration of the AAV particles described herein.

In one embodiment, the AAV particles may be delivered using an MRI-guided device. Non-limiting examples of MRI-guided devices are described in U.S. Pat. Nos. 9,055,884, 9,042,958, 8,886,288, 8,768,433, 8,396,532, 8,369,930, 8,374,677 and 8,175,677 and US Patent Application No. US20140024927 the contents of each of which are herein incorporated by reference in their entireties. As a non-limiting example, the MRI-guided device may be able to provide data in real time such as those described in U.S. Pat. Nos. 8,886,288 and 8,768,433, the contents of each of which is herein incorporated by reference in its entirety. As another non-limiting example, the MRI-guided device or system may be used with a targeting cannula such as the systems described in U.S. Pat. Nos. 8,175,677 and 8,374,677, the contents of each of which are herein incorporated by reference in their entireties. As yet another non-limiting example, the MRI-guided device includes a trajectory guide frame for guiding an interventional device as described, for example, in U.S. Pat. No. 9,055,884 and US Patent Application No. US20140024927, the contents of each of which are herein incorporated by reference in their entireties.

In one embodiment the AAV particles may be delivered using an MRI-compatible tip assembly. Non-limiting examples of MRI-compatible tip assemblies are described in US Patent Publication No. US20140275980, the contents of which is herein incorporated by reference in its entirety.

In one embodiment, the AAV particles may be delivered using an MRI compatible localization and/or guidance system such as, but not limited to, those described in US Patent Publication Nos. US20150223905 and US20150230871, the contents of each of which are herein incorporated by reference in their entireties. As a non-limiting example, the MRI compatible localization and/or guidance systems may comprise a mount adapted for fixation to a patient, a targeting cannula with a lumen configured to attach to the mount so as to be able to controllably translate in at least three dimensions, and an elongate probe configured to snugly advance via slide and retract in the targeting cannula lumen, the elongate probe comprising at least one of a stimulation or recording electrode.

In one embodiment, a subject may be administered the AAV particles described herein using a catheter. The catheter may be placed in the lumbar region or the cervical region of a subject. As a non-limiting example, the catheter may be placed in the lumbar region of the subject. As another non-limiting example, the catheter may be placed in the cervical region of the subject. As yet another non-limiting example, the catheter may be placed in the high cervical region of the subject. As used herein, the “high cervical region” refers to the region of the spinal cord comprising the cervical vertebrae C1, C2, C3 and C4 or any subset thereof.

In one embodiment, the catheter may be in located at one site in the spine for delivery. As a non-limiting example, the location may be in the cervical or the lumbar region. The AAV particles may be delivered in a continuous or bolus infusion.

In one embodiment, the catheter may be located at more than one site in the spine for multi-site delivery. The AAV particles may be delivered in a continuous and/or bolus infusion. Each site of delivery may be a different dosing regimen or the same dosing regimen may be used for each site of delivery. As a non-limiting example, the sites of delivery may be in the cervical and the lumbar region. As another non-limiting example, the sites of delivery may be in the cervical region. As another non-limiting example, the sites of delivery may be in the lumbar region.

In one embodiment, the AAV particles may be delivered using a catheter which is MRI-compatible. Non-limiting examples of MRI-compatible catheters include those taught in International Patent Publication No. WO2012116265, U.S. Pat. No. 8,825,133 and US Patent Publication No. US20140024909, the contents of each of which are herein incorporated by reference in their entireties.

In one embodiment, the catheter may be a neuromodulation catheter. Non-limiting examples of neuromodulation catheters include those taught in US Patent Application No. US20150209104 and International Publication Nos. WO2015143372, WO2015113027, WO2014189794 and WO2014150989, the contents of each of which are herein incorporated by reference in their entireties.

In one embodiment, a catheter used for administration of the AAV particles may include, but is not limited to, the SmartFlow catheter (MRI Interventions), SmartFlow Adjustable Tip Catheter (MRI Interventions), Cleveland Multiport Catheter (Infuseon Therapeutics, Inc.), MEMS catheter (Alcyone Lifesciences, Inc.), Carbothane CED cannula (Renishaw), SmartFlow (BrainLab), Smartflow Flex (BrainLab), neuro Convect (Renishaw) and/or Intracerebral Microinjection Instrument (IMI) (Atanse).

In one embodiment, the AAV particles described herein may be delivered using a micro-electro-mechanical system (MEMS) catheter from Alcyone. The MEMS catheter may include, more than one Luer connections, stop for desired depth, stiff shaft for stereotactic frames, tip-protector microtip for insertion into stereotactic frame fixtures, micro size wide tip with at least one channel/outlet, backflow stop features, and/or sensor at the tip (e.g., for monitoring pressure at the outlet, oxygen tension, pH, etc.).

In one embodiment, an intraparenchymal (IPA) catheter from Alcyone may be used to deliver the AAV particles described herein. As a non-limiting example, the catheter is the micro-electro-mechanical-system (MEMS) catheter from Alcyone.

In one embodiment, the AAV particles described herein may be delivered using an intraparenchymal catheter which may have at least one design feature such as, but not limited to, built in pressure sensor, at least one infusion level (e.g., 1, 2, 3, 4, 5, 6, 7, 8 or more than 8 individual flow channels), compatibility to stereotaxic equipment, MRI-safe with limited flare and good resolution, CED flow rates greater than 10 ul/min, reflux-resistance, and insertion should cause minimal trauma on the subject.

In another embodiment, an intraparenchymal catheter from Atanse may be used to deliver the AAV particles described herein.

In one embodiment, the catheter may be one designed and/or developed by C2C Development, LLC.

In one embodiment, the AAV particles may be delivered using a cannula which is MRI-compatible. Non-limiting examples of MRI-compatible cannulas include those taught in International Patent Publication No. WO2011130107, the contents of which are herein incorporated by reference in its entirety.

In one embodiment, the AAV particles may be delivered using a rigid cannula with an adjustable fused silica tip which can be manually or automatically extended or retracted during delivery. While not wishing to be bound by theory, the extendable feature of the tip can allow delivery of the AAV particles along the length of a surface such as, but not limited to, the length of a putamen. Optionally, the cannula may be compatible to any stereotaxic navigational system.

In one embodiment, the AAV particles may be delivered using a flexible cannula which has a rigid tip portion with a stepped design depending on the delivery site. Optionally, a skull adaptor and/or locking mechanism may be used for acute and/or multi-day applications. The cannula may also be compatible with most stereotaxic navigational systems.

In one embodiment, the AAV particles may be delivered using a rigid cannula which has a single lumen end port with a tapered step to reduce backflow. The cannula has different tip lengths to match the anatomy of the target site for delivery and different diameters to allow for higher flow rates. Optionally, the cannula may be compatible to any stereotaxic navigational system.

In one embodiment, the AAV particles may be delivered using a flexible carbothane cannula with a recessed step design. More than one cannula may be used to deliver the AAV particles to a subject. Optionally, the cannula may be compatible to any stereotaxic navigational system.

In one embodiment, the AAV particles may be delivered using a catheter with an inner drug delivery cannula that can extend to infuse at multiple sites surrounding the central catheter.

In one embodiment, the devices described herein to deliver to a subject the above-described AAV particles may also include a tip protection device (e.g., for catheters and/or stereotactic fixtures of microcatheters). Non-limiting examples of protection devices are described in US Patent Publication No. US20140371711 and International Patent Publication No. WO2014204954, the contents of each of which are herein incorporated by reference in their entireties. The tip protection device may include an elongate body having a central lumen extending longitudinally therethrough, the lumen being sized and configured to slidably receive a catheter, and a locking mechanism configured to selectively maintain the elongate body in a fixed longitudinal position relative to a catheter inserted through the central lumen.

In one embodiment, the AAV particles may be delivered using an infusion port described herein and/or one that is known in the art.

In one embodiment, the AAV particles may be delivered using an infusion pump and/or an infusion port. The infusion pump and/or the infusion port may be one described herein or one known in the art such as, but not limited to, SYNCHROMED® II by Medtronic. The infusion pump may be programmed at a fixed rate or a variable rate for controlled delivery. The stability of the AAV particles and formulations thereof as well as the leachable materials should be evaluated prior to use.

In one embodiment, to reduce peripheral organ exposure, a multi-port catheter may be used to deliver AAV particles. The device may have at least 2 ports to allow for the inflow of the AAV particles and the outflow of the CSF. As a non-limiting example, the inflow port is located near the cervical region and the outflow port is located near the sacral region. As another non-limiting example, the inflow and outflow ports are located to focus delivery to specific spinal segments and limit the distribution of the AAV particles to other CNS regions.

In one embodiment, a multi-port catheter may be used to deliver AAV particles to treat motor neuron diseases such as, but not limited to, ALS. The multi-port catheter may allow for neuraxial spread of the AAV particles in a subject. The multi-port catheter may have at least 2, 3, 4, 5, 6, 7, 8, 9 or more than 9 ports. As a non-limiting example, the multi-port catheter has 3 ports.

In one embodiment, a multi-port catheter may be used to deliver AAV particles to treat Friedreich's Ataxia. The multi-port catheter has an inflow port located in the cervical region and an outflow port located in the lumbar region. This isolated spinal cord perfusion limits the spread of the AAV particles.

In one embodiment, a multi-port catheter may be used to deliver AAV particles to treat neuropathic pain. The multi-port catheter has an inflow port located a predetermined distance from an outflow port in order to provide AAV particles to a specific region of the CNS. The distance between the inflow and outflow port may be centimeters (e.g., 1, 5, 10, 15, 20, 25, 30, 35, 40, 45, 50, 55, 60, 65, 70, 75, 80, 85, 90, 95, 100 or more than 100) or inches (¼, ½, ¾, 1, 2, 3, 4, 5, 6, 7, 8, 9, 10, 11, 12 or more than 12 inches). This isolated segmental perfusion of the AAV particles allows for a reduced dose and spread of the AAV particles.

In one embodiment, the AAV particles may be delivered using a device with an elongated tubular body and a diaphragm as described in US Patent Publication Nos. US20140276582 and US20140276614, the contents of each of which are herein incorporated by reference in their entireties.

In some embodiments, delivery of AAV particles to cells of the central nervous system (e.g., parenchyma) comprises a prolonged infusion pump or device. In some embodiments, the device may be a pump or comprise a catheter for administration of compositions of the invention across the blood brain barrier. Such devices include but are not limited to a pressurized olfactory delivery device, iontophoresis devices, multi-layered microfluidic devices, and the like. Such devices may be portable or stationary. They may be implantable or externally tethered to the body or combinations thereof.

In one embodiment, the AAV particles may be delivered to a subject using a convection-enhanced delivery device. Non-limiting examples of targeted delivery of drugs using convection are described in US Patent Publication Nos. US20100217228, US20130035574 and US20130035660 and International Patent Publication No. WO2013019830 and WO2008144585, the contents of each of which are herein incorporated by reference in their entireties. The convection-enhanced delivery device may be a microfluidic catheter device that may be suitable for targeted delivery of drugs via convection, including devices capable of multi-directional drug delivery, devices that control fluid pressure and velocity using the venturi effect, and devices that include conformable balloons. As a non-limiting example, the convention-enhanced delivery device uses the venturi effect for targeted delivery of drugs as described in US Patent Publication No. US20130035574, the contents of which are herein incorporation by reference in its entirety. As another non-limiting example, the convention-enhanced delivery device uses the conformable balloons for targeted delivery of drugs as described in US Patent Publication No. US20130035660, the contents of which are herein incorporation by reference in its entirety. As another non-limiting example, the convection enhanced delivery device may be a CED catheter from Medgenesis Therapeutix such as those described in International Patent Publication No. WO2008144585 and US Patent No. US20100217228, the contents of each of which are herein incorporated by reference in their entireties. As another non-limiting example, the AAV particles may be in a liposomal composition for convection enhanced delivery such as the liposomal compositions from Medgenesis Therapeutix described in International Patent Publication No. WO2010057317 and US Patent No. US20110274625, the contents of each of which are herein incorporated by reference in their entireties, which may comprise a molar ratio of DSPC:DSPG:CHOL of 7:2:1.

In one embodiment, the AAV particles may be delivered using an injection device which has a basic form of a stiff tube with holes of a selectable size at selectable places along the tube. This is a device which may be customized depending on the subject or the fluid being delivered. As a non-limiting example, the injection device which comprises a stiff tube with holes of a selectable size and location may be any of the devices described in U.S. Pat. Nos. 6,464,662, 6,572,579 and International Patent Publication No. WO2002007809, the contents of each of which are herein incorporated by reference in their entireties.

In one embodiment, the AAV particles may be delivered to a defined area using a medical device which comprises a sealing system proximal to the delivery end of the device. Non-limiting examples of medical device with can deliver AAV particles to a defined area includes U.S. Pat. No. 7,998,128, US Patent Application No. US20100030102 and International Patent Publication No. WO2007133776, the contents of each of which are herein incorporated by reference in their entireties.

In one embodiment, the AAV particle may be delivered over an extended period of time using an extended delivery device. Non-limiting examples of extended delivery devices are described in International Patent Publication Nos. WO2015017609 and WO2014100157, U.S. Pat. No. 8,992,458, and US Patent Publication Nos. US20150038949, US20150133887 and US20140171902, the contents of each of which are herein incorporated by reference in their entireties. As a non-limiting example, the devices used to deliver the AAV particles are CED devices with various features for reducing or preventing backflow as in International Patent Publication No. WO2015017609 and US Patent Publication No. US20150038949, the contents of each of which are herein incorporated by reference in their entireties. As another non-limiting example, the devices used to deliver the AAV particles are CED devices which include a bullet-shaped nose proximal to a distal fluid outlet where the bullet-shaped nose forms a good seal with surrounding tissue and helps reduce or prevent backflow of infused fluid as described in U.S. Pat. No. 8,992,458, US Patent Publication Nos. US20150133887 and US20140171902 and International Patent Publication No. WO2014100157, the contents of each of which are herein incorporated by reference by their entireties. As another non-limiting example, the catheter may be made using micro-electro-mechanical systems (MEMS) technology to reduce backflow as described by Brady et al. (Journal of Neuroscience Methods 229 (2014) 76-83), the contents of which are herein incorporated by reference in its entirety.

In one embodiment, the AAV particles may be delivered using an implantable delivery device. Non-limiting examples of implantable devices are described by and sold by Codman Neuro Sciences (Le Locle, CH). The implantable device may be an implantable pump such as, but not limited to, those described in U.S. Pat. Nos. 8,747,391, 7,931,642, 7,637,897, and 6,755,814 and US Patent Publication No. US20100069891, the contents of each of which are herein incorporated by reference in their entireties. The implantable device (e.g., a fluidic system) may have the flow rate accuracy of the device optimized by the methods described in U.S. Pat. Nos. 8,740,182 and 8,240,635, and US Patent Publication No. US20120283703, the contents of each of which are herein incorporated by reference in its entirety. As a non-limiting example, the duty cycle of the valve of a system may be optimized to achieve the desired flow rate. The implantable device may have an electrokinetic actuator for adjusting, controlling or programming fine titration of fluid flow through a valve mechanism without intermixing between the electrolyte and fluid. As a non-limiting example, the electrokinetic actuator may be any of those described in U.S. Pat. No. 8,231,563 and US Patent Publication No. US20120283703, the contents of which are herein incorporated by reference in its entirety. Fluids of an implantable infusion pump may be monitored using methods known in the art and those taught in U.S. Pat. No. 7,725,272, the contents of which are herein incorporated by reference in its entirety.

In one embodiment, a device may be used to deliver the AAV particles where the device creates one or more channels, tunnels or grooves in tissue in order to increase hydraulic conductivity. These channels, tunnels or grooves will allow the AAV particles to flow and produce a predictable infusion pattern. Non-limiting examples of this device is described in U.S. Pat. No. 8,083,720, US Patent Application No. US20110106009, and International Publication No. WO2009151521, the contents of each of which are herein incorporated by reference in its entirety.

In one embodiment, a pulsar intrathecal delivery device from Alcyone may be used to deliver the AAV particles described herein. The delivery device may include a pump to provide timed infusions of AAV particles to a subject based on the CSF natural pulsation connected to the cardiac cycle of a subject. The device may also include catheter to disrupt the flow of the CSF and/or a sensor (e.g., MEMS sensor, and/or a pressure, heartrate, EKG and/or respiration sensor) to ensure effective infusions. The catheter may be a single lumen catheter or a multi-lumen catheter. Additionally, the device may be connected to a programmable pump that can deliver one or more solutions to a subject.

In one embodiment, the pulsar intrathecal delivery device from Alcyone may be a multiple port device. The device may include a sensor (e.g., MEMS sensor, and/or a pressure, heartrate, EKG and/or respiration sensor) at each port to ensure effective infusions. The sensor may be the same or different for each port. Additionally, the device with multiple ports may be connected to a programmable pump that can deliver one or more solutions to a subject.

In one embodiment, an intraparenchymal delivery system from Alcyone may be used to administer the AAV particles described herein. As a non-limiting example, the system may include a distal tip to stop backflow using the properties of the tissue around the administration site.

In one embodiment, an intrathecal delivery device to deliver the AAV particles descried herein via intrathecal infusion may be a multiple port device to ensure a broad distribution of the AAV particles to the spinal cord and/or brain tissue of the subject. The device may include a sensor (e.g., MEMS sensor, and/or a pressure, heartrate, EKG and/or respiration sensor) at each port to ensure effective infusions. The sensor may be the same or different for each port. Additionally, the device with multiple ports may be connected to a programmable pump that can deliver one or more solutions to a subject.

In one embodiment, mechanical percussion (e.g., mechanical percussion jacket) on a subject may be used in combination with the administration of the AAV particles described herein. The mechanical percussion device may increase the dispersion of the AAV particles by 1%, 5%, 10%, 15%, 20%, 25%, 30%, 35%, 40%, 45%, 50%, 55%, 60%, 65%, 70%, 75%, 80%, 85%, 90%, 95%, 99% or more than 99% as compared to the distribution of the AAV particles without use of mechanical percussion.

### Spatial Orientation

### Body Angle and Position

In one embodiment, delivery of AAV particles to cells of the central nervous system (e.g., parenchyma) comprises administration to a horizontal subject. In one embodiment, delivery comprises administration to a vertical subject. In one embodiment, delivery comprises administration to a subject at an angle between approximately horizontal 0° to about vertical 90°. In one embodiment, delivery comprises administration to a subject at an angle of 0°, 1°, 2°, 3° 4°, 5°, 6°, 7°, 8°, 9°, 10°, 11°, 12°, 13°, 14°, 15°, 16°, 17°, 18°, 19°, 20°, 21°, 22°, 23°, 24°, 25°, 26°, 27°, 28°, 29°, 30°, 31°, 32°, 33°, 34°, 35°, 36°, 37°, 38°, 39°, 40°, 41°, 42°, 43°, 44°, 45°, 46°, 47°, 48°, 49°, 50°, 51°, 52°, 53°, 54°, 55°, 56°, 57°, 58°, 59°, 60°, 61°, 62°, 63°, 64°, 65°, 66°, 67°, 68°, 69°, 70°, 71°, 72°, 73°, 74°, 75°, 76°, 77°, 78°, 79°, 80°, 81°, 82°, 83°, 84°, 85°, 86°, 87°, 88°, 89°, 90°.

In one embodiment, the spine of the subject may be at an angle as compared to the ground during the delivery of the AAV particles subject. The angle of the spine of the subject as compared to the ground may be at least 10, 20, 30, 40, 50, 60, 70, 80, 90, 100, 110, 120, 130, 140, 150 or 180 degrees.

In one embodiment, delivery of AAV particles to a subject comprises administration of a hyperbaric composition while the subject is in the supine position. As a non-limiting example, the AAV particles described herein may be administered to a subject in the supine position to focus delivery of the AAV particles to the dorsal horn and provide treatment or mitigation of pain.

In one embodiment, delivery of AAV particles to a subject comprises administration of a hyperbaric composition while the subject is in the prone position. As a non-limiting example, the AAV particles described herein may be administered to a subject in the prone position to focus delivery of the AAV particles to the anterior horn and provide treatment for ALS.

In one embodiment, delivery of AAV particles to a subject comprises administration of a hyperbaric composition while the subject is in the right lateral recumbent (RLR) position. As a non-limiting example, the AAV particles described herein may be administered to a subject in the RLR position to focus delivery of the AAV particles to the dorsal root ganglion to provide treatment of FA or treatment and mitigation of pain.

In one embodiment, delivery of AAV particles to a subject comprises administration of a hyperbaric composition while the subject is in the left lateral recumbent (LLR) position. As a non-limiting example, the AAV particles described herein may be administered to a subject in the LLR position to focus delivery of the AAV particles to the dorsal root ganglion to provide treatment of FA or treatment and mitigation of pain.

In one embodiment, delivery of AAV particles to a subject comprises administration of a hyperbaric composition while the subject is in the Fowler's position. As a non-limiting example, the subject is in a high fowler's position. As another non-limiting example, the subject is in a low fowler's position.

In one embodiment, delivery of AAV particles to a subject comprises administration of a hyperbaric composition while the subject is in the Trendelenburg position. As a non-limiting example, the AAV particles described herein may be administered to a subject in the Trendelenburg position to focus delivery of the AAV particles to the cervical region of the CNS. In one embodiment, the orientation of the spine subject during delivery of the AAV particles may be vertical to the ground.

### Change in the Orientation/Slope of Subject Body Position Over Time

In one embodiment, delivery of AAV particles to cells of the central nervous system (e.g., parenchyma) comprises administration to a subject wherein the angle of the subject changes over time from horizontal to vertical head up or vertical head down. In one embodiment, delivery comprises administration to a subject wherein the angle of the subject changes over time from vertical to horizontal.

In one embodiment, delivery comprises administration to a subject wherein the angle of the subject changes over time in two planes from vertical to horizontal as well as rotation around the long axis of the body. In combination, any % angle of the body can be realized between horizontal to vertical and rotationally left or right.

### Dosing

The present invention provides methods of administering AAV particles in accordance with the invention to a subject in need thereof. AAV particle pharmaceutical, imaging, diagnostic, or prophylactic compositions thereof, may be administered to a subject using any amount and any route of administration effective for preventing, treating, diagnosing, or imaging a disease, disorder, and/or condition (e.g., a disease, disorder, and/or condition relating to working memory deficits). The exact amount required will vary from subject to subject, depending on the species, age, and general condition of the subject, the severity of the disease, the particular composition, its mode of administration, its mode of activity, and the like. Compositions in accordance with the invention are typically formulated in unit dosage form for ease of administration and uniformity of dosage. It will be understood, however, that the total daily usage of the compositions of the present invention may be decided by the attending physician within the scope of sound medical judgment. The specific therapeutically effective, prophylactically effective, or appropriate imaging dose level for any particular patient will depend upon a variety of factors including the disorder being treated and the severity of the disorder; the activity of the specific payload employed; the specific composition employed; the age, body weight, general health, sex and diet of the patient; the time of administration, route of administration, and rate of excretion of the specific payload employed; the duration of the treatment; drugs used in combination or coincidental with the specific payload employed; and like factors well known in the medical arts.

In one embodiment, delivery of the AAV particles described herein results in minimal serious adverse events (SAEs) as a result of the delivery of the AAV particles.

In one embodiment, a subject has had a low incidence of mild to moderate adverse events (AEs) near the time of the administration of the AAV particles. The subject may have had a low incidence of mild to moderate AEs within minutes (e.g., 1, 5, 10, 15, 20, 25, 30, 35, 40, 45, 50, 55 or 60 minutes), hours (1, 2, 3, 4, 5, 6, 7, 8, 9, 10, 11, 12, 13, 14, 15, 16, 17, 18, 19, 20, 21, 22, 23, or 24 hours) or days (1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 days).

In one embodiment, a subject may be administered the AAV particles described herein using sustained delivery over a period of minutes, hours or days. The infusion rate may be changed depending on the subject, distribution, formulation or another delivery parameter known to those in the art.

In certain embodiments, AAV particle pharmaceutical compositions in accordance with the present invention may be administered at dosage levels sufficient to deliver from about 0.0001 mg/kg to about 100 mg/kg, from about 0.001 mg/kg to about 0.05 mg/kg, from about 0.005 mg/kg to about 0.05 mg/kg, from about 0.001 mg/kg to about 0.005 mg/kg, from about 0.05 mg/kg to about 0.5 mg/kg, from about 0.01 mg/kg to about 50 mg/kg, from about 0.1 mg/kg to about 40 mg/kg, from about 0.5 mg/kg to about 30 mg/kg, from about 0.01 mg/kg to about 10 mg/kg, from about 0.1 mg/kg to about 10 mg/kg, or from about 1 mg/kg to about 25 mg/kg, of subject body weight per day, one or more times a day, to obtain the desired therapeutic, diagnostic, prophylactic, or imaging effect. It will be understood that the above dosing concentrations may be converted to vg or viral genomes per kg or into total viral genomes administered by one of skill in the art.

In one embodiment, the total dose of viral genomes delivered to cells of the central nervous system (e.g., parenchyma) defined by the equation [Total Dose VG=VG/mL*mL*# of doses] wherein VG is viral genomes and VG/mL is viral genome concentration. In accordance with the present invention, the total dose may be between about 1×10⁶ VG and about 1×10¹⁶ VG.

In one embodiment, delivery of AAV particles to cells of the central nervous system (e.g., parenchyma) may comprise a total dose between about 1×10⁶ VG and about 1×10¹⁶ VG. In some embodiments, delivery may comprise a total dose of about 1×10⁶, 2×10⁶, 3×10⁶, 4×10⁶, 5×10⁶, 6×10⁶, 7×10⁶, 8×10⁶, 9×10⁶, 1×10⁷, 2×10⁷, 3×10⁷, 4×10⁷, 5×10⁷, 6×10⁷, 7×10⁷, 8×10⁷, 9×10⁷, 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 1.9×10¹⁰, 2×10¹⁰, 3×10¹⁰, 3.73×10¹⁰, 4×10¹⁰, 5×10¹⁰, 6×10¹⁰, 7×10¹⁰, 8×10¹⁰, 9×10¹⁰, 1×10¹¹, 2×10¹¹, 2.5×10¹¹, 3×10¹¹, 4×10¹¹, 5×10¹¹, 6×10¹¹, 7×10¹¹, 8×10¹¹, 9×10¹¹, 1×10¹², 2×10¹², 3×10¹², 4×10¹², 5×10¹², 6×10¹², 7×10¹², 8×10¹², 9×10¹² 1×10¹³, 2×10¹³, 3×10¹³, 4×10¹³, 5×10¹³, 6×10¹³, 7×10¹³, 8×10¹³, 9×10¹³, 1×10¹⁴, 2×10¹⁴, 3×10¹⁴, 4×10¹⁴, 5×10¹⁴, 6×10¹⁴, 7×10¹⁴, 8×10¹⁴, 9×10¹⁴ 1×10¹⁵4, 1×10¹⁵, 2×10¹⁵, 3×10¹⁵, 4×10¹⁵, 5×10¹⁵, 6×10¹⁵, 7×10¹⁵, 8×10¹⁵, 9×10¹⁵, or 1×10¹⁶ VG. As a non-limiting example, the total dose is 1×10¹³ VG. As another non-limiting example, the total dose is 3×10¹³ VG. As another non-limiting example, the total dose is 3.73×10¹⁰ VG. As another non-limiting example, the total dose is 1.9×10¹⁰ VG. As another non-limiting example, the total dose is 2.5×10¹¹ VG. As another non-limiting example, the total dose is 5×10¹¹ VG. As another non-limiting example, the total dose is 1×10¹² VG. As another non-limiting example, the total dose is 5×10¹² VG.

In one embodiment, delivery of AAV particles to cells of the central nervous system (e.g., parenchyma) may comprise a composition concentration between about 1×10⁶ VG/mL and about 1×10¹⁶ VG/mL. In some embodiments, delivery may comprise a composition concentration of about 1×10⁶, 2×10⁶, 3×10⁶, 4×10⁶, 5×10⁶, 6×10⁶, 7×10⁶, 8×10⁶, 9×10⁶, 1×10⁷, 2×10⁷, 3×10⁷, 4×10⁷, 5×10⁷, 6×10⁷, 7×10⁷, 8×10⁷, 9×10⁷, 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 2×10¹⁰, 3×10¹⁰, 4×10¹⁰, 5×10¹⁰, 6×10¹⁰, 7×10¹⁰, 8×10¹⁰, 9×10¹⁰, 1×10¹¹, 2×10¹¹, 3×10¹¹, 4×10¹¹, 5×10¹¹, 6×10¹¹, 7×10¹¹, 8×10¹¹, 9×10¹¹, 1×10¹², 2×10¹², 3×10¹², 4×10¹², 5×10¹², 6×10¹², 7×10¹², 8×10¹², 9×10¹², 1×10¹³, 2×10¹³, 3×10¹³, 4×10¹³, 5×10¹³, 6×10¹³, 7×10¹³ 8×10¹³, 9×10¹³ 1×10¹⁴, 2×10¹⁴, 3×10¹⁴, 4×10¹⁴, 5×10¹⁴, 6×10¹⁴, 7×10¹⁴, 8×10¹⁴, 9×10¹⁴, 1×10¹⁵, 2×10¹⁵, 3×10¹⁵, 4×10¹⁵, 5×10¹⁵, 6×10¹⁵, 7×10¹⁵, 8×10¹⁵, 9×10¹⁵, or 1×10¹⁶ VG/mL. In one embodiment, the delivery comprises a composition concentration of 1×10¹³ VG/mL. In one embodiment, the delivery comprises a composition concentration of 3×10¹² VG/mL.

In one embodiment, delivery of AAV particles to cells of the central nervous system (e.g., parenchyma) may comprise a composition concentration between about 1×10⁶ VG/uL and about 1×10¹⁶ VG/uL. In some embodiments, delivery may comprise a composition concentration of about 1×10⁶, 2×10⁶, 3×10⁶, 4×10⁶, 5×10⁶, 6×10⁶, 7×10⁶, 8×10⁶, 9×10⁶, 1×10⁷, 2×10⁷, 3×10⁷, 4×10⁷, 5×10⁷, 6×10⁷, 7×10⁷, 8×10⁷, 9×10⁷, 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 2×10¹⁰, 3×10¹⁰, 4×10¹⁰, 5×10¹⁰, 6×10¹⁰, 7×10¹⁰, 8×10¹⁰, 9×10¹⁰, 1×10¹¹, 2×10¹¹, 3×10¹¹, 4×10¹¹, 5×10¹¹, 6×10¹¹, 7×10¹¹, 8×10¹¹, 9×10¹¹, 1×10¹², 2×10¹², 3×10¹², 4×10¹², 5×10¹², 6×10¹², 7×10¹², 8×10¹², 9×10¹², 1×10¹³, 2×10¹³, 3×10¹³, 4×10¹³, 5×10¹³, 6×10¹³, 7×10¹³, 8×10¹³, 9×10¹³, 1×10¹⁴, 2×10¹⁴ 3×10¹⁴, 4×10¹⁴, 5×10¹⁴, 6×10¹⁴, 7×10¹⁴, 8×10¹⁴, 9×10¹⁴, 1×10¹⁵, 2×10¹⁵, 3×10¹⁵, 4×10¹⁵, 5×10¹⁵, 6×10¹⁵, 7×10¹⁵, 8×10¹⁵, 9×10¹⁵, or 1×10¹⁶ VG/uL. In one embodiment, the delivery comprises a composition concentration of 1×10¹³ VG/uL. In one embodiment, the delivery comprises a composition concentration of 3×10¹² VG/uL. In one embodiment, the delivery comprises a composition concentration of 1.9×10¹⁰ VG/10 uL. In one embodiment, the delivery comprises a composition concentration of 2.5×10¹¹ VG/100 uL. In one embodiment, the delivery comprises a composition concentration of 5×10¹¹ VG/100 uL.

In one embodiment, delivery of AAV particles to cells of the central nervous system (e.g., parenchyma) may comprise a total dose between about 1×10⁶ VG and about 1×10¹⁶ VG. In some embodiments, delivery may comprise a total dose of about 1×10⁶, 2×10⁶, 3×10⁶, 4×10⁶, 5×10⁶, 6×10⁶, 7×10⁶, 8×10⁶, 9×10⁶, 1×10⁷, 2×10⁷, 3×10⁷, 4×10⁷, 5×10⁷, 6×10⁷, 7×10⁷, 8×10⁷, 9×10⁷, 1×10⁸, 2×10⁸, 3×10⁸, 4×10⁸, 5×10⁸, 6×10⁸, 7×10⁸, 8×10⁸, 9×10⁸, 1×10⁹, 1.9×10⁹, 2×10⁹, 3×10⁹, 4×10⁹, 5×10⁹, 6×10⁹, 7×10⁹, 8×10⁹, 9×10⁹, 1×10¹⁰, 2×10¹⁰, 3×10¹⁰, 4×10¹⁰, 5×10¹⁰, 6×10¹⁰, 7×10¹⁰, 8×10¹⁰, 9×10¹⁰, 1×10¹¹, 2×10¹¹, 2.5×10¹¹, 3×10¹¹, 4×10¹¹, 5×10¹¹, 6×10¹¹, 7×10¹¹, 8×10¹¹, 9×10¹¹, 1×10¹², 2×10¹², 3×10¹², 4×10¹², 5×10¹², 6×10¹², 7×10¹², 8×10¹², 9×10¹², 1×10¹³, 2×10¹³ 3×10¹³ 4 5×10¹³ 5 6×10¹³ 6 7×10¹³, 7×10¹³ 8×10¹³, 9×10¹³, 1×10¹⁴, 2×10¹⁴, 3×10¹⁴, 4×10¹⁴, 5×10¹⁴, 6×10¹⁴, 7×10¹⁴, 8×10¹⁴, 9×10¹⁴, 1×10¹⁵, 2×10¹⁵, 3×10¹⁵, 4×10¹⁵, 5×10¹⁵, 6×10¹⁵, 7×10¹⁵, 8×10¹⁵, 9×10¹⁵, or 1×10¹⁶ VG. As a non-limiting example, the total dose is 1×10¹³ VG. As another non-limiting example, the total dose is 3×10¹³ VG. As another non-limiting example, the total dose is 3.73×10¹⁰ VG. As another non-limiting example, the total dose is 1.9×10¹⁰ VG. As another non-limiting example, the total dose is 2.5×10¹¹ VG. As another non-limiting example, the total dose is 5×10¹¹ VG. As another non-limiting example, the total dose is 1×10¹² VG. As another non-limiting example, the total dose is 5×10¹² VG. As another non-limiting example, the total dose is 3×10¹⁴ VG. As another non-limiting example, the total dose is 4×10¹³ VG.

In one embodiment, delivery of AAV particles to cells of the central nervous system (e.g., parenchyma) comprises a total dose of 5×10¹⁰ VG. In one embodiment, delivery consists of a total dose of 5×10¹⁰ VG. In one embodiment, delivery comprises a total dose of 3×10¹³ VG. In one embodiment, delivery of AAV to cells of the central nervous system (e.g., parenchyma) consists of a total dose of 3×10¹³ VG.

In one embodiment, the dosage delivered to a subject may take into account the amount of backflow of the substance. As a non-limiting example, the method for determining the backflow of a substance or fluid along a track of a delivery device is described in U.S. Pat. Nos. 7,742,630, 7,715,902 and European Publication No. EP1788498, the contents of each of which is herein incorporated by reference in their entireties. As a non-limiting example, a method of reducing the amount of backflow which is described in US Patent Publication No. US20140243783, the contents of which are herein incorporated by reference in its entirety, may be used to reduce the backflow from the administration of composition comprising AAV particles described herein.

In one embodiment, the ratio of the volume of distribution and the volume infused is at least 1:1, 1:2, 1:3, 1:4, 1:5, 2:1, 2:2, 2:3, 2:4, 2:5, 3:1, 3:2, 3:3, 3:4, 3:5, 4:1, 4:2, 4:3, 4:4, 4:5, 5:1, 5:2, 5:3, 5:4, or 5:5. As a non-limiting example, the ratio of the volume of distribution is at least 3:1.

### Infusion Parameters and Volume

In some embodiments, infusion volume, duration of infusion, infusion patterns and rates for delivery of AAV particles to cells of the central nervous system (e.g., parenchyma) may be determined and regulated.

In one embodiment, delivery of AAV particles to cells of the central nervous system (e.g., parenchyma) comprises infusion of at least one dose.

In one embodiment, delivery of AAV to cells of the central nervous system (e.g., parenchyma) may comprise an infusion of 1, 2, 3, 4, 5, 6, 7, 8, 9, or 10 dose(s). The infusion may be a bolus or prolonged infusion.

In one embodiment, delivery of AAV particles to cells of the central nervous system (e.g., parenchyma) comprises infusion of up to 1 mL. The infusion may be at least 0.1 mL, 0.2 mL, 0.3 mL, 0.4 mL, 0.5 mL, 0.6 mL, 0.7 mL, 0.8 mL, 0.9 mL, 1 mL or the infusion may be 0.1-0.2 mL, 0.1-0.3 mL, 0.1-0.4 mL, 0.1-0.5 mL, 0.1-0.6 mL, 0.1-0.7 mL, 0.1-0.8 mL, 0.1-0.9 mL, 0.1-1 mL, 0.2-0.3 mL, 0.2-0.4 mL, 0.2-0.5 mL, 0.2-0.6 mL, 0.2-0.7 mL, 0.2-0.8 mL, 0.2-0.9 mL, 0.2-1 mL, 0.3-0.4 mL, 0.3-0.5 mL, 0.3-0.6 mL, 0.3-0.7 mL, 0.3-0.8 mL, 0.3-0.9 mL, 0.3-1 mL, 0.4-0.5 mL, 0.4-0.6 mL, 0.4-0.7 mL, 0.4-0.8 mL, 0.4-0.9 mL, 0.4-1 mL, 0.5-0.6 mL, 0.5-0.7 mL, 0.5-0.8 mL, 0.5-0.9 mL, 0.5-1 mL, 0.6-0.7 mL, 0.6-0.8 mL, 0.6-0.9 mL, 0.6-1 mL, 0.7-0.8 mL, 0.7-0.9 mL, 0.7-1 mL, 0.8-0.9 mL, 0.8-1 mL, or 0.9-1 mL.

In one embodiment, delivery of AAV particles to cells of the central nervous system (e.g., parenchyma) comprises infusion of between about 1 mL to about 120 mL. The infusion may be 1-5 mL, 1-10 mL, 1-15 mL, 1-20 mL, 1-25 mL, 1-30 mL, 1-35 mL, 1-40 mL, 1-45 mL, 1-50 mL, 1-55 mL, 1-60 mL, 1-65 mL, 1-70 mL, 1-75 mL, 1-80 mL, 1-85 mL, 1-90 mL, 1-95 mL, 1-100 mL, 1-105 mL, 1-110 mL, 1-115 mL, 1-120 mL, 5-10 mL, 5-15 mL, 5-20 mL, 5-25 mL, 1-30 mL, 5-35 mL, 5-40 mL, 5-45 mL, 5-50 mL, 5-55 mL, 5-60 mL, 5-65 mL, 5-70 mL, 5-75 mL, 5-80 mL, 5-85 mL, 5-90 mL, 5-95 mL, 5-100 mL, 5-105 mL, 5-110 mL, 5-115 mL, 1-120 mL, 10-15 mL, 10-20 mL, 10-25 mL, 10-30 mL, 10-35 mL, 10-40 mL, 10-45 mL, 10-50 mL, 10-55 mL, 10-60 mL, 10-65 mL, 10-70 mL, 10-75 mL, 10-80 mL, 10-85 mL, 10-90 mL, 10-95 mL, 10-100 mL, 10-105 mL, 10-110 mL, 10-115 mL, 10-120 mL 15-20 mL, 15-25 mL, 15-30 mL, 15-35 mL, 15-40 mL, 15-45 mL, 15-50 mL, 15-55 mL, 15-60 mL, 15-65 mL, 15-70 mL, 15-75 mL, 15-80 mL, 15-85 mL, 15-90 mL, 15-95 mL, 15-100 mL, 15-105 mL, 15-110 mL, 15-115 mL, 15-120 mL, 20-25 mL, 20-30 mL, 20-35 mL, 20-40 mL, 20-45 mL, 20-50 mL, 20-55 mL, 20-60 mL, 20-65 mL, 20-70 mL, 20-75 mL, 20-80 mL, 20-85 mL, 20-90 mL, 20-95 mL, 20-100 mL, 20-105 mL, 20-110 mL, 20-115 mL, 20-120 mL, 25-30 mL, 25-35 mL, 25-40 mL, 25-45 mL, 25-50 mL, 25-55 mL, 25-60 mL, 25-65 mL, 25-70 mL, 25-75 mL, 25-80 mL, 25-85 mL, 25-90 mL, 25-95 mL, 25-100 mL, 25-105 mL, 25-110 mL, 25-115 mL, 25-120 mL, 30-35 mL, 30-40 mL, 30-45 mL, 30-50 mL, 30-55 mL, 30-60 mL, 30-65 mL, 30-70 mL, 30-75 mL, 30-80 mL, 30-85 mL, 30-90 mL, 30-95 mL, 30-100 mL, 30-105 mL, 30-110 mL, 30-115 mL, 30-120 mL, 35-40 mL, 35-45 mL, 35-50 mL, 35-55 mL, 35-60 mL, 35-65 mL, 35-70 mL, 35-75 mL, 35-80 mL, 35-85 mL, 35-90 mL, 35-95 mL, 35-100 mL, 35-105 mL, 35-110 mL, 35-115 mL, 35-120 mL, 40-45 mL, 40-50 mL, 40-55 mL, 40-60 mL, 40-65 mL, 40-70 mL, 40-75 mL, 40-80 mL, 40-85 mL, 40-90 mL, 40-95 mL, 40-100 mL, 40-105 mL, 40-110 mL, 40-115 mL, 40-120 mL, 45-50 mL, 45-55 mL, 45-60 mL, 45-65 mL, 45-70 mL, 45-75 mL, 45-80 mL, 45-85 mL, 45-90 mL, 45-95 mL, 45-100 mL, 45-105 mL, 45-110 mL, 45-115 mL, 45-120 mL, 50-55 mL, 50-60 mL, 50-65 mL, 50-70 mL, 50-75 mL, 50-80 mL, 50-85 mL, 50-90 mL, 50-95 mL, 50-100 mL, 50-105 mL, 50-110 mL, 50-115 mL, 50-120 mL, 55-60 mL, 55-65 mL, 55-70 mL, 55-75 mL, 55-80 mL, 55-85 mL, 55-90 mL, 55-95 mL, 55-100 mL, 55-105 mL, 55-110 mL, 55-115 mL, 55-120 mL, 60-65 mL, 60-70 mL, 60-75 mL, 60-80 mL, 60-85 mL, 60-90 mL, 60-95 mL, 60-100 mL, 60-105 mL, 60-110 mL, 60-115 mL, 60-120 mL, 65-70 mL, 65-75 mL, 65-80 mL, 65-85 mL, 65-90 mL, 65-95 mL, 65-100 mL, 65-105 mL, 65-110 mL, 65-115 mL, 65-120 mL, 70-75 mL, 70-80 mL, 70-85 mL, 70-90 mL, 70-95 mL, 70-100 mL, 70-105 mL, 70-110 mL, 70-115 mL, 70-120 mL, 75-80 mL, 75-85 mL, 75-90 mL, 75-95 mL, 75-100 mL, 75-105 mL, 75-110 mL, 75-115 mL, 75-120 mL, 80-85 mL, 80-90 mL, 80-95 mL, 80-100 mL, 80-105 mL, 80-110 mL, 80-115 mL, 80-120 mL, 85-90 mL, 85-95 mL, 85-100 mL, 85-105 mL, 85-110 mL, 85-115 mL, 85-120 mL, 90-95 mL, 90-100 mL, 90-105 mL, 90-110 mL, 90-115 mL, 90-120 mL, 95-100 mL, 95-105 mL, 95-110 mL, 95-115 mL, 95-120 mL, 100-105 mL, 100-110 mL, 100-115 mL, 100-120 mL, 105-110 mL, 105-115 mL, 105-120 mL, 110-115 mL, 110-120 mL, or 115-120 mL.

In one embodiment, delivery of AAV particles to cells of the central nervous system (e.g., parenchyma) may comprise an infusion of about 0.1, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, or 120 mL. In one embodiment, delivery of AAV particles to cells of the central nervous system (e.g., parenchyma) comprises of infusion of 1 mL.

In one embodiment, delivery of AAV particles to cells of the central nervous system (e.g., parenchyma) comprises of infusion of at least 1 mL. In one embodiment, delivery of AAV particles to cells of the central nervous system (e.g., parenchyma) comprises infusion of at least 3 mL. In one embodiment, delivery of AAV particles to cells of the central nervous system (e.g., parenchyma) comprises of infusion of 3 mL. In one embodiment, delivery of AAV particles to cells of the central nervous system (e.g., parenchyma) comprises infusion of at least 10 mL. In one embodiment, delivery of AAV particles to cells of the central nervous system (e.g., parenchyma) consists of infusion of 10 mL.

### Infusion Compositions

In some embodiments, a composition comprising AAV particles delivered to cells of the central nervous system (e.g., parenchyma) may have a certain range of concentrations, pH, baricity (i.e. density of solution), osmolarity, temperature, and other physiochemical and biochemical properties that benefit the delivery of AAV particles to cells of the central nervous system (e.g., parenchyma).

### Duration of Infusion

### Bolus Infusion

In one embodiment, a subject may be administered the AAV particles described herein using a bolus infusion. As used herein, a “bolus infusion” means a single and rapid infusion of a substance or composition.

In one embodiment, delivery of AAV particles to cells of the central nervous system (e.g., parenchyma) comprises infusion by bolus injection with a duration of less than 30 minutes. In one embodiment, infusion by bolus injection comprises injection with a duration of less than 20 minutes. In one embodiment, infusion by bolus injection comprises injection with a duration of less than 10 minutes. In one embodiment, infusion by bolus injection comprises injection with a duration of less than 10 seconds. In one embodiment, infusion by bolus injection comprises injection with a duration of between 10 seconds to 10 minutes. In one embodiment, infusion by bolus injection comprises injection with a duration of 10 minutes. In one embodiment, infusion by bolus injection consists of injection with a duration of 10 minutes.

In one embodiment, delivery of AAV particles to cells of the central nervous system (e.g., parenchyma) comprises infusion by at least one bolus injection. In one embodiment, delivery may comprise infusion by 1, 2, 3, 4, 5, 6, 7, 8, 9, or 10 bolus injections. In one embodiment, delivery may comprise infusion by at least three bolus injections. In one embodiment, delivery comprises infusion by three bolus injections. In one embodiment, delivery of AAV to cells of the central nervous system (e.g., parenchyma) consists of infusion by three bolus injections.

In one embodiment, delivery of AAV particles to cells of the central nervous system (e.g., parenchyma) comprising infusion of more than one bolus injection further comprises an interval of at least one hour between injections. The interval may be 1, 2, 3, 4, 5, 6, 7, 8, 9, 10, 11, 12, 13, 14, 15, 16, 17, 18, 19, 20, 21, 22, 23, 24, 30, 36, 42, 48, 54, 60, 66, 72, 78, 84, 90, 96, 108, or 120 hour(s) between injections.

In one embodiment, delivery comprising infusion of more than one bolus injection further comprises an interval of one hour between injections.

In one embodiment, delivery consists of infusion by three bolus injections at an interval of one hour.

In one embodiment, delivery of the AAV particles described herein is a multi-level bolus with a controlled withdrawal of the catheter. As a non-limiting example, the initial administration of the AAV particles occurs at C2 and the final administration occurs at L5. As a non-limiting example, the administration of the AAV particles occurs at C2, C6, T6, L1 and the final administration occurs at L5.

### Prolonged or Continuous Infusion

In one embodiment, delivery of AAV particles to cells of the central nervous system (e.g., parenchyma) comprises prolonged or continuous infusion of pharmaceutically acceptable composition comprising AAV particles.

In one embodiment, delivery comprises prolonged infusion of one dose. In another embodiment, delivery comprises prolonged infusion of two or more doses.

In one embodiment, delivery of AAV particles to cells of the central nervous system (e.g., parenchyma) comprises prolonged or continuous infusion of pharmaceutically acceptable composition comprising AAV particles over a duration of at least 10 minutes. As used herein, continuous infusion, also referred to as prolonged infusion and prolonged continuous infusion, refer to a single infusion of a substance or composition over a period of time of at least 10 minutes.

In one embodiment, delivery comprises prolonged infusion over a duration of between 30 minutes and 60 minutes.

In one embodiment, delivery comprises prolonged infusion over a duration of one hour.

In one embodiment, delivery of AAV particles to cells of the central nervous system (e.g., parenchyma) consists of prolonged infusion over a duration of one hour.

In one embodiment, delivery may comprise prolonged infusion of over a duration of 0.17, 0.33, 0.5, 0.67, 0.83, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125 or more than 125 hour(s).

In one embodiment, delivery of AAV particles to cells of the central nervous system (e.g., parenchyma) comprises prolonged infusion over a duration of 10 hours. In one embodiment, delivery of AAV particles to cells of the central nervous system (e.g., parenchyma) consists of prolonged infusion over a duration of 10 hours. In one embodiment, prolonged infusion may yield more homogenous levels of protein expression across the spinal cord, as compared to bolus dosing at one or multiple sites. In one embodiment, dentate nucleus expression may increase with prolonged infusions.

In one embodiment, delivery of AAV particles to cells of the central nervous system (e.g., parenchyma) comprises prolonged infusion of at least one dose, or two or more doses. The interval between doses may be at least one hour, or between 1 hour and 120 hours.

In one embodiment, delivery of AAV particles to cells of the central nervous system (e.g., parenchyma) comprising prolonged infusion of more than one dose further comprises an interval of at least one hour between doses. In one embodiment, delivery may comprise an interval of 1, 2, 3, 4, 5, 6, 7, 8, 9, 10, 11, 12, 13, 14, 15, 16, 17, 18, 19, 20, 21, 22, 23, 24, 30, 36, 42, 48, 54, 60, 66, 72, 78, 84, 90, 96, 108, or 120 hour(s) between doses. In one embodiment, delivery comprises an interval of 24 hours between doses. In one embodiment, delivery consists of three prolonged infusion doses at an interval of 24 hours.

In one embodiment, delivery of AAV particles to cells of the central nervous system (e.g., parenchyma) comprises a rate of delivery may be defined by [VG/hour=mL/hour*VG/mL] wherein VG is viral genomes, VG/mL is composition concentration, and mL/hour is rate of prolonged infusion. In accordance with the present invention, the

In one embodiment, delivery of AAV to cells of the central nervous system (e.g., parenchyma) may comprise a rate of prolonged infusion between about 0.1 mL/hour and about 25.0 mL/hour (or higher if CSF pressure does not increase to dangerous levels). In some embodiments, delivery may comprise a rate of prolonged infusion of about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 10.1, 10.2, 10.3, 10.4, 10.5, 10.6, 10.7, 10.8, 10.9, 11.0, 11.1, 11.2, 11.3, 11.4, 11.5, 11.6, 11.7, 11.8, 11.9, 12.0, 12.1, 12.2, 12.3, 12.4, 12.5, 12.6, 12.7, 12.8, 12.9, 13.0, 13.1, 13.2, 13.3, 13.4, 13.5, 13.6, 13.7, 13.8, 13.9, 14.0, 14.1, 14.2, 14.3, 14.4, 14.5, 14.6, 14.7, 14.8, 14.9, 15.0, 15.1, 15.2, 15.3, 15.4, 15.5, 15.6, 15.7, 15.8, 15.9, 16.0, 16.1, 16.2, 16.3, 16.4, 16.5, 16.6, 16.7, 16.8, 16.9, 17.0, 17.1, 17.2, 17.3, 17.4, 17.5, 17.6, 17.7, 17.8, 17.9, 18.0, 18.1, 18.2, 18.3, 18.4, 18.5, 18.6, 18.7, 18.8, 18.9, 19.0, 19.1, 19.2, 19.3, 19.4, 19.5, 19.6, 19.7, 19.8, 19.9, 20.0, 20.1, 20.2, 20.3, 20.4, 20.5, 20.6, 20.7, 20.8, 20.9, 21.0, 21.1, 21.2, 21.3, 21.4, 21.5, 21.6, 21.7, 21.8, 21.9, 22.0, 22.1, 22.2, 22.3, 22.4, 22.5, 22.6, 22.7, 22.8, 22.9, 23.0, 23.1, 23.2, 23.3, 23.4, 23.5, 23.6, 23.7, 23.8, 23.9, 24.0, 24.1, 24.2, 24.3, 24.4, 24.5, 24.6, 24.7, 24.8, 24.9, or 25.0 mL/hour. In some embodiments, delivery may comprise a rate of prolonged infusion of about 10, 20 30, 40, or 50 mL/hr. In one embodiment, delivery of AAV particles to cells of the central nervous system (e.g., parenchyma) comprises a rate of prolonged infusion of 1.0 mL/hour. In one embodiment, delivery consists of a rate of prolonged infusion of 1.0 mL/hour. In one embodiment, delivery of AAV to cells of the central nervous system (e.g., parenchyma) comprises a rate of prolonged infusion of 1.5 mL/hour. In one embodiment, delivery of AAV particles to cells of the central nervous system (e.g., parenchyma) consists of a rate of prolonged infusion of 1.5 mL/hour.

In one embodiment, delivery of AAV particles to cells of the central nervous system (e.g., parenchyma) may comprise a constant rate of prolonged infusion. As used herein, a “constant rate” is a rate that stays about the same during the prolonged infusion.

In one embodiment, delivery of AAV particles to cells of the central nervous system (e.g., parenchyma) may comprise a ramped rate of prolonged infusion where the rate either increases or decreases over time. As a non-limiting example, the rate of prolonged infusion increases over time. As another non-limiting example, the rate of prolonged infusion decreases over time.

In one embodiment, delivery of AAV particles to cells of the central nervous system (e.g., parenchyma) may comprise a complex rate of prolonged infusion wherein the rate of prolonged infusion alternates between high and low rates of prolonged infusion over time.

### CSF Adsorption and Intercranial Pressure

In one embodiment, delivery of AAV particles to cells of the central nervous system (e.g., parenchyma) may comprise a rate of prolonged infusion wherein the rate of prolonged infusion exceeds the rate of CSF absorption. In some embodiments, CSF pressure may increase wherein the rate of delivery is greater than the rate of clearance. In one embodiment, increased CSF pressure may increase delivery of AAV particles to cells of the central nervous system (e.g., parenchyma of brain and spinal cord). In one embodiment, delivery of AAV to cells of the central nervous system (e.g., parenchyma) may comprise an increase in sustained CSF pressure between about 1% and about 25%. In some embodiments, delivery may comprise an increase in sustained CSF pressure of about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, or 25%.

In one embodiment, the intracranial pressure may be evaluated and adjusted (e.g., increased or decreased) prior to administration. The route, volume, AAV particle concentration, infusion duration and/or vector titer may be optimized based on the intracranial pressure of a subject.

### Combinations

The AAV particles may be used in combination with one or more other therapeutic, prophylactic, diagnostic, or imaging agents. By “in combination with,” it is not intended to imply that the agents must be administered at the same time and/or formulated for delivery together, although these methods of delivery are within the scope of the present disclosure. Compositions can be administered concurrently with, prior to, or subsequent to, one or more other desired therapeutics or medical procedures. In general, each agent will be administered at a dose and/or on a time schedule determined for that agent. In some embodiments, the present disclosure encompasses the delivery of pharmaceutical, prophylactic, diagnostic, or imaging compositions in combination with agents that may improve their bioavailability, reduce and/or modify their metabolism, inhibit their excretion, and/or modify their distribution within the body.

### Measurement of Expression

In one embodiment, the expression of the viral genomes, and/or payloads from the viral genomes described herein may be determined using various methods known in the art such as, but not limited to, immunochemistry (e.g., IHC), in situ hybridization (ISH), laser capture, qRT-PCR, ELISA, western blot, LCMS, Vg levels, Vg ISH, IHC/IF, or any combination thereof.

Expression of payloads from viral genomes may be determined using various methods known in the art such as, but not limited to immunochemistry (e.g., IHC) or in situ hybridization (ISH). In one embodiment, transgenes delivered in different AAV capsids may have different expression levels in Dorsal Root Ganglion (DRG). As a non-limiting example, the expression of FXN in DRG may be greatest in AAVDJ8 and lowest in AAV2 (AAVDJ8>AAVDJ>AAV6>scAAVrh10>ssAAVrh10>AAV9>AAV5>AAV2).

### Methods of the Present Invention

The present disclosure provides a method for treating a disease, disorder and/or condition in a mammalian subject, including a human subject, comprising administering to the subject any of the viral particles e.g., AAV, AAV particles or AAV genomes described herein (i.e., viral genomes or “VG”) or administering to the subject a particle comprising said AAV particle or AAV genome, or administering to the subject any of the described compositions, including pharmaceutical compositions. In one embodiment, the disease, disorder and/or condition is a neurological disease, disorder and/or condition. The CNS diseases may be diseases that affect any component of the brain (including the cerebral hemispheres, diencephalon, brain stem, and cerebellum) or the spinal cord.

In some embodiments, AAV particles of the present invention, through delivery of a function payload that is a therapeutic product that can modulate the level or function of a gene product in the CNS, may be used to treat a neurodegenerative diseases and/or diseases or disorders that are characteristic with neurodegeneration, neuromuscular diseases, lysosomal diseases, trauma, bone marrow injuries, pain (including neuropathic pain), cancers of the nervous system, demyelinating diseases, autoimmune diseases of the nervous system, neurotoxic syndromes, sleeping disorders genetic brain disorders and developmental CNS disorders. A functional payload may alleviate or reduce symptoms that result from abnormal level and/or function of a gene product (e.g., an absence or defect in a protein) in a subject in need thereof or that otherwise confers a benefit to a CNS disorder in a subject in need thereof.

As non-limiting examples, therapeutic products delivered by AAV particles of the present invention may include, but are not limited to, growth and trophic factors, cytokines, hormones, neurotransmitters, enzymes, anti-apoptotic factors, angiogenic factors, and any protein known to be mutated in pathological disorders such as the “survival of motor neuron” protein (SMN); antisense RNA or RNAi targeting messenger RNAs coding for proteins having a therapeutic interest in any of CNS diseases discussed herein; or microRNAs that function in gene silencing and post-transcriptionally regulation of gene expression in the CNS (e.g., brain specific Mir-128a, See Adlakha and Saini, Molecular cancer, 2014, 13:33).

The growth and trophic factors may include, but are not limited to brain-derived growth factor (BDNF), epidermal growth factor (EGF), basic Fibroblast growth factor (bFGF), Ciliary neurotrophic factor (CNTF), corticotropin-releasing factor (CRF), Glial cell line derived growth factor (GDNF), Insulin-like growth factor-1 (IGF-1), nerve growth factor (NGF), neurotrophin-3 (NT-3), neurotrophin-4 (NT-4), and vascular endothelial growth factor (VEGF). Cytokines may include interleukin-10 (IL-10), interleukin-6, Interleukin-8, chemokine CXCL12 (SDF-1), TGF-beta, and Growth and differentiation factor (GDF-1/10).

In some embodiments, the neurological disorders may be neurodegenerative disorders including, but not limited to, Alzheimer's Diseases (AD), Amyotrophic lateral sclerosis (ALS), Creutzfeldt-Jakob Disease, Huntingtin's disease (HD), Friedreich's ataxia (FA), Parkinson Disease (PD), Multiple System Atrophy (MSA), Spinal Muscular Atrophy (SMA), Multiple Sclerosis (MS), Primary progressive aphasia, Progressive supranuclear palsy, Dementia, Brain Cancer, Degenerative Nerve Diseases, Encephalitis, Epilepsy, Genetic Brain Disorders that cause neurodegeneration, Retinitis pigmentosa (RP), Head and Brain Malformations, Hydrocephalus, Stroke, Prion disease, Infantile neuronal ceroid lipofuscinosis (INCL) (a neurodegenerative disease of children caused by a deficiency in the lysosomal enzyme palmitoyl protein thioesterase-1 (PPT1)).

In some embodiments, AAV particles of the present invention may be used to treat diseases that are associated with impairments of the growth and development of the CNS, i.e., neurodevelopmental disorders. In some aspects, such neurodevelopmental disorders may be caused by genetic mutations, including but not limited to, Fragile X syndrome (caused by mutations in FMR1 gene), Down syndrome (caused by trisomy of chromosome 21), Rett syndrome, Williams syndrome, Angelman syndrome, Smith-Magenis syndrome, ATR-X syndrome, Barth syndrome, Immune dysfunction and/or infectious diseases during infancy such as Sydenham's chorea, Schizophrenia Congenital toxoplasmosis, Congenital rubella syndrome, Metabolic disorders such as diabetes mellitus and phenylketonuria; nutritional defects and/or brain trauma, Autism and autism spectrum.

In some embodiments, AAV particles of the present invention, may be used to treat a tumor in the CNS, including but not limited to, acoustic neuroma, Astrocytoma (Grades I, II, III and IV), Chordoma, CNS Lymphoma, Craniopharyngioma, Gliomas (e.g., brain stem glioma, ependymoma, optical nerve glioma, subependymoma), Medulloblastoma, Meningioma, Metastatic brain tumors, Oligodendroglioma, Pituitary Tumors, Primitive neuroectodermal (PNET), and Schwannoma.

In some embodiments, the neurological disorders may be functional neurological disorders with motor and/or sensory symptoms which have neurological origin in the CNS. As non-limiting examples, functional neurological disorders may be chronic pain, seizures, speech problems, involuntary movements, and sleep disturbances.

In some embodiments, the neurological disorders may be white matter disorders (a group of diseases that affects nerve fibers in the CNS) including but not limited to, Pelizaeus-Merzbacher disease, Hypomyelination with atrophy of basal ganglia and cerebellum, Aicardi-Goutières syndrome, Megalencephalic leukoencephalopathy with subcortical cysts, Congenital muscular dystrophies, Myotonic dystrophy, Wilson disease, Lowe syndrome, Sjögren-Larsson syndrome, PIBD or Tay syndrome, Cockayne's disease, erebrotendinous xanthomatosis, Zellweger syndrome, Neonatal adrenoleukodystrophy, Infantile Refsum disease, Zellweger-like syndrome, Pseudo-Zellweger syndrome, Pseudo-neonatal adrenoleukodystrophy, Bifunctional protein deficiency, X-linked adrenoleukodystrophy and adrenomyeloneuropathy and Refsum disease.

In some embodiments, the neurological disorders may be lysosomal storage disorders (LSDs) caused by the inability of cells in the CNS to break down metabolic end products, including but not limited to Niemann-Pick disease (a LSD resulting from inherited deficiency in acid sphingomyelinase (ASM); Metachromatic leukodystrophy (MLD) (a LSD characterized by accumulation of sulfatides in glial cells and neurons, the result of an inherited deficiency of arylsulfatase A (ARSA)); Globoid-cell leukodystrophy (GLD) (a LSD caused by mutations in galactosylceramidase); Fabry disease (a LSD caused by mutations in the alpha-galactosidase A (GLA) gene); Gaucher disease (caused by mutations in the beta-glucocerebrosidase (GBA) gene); GM1/GM2 gangliosidosis; Mucopolysaccharidoses disorder; Pompe disease; and Neuronal ceroid lipofuscinosis.

In one embodiment, the neurological disease, disorder and/or condition is Parkinson's disease. In one embodiment the polynucleotide used to treat Parkinson's disease comprises any one of SEQ ID NOs 570-662 wherein the payload is replaced by AADC or any other payload known in the art for treating Parkinson's disease. As a non-limiting example, the payload may be a sequence such as NM_001082971.1 (GI: 132814447), NM_000790.3 (GI: 132814459), NM_001242886.1 (GI: 338968913), NM_001242887.1 (GI: 338968916), NM_001242888.1 (GI: 338968918), NM_001242889.1 (GI: 338968920), NM_001242890.1 (GI: 338968922) and fragment or variants thereof.

In another embodiment, the neurological disease, disorder and/or condition is Friedreich's Ataxia. In one embodiment, the delivery of the AAV particles may halt or slow the disease progression of Friedreich's Ataxia by 10%, 20%, 30%, 40%, 50%, 60%, 70%, 80%, 90%, 95% or more than 95% using a known analysis method and comparator group for Friedreich's Ataxia. As a non-limiting example, the delivery of the AAV particles may halt or slow progression of Friedreich's Ataxia progression as measured by mFARS/SARA by 50% relative to a comparator group. In one embodiment the polynucleotide used to treat Friedreich's Ataxia comprises any one of SEQ ID NOs 570-662 wherein the payload is replaced by Frataxin or any other payload known in the art for treating Friedreich's Ataxia. As a non-limiting example, the payload may be a sequence such as NM_000144.4 (GI: 239787167), NM_181425.2 (GI: 239787185), NM_001161706.1 (GI: 239787197) and fragment or variants thereof.

In another embodiment, the neurological disease, disorder and/or condition is Amyotrophic lateral sclerosis (ALS). In one embodiment, the delivery of the AAV particles may halt or slow the disease progression of ALS by 10%, 20%, 30%, 40%, 50%, 60%, 70%, 80%, 90%, 95% or more than 95% using a known analysis method and comparator group for ALS. In one embodiment the polynucleotide used to treat ALS comprises any one of SEQ ID NOs 570-662 wherein the payload is replaced by an shRNA, miRNA, siRNA, RNAi for SOD1 or any other payload known in the art for treating ALS.

In another embodiment, the neurological disease, disorder and/or condition is Huntington's disease. In one embodiment, the delivery of the AAV particles may halt or slow the disease progression of Huntington's disease by 10%, 20%, 30%, 40%, 50%, 60%, 70%, 80%, 90%, 95% or more than 95% using a known analysis method and comparator group for Huntington's disease. In one embodiment the polynucleotide used to treat Huntington's disease comprises any one of SEQ ID NOs 570-662 wherein the payload is replaced by an shRNA, miRNA, siRNA, RNAi for Htt or any other payload known in the art for treating Huntington's disease.

In another embodiment, the neurological disease, disorder or condition is spinal muscular atrophy (SMA). In one embodiment, the delivery of the AAV particles may halt or slow the disease progression of SMA by 10%, 20%, 30%, 40%, 50%, 60%, 70%, 80%, 90%, 95% or more than 95% using a known analysis method and comparator group for SMA. In one embodiment the polynucleotide used to treat SMA comprises any one of SEQ ID NOs 570-662 wherein the payload is replaced by SMN or any other payload known in the art for treating SMA. As a non-limiting example, the payload may be a sequence such as NM_001297715.1 (GI: 663070993), NM_000344.3 (GI: 196115055), NM_022874.2 (GI: 196115040) and fragment or variants thereof.

In one embodiment, the AAV particle encoding a payload may increase the amount of protein encoded by the payload (e.g., transgene) by 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, 97%, 99% or more than 100%.

In one embodiment, the AAV particle encoding a payload may increase the amount of protein encoded by the payload (e.g., transgene) by 1-5%, 1-10%, 1-15%, 1-20%, 5-10%, 5-15%, 5-20%, 5-25%, 10-20%, 10-30%, 15-35%, 20-40%, 20-50%, 30-50%, 30-60%, 40-60%, 40-70%, 50-60%, 50-70%, 60-80%, 60-90%, 70-80%, 70-90%, 80-90%, 80-99% or 90-100%.

In one embodiment, the AAV particles may be delivered to a subject to improve and/or correct mitochondrial dysfunction.

In one embodiment, the AAV particles may be delivered to a subject to preserve neurons. The neurons may be primary and/or secondary sensor neurons.

In one embodiment, administration of the AAV particles may preserve and/or correct function in the sensory pathways.

In one embodiment, administration of the AAV particles may protect central pathways from degeneration. As a non-limiting example, the degeneration is later onset degeneration of auditory pathways.

### Definitions

At various places in the present specification, substituents of compounds of the present disclosure are disclosed in groups or in ranges. It is specifically intended that the present disclosure include each and every individual sub-combination of the members of such groups and ranges. The following is a non-limiting list of term definitions.

Adeno-associated virus: The term “adeno-associated virus” or “AAV” as used herein refers to members of the dependovirus genus comprising any particle, sequence, gene, protein, or component derived therefrom. The term “AAV particle” as used herein comprises a capsid and a polynucleotide referred to as the AAV genome or viral genome (VG). The AAV particle may be derived from any serotype, described herein or known in the art, including combinations of serotypes (i.e., “pseudotyped” AAV) or from various genomes (e.g., single stranded or self-complementary). In addition, the AAV particle may be replication defective and/or targeted.

Activity: As used herein, the term “activity” refers to the condition in which things are happening or being done. Compositions of the invention may have activity and this activity may involve one or more biological events.

Administered in combination: As used herein, the term “administered in combination” or “combined administration” refers to simultaneous exposure to two or more agents (e.g., AAV) administered at the same time or within an interval such that the subject is at some point in time simultaneously exposed to both and/or such that there may be an overlap in the effect of each agent on the patient. In some embodiments, at least one dose of one or more agents is administered within about 24 hours, 12 hours, 6 hours, 3 hours, 1 hour, 30 minutes, 15 minutes, 10 minutes, 5 minutes, or 1 minute of at least one dose of one or more other agents. In some embodiments, administration occurs in overlapping dosage regimens. As used herein, the term “dosage regimen” refers to a plurality of doses spaced apart in time. Such doses may occur at regular intervals or may include one or more hiatus in administration. In some embodiments, the administration of individual doses of one or more compounds and/or compositions of the present invention, as described herein, are spaced sufficiently closely together such that a combinatorial (e.g., a synergistic) effect is achieved.

Amelioration: As used herein, the term “amelioration” or “ameliorating” refers to a lessening of severity of at least one indicator of a condition or disease. For example, in the context of neurodegeneration disorder, amelioration includes the reduction of neuron loss.

Animal: As used herein, the term “animal” refers to any member of the animal kingdom. In some embodiments, “animal” refers to humans at any stage of development. In some embodiments, “animal” refers to non-human animals at any stage of development. In certain embodiments, the non-human animal is a mammal (e.g., a rodent, a mouse, a rat, a rabbit, a monkey, a dog, a cat, a sheep, cattle, a primate, or a pig). In some embodiments, animals include, but are not limited to, mammals, birds, reptiles, amphibians, fish, and worms. In some embodiments, the animal is a transgenic animal, genetically-engineered animal, or a clone.

Antisense strand: As used herein, the term “the antisense strand” or “the first strand” or “the guide strand” of a siRNA molecule refers to a strand that is substantially complementary to a section of about 10-50 nucleotides, e.g., about 15-30, 16-25, 18-23 or 19-22 nucleotides of the mRNA of the gene targeted for silencing. The antisense strand or first strand has sequence sufficiently complementary to the desired target mRNA sequence to direct target-specific silencing, e.g., complementarity sufficient to trigger the destruction of the desired target mRNA by the RNAi machinery or process.

Approximately: As used herein, the term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Associated with: As used herein, the terms “associated with,” “conjugated,” “linked,” “attached,” and “tethered,” when used with respect to two or more moieties, mean that the moieties are physically associated or connected with one another, either directly or via one or more additional moieties that serve as linking agents, to form a structure that is sufficiently stable so that the moieties remain physically associated under the conditions in which the structure is used, e.g., physiological conditions. An “association” need not be strictly through direct covalent chemical bonding. It may also suggest ionic or hydrogen bonding or a hybridization based connectivity sufficiently stable such that the “associated” entities remain physically associated.

Biomolecule: As used herein, the term “biomolecule” is any natural molecule which is amino acid-based, nucleic acid-based, carbohydrate-based or lipid-based, and the like.

Biologically active: As used herein, the phrase “biologically active” refers to a characteristic of any substance (e.g., an AAV) that has activity in or on a biological system and/or organism. For instance, a substance that, when administered to an organism, has a biological effect on that organism, is considered to be biologically active. In particular embodiments, a compounds and/or compositions of the present invention may be considered biologically active if even a portion of is biologically active or mimics an activity considered to biologically relevant.

Biological system: As used herein, the term “biological system” refers to a group of organs, tissues, cells, intracellular components, proteins, nucleic acids, molecules (including, but not limited to biomolecules) that function together to perform a certain biological task within cellular membranes, cellular compartments, cells, tissues, organs, organ systems, multicellular organisms, or any biological entity. In some embodiments, biological systems are cell signaling pathways comprising intracellular and/or extracellular cell signaling biomolecules. In some embodiments, biological systems comprise growth factor signaling events within the extracellular/cellular matrix and/or cellular niches.

Central Nervous System or CNS: As used herein, “Central Nervous System” or “CNS” refers to one of the two major subdivisions of the nervous system, which in vertebrates includes of the brain and spinal cord. The central nervous system coordinates the activity of the entire nervous system.

CNS tissue: As used herein, “CNS tissue” or “CNS tissues” refers to the tissues of the central nervous system, which in vertebrates, include the brain and spinal cord and sub-structures thereof.

CNS structures: As used herein, “CNS structures” refers to structures of the central nervous system and sub-structures thereof. Non-limiting examples of structures in the spinal cord may include, ventral horn, dorsal horn, white matter, and nervous system pathways or nuclei within. Non limiting examples of structures in the brain include, forebrain, midbrain, hindbrain, diencephalon, telencephalon, myelencepphalon, metencephalon, mesencephalon, prosencephalon, rhombencephalon, cortices, frontal lobe, parietal lobe, temporal lobe, occipital lobe, cerebrum, thalamus, hypothalamus, tectum, tegmentum, cerebellum, pons, medulla, amygdala, hippocampus, basal ganglia, corpus callosum, pituitary gland, ventricles and sub-structures thereof.

CNS Cells: As used herein, “CNS Cells” refers to cells of the central nervous system and sub-structures thereof. Non-limiting examples of CNS cells include, neurons and sub-types thereof, glia, microglia, oligodendrocytes, ependymal cells and astrocytes. Non-limiting examples of neurons include sensory neurons, motor neurons, interneurons, unipolar cells, bipolar cells, multipolar cells, psuedounipolar cells, pyramidal cells, basket cells, stellate cells, purkinje cells, betz cells, amacrine cells, granule cell, ovoid cell, medium aspiny neurons and large aspiny neurons.

Complementary and substantially complementary: As used herein, the term “complementary” refers to the ability of polynucleotides to form base pairs with one another. Base pairs are typically formed by hydrogen bonds between nucleotide units in antiparallel polynucleotide strands. Complementary polynucleotide strands can form base pair in the Watson-Crick manner (e.g., A to T, A to U, C to G), or in any other manner that allows for the formation of duplexes. As persons skilled in the art are aware, when using RNA as opposed to DNA, uracil rather than thymine is the base that is considered to be complementary to adenosine. However, when a U is denoted in the context of the present invention, the ability to substitute a T is implied, unless otherwise stated. Perfect complementarity or 100% complementarity refers to the situation in which each nucleotide unit of one polynucleotide strand can form hydrogen bond with a nucleotide unit of a second polynucleotide strand. Less than perfect complementarity refers to the situation in which some, but not all, nucleotide units of two strands can form hydrogen bond with each other. For example, for two 20-mers, if only two base pairs on each strand can form hydrogen bond with each other, the polynucleotide strands exhibit 10% complementarity. In the same example, if 18 base pairs on each strand can form hydrogen bonds with each other, the polynucleotide strands exhibit 90% complementarity. As used herein, the term “substantially complementary” means that the siRNA has a sequence (e.g., in the antisense strand) which is sufficient to bind the desired target mRNA, and to trigger the RNA silencing of the target mRNA.

Composition: As used herein, the term “composition” comprises a polynucleotide, viral genome or AAV particle and at least one excipient.

Compound: As used herein, the term “compound,” refers to a distinct chemical entity. In some embodiments, a particular compound may exist in one or more isomeric or isotopic forms (including, but not limited to stereoisomers, geometric isomers and isotopes). In some embodiments, a compound is provided or utilized in only a single such form. In some embodiments, a compound is provided or utilized as a mixture of two or more such forms (including, but not limited to a racemic mixture of stereoisomers). Those of skill in the art appreciate that some compounds exist in different such forms, show different properties and/or activities (including, but not limited to biological activities). In such cases it is within the ordinary skill of those in the art to select or avoid particular forms of the compound for use in accordance with the present invention. For example, compounds that contain asymmetrically substituted carbon atoms can be isolated in optically active or racemic forms. Methods on how to prepare optically active forms from optically active starting materials are known in the art, such as by resolution of racemic mixtures or by stereoselective synthesis.

Conserved: As used herein, the term “conserved” refers to nucleotides or amino acid residues of polynucleotide or polypeptide sequences, respectively, that are those that occur unaltered in the same position of two or more sequences being compared. Nucleotides or amino acids that are relatively conserved are those that are conserved among more related sequences than nucleotides or amino acids appearing elsewhere in the sequences.

In some embodiments, two or more sequences are said to be “completely conserved” if they are 100% identical to one another. In some embodiments, two or more sequences are said to be “highly conserved” if they are at least 70% identical, at least 80% identical, at least 90% identical, or at least 95% identical to one another. In some embodiments, two or more sequences are said to be “highly conserved” if they are about 70% identical, about 80% identical, about 90% identical, about 95%, about 98%, or about 99% identical to one another. In some embodiments, two or more sequences are said to be “conserved” if they are at least 30% identical, at least 40% identical, at least 50% identical, at least 60% identical, at least 70% identical, at least 80% identical, at least 90% identical, or at least 95% identical to one another. In some embodiments, two or more sequences are said to be “conserved” if they are about 30% identical, about 40% identical, about 50% identical, about 60% identical, about 70% identical, about 80% identical, about 90% identical, about 95% identical, about 98% identical, or about 99% identical to one another. Conservation of sequence may apply to the entire length of an oligonucleotide or polypeptide or may apply to a portion, region or feature thereof.

In one embodiment, conserved sequences are not contiguous. Those skilled in the art are able to appreciate how to achieve alignment when gaps in contiguous alignment are present between sequences, and to align corresponding residues not withstanding insertions or deletions present.

Delivery: As used herein, “delivery” refers to the act or manner of delivering a parvovirus e.g., AAV compound, substance, entity, moiety, cargo or payload to a target. Such target may be a cell, tissue, organ, organism, or system (whether biological or production).

Delivery Agent: As used herein, “delivery agent” refers to any agent which facilitates, at least in part, the delivery of one or more substances (including, but not limited to a compounds and/or compositions of the present invention, e.g., viral particles or AAV particles) to targeted cells.

Destabilized: As used herein, the term “destable,” “destabilize,” or “destabilizing region” means a region or molecule that is less stable than a starting, reference, wild-type or native form of the same region or molecule.

Detectable label: As used herein, “detectable label” refers to one or more markers, signals, or moieties which are attached, incorporated or associated with another entity, which markers, signals or moieties are readily detected by methods known in the art including radiography, fluorescence, chemiluminescence, enzymatic activity, absorbance, immunological detection and the like. Detectable labels may include radioisotopes, fluorophores, chromophores, enzymes, dyes, metal ions, ligands, biotin, avidin, streptavidin and haptens, quantum dots, polyhistidine tags, myc tags, flag tags, human influenza hemagglutinin (HA) tags and the like. Detectable labels may be located at any position in the entity with which they are attached, incorporated or associated. For example, when attached, incorporated in or associated with a peptide or protein, they may be within the amino acids, the peptides, or proteins, or located at the N- or C-termini.

Effective amount: As used herein, the term “effective amount” of an agent is that amount sufficient to effect beneficial or desired results, for example, upon single or multiple dose administration to a subject or a cell, in curing, alleviating, relieving or improving one or more symptoms of a disorder and, as such, an “effective amount” depends upon the context in which it is being applied. For example, in the context of administering an agent that treats ALS, an effective amount of an agent is, for example, an amount sufficient to achieve treatment, as defined herein, of ALS, as compared to the response obtained without administration of the agent.

Engineered: As used herein, embodiments of the invention are “engineered” when they are designed to have a feature or property, whether structural or chemical, that varies from a starting point, wild-type or native molecule. Thus, engineered agents or entities are those whose design and/or production include an act of the hand of man.

Epitope: As used herein, an “epitope” refers to a surface or region on a molecule that is capable of interacting with a biomolecule. For example a protein may contain one or more amino acids, e.g., an epitope, which interacts with an antibody, e.g., a biomolecule. In some embodiments, when referring to a protein or protein module, an epitope may comprise a linear stretch of amino acids or a three dimensional structure formed by folded amino acid chains.

Expression: As used herein, “expression” of a nucleic acid sequence refers to one or more of the following events: (1) production of an RNA template from a DNA sequence (e.g., by transcription); (2) processing of an RNA transcript (e.g., by splicing, editing, 5′ cap formation, and/or 3′ end processing); (3) translation of an RNA into a polypeptide or protein; (4) folding of a polypeptide or protein; and (5) post-translational modification of a polypeptide or protein.

Feature: As used herein, a “feature” refers to a characteristic, a property, or a distinctive element.

Formulation: As used herein, a “formulation” includes at least a compound and/or composition of the present invention (e.g., a vector, AAV particle, etc.) and a delivery agent.

Fowler's Position: As used herein, a subject tin the “Fowler's position” is sitting straight up or leaning slightly back with legs which may be straight or bent. A “high fowlers” position is somewhat who is sitting upright. A “low fowlers” position is someone whose head is only slightly elevated.

Fragment: A “fragment,” as used herein, refers to a contiguous portion of a whole. For example, fragments of proteins may comprise polypeptides obtained by digesting full-length protein isolated from cultured cells. In some embodiments, a fragment of a protein includes at least 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 150, 200, 250 or more amino acids.

Functional: As used herein, a “functional” biological molecule is a biological entity with a structure and in a form in which it exhibits a property and/or activity by which it is characterized.

Gene expression: The term “gene expression” refers to the process by which a nucleic acid sequence undergoes successful transcription and in most instances translation to produce a protein or peptide. For clarity, when reference is made to measurement of “gene expression”, this should be understood to mean that measurements may be of the nucleic acid product of transcription, e.g., RNA or mRNA or of the amino acid product of translation, e.g., polypeptides or peptides. Methods of measuring the amount or levels of RNA, mRNA, polypeptides and peptides are well known in the art.

High Cervical Region: As used herein, the term “high cervical region” refers to the region of the spinal cord comprising the cervical vertebrae C1, C2, C3 and C4 or any subset thereof.

Homology: As used herein, the term “homology” refers to the overall relatedness between polymeric molecules, e.g. between nucleic acid molecules (e.g. DNA molecules and/or RNA molecules) and/or between polypeptide molecules. In some embodiments, polymeric molecules are considered to be “homologous” to one another if their sequences are at least 25%, 30%, 35%, 40%, 45%, 50%, 55%, 60%, 65%, 70%, 75%, 80%, 85%, 90%, 95%, or 99% identical or similar. The term “homologous” necessarily refers to a comparison between at least two sequences (polynucleotide or polypeptide sequences). In accordance with the invention, two polynucleotide sequences are considered to be homologous if the polypeptides they encode are at least about 50%, 60%, 70%, 80%, 90%, 95%, or even 99% for at least one stretch of at least about 20 amino acids. In some embodiments, homologous polynucleotide sequences are characterized by the ability to encode a stretch of at least 4-5 uniquely specified amino acids. For polynucleotide sequences less than 60 nucleotides in length, homology is typically determined by the ability to encode a stretch of at least 4-5 uniquely specified amino acids. In accordance with the invention, two protein sequences are considered to be homologous if the proteins are at least about 50%, 60%, 70%, 80%, or 90% identical for at least one stretch of at least about 20 amino acids. In many embodiments, homologous protein may show a large overall degree of homology and a high degree of homology over at least one short stretch of at least 3, 4, 5, 6, 7, 8, 9, 10, 11, 12, 13, 14, 15, 16, 17, 18, 19, 20, 25, 30, 35, 40, 45, 50 or more amino acids. In many embodiments, homologous proteins share one or more characteristic sequence elements. As used herein, the term “characteristic sequence element” refers to a motif present in related proteins. In some embodiments, the presence of such motifs correlates with a particular activity (such as biological activity).

Identity: As used herein, the term “identity” refers to the overall relatedness between polymeric molecules, e.g., between oligonucleotide molecules (e.g. DNA molecules and/or RNA molecules) and/or between polypeptide molecules. Calculation of the percent identity of two polynucleotide sequences, for example, may be performed by aligning the two sequences for optimal comparison purposes (e.g., gaps can be introduced in one or both of a first and a second nucleic acid sequences for optimal alignment and non-identical sequences can be disregarded for comparison purposes). In certain embodiments, the length of a sequence aligned for comparison purposes is at least 30%, at least 40%, at least 50%, at least 60%, at least 70%, at least 80%, at least 90%, at least 95%, or 100% of the length of the reference sequence. The nucleotides at corresponding nucleotide positions are then compared. When a position in the first sequence is occupied by the same nucleotide as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a mathematical algorithm. For example, the percent identity between two nucleotide sequences can be determined using methods such as those described in Computational Molecular Biology, Lesk, A. M., ed., Oxford University Press, New York, 1988; Biocomputing: Informatics and Genome Projects, Smith, D. W., ed., Academic Press, New York, 1993; Sequence Analysis in Molecular Biology, von Heinje, G., Academic Press, 1987; Computer Analysis of Sequence Data, Part I, Griffin, A. M., and Griffin, H. G., eds., Humana Press, New Jersey, 1994; and Sequence Analysis Primer, Gribskov, M. and Devereux, J., eds., M Stockton Press, New York, 1991; each of which is incorporated herein by reference in its entirety. For example, the percent identity between two nucleotide sequences can be determined, for example using the algorithm of Meyers and Miller (CABIOS, 1989, 4:11-17), which has been incorporated into the ALIGN program (version 2.0) using a PAM120 weight residue table, a gap length penalty of 12 and a gap penalty of 4. The percent identity between two nucleotide sequences can, alternatively, be determined using the GAP program in the GCG software package using an NWSgapdna.CMP matrix. Methods commonly employed to determine percent identity between sequences include, but are not limited to those disclosed in Carillo, H., and Lipman, D., SIAM J Applied Math., 48:1073 (1988); incorporated herein by reference in its entirety. Techniques for determining identity are codified in publicly available computer programs. Computer software to determine homology between two sequences include, but are not limited to, GCG program package, Devereux, J., et al., Nucleic Acids Research, 12(1), 387 (1984)), BLASTP, BLASTN, and FASTA Altschul, S. F. et al., J. Molec. Biol., 215, 403 (1990)).

Inhibit expression of a gene: As used herein, the phrase “inhibit expression of a gene” means to cause a reduction in the amount of an expression product of the gene. The expression product may be RNA transcribed from the gene (e.g. mRNA) or a polypeptide translated from mRNA transcribed from the gene. Typically a reduction in the level of mRNA results in a reduction in the level of a polypeptide translated therefrom. The level of expression may be determined using standard techniques for measuring mRNA or protein.

In vitro: As used herein, the term “in vitro” refers to events that occur in an artificial environment, e.g., in a test tube or reaction vessel, in cell culture, in a Petri dish, etc., rather than within an organism (e.g., animal, plant, or microbe).

In vivo: As used herein, the term “in vivo” refers to events that occur within an organism (e.g., animal, plant, or microbe or cell or tissue thereof).

Isolated: As used herein, the term “isolated” is synonymous with “separated”, but carries with it the inference separation was carried out by the hand of man. In one embodiment, an isolated substance or entity is one that has been separated from at least some of the components with which it was previously associated (whether in nature or in an experimental setting). Isolated substances may have varying levels of purity in reference to the substances from which they have been associated. Isolated substances and/or entities may be separated from at least about 10%, about 20%, about 30%, about 40%, about 50%, about 60%, about 70%, about 80%, about 90%, or more of the other components with which they were initially associated. In some embodiments, isolated agents are more than about 80%, about 85%, about 90%, about 91%, about 92%, about 93%, about 94%, about 95%, about 96%, about 97%, about 98%, about 99%, or more than about 99% pure. As used herein, a substance is “pure” if it is substantially free of other components.

Substantially isolated: By “substantially isolated” is meant that the compound is substantially separated from the environment in which it was formed or detected. Partial separation can include, for example, a composition enriched in the compound of the present disclosure. Substantial separation can include compositions containing at least about 50%, at least about 60%, at least about 70%, at least about 80%, at least about 90%, at least about 95%, at least about 97%, or at least about 99% by weight of the compound of the present disclosure, or salt thereof. Methods for isolating compounds and their salts are routine in the art. In some embodiments, isolation of a substance or entity includes disruption of chemical associations and/or bonds. In some embodiments, isolation includes only the separation from components with which the isolated substance or entity was previously combined and does not include such disruption.

Left Lateral Recumbent Position: As used herein, “Left Lateral Recumbent” or LLR position refers to a subject laying on their left side.

Lumbar Region: As used herein, the term “lumbar region” refers to the region of the spinal cord comprising the lumbar vertebrae L1, L2, L3, L4, and L5.

Modified: As used herein, the term “modified” refers to a changed state or structure of a molecule or entity as compared with a parent or reference molecule or entity. Molecules may be modified in many ways including chemically, structurally, and functionally. In some embodiments, compounds and/or compositions of the present invention are modified by the introduction of non-natural amino acids, or non-natural nucleotides.

Mutation: As used herein, the term “mutation” refers to a change and/or alteration. In some embodiments, mutations may be changes and/or alterations to proteins (including peptides and polypeptides) and/or nucleic acids (including polynucleic acids). In some embodiments, mutations comprise changes and/or alterations to a protein and/or nucleic acid sequence. Such changes and/or alterations may comprise the addition, substitution and or deletion of one or more amino acids (in the case of proteins and/or peptides) and/or nucleotides (in the case of nucleic acids and or polynucleic acids). In embodiments wherein mutations comprise the addition and/or substitution of amino acids and/or nucleotides, such additions and/or substitutions may comprise 1 or more amino acid and/or nucleotide residues and may include modified amino acids and/or nucleotides.

Naturally occurring: As used herein, “naturally occurring” or “wild-type” means existing in nature without artificial aid, or involvement of the hand of man.

Non-human vertebrate: As used herein, a “non-human vertebrate” includes all vertebrates except Homo sapiens, including wild and domesticated species. Examples of non-human vertebrates include, but are not limited to, mammals, such as alpaca, banteng, bison, camel, cat, cattle, deer, dog, donkey, gayal, goat, guinea pig, horse, llama, mule, pig, rabbit, reindeer, sheep water buffalo, and yak.

Nucleic acid: As used herein, the term “nucleic acid”, “polynucleotide” and ‘oligonucleotide” refer to any nucleic acid polymers composed of either polydeoxyribonucleotides (containing 2-deoxy-D-ribose), or polyribonucleotides (containing D-ribose), or any other type of polynucleotide which is an N glycoside of a purine or pyrimidine base, or modified purine or pyrimidine bases. There is no intended distinction in length between the term “nucleic acid”, “polynucleotide” and “oligonucleotide”, and these terms will be used interchangeably. These terms refer only to the primary structure of the molecule. Thus, these terms include double- and single-stranded DNA, as well as double- and single stranded RNA.

Off-target: As used herein, “off target” refers to any unintended effect on any one or more target, gene and/or cellular transcript.

Operably linked: As used herein, the phrase “operably linked” refers to a functional connection between two or more molecules, constructs, transcripts, entities, moieties or the like.

Particle: As used herein, a “particle” is a virus comprised of at least two components, a protein capsid and a polynucleotide sequence enclosed within the capsid.

Patient: As used herein, “patient” refers to a subject who may seek or be in need of treatment, requires treatment, is receiving treatment, will receive treatment, or a subject who is under care by a trained (e.g., licensed) professional for a particular disease or condition.

Payload: As used herein, “payload” refers to one or more polynucleotides or polynucleotide regions encoded by or within a viral genome or an expression product of such polynucleotide or polynucleotide region, e.g., a transgene, a polynucleotide encoding a polypeptide or multi-polypeptide or a modulatory nucleic acid or regulatory nucleic acid.

Payload construct: As used herein, “payload construct” is one or more polynucleotide regions encoding or comprising a payload that is flanked on one or both sides by an inverted terminal repeat (ITR) sequence. The payload construct is a template that is replicated in a viral production cell to produce a viral genome.

Payload construct vector: As used herein, “payload construct vector” is a vector encoding or comprising a payload construct, and regulatory regions for replication and expression in bacterial cells. The payload construct vector may also comprise component for viral expression in a viral replication cell.

Peptide: As used herein, the term “peptide” refers to a chain of amino acids that is less than or equal to about 50 amino acids long, e.g., about 5, 10, 15, 20, 25, 30, 35, 40, 45, or 50 amino acids long.

Pharmaceutically acceptable: The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

Pharmaceutically acceptable excipients: As used herein, the term “pharmaceutically acceptable excipient,” as used herein, refers to any ingredient other than active agents (e.g., as described herein) present in pharmaceutical compositions and having the properties of being substantially nontoxic and non-inflammatory in subjects. In some embodiments, pharmaceutically acceptable excipients are vehicles capable of suspending and/or dissolving active agents. Excipients may include, for example: antiadherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspending or dispersing agents, sweeteners, and waters of hydration. Excipients include, but are not limited to: butylated hydroxytoluene (BHT), calcium carbonate, calcium phosphate (dibasic), calcium stearate, croscarmellose, cross-linked polyvinyl pyrrolidone, citric acid, crospovidone, cysteine, ethylcellulose, gelatin, hydroxypropyl cellulose, hydroxypropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, sucrose, talc, titanium dioxide, vitamin A, vitamin E, vitamin C, and xylitol.

Pharmaceutically acceptable salts: Pharmaceutically acceptable salts of the compounds described herein are forms of the disclosed compounds wherein the acid or base moiety is in its salt form (e.g., as generated by reacting a free base group with a suitable organic acid). Examples of pharmaceutically acceptable salts include, but are not limited to, mineral or organic acid salts of basic residues such as amines; alkali or organic salts of acidic residues such as carboxylic acids; and the like. Representative acid addition salts include acetate, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, and the like. Representative alkali or alkaline earth metal salts include sodium, lithium, potassium, calcium, magnesium, and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like. Pharmaceutically acceptable salts include the conventional non-toxic salts, for example, from non-toxic inorganic or organic acids. In some embodiments a pharmaceutically acceptable salt is prepared from a parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17^{th} ed., Mack Publishing Company, Easton, Pa., 1985, p. 1418, Pharmaceutical Salts: Properties, Selection, and Use, P. H. Stahl and C. G. Wermuth (eds.), Wiley-VCH, 2008, and Berge et al., Journal of Pharmaceutical Science, 66, 1-19 (1977), each of which is incorporated herein by reference in its entirety. Pharmaceutically acceptable solvate: The term “pharmaceutically acceptable solvate,” as used herein, refers to a crystalline form of a compound wherein molecules of a suitable solvent are incorporated in the crystal lattice. For example, solvates may be prepared by crystallization, recrystallization, or precipitation from a solution that includes organic solvents, water, or a mixture thereof. Examples of suitable solvents are ethanol, water (for example, mono-, di-, and tri-hydrates), N-methylpyrrolidinone (NMP), dimethyl sulfoxide (DMSO), N,N′-dimethylformamide (DMF), N,N′-dimethylacetamide (DMAC), 1,3-dimethyl-2-imidazolidinone (DMEU), 1,3-dimethyl-3,4,5,6-tetrahydro-2-(1H)-pyrimidinone (DMPU), acetonitrile (ACN), propylene glycol, ethyl acetate, benzyl alcohol, 2-pyrrolidone, benzyl benzoate, and the like. When water is the solvent, the solvate is referred to as a “hydrate.” In some embodiments, the solvent incorporated into a solvate is of a type or at a level that is physiologically tolerable to an organism to which the solvate is administered (e.g., in a unit dosage form of a pharmaceutical composition).

Pharmaceutical Composition: As used herein, the term “pharmaceutical composition” or pharmaceutically acceptable composition” comprises an AAV polynucleotides, AAV genomes or AAV particle and one or more pharmaceutically acceptable excipients.

Pharmacokinetic: As used herein, “pharmacokinetic” refers to any one or more properties of a molecule or compound as it relates to the determination of the fate of substances administered to living organisms. Pharmacokinetics are divided into several areas including the extent and rate of absorption, distribution, metabolism and excretion. This is commonly referred to as ADME where: (A) Absorption is the process of a substance entering the blood circulation; (D) Distribution is the dispersion or dissemination of substances throughout the fluids and tissues of the body; (M) Metabolism (or Biotransformation) is the irreversible transformation of parent compounds into daughter metabolites; and (E) Excretion (or Elimination) refers to the elimination of the substances from the body. In rare cases, some drugs irreversibly accumulate in body tissue.

Physicochemical: As used herein, “physicochemical” means of or relating to a physical and/or chemical property.

Preventing: As used herein, the term “preventing” refers to partially or completely delaying onset of an infection, disease, disorder and/or condition; partially or completely delaying onset of one or more symptoms, features, or clinical manifestations of a particular infection, disease, disorder, and/or condition; partially or completely delaying onset of one or more symptoms, features, or manifestations of a particular infection, disease, disorder, and/or condition; partially or completely delaying progression from an infection, a particular disease, disorder and/or condition; and/or decreasing the risk of developing pathology associated with the infection, the disease, disorder, and/or condition.

Proliferate: As used herein, the term “proliferate” means to grow, expand, replicate or increase or cause to grow, expand, replicate or increase. “Proliferative” means having the ability to proliferate. “Anti-proliferative” means having properties counter to or in opposition to proliferative properties.

Prone position: As used herein, “prone position” refers to a subject lying face down.

Protein of interest: As used herein, the terms “proteins of interest” or “desired proteins” include those provided herein and fragments, mutants, variants, and alterations thereof.

Purified: As used herein, the term “purify” means to make substantially pure or clear from unwanted components, material defilement, admixture or imperfection. “Purified” refers to the state of being pure. “Purification” refers to the process of making pure.

Region: As used herein, the term “region” refers to a zone or general area. In some embodiments, when referring to a protein or protein module, a region may comprise a linear sequence of amino acids along the protein or protein module or may comprise a three dimensional area, an epitope and/or a cluster of epitopes. In some embodiments, regions comprise terminal regions. As used herein, the term “terminal region” refers to regions located at the ends or termini of a given agent. When referring to proteins, terminal regions may comprise N- and/or C-termini. N-termini refer to the end of a protein comprising an amino acid with a free amino group. C-termini refer to the end of a protein comprising an amino acid with a free carboxyl group. N- and/or C-terminal regions may there for comprise the N- and/or C-termini as well as surrounding amino acids. In some embodiments, N- and/or C-terminal regions comprise from about 3 amino acid to about 30 amino acids, from about 5 amino acids to about 40 amino acids, from about 10 amino acids to about 50 amino acids, from about 20 amino acids to about 100 amino acids and/or at least 100 amino acids. In some embodiments, N-terminal regions may comprise any length of amino acids that includes the N-terminus, but does not include the C-terminus. In some embodiments, C-terminal regions may comprise any length of amino acids, which include the C-terminus, but do not comprise the N-terminus.

In some embodiments, when referring to a polynucleotide, a region may comprise a linear sequence of nucleic acids along the polynucleotide or may comprise a three dimensional area, secondary structure, or tertiary structure. In some embodiments, regions comprise terminal regions. As used herein, the term “terminal region” refers to regions located at the ends or termini of a given agent. When referring to polynucleotides, terminal regions may comprise 5′ and 3′ termini. 5′ termini refer to the end of a polynucleotide comprising a nucleic acid with a free phosphate group. 3′ termini refer to the end of a polynucleotide comprising a nucleic acid with a free hydroxyl group. 5′ and 3′ regions may there for comprise the 5′ and 3′ termini as well as surrounding nucleic acids. In some embodiments, 5′ and 3′ terminal regions comprise from about 9 nucleic acids to about 90 nucleic acids, from about 15 nucleic acids to about 120 nucleic acids, from about 30 nucleic acids to about 150 nucleic acids, from about 60 nucleic acids to about 300 nucleic acids and/or at least 300 nucleic acids. In some embodiments, 5′ regions may comprise any length of nucleic acids that includes the 5′ terminus, but does not include the 3′ terminus. In some embodiments, 3′ regions may comprise any length of nucleic acids, which include the 3′ terminus, but does not comprise the 5′ terminus.

Right Lateral Recumbent Position: As used herein, “Right Lateral Recumbent” or RLR position refers to a subject laying on their right side.

RNA or RNA molecule: As used herein, the term “RNA” or “RNA molecule” or “ribonucleic acid molecule” refers to a polymer of ribonucleotides; the term “DNA” or “DNA molecule” or “deoxyribonucleic acid molecule” refers to a polymer of deoxyribonucleotides. DNA and RNA can be synthesized naturally, e.g., by DNA replication and transcription of DNA, respectively; or be chemically synthesized. DNA and RNA can be single-stranded (i.e., ssRNA or ssDNA, respectively) or multi-stranded (e.g., double stranded, i.e., dsRNA and dsDNA, respectively). The term “mRNA” or “messenger RNA”, as used herein, refers to a single stranded RNA that encodes the amino acid sequence of one or more polypeptide chains.

RNA interference: As used herein, the term “RNA interference” or “RNAi” refers to a sequence specific regulatory mechanism mediated by RNA molecules which results in the inhibition or interference or “silencing” of the expression of a corresponding protein-coding gene.

Sacral Region: As used herein, the term “sacral region” refers to the region of the spinal cord comprising the sacral vertebrae S1, S2, S3, S4, and S5.

Sample: As used herein, the term “sample” refers to an aliquot or portion taken from a source and/or provided for analysis or processing. In some embodiments, a sample is from a biological source such as a tissue, cell or component part (e.g. a body fluid, including but not limited to blood, mucus, lymphatic fluid, synovial fluid, cerebrospinal fluid, saliva, amniotic fluid, amniotic cord blood, urine, vaginal fluid and semen). In some embodiments, a sample may be or comprise a homogenate, lysate or extract prepared from a whole organism or a subset of its tissues, cells or component parts, or a fraction or portion thereof, including but not limited to, for example, plasma, serum, spinal fluid, lymph fluid, the external sections of the skin, respiratory, intestinal, and genitourinary tracts, tears, saliva, milk, blood cells, tumors, organs. In some embodiments, a sample is or comprises a medium, such as a nutrient broth or gel, which may contain cellular components, such as proteins or nucleic acid molecule. In some embodiments, a “primary” sample is an aliquot of the source. In some embodiments, a primary sample is subjected to one or more processing (e.g., separation, purification, etc.) steps to prepare a sample for analysis or other use.

Self-complementary viral particle: As used herein, a “self-complementary viral particle” is a particle comprised of at least two components, a protein capsid and a polynucleotide sequence encoding a self-complementary genome enclosed within the capsid.

Sense strand: As used herein, the term “the sense strand” or “the second strand” or “the passenger strand” of a siRNA molecule refers to a strand that is complementary to the antisense strand or first strand. The antisense and sense strands of a siRNA molecule are hybridized to form a duplex structure. As used herein, a “siRNA duplex” includes a siRNA strand having sufficient complementarity to a section of about 10-50 nucleotides of the mRNA of the gene targeted for silencing and a siRNA strand having sufficient complementarity to form a duplex with the siRNA strand.

Signal Sequences: As used herein, the phrase “signal sequences” refers to a sequence which can direct the transport or localization.

Single unit dose: As used herein, a “single unit dose” is a dose of any therapeutic administered in one dose/at one time/single route/single point of contact, i.e., single administration event. In some embodiments, a single unit dose is provided as a discrete dosage form (e.g., a tablet, capsule, patch, loaded syringe, vial, etc.).

Similarity: As used herein, the term “similarity” refers to the overall relatedness between polymeric molecules, e.g. between polynucleotide molecules (e.g. DNA molecules and/or RNA molecules) and/or between polypeptide molecules. Calculation of percent similarity of polymeric molecules to one another can be performed in the same manner as a calculation of percent identity, except that calculation of percent similarity takes into account conservative substitutions as is understood in the art.

Small/short interfering RNA: As used herein, the term “small/short interfering RNA” or “siRNA” refers to an RNA molecule (or RNA analog) comprising between about 5-60 nucleotides (or nucleotide analogs) which is capable of directing or mediating RNAi. Preferably, a siRNA molecule comprises between about 15-30 nucleotides or nucleotide analogs, more preferably between about 16-25 nucleotides (or nucleotide analogs), even more preferably between about 18-23 nucleotides (or nucleotide analogs), and even more preferably between about 19-22 nucleotides (or nucleotide analogs) (e.g., 19, 20, 21 or 22 nucleotides or nucleotide analogs). The term “short” siRNA refers to a siRNA comprising 5-23 nucleotides, preferably 21 nucleotides (or nucleotide analogs), for example, 19, 20, 21 or 22 nucleotides. The term “long” siRNA refers to a siRNA comprising 24-60 nucleotides, preferably about 24-25 nucleotides, for example, 23, 24, 25 or 26 nucleotides. Short siRNAs may, in some instances, include fewer than 19 nucleotides, e.g., 16, 17 or 18 nucleotides, or as few as 5 nucleotides, provided that the shorter siRNA retains the ability to mediate RNAi. Likewise, long siRNAs may, in some instances, include more than 26 nucleotides, e.g., 27, 28, 29, 30, 35, 40, 45, 50, 55, or even 60 nucleotides, provided that the longer siRNA retains the ability to mediate RNAi or translational repression absent further processing, e.g., enzymatic processing, to a short siRNA. siRNAs can be single stranded RNA molecules (ss-siRNAs) or double stranded RNA molecules (ds-siRNAs) comprising a sense strand and an antisense strand which hybridized to form a duplex structure called siRNA duplex.

Split dose: As used herein, a “split dose” is the division of single unit dose or total daily dose into two or more doses.

Stable: As used herein “stable” refers to a compound or entity that is sufficiently robust to survive isolation to a useful degree of purity from a reaction mixture, and preferably capable of formulation into an efficacious therapeutic agent.

Stabilized: As used herein, the term “stabilize”, “stabilized,” “stabilized region” means to make or become stable. In some embodiments, stability is measured relative to an absolute value. In some embodiments, stability is measured relative to a reference compound or entity.

Subject: As used herein, the term “subject” or “patient” refers to any organism to which a composition in accordance with the invention may be administered, e.g., for experimental, diagnostic, prophylactic, and/or therapeutic purposes. Typical subjects include animals (e.g., mammals such as mice, rats, rabbits, non-human primates, and humans).

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

Substantially equal: As used herein as it relates to time differences between doses, the term means plus/minus 2%.

Substantially simultaneously: As used herein and as it relates to plurality of doses, the term typically means within about 2 seconds.

Suffering from: An individual who is “suffering from” a disease, disorder, and/or condition has been diagnosed with or displays one or more symptoms of a disease, disorder, and/or condition.

Supine position: As used herein, “supine position” refers to a subject lying on his or her back.

Susceptible to: An individual who is “susceptible to” a disease, disorder, and/or condition has not been diagnosed with and/or may not exhibit symptoms of the disease, disorder, and/or condition but harbors a propensity to develop a disease or its symptoms. In some embodiments, an individual who is susceptible to a disease, disorder, and/or condition (for example, cancer) may be characterized by one or more of the following: (1) a genetic mutation associated with development of the disease, disorder, and/or condition; (2) a genetic polymorphism associated with development of the disease, disorder, and/or condition; (3) increased and/or decreased expression and/or activity of a protein and/or nucleic acid associated with the disease, disorder, and/or condition; (4) habits and/or lifestyles associated with development of the disease, disorder, and/or condition; (5) a family history of the disease, disorder, and/or condition; and (6) exposure to and/or infection with a microbe associated with development of the disease, disorder, and/or condition. In some embodiments, an individual who is susceptible to a disease, disorder, and/or condition will develop the disease, disorder, and/or condition. In some embodiments, an individual who is susceptible to a disease, disorder, and/or condition will not develop the disease, disorder, and/or condition.

Synthetic: The term “synthetic” means produced, prepared, and/or manufactured by the hand of man. Synthesis of polynucleotides or polypeptides or other molecules of the present invention may be chemical or enzymatic.

Targeting: As used herein, “targeting” means the process of design and selection of nucleic acid sequence that will hybridize to a target nucleic acid and induce a desired effect.

Targeted Cells: As used herein, “targeted cells” refers to any one or more cells of interest. The cells may be found in vitro, in vivo, in situ or in the tissue or organ of an organism. The organism may be an animal, preferably a mammal, more preferably a human and most preferably a patient.

Therapeutic Agent: The term “therapeutic agent” refers to any agent that, when administered to a subject has a therapeutic, diagnostic, and/or prophylactic effect and/or elicits a desired biological and/or pharmacological effect.

Therapeutically effective amount: As used herein, the term “therapeutically effective amount” means an amount of an agent to be delivered (e.g., nucleic acid, drug, therapeutic agent, diagnostic agent, prophylactic agent, etc.) that is sufficient, when administered to a subject suffering from or susceptible to an infection, disease, disorder, and/or condition, to treat, improve symptoms of, diagnose, prevent, and/or delay the onset of the infection, disease, disorder, and/or condition. In some embodiments, a therapeutically effective amount is provided in a single dose. In some embodiments, a therapeutically effective amount is administered in a dosage regimen comprising a plurality of doses. Those skilled in the art will appreciate that in some embodiments, a unit dosage form may be considered to comprise a therapeutically effective amount of a particular agent or entity if it comprises an amount that is effective when administered as part of such a dosage regimen.

Therapeutically effective outcome: As used herein, the term “therapeutically effective outcome” means an outcome that is sufficient in a subject suffering from or susceptible to an infection, disease, disorder, and/or condition, to treat, improve symptoms of, diagnose, prevent, and/or delay the onset of the infection, disease, disorder, and/or condition.

Thoracic Region: As used herein, a “thoracic region” refers to a region of the spinal cord comprising the thoracic vertebrae T1, T2, T3, T4, T5, T6, T7, T8, T9, T10, T11, and T12.

Total daily dose: As used herein, a “total daily dose” is an amount given or prescribed in a 24 hour period. It may be administered as a single unit dose.

Treating: As used herein, the term “treating” refers to partially or completely alleviating, ameliorating, improving, relieving, delaying onset of, inhibiting progression of, reducing severity of, and/or reducing incidence of one or more symptoms or features of a particular infection, disease, disorder, and/or condition. For example, “treating” cancer may refer to inhibiting survival, growth, and/or spread of a tumor. Treatment may be administered to a subject who does not exhibit signs of a disease, disorder, and/or condition and/or to a subject who exhibits only early signs of a disease, disorder, and/or condition for the purpose of decreasing the risk of developing pathology associated with the disease, disorder, and/or condition.

Trendelenburg Position: As used herein, a subject tin the “Trendelenburg position” is lying supine with their head slightly lower than their feet.

Unmodified: As used herein, “unmodified” refers to any substance, compound or molecule prior to being changed in any way. Unmodified may, but does not always, refer to the wild-type or native form of a biomolecule or entity. Molecules or entities may undergo a series of modifications whereby each modified product may serve as the “unmodified” starting molecule or entity for a subsequent modification.

Vector: As used herein, a “vector” is any molecule or moiety which transports, transduces or otherwise acts as a carrier of a heterologous molecule. Vectors of the present invention may be produced recombinantly and may be based on and/or may comprise adeno-associated virus (AAV) parent or reference sequence. Such parent or reference AAV sequences may serve as an original, second, third or subsequent sequence for engineering vectors. In non-limiting examples, such parent or reference AAV sequences may comprise any one or more of the following sequences: a polynucleotide sequence encoding a polypeptide or multi-polypeptide, which sequence may be wild-type or modified from wild-type and which sequence may encode full-length or partial sequence of a protein, protein domain, or one or more subunits of a protein; a polynucleotide comprising a modulatory or regulatory nucleic acid which sequence may be wild-type or modified from wild-type; and a transgene that may or may not be modified from wild-type sequence. These AAV sequences may serve as either the “donor” sequence of one or more codons (at the nucleic acid level) or amino acids (at the polypeptide level) or “acceptor” sequences of one or more codons (at the nucleic acid level) or amino acids (at the polypeptide level).

Viral construct vector: As used herein, a “viral construct vector” is a vector which comprises one or more polynucleotide regions encoding or comprising Rep and or Cap protein. A viral construct vector may also comprise one or more polynucleotide region encoding or comprising components for viral expression in a viral replication cell.

Viral genome: As used herein, a “viral genome” is a polynucleotide encoding at least one inverted terminal repeat (ITR), at least one regulatory sequence, and at least one payload. The viral genome is derived by replication of a payload construct from the payload construct vector. A viral genome encodes at least one copy of the payload construct.

### EQUIVALENTS AND SCOPE

Those skilled in the art will recognize, or be able to ascertain using no more than routine experimentation, many equivalents to the specific embodiments in accordance with the invention described herein. The scope of the present invention is not intended to be limited to the above Description, but rather is as set forth in the appended claims.

In the claims, articles such as “a,” “an,” and “the” may mean one or more than one unless indicated to the contrary or otherwise evident from the context. claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or the entire group members are present in, employed in, or otherwise relevant to a given product or process.

It is also noted that the term “comprising” is intended to be open and permits but does not require the inclusion of additional elements or steps. When the term “comprising” is used herein, the term “consisting of” is thus also encompassed and disclosed.

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise.

In addition, it is to be understood that any particular embodiment of the present invention that falls within the prior art may be explicitly excluded from any one or more of the claims. Since such embodiments are deemed to be known to one of ordinary skill in the art, they may be excluded even if the exclusion is not set forth explicitly herein. Any particular embodiment of the compositions of the invention (e.g., any antibiotic, therapeutic or active ingredient; any method of production; any method of use; etc.) can be excluded from any one or more claims, for any reason, whether or not related to the existence of prior art.

It is to be understood that the words which have been used are words of description rather than limitation, and that changes may be made within the purview of the appended claims without departing from the true scope and spirit of the invention in its broader aspects.

While the present invention has been described at some length and with some particularity with respect to the several described embodiments, it is not intended that it should be limited to any such particulars or embodiments or any particular embodiment, but it is to be construed with references to the appended claims so as to provide the broadest possible interpretation of such claims in view of the prior art and, therefore, to effectively encompass the intended scope of the invention.

### EXAMPLES

### Example 1. Design of the Payload Construct

Payload constructs were designed to comprise at a minimum a nucleic acid sequence encoding a frataxin protein.

Once designed, the sequence was engineered or synthesized or inserted in a plasmid or vector and administered to a cell or organism. Suitable plasmids or vectors were any which transduce or transfect the target cell.

Adeno-associated viral vectors (AAV), viral particles or entire viruses may be used.

Administration resulted in the processing of the payload construct to generate the frataxin protein which alters the etiology of the disease, in this case Friedreich's Ataxia.

AAV constructs were designed and built using standard molecular cloning techniques. FXN-tag transgenes were cloned into either pAAVss, pAAVsc, or pcDNA3.1 plasmid and the resulting clones were further sequenced to confirm the correctness of all elements such as ITRs, promoters, and tags.

In one non-limiting example, plasmids containing a payload construct are described herein and some are described in Table 4. These AAV particles in Table 4 may comprise a pCDNA3.1, pAAVss, or pAAVsc vectors and may contain the following components: a CMV, CB6, CB7, PGK, GFAP, hSYN, mCMVe-hEF1p, SV40, CBA or FXN promoter; an intron such as SV40 or MVM/CBA; a full or partial Kozak sequence; a FXN (Frataxin), CS (citrate synthase), RPL (ribosomal protein), SOD2 (superoxide dismutase), or AH (aconitate hydratase) signal peptide, also known as a mitochondrial targeting sequence (MTS); a cmyc, flag, cmycflag3, 3flag, 3flagcmyc, HA long or HA short tag; a SV40, rabbit beta-globin, or bGH poly (A) signal, 3′ and/or 5′ ITR sequences derived from any AAV genome comprising a partial and/or wild type sequence; and either wild type Frataxin or codon optimized Frataxin.

TABLE 4
AAV constructs.

| | | | | 0.5 | Signal | | | | | | SEQ ID |
|---|---|---|---|---|---|---|---|---|---|---|---|
| Vector | Promoter | Intron | Kozak | Kozak | Peptide | Payload | Tag | 5′ITR | Poly(A) | 3′ITR | NO |
| pCDNA3.1(+) | CMV | N/A | − | − | FXN | FXN | cmyc3flag | − | bGH | − | 570 |
| pCDNA3.1(+) | CMV | N/A | − | − | FXN | FXN | — | − | bGH | − | 571 |
| pCDNA3.1(+) | CMV | N/A | − | − | FXN | FXN | 3flag | − | bGH | − | 572 |
| pCDNA3.1(+) | CMV | N/A | − | − | FXN | FXN | 3flagcmyc | − | bGH | − | 573 |
| pCDNA3.1(+) | CMV | N/A | − | − | FXN | FXN | cmyc | − | bGH | − | 574 |
| pCDNA3.1(+) | CMV | N/A | − | − | FXN | FXN | HA(L) | − | bGH | − | 575 |
| pCDNA3.1(+) | CMV | N/A | − | − | FXN | FXN | HA(S) | − | bGH | − | 576 |
| pCDNA3.1(+) | CMV | N/A | + | − | CS | FXN | — | − | bGH | − | 577 |
| pCDNA3.1(+) | CMV | N/A | − | + | FXN | FXN | — | − | bGH | − | 578 |
| pAAVss | CB6 | N/A | − | + | FXN | FXN | — | + | SV40 | + | 579 |
| pAAVss | CB6 | MVM/CBA | − | + | FXN | FXN | — | + | SV40 | + | 580 |
| pAAVss | CB6 | SV40 | − | − | FXN | FXN | — | + | SV40 | + | 581 |
| pAAVss | CB6 | SV40 | − | − | FXN | CodOp | — | + | SV40 | + | 582 |
| | | | | | | FXN | | | | | |
| pAAVss | CB6 | SV40 | − | − | FXN | CodOp | — | + | SV40 | + | 583 |
| | | | | | | FXN | | | | | |
| pAAVss | CB6 | SV40 | − | − | FXN | CodOp | — | + | SV40 | + | 584 |
| | | | | | | FXN | | | | | |
| pAAVss | CB6 | SV40 | − | − | FXN | CodOp | — | + | SV40 | + | 585 |
| | | | | | | FXN | | | | | |
| pAAVss | CB6 | SV40 | − | − | FXN | CodOp | — | + | SV40 | + | 586 |
| | | | | | | FXN | | | | | |
| pAAVss | CB6 | SV40 | − | − | FXN | CodOp | — | + | SV40 | + | 587 |
| | | | | | | FXN | | | | | |
| pAAVss | CB6 | SV40 | − | − | FXN | CodOp | — | + | SV40 | + | 588 |
| | | | | | | FXN | | | | | |
| pAAVss | CB6 | SV40 | − | − | FXN | CodOp | — | + | SV40 | + | 589 |
| | | | | | | FXN | | | | | |
| pAAVss | CB6 | SV40 | − | − | FXN | CodOp | — | + | SV40 | + | 590 |
| | | | | | | FXN | | | | | |
| pAAVss | CB6 | SV40 | − | − | FXN | CodOp | — | + | SV40 | + | 591 |
| | | | | | | FXN | | | | | |
| pAAVss | CB6 | SV40 | − | − | FXN | CodOp | — | + | SV40 | + | 592 |
| | | | | | | FXN | | | | | |
| pAAVss | CB6 | SV40 | − | − | FXN | CodOp | — | + | SV40 | + | 593 |
| | | | | | | FXN | | | | | |
| pAAVss | CB6 | SV40 | + | − | AH | FXN | — | + | SV40 | + | 594 |
| pAAVss | CB6 | SV40 | + | − | CS | FXN | — | + | SV40 | + | 595 |
| pAAVss | CB6 | SV40 | + | − | CS | FXN | HA(L) | + | SV40 | + | 596 |
| pAAVss | CB6 | SV40 | + | − | CS | FXN | HA(S) | + | SV40 | + | 597 |
| pAAVss | CB6 | SV40 | − | + | FXN | FXN | — | + | SV40 | + | 598 |
| pAAVss | CB6 | SV40 | − | + | FXN | CodOp | — | + | SV40 | + | 599 |
| | | | | | | FXN | | | | | |
| pAAVss | CB6 | SV40 | − | + | FXN | CodOp | — | + | SV40 | + | 600 |
| | | | | | | FXN | | | | | |
| pAAVss | CB6 | SV40 | − | + | FXN | CodOp | — | + | SV40 | + | 601 |
| | | | | | | FXN | | | | | |
| pAAVss | CB6 | SV40 | − | + | FXN | CodOp | — | + | SV40 | + | 602 |
| | | | | | | FXN | | | | | |
| pAAVss | CB6 | SV40 | − | + | FXN | CodOp | — | + | SV40 | + | 603 |
| | | | | | | FXN | | | | | |
| pAAVss | CB6 | SV40 | − | + | FXN | CodOp | — | + | SV40 | + | 604 |
| | | | | | | FXN | | | | | |
| pAAVss | CB6 | SV40 | − | + | FXN | CodOp | — | + | SV40 | + | 605 |
| | | | | | | FXN | | | | | |
| pAAVss | CB6 | SV40 | + | − | RPL | FXN | — | + | SV40 | + | 606 |
| pAAVss | CB6 | SV40 | + | − | RPL | CodOp | — | + | SV40 | + | 607 |
| | | | | | | FXN | | | | | |
| pAAVss | CB6 | SV40 | − | + | S0D2 | CodOp | — | + | SV40 | + | 608 |
| | | | | | | FXN | | | | | |
| pAAVss | CB7 | SV40 | − | − | FXN | FXN | — | + | SV40 | + | 609 |
| pAAVss | CMV | N/A | − | + | FXN | FXN | — | + | SV40 | + | 610 |
| pAAVss | CMV | SV40 | − | − | FXN | FXN | — | + | SV40 | + | 611 |
| pAAVss | CMV | SV40 | − | − | FXN | CodOp | — | + | SV40 | + | 612 |
| | | | | | | FXN | | | | | |
| pAAVss | CMV | SV40 | − | − | FXN | CodOp | — | + | SV40 | + | 613 |
| | | | | | | FXN | | | | | |
| pAAVss | CMV | SV40 | − | − | FXN | CodOp | — | + | SV40 | + | 614 |
| | | | | | | FXN | | | | | |
| pAAVss | CMV | SV40 | − | − | FXN | CodOp | — | + | SV40 | + | 615 |
| | | | | | | FXN | | | | | |
| pAAVss | CMV | SV40 | − | + | FXN | FXN | — | + | SV40 | + | 616 |
| pAAVss | FXNp | SV40 | − | − | FXN | FXN | — | + | SV40 | + | 617 |
| pAAVss | FXNp | SV40 | − | − | FXN | CodOp | — | + | SV40 | + | 618 |
| | | | | | | FXN | | | | | |
| pAAVss | FXNp | SV40 | − | − | FXN | CodOp | — | + | SV40 | + | 619 |
| | | | | | | FXN | | | | | |
| pAAVss | FXNp | SV40 | − | − | FXN | CodOp | — | + | SV40 | + | 620 |
| | | | | | | FXN | | | | | |
| pAAVss | FXNp | SV40 | − | − | FXN | CodOp | — | + | SV40 | + | 621 |
| | | | | | | FXN | | | | | |
| pAAVss | GFAP | SV40 | − | − | FXN | FXN | — | + | SV40 | + | 622 |
| pAAVss | hSYN | SV40 | − | − | FXN | FXN | — | + | SV40 | + | 623 |
| pAAVss | mCMVe- | SV40 | − | − | FXN | FXN | — | + | SV40 | + | 624 |
| | hEF1p | | | | | | | | | | |
| pAAVss | mCMVe- | SV40 | − | − | FXN | FXN | — | + | SV40 | + | 625 |
| | hEF1p | | | | | | | | | | |
| pAAVss | PGK | SV40 | − | − | FXN | FXN | — | + | SV40 | + | 626 |
| pAAVss | PGK | SV40 | − | − | FXN | CodOp | — | + | SV40 | + | 627 |
| | | | | | | FXN | | | | | |
| pAAVss | PGK | SV40 | − | − | FXN | CodOp | — | + | SV40 | + | 628 |
| | | | | | | FXN | | | | | |
| pAAVss | PGK | SV40 | − | − | FXN | CodOp | — | + | SV40 | + | 629 |
| | | | | | | FXN | | | | | |
| pAAVss | PGK | SV40 | − | − | FXN | CodOp | — | + | SV40 | + | 630 |
| | | | | | | FXN | | | | | |
| pAAVss | SV40 | SV40 | − | − | FXN | FXN | — | + | SV40 | + | 631 |
| pAAVsc | CBA | SV40 | − | − | FXN | FXN | — | + | SV40 | + | 632 |
| pAAVsc | CBA | SV40 | − | − | FXN | FXN | HA(S) | + | SV40 | + | 633 |
| pAAVsc | CBA | SV40 | − | − | FXN | FXN | 3flag | + | SV40 | + | 634 |
| pAAVsc | CBA | SV40 | − | − | FXN | FXN | 3flagcmyc | + | SV40 | + | 635 |
| pAAVsc | CBA | SV40 | − | − | FXN | FXN | cmyc | + | SV40 | + | 636 |
| pAAVsc | CBA | SV40 | − | − | FXN | FXN | cmyc3flag | + | SV40 | + | 637 |
| pAAVsc | CBA | SV40 | − | − | FXN | CodOp | — | + | SV40 | + | 638 |
| | | | | | | FXN | | | | | |
| pAAVsc | CBA | SV40 | − | − | FXN | CodOp | — | + | SV40 | + | 639 |
| | | | | | | FXN | | | | | |
| pAAVsc | CBA | SV40 | − | − | FXN | CodOp | — | + | SV40 | + | 640 |
| | | | | | | FXN | | | | | |
| pAAVsc | CBA | SV40 | − | − | FXN | CodOp | — | + | SV40 | + | 641 |
| | | | | | | FXN | | | | | |
| pAAVsc | CMV | SV40 | − | − | FXN | FXN | — | + | SV40 | + | 642 |
| pAAVsc | CMV | SV40 | − | − | FXN | CodOp | — | + | SV40 | + | 643 |
| | | | | | | FXN | | | | | |
| pAAVsc | CMV | SV40 | − | − | FXN | CodOp | — | + | SV40 | + | 644 |
| | | | | | | FXN | | | | | |
| pAAVsc | CMV | SV40 | − | − | FXN | CodOp | — | + | SV40 | + | 645 |
| | | | | | | FXN | | | | | |
| pAAVsc | CMV | SV40 | − | − | FXN | CodOp | — | + | SV40 | + | 646 |
| | | | | | | FXN | | | | | |
| pAAVsc | CMV | SV40 | − | + | FXN | FXN | — | + | SV40 | + | 647 |
| pAAVsc | FXNp | SV40 | − | − | FXN | FXN | — | + | SV40 | + | 648 |
| pAAVsc | FXNp | SV40 | − | − | FXN | CodOp | — | + | SV40 | + | 649 |
| | | | | | | FXN | | | | | |
| pAAVsc | FXNp | SV40 | − | − | FXN | CodOp | — | + | SV40 | + | 650 |
| | | | | | | FXN | | | | | |
| pAAVsc | FXNp | SV40 | − | − | FXN | CodOp | — | + | SV40 | + | 651 |
| | | | | | | FXN | | | | | |
| pAAVsc | FXNp | SV40 | − | − | FXN | CodOp | — | + | SV40 | + | 652 |
| | | | | | | FXN | | | | | |
| pAAVsc | GFAP | SV40 | − | − | FXN | FXN | — | + | SV40 | + | 653 |
| pAAVsc | PGK | SV40 | − | − | FXN | FXN | — | + | SV40 | + | 654 |
| pAAVsc | PGK | SV40 | − | − | FXN | CodOp | — | + | SV40 | + | 655 |
| | | | | | | FXN | | | | | |
| pAAVsc | PGK | SV40 | − | − | FXN | CodOp | — | + | SV40 | + | 656 |
| | | | | | | FXN | | | | | |
| pAAVsc | PGK | SV40 | − | − | FXN | CodOp | — | + | SV40 | + | 657 |
| | | | | | | FXN | | | | | |
| pAAVsc | PGK | SV40 | − | − | FXN | CodOp | — | + | SV40 | + | 658 |
| | | | | | | FXN | | | | | |
| pAAVss | CB6 | SV40 | − | + | FXN | FXN | — | + | SV40 | + | 659 |
| pAAVsc | CBA | SV40 | − | + | FXN | FXN | — | + | SV40 | + | 660 |
| pAAVss | N/A | SV40 | − | − | FXN | FXN | — | + | SV41 | + | 661 |
| pAAVss | CBA | SV40 | − | − | FXN | FXN | — | + | SV42 | + | 662 |

Plasmid constructs suitable for use in AAV particles include those in the sequence listing.

### Example 2. ELISA Assay for Detecting Differential Payload Expression from Regulatory Elements in Various Cell Types

The HEK293 cell line was transfected with AAV constructs, SEQ ID Nos. 582-591, 609, 617, 623-625, 631, 632, 639, 642, 644, 648, 650, 654, 656, 661, and 662 to assay the level of expression of a Frataxin payload sequence under control of various regulatory elements in human embryonic kidney 293 (HEK293), primary human fibroblast (FA), rat primary dorsal root ganglia (DRG) neurons (rDRG), or human induced pluripotent stem cell (iPSC) derived neural stem cells (hNSC) cell types.

HEK 293 cells were co-transfected in triplicate using FUGENER HD reagent with each construct (0.5 μg) and the gWiz-GFP plasmid (100 ng) as an internal transfection efficiency control. The transfected 293FT cells were harvested 30-36 hours post-transfection, lysed using the THERMO SCIENTIFIC™ PIERCE™ M-PER™ Mammalian Protein Extraction Reagent, and resuspended in 200 ul of lysis buffer. Protein concentration in each of the samples was measured using the Thermo Scientific™ Pierce™ BCA™ Protein Assay.

Table 5 shows the titer and the volume of self-complementary AAV (scAAV) constructs comprising frataxin (FXN), PGK, CBA, or CMV promoter sequences and constructs encoding codon-optimized FXN, expressed in HEK-293 cells by three-plasmid transfection method. Lower titers were obtained with FXN and PGK promoters.

TABLE 5
AAV vector titers in HEK293 cells

| | Titer | Volume |
|---|---|---|
| Vector | (GC/ml) | (ml) |
| scAAVrh10.CBA-SV40-FXN | 1.00E+13 | 3.5 |
| scAAVrh10.CBA-SV40-FXNOpti10 | 1.00E+13 | 3.8 |
| scAAVrh10.CMV-SV40-FXN | 1.00E+13 | 4.3 |
| scAAVrh10.CMV-SV40-FXNOpti10 | 1.00E+13 | 3.2 |
| scAAVrh10.FXNpro-SV40-FXN | 1.50E+12 | 3.1 |
| scAAVrh10.FXNpro-SV40-FXNOpti10 | 4.00E+12 | 2.8 |
| scAAVrh10.PGK-SV40-FXN | 1.50E+12 | 3.0 |
| scAAVrh10.PGK-SV40-FXNOpti10 | 5.00E+12 | 3.2 |

Silver stained SDS-page of vectors listed in Table 5 subsequent to expression in HEK293 cells shows detection of capsid proteins VP1, VP2 and VP3 in proper 1:1:10 ratio.

Analysis of DNAs within rAAV capsids of Table 5 by native agarose gel electrophoresis and alkaline agarose gel electrophoresis show predominance of self-complementary genomes for CBA, CMV and PGK promoters and empty particles (limited DNA) generated by the FXNpro constructs.

### Example 3. Summary of mRNA Expression of AAV Constructs

Expression of the transgene was dependent on the capsid and if expression was being evaluated in the motor neurons of the spinal cord or the DRG sensory neurons. Table 6 provides a summary of the expression for each capsid. In Table 6, “NT” means not tested.

TABLE 6
Expression

| | Transgene expression | | FXN-IHC Staining | |
|---|---|---|---|---|
| Capsid | Motor Neuron | DRG | Motor Neuron | DRG |
| AAV2 | +++ | + | NT | Low |
| AAVDJ | +++ | ++ | High | High |
| AAVDJ8 | ++ | + | NT | High |
| AAV6 | ++ | +++ | NT | NT |
| AAVrh10 | +++ | +++ | High for sc | NT |
| AAV9 | + | + | NT | NT |
| AAV5 | +++ | ++ | NT | NT |

The AAV9 capsid showed the lowest transgene expression in the spinal cord. AAVDJ and AAVrh10 gave the strongest and consistent transgene expression by immunochemistry (IHC) for motor neuron transduction. AAVDJ and AAVDJ8 gave the strongest and consistent transgene expression by IHC for DRG transduction.

### Example 4. Comparison of Human FXN Expression Following Intrastriatal Delivery in Mice of AAV Constructs Containing Three Different Promoters

To compare human FXN expression driven by PGK, CMV and CBA and FXN promoters, more than 93 wild type mice (C57BL/6), 6-8 weeks old, were administered an AAV with dose levels shown in Tables 7-9. The AAVs were formulated in PBS and 0.001% F-68, and 5 uL administered via intrastriatal (IS) injection. Human FXN (hFXN) protein levels in striatum were quantified after 7 days (Tables 7 and 8) or 28 days (Table 9) by ELISA with an assay (Abcam) specific for human FXN (no detection of mouse FXN).

Seven days after AAV intrastriatal administration (5E9 VG), all AAV constructs resulted in human frataxin expression. The CBA promoter drove the highest expression, followed by PGK and CMV promoters. The same rank order of promoter-driven expression (CBA>PGK>CMV) was observed with constructs expressing wild-type human frataxin (Table 7) and codon optimized human frataxin (Table 8) at 7 days post-administration. With the CBA promoter, wild-type frataxin (scAAVrh10-CBA-FXN) and codon-optimized frataxin (scAAVrh10-CBA-Opti10FXN) resulted in similar levels of human frataxin protein in the striatum at this time point (Table 8).

TABLE 7
Striatum Levels of human FXN at 7 days Following
Intrastriatal Injection of Wild-Type Frataxin Constructs

| | AAV | | Inj. Site |
|---|---|---|---|
| Test Article | Genome | Dose (VG) | AVG ± SEM |
| scAAVrh10-CBA-FXN | SC | 5 × 10⁹ | 120.1 ± 20.18 |
| scAAVrh10-CMV-FXN | SC | 5 × 10⁹ | 31.20 ± 12.18 |
| scAAVrh10-PGK-FXN | SC | 5 × 10⁹ | 55.49 ± 4.69 |
| Vehicle | — | — | 2.35 ± 1.33 |

TABLE 8
Striatum Levels of human FXN at 7 days Following Intrastriatal
Injection of Codon-Optimized Frataxin (Opti10FXN) Constructs

| | AAV | | Inj. Site |
|---|---|---|---|
| Test Article | Genome | Dose (VG) | AVG ± SEM |
| scAAVrh10-CBA-Opti10FXN | SC | 5 × 10⁹ | 64.12 ± 15.45 |
| scAAVrh10-CMV-Opti10FXN | SC | 5 × 10⁹ | 18.85 ± 3.93 |
| scAAVrh10-PGK-Opti10FXN | SC | 5 × 10⁹ | 29.96 ± 6.16 |
| scAAVrh10-CBA-FXN | SC | 5 × 10⁹ | 61.89 ± 3.77 |
| Vehicle | — | — | 2.00 ± 0.09 |

Twenty-eight days after AAV intrastriatal administration (5E8, 5E9 or 5E10 VG), all AAV constructs resulted in human frataxin expression in the striatum (Table 9). For all 3 promoters (CBA, CMV, PGK), each log increase in dose resulted in an approximately 6-8 fold increase in human frataxin protein levels in the striatum. The CBA promoter drives the highest level of expression in the striatum, followed by the CMV and PGK promoters. The rank order of promoter-driven expression (CBA>CMV>PGK) was observed at 28 days post-administration, with the CBA promoter resulting in approximately 3-fold higher levels of human FXN protein expression than the CMV promoter, and the CMV promoter resulting in approximately 3-fold higher levels of human FXN protein expression than the PGK promoter, across the dose levels used.

TABLE 9
Striatum Levels of human FXN at 28 days Following Intrastriatal
Injection of Wild-Type and Codon-Optimized Frataxin Constructs

| | AAV | Dose | | Inj. Site | |
|---|---|---|---|---|---|
| Test Article | Genome | (VG) | | AVG ± SEM | |
| scAAVrh10-CBA-FXN | SC | 5 × | 10⁸ | 113.51 ± | 10.32 |
| scAAVrh10-CBA-FXN | SC | 5 × | 10⁹ | 748.53 ± | 120.54 |
| scAAVrh10-CBA-FXN | SC | 5 × | 10¹⁰ | 4915.25 ± | 896.59 |
| scAAVrh10-CMV-FXN | SC | 5 × | 10⁸ | 33.37 ± | 4.91 |
| scAAVrh10-CMV-FXN | SC | 5 × | 10⁹ | 260.67 ± | 12.61 |
| scAAVrh10-CMV-FXN | SC | 5 × | 10¹⁰ | 1687.10 ± | 278.23 |
| scAAVrh10-PGK-FXN | SC | 5 × | 10⁸ | 12.49 ± | 1.02 |
| scAAVrh10-PGK-FXN | SC | 5 × | 10⁹ | 79.93 ± | 1.60 |
| scAAVrh10-PGK-FXN | SC | 5 × | 10¹⁰ | 515.81 ± | 29.32 |
| scAAVrh10-CBA-Opti10FXN | SC | 5 × | 10⁹ | 777.94 ± | 176.08 |
| Vehicle | | | | 5.47 ± | 2.76 |

### Example 5. Comparison of Capsids and Mammals

### A. Rodents Comparison

Mice (normal e.g., C57BL/6; ˜25 g; n=8) and Rats (e.g., Sprague-Dawley; ˜300 g; n=8) are administered via intrathecal and/or intrastriatal administration either a control of vehicle only (Tributyl citrate (TBC)-180 mM sodium chloride, 10 mM sodium phosphate and 0.001% pluronic acid) or AAVrh10 or AAVDJ serotypes which were packaged with a transgene (FXN).

During the study, the body weight of each animal is taken weekly, daily observations of the behavior of each animal are recorded and blood and CSF samples pre-dose and post-AAV infusion are taken for analysis. After 6 weeks, the animals are compared for distribution and level of transgene (FXN) expression in spinal cord and DRGs as well as the percent target cell transduction and distribution, relative transduction of peripheral organs. The transduction pattern is evaluated using a method known in the art, and cell tropism using double label against neurological marks.

### B. Rodents Serotype Study-Intrathecal Administration

Rodents (Sprague-Dawley Rat; n=2 for control and 4 for serotypes) are administered via slow intrathecal administration a bolus (10 ul) of either a control of vehicle only (PBS, 0.001% F-68) or AAV1, AAV2, AAV6, AAV9, AAVrh10, AAVDJ or AAVDJ8 serotypes which were packaged with a transgene (GFP) as outlined in Table 10.

TABLE 10
IT Study Design

| Group | Serotype | Dose (vg) |
|---|---|---|
| 1 | AAV1 | 3.73 × 10¹⁰ |
| 2 | AAV2 | TBD |
| 3 | AAV6 | 3.73 × 10¹⁰ |
| 4 | AAVrh10 | 3.73 × 10¹⁰ |
| 5 | AAVDJ | 3.73 × 10¹⁰ |
| 6 | AAVDJ8 | 3.73 × 10¹⁰ |
| 7 | AAV9 | 3.73 × 10¹⁰ |
| 8 | N/A - Vehicle only | 0 |

During the study, the body weight of each animal is taken weekly, daily observations of the behavior of each animal are recorded. After 28 days the brain, spinal cord, DRGs, sciatic nerve and sympathetic ganglia, SGC, hind paw skin, liver and heart may be analyzed by methods known in the art such as PFA transcardiac perfusion, IHC and microscope analysis.

### C. Rodents Serotype Study-Intrathecal Administration

Rodents (Sprague-Dawley Rat; n=2 for control and 4 for serotypes) are administered via slow intrastriatal administration (10 ul at 0.5 ul/min) of either a control of vehicle only (PBS, 0.001% F-68) or AAV1, AAV2, AAV6, AAV9, AAVrh10, AAVDJ or AAVDJ8 serotypes which were packaged with a transgene (GFP) as outlined in Table 11.

TABLE 11
IS Study Design

| Group | Serotype | Dose (vg) |
|---|---|---|
| 1 | AAV1 | 1.9 × 10¹⁰ |
| 2 | AAV2 | TBD |
| 3 | AAV6 | 1.9 × 10¹⁰ |
| 4 | AAVrh10 | 1.9 × 10¹⁰ |
| 5 | AAVDJ | 1.9 × 10¹⁰ |
| 6 | AAVDJ8 | 1.9 × 10¹⁰ |
| 7 | AAV9 | 1.9 × 10¹⁰ |
| 8 | N/A - Vehicle only | 0 |

During the study, the body weight of each animal is taken weekly, daily observations of the behavior of each animal are recorded. After 28 days the brain, spinal cord, DRGs, sciatic nerve and sympathetic ganglia, SGC, hind paw skin, liver and heart may be analyzed by methods known in the art such as PFA transcardiac perfusion, IHC and microscope analysis.

### D. Non-Human Primates Serotype Study

Non-human primates (cynomolgus adult male or female prescreen for capsid-specific low anti-AAV antibodies; n=4 per group and 2 control) are administered via intrathecal (L1) administration either a control of vehicle only (Tributyl citrate (TBC)-180 mM sodium chloride, 10 mM sodium phosphate and 0.001% pluronic acid) or AAV2, AAVDJ, AAVDJ8 or AAV1 serotypes which are packaged with a transgene (GFP) at three doses of 1×10¹³ at a rate of 1 ml bolus/hour (total volume 3 ml).

During the study, the body weight of each animal is taken weekly, daily observations of the behavior of each animal are recorded and blood and CSF samples pre-dose and post-AAV infusion are taken for analysis. Approximately 3 weeks after administration the animals are compared for distribution and level of transgene (GFP) expression in NHP spinal cord and DRGs as well as the percent target cell transduction and distribution, relative transduction of peripheral organs. The transduction patter is evaluated using GFP-IHC and/or GFP-ISH, and cell tropism using double label against neurological marks.

### E. Non-Human Primates Expanded Serotype Study

Non-human primates are administered via intrathecal and/or intrastriatal administration either AAV1, AAV2, AAV6, AAV9, AAVDJ or AAVDJ8 serotypes which are packaged with a transgene (GFP). Approximately 2-3 weeks after administration the animals are compared for distribution and level of transgene (GFP) expression in NHP spinal cord and DRGs as well as the percent target cell transduction and distribution, relative transduction of peripheral organs. The transduction patter is evaluated using GFP-IHC and/or GFP-ISH, and cell tropism using double label against neurological marks.

### Example 6. Comparison of Capsids and Transgenes

Non-human primates (n=4; 7 groups; Cynomolgus, pre-screened for capsid-specific low anti-AAV antibodies) are administered by intrathecal administration (L1) at 1 ml bolus/hour, either AAVDJ or AAVrh10 serotypes which are packaged with human or cynomolgus (Cyno) forms of FXN in a delivery vehicle (Tributyl citrate (TBC)-180 mM sodium chloride, 10 mM sodium phosphate and 0.001% pluronic acid). Empty AAVDJ and/or AAVrh10 capsids will be used a controls. The study design is shown in Table 12.

TABLE 12
Expression and Pathology

| Group | Capsid | Genome | Transgene | Dose | Total Volume |
|---|---|---|---|---|---|
| 1 | AAVDJ | SC | Human FXN | 1 × 10¹³ | 3 ml |
| 2 | AAVrh10 | SC | Human FXN | 1 × 10¹³ | 3 ml |
| 3 | AAVrh10 | SC | Cyno FXN | 1 × 10¹³ | 3 ml |
| 4 | AAVrh10 | SC | Cyno FXN | 1 × 10¹² | 3 ml |
| 5 | AAVrh10 | SC | Cyno FXN | TBD | 3 ml |
| 6 | AAVDJ | — | — | 0 | 3 ml |
| 7 | AAVrh10 | — | — | 0 | 3 ml |

During the study, the body weight of each animal is taken weekly, daily observations of the behavior of each animal are recorded and blood and CSF samples pre-dose and post-AAV infusion are taken for analysis. After 6 weeks the expression of the transgene and pathology will be conducted on all NHPs to determine the effect of the capsid, transgene (human (non-self) vs. cynomolgus (self)), dose and immune reaction to the capsid and/or transgene.

### Example 7. Comparison Systemic Delivery of Capsids

### A. Intravascular Injection

C57BL/6 mice (n=1-5) were administered by bolus tail vein intravascular injection administration a dose as outlined in Table 13 of either AAV9, AAV2, AAV5, AAV6, AAVrh10, AAVDJ8 or AAVDJ serotypes which were packaged with FXN in a delivery vehicle (PBS, 5% sorbitol and 0.001% F-68) or the delivery vehicle alone. The study design is shown in Table 13.

TABLE 13
Study Design

| | | | | | | Total Volume |
|---|---|---|---|---|---|---|
| Test Article | Capsid | Genome | n | Dose (vg) | | (ul) |
| VCAV-01801-B | AAV9 | SC | 5 | 5 × | 10¹¹ | 100 |
| VCAV-01791 | AAV9 | SS | 5 | 5 × | 10¹¹ | 100 |
| VCAV-01870 | AAV2 | SS | 5 | 5 × | 10¹¹ | 100 |
| VCAV-01871 | AAV5 | SS | 5 | 5 × | 10¹¹ | 100 |
| VCAV-01851 | AAV6 | SS | 5 | 5 × | 10¹¹ | 100 |
| VCAV-01842 | AAVrh10 | SS | 5 | 5 × | 10¹¹ | 100 |
| VCAV-01962 | AAVrh10 | SC | 2 | 5 × | 10¹¹ | 100 |
| | | | 1 | 2.5 × | 10¹¹ | 100 |
| VCAV-01888 | AAVDJ8 | SS | 4 | 5 × | 10¹¹ | 100 |
| | | | 1 | 2.5 × | 10¹¹ | 100 |
| VCAV-01858 | AAVDJ | SS | 4 | 5 × | 10¹¹ | 100 |
| | | | 1 | 2.5 × | 10¹¹ | 100 |

| | | | | | |
|---|---|---|---|---|---|
| Vehicle | — | — | 5 | — | 100 |

During the study (4 weeks), the body weight of each animal was taken prior to administration and weekly during the study, daily observations of the behavior of each animal were recorded and blood and CSF samples pre-dose and post-AAV infusion are taken for analysis. After 4 weeks, serum was collected as well as samples of the liver (L), cortex (Ctx), Striatum (Cpu), Hippocampus (Hipp), Thalamus (TH), Hemisphere (HP), Cervical spinal cord (SC-C), Thoracic spinal cord (SC-T), Lumbar spinal cord (SC-L), Brainstem (Br), Cerebellum (Cb), Heart (H), Lung, Kidney (K), Spleen (S), Gastrocnemius muscle (SM), and DRG.

Most of the groups continued to gain body weight during the study period. The scAAVrh10 group started to lose body weight four weeks after injection.

Levels of human frataxin in mouse liver on day 28 are shown in Table 14. scAAVrh10 resulted in the highest frataxin expression in the liver (scAAVrh10>scAAV9>ssAAVDJ8>ssAAVrh10>ssAAVDJ>ssAAV6>ssAAV9>ssAAV5>ssAAV2). Self-complementary AAV9 and AAVrh10 resulted in 26 and 18 fold higher expression in the liver than single-stranded vectors, respectfully. Of all the single-stranded vectors, ssAAVDJ8 resulted in the highest liver levels.

TABLE 14
Human Frataxin Levels

| Vector | Frataxin (ng/mg) |
|---|---|
| scAAVrh10 | 19,428 |
| scAAV9 | 8,883 |
| ssAAVDJ8 | 1,849 |
| ssAAVrh10 | 1,057 |
| ssAAVDJ | 811 |
| ssAAV6 | 569 |
| ssAAV9 | 340 |
| ssAAV5 | 90 |
| ssAAV2 | 81 |

Human frataxin was detected in both the mouse cortex and striatum for scAAV9, scAAVrh10 and ssAAVDJ8. scAAV9 injection resulted in the highest frataxin expression (˜2 ng frataxin/mg protein) in both the cortex and striatum (scAAV9>scAAVrh10>ssAAVDJ8).

Human frataxin was detected in serum on day 28 in the scAAVrh10 and scAAV9 groups.

### B. Intravascular and Intrastriatal Injection

C57BL/6 mice (n=2-5) were administered by bolus tail vein intravascular injection (IV) (1×10¹² vg/100 ul) or intrastriatal CM infusion (IS) (5×10¹⁰ vg/4 ul over 10 minute infusion) as outlined in Table 15 of either AAV9, AAVrh10, or AAVDJ8 serotypes which were packaged with FXN in a delivery vehicle (PBS, 5% sorbitol and 0.001% F-68) or the delivery vehicle alone. The study design is shown in Table 15.

TABLE 15
Study Design

| Test Article | Capsid | Genome | n | Route | Dose (vg) |
|---|---|---|---|---|---|
| VCAV-01801-B | AAV9 | SC | 4 | IV | 1 × 10¹² |
| VCAV-01962 | AAVrh10 | SC | 4 | IV | 1 × 10¹² |
| VCAV-01888 | AAVDJ8 | SS | 5 | IV | 1 × 10¹² |
| VCAV-01801-B | AAV9 | SC | 3 | IS | 5 × 10¹⁰ |
| VCAV-01962 | AAVrh10 | SC | 2 | IS | 5 × 10¹⁰ |
| VCAV-01888 | AAVDJ8 | SS | 3 | IS | 5 × 10¹⁰ |

During the study (4 weeks), the body weight of each animal was taken prior to administration and weekly during the study, daily observations of the behavior of each animal were recorded and blood and CSF samples pre-dose and post-AAV infusion are taken for analysis. After 4 weeks, serum was collected as well as samples of the liver (L), cortex (Ctx), Striatum (Cpu), Hippocampus (Hipp), Thalamus (TH), Hemisphere (HP), Cervical spinal cord (SC-C), Thoracic spinal cord (SC-T), Lumbar spinal cord (SC-L), Brainstem (Br), Cerebellum (Cb), Heart (H), Lung, Kidney (K), Spleen (S), Gastrocnemius muscle (SM), and DRG.

Most of the groups continued to gain body weight during the study period. The scAAVrh10 group started to lose body weight three weeks after injection.

For IV injection, the greatest expression was seen in the liver and the lowest was seen in the striatum and cortex (Liver>DRG>spinal cord>cortex and striatum).

Levels of human frataxin in mouse liver on day 28 are shown in Table 16 for IV injection. scAAVrh10 resulted in the highest frataxin expression in the liver after IV injection (scAAVrh10>scAAV9>ssAAVDJ8).

TABLE 16
Human Frataxin Levels in the Liver After IV Administration

| Vector | Frataxin (ng/mg) |
|---|---|
| scAAVrh10 | 34,303 |
| scAAV9 | 11,939 |
| ssAAVDJ8 | 3,285 |

Human frataxin was detected in both the mouse cortex and striatum on day 28 following IV injection and in the striatum for IS administration as shown in Tables 17 and 18. scAAV9 and scAAVrh10 showed the highest expression for IV and IS administration for the striatum and in the cortex for IV administration scAAV9 and scAAVrh10 also showed the highest expression.

TABLE 17
Human Frataxin Levels in the Striatum

| Route of | | |
|---|---|---|
| Administration | Vector | Frataxin (ng/mg) |
| IV | scAAV9 | 1 |
| | scAAVrh10 | 1 |
| | ssAAVDJ8 | 0.3 |
| IS | scAAV9 | 4,713 |
| | scAAVrh10 | 3,793 |
| | ssAAVDJ8 | 655 |

TABLE 18
Human Frataxin Levels in the Cortex

| Route of | | |
|---|---|---|
| Administration | Vector | Frataxin (ng/mg) |
| IV | scAAV9 | 1 |
| | scAAVrh10 | 1 |
| | ssAAVDJ8 | 0.4 |

Human frataxin was detected in the spinal cord and DRG on day 28 following IV injection as shown in Table 19. scAAVrh10 showed the highest expression in the spinal cord and DRG. ssAAVDJ8 had the second highest in the spinal cord and was the lowest in DRG. scAAV9 was the second highest in DRG and the lowest in the spinal cord.

TABLE 19
Human Frataxin Levels in the Striatum

| Route of | | Frataxin (ng/mg) | |
|---|---|---|---|
| Administration | Vector | Spinal Cord | DRG |
| IV | scAAV9 | 2 | 12 |
| | scAAVrh10 | 5 | 16 |
| | ssAAVDJ8 | 3 | 5 |

### Example 8. Non-Human Primate Intrathecal Delivery Study

Non-human primates (NHPs) (n=24 male Cynomolgus with low serum anti-AAV antibody titers) were administered an AAV particle, serotype rh.10 and self-complementary (SC), via bolus or continuous intrathecal (IT) delivery using implanted chronic catheters with tips at cervical and/or lumbar levels as outlined in Table 20.

TABLE 20
Study Design

| Group | Description | Site(s) | Rate | Vol. | | Conc. (vg/ml) | Dose (vg) | N |
|---|---|---|---|---|---|---|---|---|
| 1 | Bolus; IT-lumbar | L1 | Bolus | 1 | ml | 1 × 10¹³ | 1 × 10¹³ | 4 |
| 2 | Bolus; IT-cervical | C1 | Bolus | 1 | ml | 1 × 10¹³ | 1 × 10¹³ | 4 |
| 3 | 10 h infusion; high | L1 | 0.1 ml/h | 1 | ml | 1 × 10¹³ | 1 × 10¹³ | 4 |
| | titer/low volume | | | | | | | |
| 4 | 10 h infusion; high | C1 | 0.1 ml/h | 1 | ml | 1 × 10¹³ | 1 × 10¹³ | 4 |
| | titer/low volume | | | | | | | |
| 5 | 10 h infusion; low | L1 | 1.0 ml/h | 10 | ml | 1 × 10¹³ | 1 × 10¹³ | 4 |
| | titer/high volume | | | | | | | |
| 6 | 10 h infusion; low | C1 | 1.0 ml/h | 10 | ml | 0.1 × 10¹³ | 1 × 10¹³ | 4 |
| | titer/high volume | | | | | | | |

The study compared the location of administration as well as infusion rate and volume. The severity of ganglion infiltrates and neuronal degeneration in DRG and the spinal cord varied among the groups. Lumbar was greater than cervical groups and the slow 1 mL infusion was greater than the 10 mL infusion which was greater than the 1 mL bolus.

### Example 9. Intrathecal Delivery Study

Pigs (n=6 Gottingen minipigs) were administered an AAV particle, serotype rh.10, via bolus or continuous intrathecal (IT) delivery using implanted chronic catheters with tips at cervical, thoracic and/or lumbar levels as outlined in Table 21.

TABLE 21
Study Design

| Group | Description | Site(s) | Rate | Vol. | | Conc. (vg/ml) | Dose (vg) | N |
|---|---|---|---|---|---|---|---|---|
| 1 | 1-site bolus | Cervical | Slow Bolus | 3 | ml | 1 × 10¹³ | 3 × 10¹³ | 2 |
| 2 | 3-site bolus | Cervical | Slow Bolus | 3 × 1 | ml | 1 × 10¹³ | 3 × 10¹³ | 2 |
| | | Thoracic | | | | | | |
| | | Lumbar | | | | | | |
| 3 | 1-site 10-hour | Cervical | 1.0 ml/h | 10 | ml | 0.3 × 10¹³ | 3 × 10¹³ | 2 |
| | infusion | | | | | | | |

The study compared the location of administration as well as infusion rate and volume. There was successful expression of the protein in both the spinal cord and DRG after intrathecal delivery. Greater transduction/distribution S1>L1>C4 spinal cord and overall greater transduction in spinal cord from 3 site injections and in DRG from cervical bolus. There were no adverse pathology findings in this study.

### Example 10. Animal Models

AAV9, AAV2, AAV1, AAV5, AAV6, AAVrh10, AAVDJ8 or AAVDJ serotypes which were packaged with FXN having either a full length or functional deleted mutant of CBA, CMV, PGK or FXN promoter. The AAV particles are administered in a delivery vehicle (PBS, 5% sorbitol and 0.001% F-68) or the delivery vehicle alone is administered by intraparenchymal administration, intracerebroventricular infusion or intrathecal infusion to at least one set of mice from the mouse models described in Table 22 (From Table 1 of Martelli et al. 2012, the contents of which is herein incorporated by reference). To evaluate the effect of different dosages, a low, medium and high vector dose is administered with a 10-fold dose escalation between the dose levels.

TABLE 22
Mouse Models

| Model | Model/ | |
|---|---|---|
| Type | Genotype | Notes/Phenotype |
| Knockout | FXN- | Embryonic lethality during gastrulation |
| | knockout | (Cossee et al. 2000) |
| | mouse | |
| Conditional | MCK- | Muscle creatine promoter. FXN deletion in heart |
| mouse | Cre | and skeletal muscle. Reduced lifespan (76 +/− |
| models | | 10 days) and hyper trophic cardiomyopathy but |
| of FXN | | no skeletal muscle phenotype. Early Fe—S |
| deletion | | cluster deficit and late mitochondrial iron |
| | | accumulation. |
| | | No sign of oxidative stress (Puccio et al. 2001) |
| | NSE-Cre | Neuron-specific enolase promoter. FXN deletion |
| | | in nervous system, heart and liver. Reduced |
| | | lifespan (29 ± 9 days). Severe neuronal and |
| | | cardiac phenotype (Puccio et al. 2001) |
| | Prp- | Tamoxifen-inducible Cre, prion promoter. Fxn |
| | CreER | deletion in DRG and cerebellum. Progressive |
| | | spinocerebellar and sensory ataxia. |
| | | Neurodegeneration of sensory neurons in DRG |
| | | and granular layer in cerebellum. Abnormal |
| | | autophagy in DRG. (Simon et al. 2004) |
| | Ins2-Cre | Insulin promoter. Fxn deletion in pancreatic β- |
| | | cells; diabetes mellitus (Ristow et al. 2003) |
| | ALB-Cre | Albumin promoter. Fxn deletion in hepatocytes. |
| | | Tumor formation or liver regeneration. |
| | | (Thierbach et al. 2005) |
| Mouse | KIKI | Double knock-in with 230 GAA repeats. No |
| models | | overt phenotype. Transcriptional deregulation |
| with GAA | | involving the PPAR pathway. Markers of |
| expansions | | heterochromatin on the GAA tract. (Miranda et |
| in FXN | | al. 2002) |
| | KIKO | Simple knock-in crossed with knockout mouse. |
| | | 26-32% residual frataxin expression. No overt |
| | | phenotype. Transcriptional deregulation |
| | | involving the PPAR pathway. (Miranda et al. |
| | | 2002) |
| | YG8R | YAC containing the full human FXN locus with |
| | | a GAA expansion and deleted for endogenous |
| | | murine frataxin. Progressive ataxia with affected |
| | | DRG. No cardiopathy but mitochondrial iron |
| | | accumulation and lipid peroxidation. Markers of |
| | | heterochromatin on the GAA tract. Tissue |
| | | dependent GAA instability. (Al-Mahdawi et al. |
| | | 2006) |

During the study, the body weight of each animal is taken weekly, daily observations of the behavior of each animal are recorded and blood and CSF samples pre-dose and post-AAV infusion are taken for analysis. After about 6-8 weeks, the animals are compared for distribution and level of transgene (FXN) expression in neural tissues (e.g., brain, spinal cord, DRGs) as well as the percent target cell transduction and distribution, relative transduction of peripheral organs (e.g., liver, heart and pancreas). The transduction pattern is evaluated using a method known in the art, and cell tropism using double label against neurological marks.

### Example 11. Non Human Primate Study

5 groups of adult non-human primates (n=2-3 per group; Cynomolgus) are pre-screened for serotype-specific low anti-AAV antibody levels. The non-human primates will be pre-implanted with catheters and left in place after delivery for CSF sampling. Delivery of the AAV particles (capsids may be selected from AAV9, AAV2, AAV1, AAV5, AAV6, AAVrh10, AAVDJ8 or AAVDJ serotypes; promoter may be either a full length or functional deleted mutant of CBA, CMV, PGK or FXN; payload is either wild type or non-wild type FXN) in a delivery vehicle (e.g., PBS, 5% sorbitol and 0.001% F-68) or delivery vehicle alone to the non-human primates will be by intrathecal lumbar administration by continuous infusion at a rate of 1 ml over 1 hour. Study design is shown in Table 23 where subscript X and Y refer to different capsids and subscript 1 and 2 refer to different promoters.

To evaluate the effect of different dosages, a low, medium and high vector dose is administered with a 10-fold dose escalation between the dose levels.

TABLE 23
Study Design

| | Vector Dose | | |
|---|---|---|---|
| Treatment Group | Low | Medium | High |
| AAV_{X}-Promoter₁-hFXN | n = 2 | n = 2 | n = 3 |
| AAV_{X}-Promoter₂-hFXN | n = 2 | n = 2 | n = 3 |
| AAV_{Y}-Promoter₁-hFXN | n = 2 | n = 2 | n = 3 |
| AAV_{Y}-Promoter₂-hFXN | n = 2 | n = 2 | n = 3 |
| Vehicle Control (n = 2) | n/a | n/a | n/a |

During the study, daily observations of the behavior of each animal are recorded and blood and (baseline, 1-7 days post infusion and biweekly thereafter) and CSF samples (baseline, weekly post infusion if catheters remain usable and at necropsy) are taken for analysis. After about 8 weeks, blood and CSF analysis for the AAV levels (acute time points) and anti-AAV antibodies is gathered for each animal. Animals are perfused with heparinized saline and the distribution and level of transgene (FXN) expression in neural tissues (e.g., brain, spinal cord, DRGs) as well as the percent target cell transduction and distribution, relative transduction of peripheral organs (e.g., liver, heart and pancreas) is determined. Tissue samples will be frozen for molecular and biochemical analysis and/or placed in ice-cold paraformaldehyde for histological evaluation for molecular biology aspects (e.g., vector DNA, mRNA (PCR)), biochemistry (FXN protein (Mass spectrometry, ELISA)) and neurohistology (FXN immunochemistry and in situ hybridization). One set of fixed specimens can also be sent for independent, blinded histopathological evaluation.

### Example 12. CSF Flow Dynamics Studies

The CSF flow of non-human primates (N=8; adult cynomolgus) is studied by MRI imaging. The study is conducted with and without implant IT catheter (cervical N=4; lumbar N=4).

Particle distribution in the CSF is studied by administering non-human primates (N=8; adult cynomolgus) Gadoluminate via implant IT catheter (cervical N=4; lumbar N=4). The Gadoluminate dosing is bolus (1 ml) and 10 hour infusion (1 ml/h). The particle distribution is monitored by MRI imaging.

Particle distribution in the CSF compared to AAV expression is studied by administering non-human primates (N=8; adult cynomolgus) Gadoluminate and AAV serotype packaged with GFP via implant IT catheter (cervical N=4; lumbar N=4). The particle distribution is monitored by MRI imaging. The AAV expression is analyzed by immunohistochemistry after necropsy.

### Example 13. Dose Response Study

Non-human primates (n=4; 6 groups; cynomolgus 3-years old, pre-screened for capsid-specific low anti-AAV antibodies) are administered using an IT-lumbar implanted catheter at L1, two 1 ml bolus/hour infusions of self-complementary CBA-hFXN vector (vehicle—PBS with 0.001% F-68) via intrathecal (IT) administration. Empty capsids and vehicle alone will be used a controls. The study design is shown in Table 24.

TABLE 24
Study Design

| Group | Capsid | Genome | Transgene | Dose (vg) |
|---|---|---|---|---|
| 1 | Vehicle | — | — | N/A |
| | (PBS) | | | |
| 2 | Empty | — | — | 2 × 10¹³ |
| | Vector | | | |
| 3 | AAVDJ | SC | Human FXN | 2 × 10¹³ |
| 4 | AAVDJ | SC | Human FXN | 2 × 10¹² |
| 5 | AAVrh10 | SC | Human FXN | 2 × 10¹¹ |
| 6 | AAVDJ | SC | Human FXN | 2 × 10¹⁰ |

During the study, the body weight of each animal is taken weekly, daily observations of the behavior of each animal are recorded and blood and CSF samples pre-dose and post-AAV infusion are taken for analysis. At the end of the study, the expression of the transgene and pathology will be conducted on all NHPs to determine the effect of the capsid, dose and immune reaction to the capsid.

### Example 14. Comparison of Capsids

Non-human primates (n=4; 6 groups; cynomolgus, pre-screened for capsid-specific low anti-AAV antibodies) are administered by intrathecal administration (L1) at 1 ml bolus/hour, either an AAV particle (self-complementary and CBA promoter) described in Table 25 in a delivery vehicle. The study design is shown in Table 25.

TABLE 25
Expression and Pathology

| Group | Capsid | Transgene | Dose (vg) | Total Volume |
|---|---|---|---|---|
| 1 | AAV2 | GFP | ≤3 × 10¹³ | Up to 3 ml |
| 2 | AAVDJ | GFP | ≤3 × 10¹³ | Up to 3 ml |
| 3 | AAVDJ8 | GFP | ≤3 × 10¹³ | Up to 3 ml |
| 4 | AAV1 | GFP | ≤3 × 10¹³ | Up to 3 ml |
| 5 | AAV6 | GFP | ≤3 × 10¹³ | Up to 3 ml |
| 6 | AAV9 | GFP | ≤3 × 10¹³ | Up to 3 ml |

During the study, the body weight of each animal is taken weekly, daily observations of the behavior of each animal are recorded and blood and CSF samples pre-dose and post-AAV infusion are taken for analysis. At the end of the study (approximately 3 weeks) the expression of the transgene and pathology will be conducted on all NHPs to determine the effect of the capsid, and immune reaction to the capsid.

### Example 15. IT Volume Comparison

Rodents (n=8; 6 groups) are administered by intrathecal administration a slow small volume or a rapid large volume in the Trendelenburg position a composition of AAV particle (self-complementary, CBA promoter, rh10 capsid and an HA (human influenza hemagglutinin) tag) described in Table 26 in a delivery vehicle at a dose of 9×10¹⁰ vg (0.7 ml at 6.7×10¹² vg/ml). The study design is shown in Table 26.

TABLE 26
Expression and Pathology

| | | | Saline | | | | Inj. |
|---|---|---|---|---|---|---|---|
| | | Injection | Flush Vol. | Total Vol. | Inj. Vol. | Flow Rate | Duration |
| Group | Site | Vol. (ul) | (ul) | (ul) | (ul) | (ul/min) | (min) |
| 1 | L1 (CM entry) | 15 | 5 | 21 | 15 | 1 | 21 |
| 2 | C5 (CM entry) | 15 | 5 | 21 | 15 | 1 | 21 |
| 3 | L1 (L5 or | 30 | 45 | 76 | 70 | 150 | 0.5 |
| | CM entry) | | | | | | |
| 4 | L1 (L5 or | 15 | 5 | 21 | 15 | 1 | 21 (in head, |
| | CM entry) | | | | | | down tilt) |
| 5 | C5 (CM entry) | 15 | 5 | 21 | 15 | 1 | 21 (in head, |
| | | | | | | | down tilt) |
| 6 | C5, T1, L1 | 15 each | 5 once | 51 | 45 | 1 | 51 |
| | (CM entry) | (C, T, L) | | | | | |

During the study, the body weight of each animal is taken weekly, daily observations of the behavior of each animal are recorded and blood and CSF samples pre-dose and post-AAV infusion are taken for analysis. At the end of the study the expression of the transgene (FXN) and pathology will be conducted on all rodents to determine the effect of the capsid, and immune reaction to the capsid in the cervical, thoracic, lumbar, spinal cord and DRGs.

While the present invention has been described at some length and with some particularity with respect to the several described embodiments, it is not intended that it should be limited to any such particulars or embodiments or any particular embodiment, but it is to be construed with references to the appended claims so as to provide the broadest possible interpretation of such claims in view of the prior art and, therefore, to effectively encompass the intended scope of the invention.

All publications, patent applications, patents, and other references mentioned herein are incorporated by reference in their entirety. In case of conflict, the present specification, including definitions, will control. In addition, section headings, the materials, methods, and examples are illustrative only and not intended to be limiting.

We claim:

1. A method of increasing the level of a protein in the CNS of a subject in need thereof comprising administering to said subject an effective amount of an AAV particle comprising a vector genome packaged in a capsid, said capsid having a serotype selected from the group consisting of AAVrh.10 (AAVrh10), AAV-DJ (AAVDJ), AAV-DJ8 (AAVDJ8), AAV1, AAV2, AAV2G9, AAV3, AAV3a, AAV3b, AAV3-3, AAV4, AAV4-4, AAV5, AAV6, AAV6.1, AAV6.2, AAV6.1.2, AAV7, AAV7.2, AAV8, AAV9, AAV9.11, AAV9.13, AAV9.16, AAV9.24, AAV9.45, AAV9.47, AAV9.61, AAV9.68, AAV9.84, AAV9.9, AAV10, AAV11, AAV12, AAV16.3, AAV24.1, AAV27.3, AAV42.12, AAV42-1b, AAV42-2, AAV42-3a, AAV42-3b, AAV42-4, AAV42-5a, AAV42-5b, AAV42-6b, AAV42-8, AAV42-10, AAV42-11, AAV42-12, AAV42-13, AAV42-15, AAV42-aa, AAV43-1, AAV43-12, AAV43-20, AAV43-21, AAV43-23, AAV43-25, AAV43-5, AAV44.1, AAV44.2, AAV44.5, AAV223.1, AAV223.2, AAV223.4, AAV223.5, AAV223.6, AAV223.7, AAV1-7/rh.48, AAV1-8/rh.49, AAV2-15/rh.62, AAV2-3/rh.61, AAV2-4/rh.50, AAV2-5/rh.51, AAV3.1/hu.6, AAV3.1/hu.9, AAV3-9/rh.52, AAV3-11/rh.53, AAV4-8/r11.64, AAV4-9/rh.54, AAV4-19/rh.55, AAV5-3/rh.57, AAV5-22/rh.58, AAV7.3/hu.7, AAV16.8/hu.10, AAV16.12/hu.11, AAV29.3/bb.1, AAV29.5/bb.2, AAV106.1/hu.37, AAV114.3/hu.40, AAV127.2/hu.41, AAV127.5/hu.42, AAV128.3/hu.44, AAV130.4/hu.48, AAV145.1/hu.53, AAV145.5/hu.54, AAV145.6/hu.55, AAV161.10/hu.60, AAV161.6/hu.61, AAV33.12/hu.17, AAV33.4/hu.15, AAV33.8/hu.16, AAV52/hu.19, AAV52.1/hu.20, AAV58.2/hu.25, AAVA3.3, AAVA3.4, AAVA3.5, AAVA3.7, AAVC1, AAVC2, AAVC5, AAVF3, AAVF5, AAVH2, AAVrh.72, AAVhu.8, AAVrh.68, AAVrh.70, AAVpi.1, AAVpi.3, AAVpi.2, AAVrh.60, AAVrh.44, AAVrh.65, AAVrh.55, AAVrh.47, AAVrh.69, AAVrh.45, AAVrh.59, AAVhu.12, AAVH6, AAVLK03, AAVH-1/hu.1, AAVH-5/hu.3, AAVLG-10/rh.40, AAVLG-4/rh.38, AAVLG-9/hu.39, AAVN721-8/rh.43, AAVCh.5, AAVCh.5R1, AAVcy.2, AAVcy.3, AAVcy.4, AAVcy.5, AAVCy.5R1, AAVCy.5R2, AAVCy.5R3, AAVCy.5R4, AAVcy.6, AAVhu.1, AAVhu.2, AAVhu.3, AAVhu.4, AAVhu.5, AAVhu.6, AAVhu.7, AAVhu.9, AAVhu.10, AAVhu.11, AAVhu.13, AAVhu.15, AAVhu.16, AAVhu.17, AAVhu.18, AAVhu.20, AAVhu.21, AAVhu.22, AAVhu.23.2, AAVhu.24, AAVhu.25, AAVhu.27, AAVhu.28, AAVhu.29, AAVhu.29R, AAVhu.31, AAVhu.32, AAVhu.34, AAVhu.35, AAVhu.37, AAVhu.39, AAVhu.40, AAVhu.41, AAVhu.42, AAVhu.43, AAVhu.44, AAVhu.44R1, AAVhu.44R2, AAVhu.44R3, AAVhu.45, AAVhu.46, AAVhu.47, AAVhu.48, AAVhu.48R1, AAVhu.48R2, AAVhu.48R3, AAVhu.49, AAVhu.51, AAVhu.52, AAVhu.54, AAVhu.55, AAVhu.56, AAVhu.57, AAVhu.58, AAVhu.60, AAVhu.61, AAVhu.63, AAVhu.64, AAVhu.66, AAVhu.67, AAVhu.14/9, AAVhu.t 19, AAVrh.2, AAVrh.2R, AAVrh.8, AAVrh.8R, AAVrh.12, AAVrh.13, AAVrh.13R, AAVrh.14, AAVrh.17, AAVrh.18, AAVrh.19, AAVrh.20, AAVrh.21, AAVrh.22, AAVrh.23, AAVrh.24, AAVrh.25, AAVrh.31, AAVrh.32, AAVrh.33, AAVrh.34, AAVrh.35, AAVrh.36, AAVrh.37, AAVrh.37R2, AAVrh.38, AAVrh.39, AAVrh.40, AAVrh.46, AAVrh.48, AAVrh.48.1, AAVrh.48.1.2, AAVrh.48.2, AAVrh.49, AAVrh.51, AAVrh.52, AAVrh.53, AAVrh.54, AAVrh.56, AAVrh.57, AAVrh.58, AAVrh.61, AAVrh.64, AAVrh.64R1, AAVrh.64R2, AAVrh.67, AAVrh.73, AAVrh.74, AAVrh8R, AAVrh8R A586R mutant, AAVrh8R R533A mutant, AAAV, BAAV, caprine AAV, bovine AAV, AAVhE1.1, AAVhEr1.5, AAVhER1.14, AAVhEr1.8, AAVhEr1.16, AAVhEr1.18, AAVhEr1.35, AAVhEr1.7, AAVhEr1.36, AAVhEr2.29, AAVhEr2.4, AAVhEr2.16, AAVhEr2.30, AAVhEr2.31, AAVhEr2.36, AAVhER1.23, AAVhEr3.1, AAV2.5T, AAV-PAEC, AAV-LK01, AAV-LK02, AAV-LK03, AAV-LK04, AAV-LK05, AAV-LK06, AAV-LK07, AAV-LK08, AAV-LK09, AAV-LK10, AAV-LK11, AAV-LK12, AAV-LK13, AAV-LK14, AAV-LK15, AAV-LK16, AAV-LK17, AAV-LK18, AAV-LK19, AAV-PAEC2, AAV-PAEC4, AAV-PAEC6, AAV-PAEC7, AAV-PAEC8, AAV-PAEC 11, AAV-PAEC12, AAV-2-pre-miRNA-101, AAV-8h, AAV-8b, AAV-h, AAV-b, AAV SM 10-2, AAV Shuffle 100-1, AAV Shuffle 100-3, AAV Shuffle 100-7, AAV Shuffle 10-2, AAV Shuffle 10-6, AAV Shuffle 10-8, AAV Shuffle 100-2, AAV SM 10-1, AAV SM 10-8, AAV SM 100-3, AAV SM 100-10, BNP61 AAV, BNP62 AAV, BNP63 AAV, AAVrh.50, AAVrh.43, AAVrh.62, AAVrh.48, AAVhu.19, AAVhu.11, AAVhu.53, AAV4-8/rh.64, AAVLG-9/hu.39, AAV54.5/hu.23, AAV54.2/hu.22, AAV54.7/hu.24, AAV54.1/hu.21, AAV54.4R/hu.27, AAV46.2/hu.28, AAV46.6/hu.29, AAV128.1/hu.43, true type AAV (ttAAV), UPENN AAV 10 and/or Japanese AAV 10 serotypes, and variants thereof.

2. The method of claim 1, wherein the capsid is AAVrh10.

3. The method of claim 1, wherein the capsid is AAV-DJ.

4. The method of claim 1, wherein the capsid is AAV-DJ8.

5. The method of claim 1, wherein the vector genome comprises a promoter, and wherein said promoter is selected from the group consisting of CBA, CMV, PGK, FXN, H1, and fragments or variants thereof.

6. The method of claim 5, wherein the promoter is CBA.

7. The method of claim 5, wherein the promoter is CMV.

8. The method of claim 5, wherein the promoter is FXN.

9. The method of claim 5, wherein the promoter is H1.

10. The method of any of claims 1-9, wherein the administration is a route selected from the group consisting of intrathecal (IT) administration, intraparenchymal (IPa) administration, and intracerebroventricular (ICV) administration.

11. The method of claim 10, wherein the route is IT administration.

12. The method of claim 11, wherein IT administration occurs in at least one location in at least one region of the spine of the subject, and wherein the at least one region of the spine of the subject is selected from the group consisting of cervical, thoracic, lumbar and sacral region.

13. The method of claim 11, wherein IT administration occurs in the cervical region, and wherein IT administration to the cervical region occurs in at least one location selected from the group consisting of C1, C2, C3, C4, C5, C6, and C7.

14. The method of claim 11, wherein IT administration occurs in the thoracic region, and wherein IT administration to the thoracic region occurs in at least one location selected from the group consisting of T1, T2, T3, T3, T4, T5, T6, T7, T8, T9, T10, T11, and T12.

15. The method of claim 11, wherein the IT administration occurs in the lumbar region, and wherein IT administration to the lumbar region occurs in at least one location selected from the group consisting of L1, L2, L3, L4, and L5.

16. The method of claim 11, wherein IT administration occurs in the lumbar region, and wherein IT administration to the sacral region occurs in at least one location selected from the group consisting of S1, S2, S3, S4, and S5.

17. The method of any of claims 12-16, wherein IT administration occurs in one location.

18. The method of claim 17, wherein the location is C1.

19. The method of claim 17, wherein the location is C5.

20. The method of claim 17, wherein the location is T1.

21. The method of claim 17, wherein the location is L1.

22. The method of claim 17, wherein the location is L5.

23. The method of any of claims 12-16, wherein IT administration occurs in three locations.

24. The method of claim 23, wherein the locations are L1, T1 and C5.

25. The method of claim 11, wherein the volume of IT administration is less than 1 mL.

26. The method of claim 11, wherein the volume of IT administration is between about 0.1 mL to about 120 mL.

27. The method of any of claims 11-24, wherein the IT administration is via bolus infusion.

28. The method of any of claims 11-24, wherein the IT administration is via prolonged infusion.

29. The method of claim 28, wherein the prolonged infusion occurs at a volume of more than 1 mL.

30. The method of claim 29, wherein the prolonged infusion occurs at a volume of at least 3 mL.

31. The method of claim 29, wherein the prolonged infusion occurs at a volume of 3 mL.

32. The method of claim 29, wherein the prolonged infusion occurs at a volume of at least 10 mL.

33. The method of claim 29, wherein the prolonged infusion occurs at a volume of 10 mL.

34. The method of claim 28, wherein the prolonged infusion occurs for at least a duration selection from the group consisting of 0.17, 0.33, 0.5, 0.67, 0.83, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, and 36 hour(s).

35. The method of claim 34, wherein the duration is at least one hour.

36. The method of claim 34, wherein the duration is at least 10 hours.

37. The method of claim 28, wherein the prolonged infusion occurs at a constant rate.

38. The method of claim 28, wherein the prolonged infusion occurs at a ramped rate.

39. The method of claim 38, wherein the ramped rate increases over the duration of the prolonged infusion.

40. The method of claim 28, wherein the prolonged infusion occurs at a complex rate alternating between high and low rates over the duration of the prolonged infusion.

41. The method of any one of claims 29-40, wherein the rate of prolonged infusion is between about 0.1 mL/hour and about 25.0 mL/hour.

42. The method of claim 41, wherein the rate of prolonged infusion is selected from the group consisting of 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 10.1, 10.2, 10.3, 10.4, 10.5, 10.6, 10.7, 10.8, 10.9, 11.0, 11.1, 11.2, 11.3, 11.4, 11.5, 11.6, 11.7, 11.8, 11.9, 12.0, 12.1, 12.2, 12.3, 12.4, 12.5, 12.6, 12.7, 12.8, 12.9, 13.0, 13.1, 13.2, 13.3, 13.4, 13.5, 13.6, 13.7, 13.8, 13.9, 14.0, 14.1, 14.2, 14.3, 14.4, 14.5, 14.6, 14.7, 14.8, 14.9, 15.0, 15.1, 15.2, 15.3, 15.4, 15.5, 15.6, 15.7, 15.8, 15.9, 16.0, 16.1, 16.2, 16.3, 16.4, 16.5, 16.6, 16.7, 16.8, 16.9, 17.0, 17.1, 17.2, 17.3, 17.4, 17.5, 17.6, 17.7, 17.8, 17.9, 18.0, 18.1, 18.2, 18.3, 18.4, 18.5, 18.6, 18.7, 18.8, 18.9, 19.0, 19.1, 19.2, 19.3, 19.4, 19.5, 19.6, 19.7, 19.8, 19.9, 20.0, 20.1, 20.2, 20.3, 20.4, 20.5, 20.6, 20.7, 20.8, 20.9, 21.0, 21.1, 21.2, 21.3, 21.4, 21.5, 21.6, 21.7, 21.8, 21.9, 22.0, 22.1, 22.2, 22.3, 22.4, 22.5, 22.6, 22.7, 22.8, 22.9, 23.0, 23.1, 23.2, 23.3, 23.4, 23.5, 23.6, 23.7, 23.8, 23.9, 24.0, 24.1, 24.2, 24.3, 24.4, 24.5, 24.6, 24.7, 24.8, 24.9, and 25.0 mL/hour.

43. The method of claim 42, wherein the rate of prolonged infusion is 1.0 mL/hour.

44. The method of claim 42, wherein the rate of prolonged infusion is 1.5 mL/hour.

45. The method of any one of claims 29-40, wherein the rate of prolonged infusion exceeds the rate of cerebrospinal fluid (CSF) absorption.

46. The method of any one of claims 11-28, wherein the IT administration comprises a total dose between about 1×10⁶ VG and about 1×10¹⁶ VG.

47. The method of claim 46, wherein the total dose is selected from the group consisting of about 1×10⁶, 2×10⁶, 3×10⁶, 4×10⁶, 5×10⁶, 6×10⁶, 7×10⁶, 8×10⁶, 9×10⁶, 1×10⁷, 2×10⁷, 3×10⁷, 4×10⁷, 5×10⁷, 6×10⁷, 7×10⁷, 8×10⁷, 9×10⁷, 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 2×10¹⁰, 3×10¹⁰, 4×10¹⁰, 5×10¹⁰, 6×10¹⁰, 7×10¹⁰, 8×10¹⁰, 9×10¹⁰, 1×10¹¹, 2×10¹¹, 3×10¹¹, 4×10¹¹, 5×10¹¹, 6×10¹¹, 7×10¹¹, 8×10¹¹, 9×10¹¹, 1×10¹², 2×10¹², 3×10¹², 4×10¹², 5×10¹², 6×10¹², 7×10¹², 8×10¹², 9×10¹², 1×10¹³, 2×10¹³, 3×10¹³, 4×10¹³, 5×10¹³, 6×10¹³, 7×10¹³, 8×10¹³, 9×10¹³, 1×10¹⁴, 2×10¹⁴, 3×10¹⁴, 4×10¹⁴, 5×10¹⁴, 6×10¹⁴, 7×10¹⁴, 8×10¹⁴, 9×10¹⁴, 1×10¹⁵, 2×10¹⁵, 3×10¹⁵, 4×10¹⁵, 5×10¹⁵, 6×10¹⁵, 7×10¹⁵, 8×10¹⁵, 9×10¹⁵, and 1×10¹⁶ VG.

48. The method of any one of claims 11-28, wherein the concentration of the AAV particles in the IT administration is selected from the group consisting of 1×10⁶, 2×10⁶, 3×10⁶, 4×10⁶, 5×10⁶, 6×10⁶, 7×10⁶, 8×10⁶, 9×10⁶, 1×10⁷, 2×10⁷, 3×10⁷, 4×10⁷, 5×10⁷, 6×10⁷, 7×10⁷, 8×10⁷, 9×10⁷, 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 2×10¹⁰, 3×10¹⁰, 4×10¹⁰, 5×10¹⁰, 6×10¹⁰, 7×10¹⁰, 8×10¹⁰, 9×10¹⁰, 1×10¹¹, 2×10¹¹, 3×10¹¹, 4×10¹¹, 5×10¹¹, 6×10¹¹, 7×10¹¹, 8×10¹¹, 9×10¹¹, 1×10¹², 2×10¹², 3×10¹², 4×10¹², 5×10¹², 6×10¹², 7×10¹², 8×10¹², 9×10¹², 1×10¹³, 2×10¹³, 3×10¹³, 4×10¹³, 5×10¹³, 6×10¹³, 7×10¹³, 8×10¹³, 9×10¹³, 1×10¹⁴, 2×10¹⁴, 3×10¹⁴, 4×10¹⁴, 5×10¹⁴, 6×10¹⁴, 7×10¹⁴, 8×10¹⁴, 9×10¹⁴, 1×10¹⁵, 2×10¹⁵, 3×10¹⁵, 4×10¹⁵, 5×10¹⁵, 6×10¹⁵, 7×10¹⁵, 8×10¹⁵, 9×10¹⁵, and 1×10¹⁶ VG/mL.

49. The method of any one of claims 11-48, wherein during IT administration the subject is in a position selected from the group consisting of, supine, prone, right lateral recumbent (RLR), left lateral recumbent (LLR), Fowler's, and Trendelenburg.

50. The method of any one of claims 11-48, wherein during IT administration the subject is at an angle between approximately horizontal 00 to about vertical 90° for the duration of the administration.

51. The method of claim 50, wherein the subject is at an angle selected from the group consisting of 0°, 1°, 2°, 3°, 4°, 5°, 6°, 7°, 8°, 9°, 10°, 11°, 12°, 13°, 14°, 15°, 16°, 17°, 18°, 19°, 20°, 21°, 22°, 23°, 24°, 25°, 26°, 27°, 28°, 29°, 30°, 31°, 32°, 33°, 34°, 35°, 36°, 37°, 38°, 39°, 40°, 41°, 42°, 43°, 44°, 45°, 46°, 47°, 48°, 49°, 50°, 51°, 52°, 53°, 54°, 55°, 56°, 57°, 58°, 59°, 60°, 61°, 62°, 63°, 64°, 65°, 66°, 67°, 68°, 69°, 70°, 71°, 72°, 73°, 74° 75°, 76°, 77°, 78°, 79°, 80°, 81°, 82°, 83°, 84°, 85°, 86°, 87°, 88°, 89°, and 90°.

52. The method of any one of claims 11-51, wherein the IT administration is by an infusion pump or device, and wherein said infusion pump or devices uses a catheter.

53. The method of claim 52, wherein the catheter is a single port catheter.

54. The method of claim 52, wherein the catheter is a multi-port catheter.

55. The method of claim 52, wherein the catheter is a flexible catheter.

56. The method of claim 52, wherein the catheter is a rigid catheter.

57. The method of claim 52, wherein the catheter is a retractable catheter.

58. A method of increasing distribution of AAV particles in the CNS of a subject in need thereof comprising administering to said subject an effective amount of said AAV particle comprising a vector genome packaged in a capsid.

59. The method of claim 58, wherein the capsid has a serotype selected from the group consisting of AAVrh.10 (AAVrh10), AAV-DJ (AAVDJ), AAV-DJ8 (AAVDJ8), AAV1, AAV2, AAV2G9, AAV3, AAV3a, AAV3b, AAV3-3, AAV4, AAV4-4, AAV5, AAV6, AAV6.1, AAV6.2, AAV6.1.2, AAV7, AAV7.2, AAV8, AAV9, AAV9.11, AAV9.13, AAV9.16, AAV9.24, AAV9.45, AAV9.47, AAV9.61, AAV9.68, AAV9.84, AAV9.9, AAV10, AAV11, AAV12, AAV16.3, AAV24.1, AAV27.3, AAV42.12, AAV42-1b, AAV42-2, AAV42-3a, AAV42-3b, AAV42-4, AAV42-5a, AAV42-5b, AAV42-6b, AAV42-8, AAV42-10, AAV42-11, AAV42-12, AAV42-13, AAV42-15, AAV42-aa, AAV43-1, AAV43-12, AAV43-20, AAV43-21, AAV43-23, AAV43-25, AAV43-5, AAV44.1, AAV44.2, AAV44.5, AAV223.1, AAV223.2, AAV223.4, AAV223.5, AAV223.6, AAV223.7, AAV1-7/rh.48, AAV1-8/rh.49, AAV2-15/rh.62, AAV2-3/rh.61, AAV2-4/rh.50, AAV2-5/rh.51, AAV3.1/hu.6, AAV3.1/hu.9, AAV3-9/rh.52, AAV3-11/rh.53, AAV4-8/r11.64, AAV4-9/rh.54, AAV4-19/rh.55, AAV5-3/rh.57, AAV5-22/rh.58, AAV7.3/hu.7, AAV16.8/hu.10, AAV16.12/hu.11, AAV29.3/bb.1, AAV29.5/bb.2, AAV106.1/hu.37, AAV114.3/hu.40, AAV127.2/hu.41, AAV127.5/hu.42, AAV128.3/hu.44, AAV130.4/hu.48, AAV145.1/hu.53, AAV145.5/hu.54, AAV145.6/hu.55, AAV161.10/hu.60, AAV161.6/hu.61, AAV33.12/hu.17, AAV33.4/hu.15, AAV33.8/hu.16, AAV52/hu.19, AAV52.1/hu.20, AAV58.2/hu.25, AAVA3.3, AAVA3.4, AAVA3.5, AAVA3.7, AAVC1, AAVC2, AAVC5, AAVF3, AAVF5, AAVH2, AAVrh.72, AAVhu.8, AAVrh.68, AAVrh.70, AAVpi.1, AAVpi.3, AAVpi.2, AAVrh.60, AAVrh.44, AAVrh.65, AAVrh.55, AAVrh.47, AAVrh.69, AAVrh.45, AAVrh.59, AAVhu.12, AAVH6, AAVLK03, AAVH-1/hu.1, AAVH-5/hu.3, AAVLG-10/rh.40, AAVLG-4/rh.38, AAVLG-9/hu.39, AAVN721-8/rh.43, AAVCh.5, AAVCh.5R1, AAVcy.2, AAVcy.3, AAVcy.4, AAVcy.5, AAVCy.5R1, AAVCy.5R2, AAVCy.5R3, AAVCy.5R4, AAVcy.6, AAVhu.1, AAVhu.2, AAVhu.3, AAVhu.4, AAVhu.5, AAVhu.6, AAVhu.7, AAVhu.9, AAVhu.10, AAVhu.11, AAVhu.13, AAVhu.15, AAVhu.16, AAVhu.17, AAVhu.18, AAVhu.20, AAVhu.21, AAVhu.22, AAVhu.23.2, AAVhu.24, AAVhu.25, AAVhu.27, AAVhu.28, AAVhu.29, AAVhu.29R, AAVhu.31, AAVhu.32, AAVhu.34, AAVhu.35, AAVhu.37, AAVhu.39, AAVhu.40, AAVhu.41, AAVhu.42, AAVhu.43, AAVhu.44, AAVhu.44R1, AAVhu.44R2, AAVhu.44R3, AAVhu.45, AAVhu.46, AAVhu.47, AAVhu.48, AAVhu.48R1, AAVhu.48R2, AAVhu.48R3, AAVhu.49, AAVhu.51, AAVhu.52, AAVhu.54, AAVhu.55, AAVhu.56, AAVhu.57, AAVhu.58, AAVhu.60, AAVhu.61, AAVhu.63, AAVhu.64, AAVhu.66, AAVhu.67, AAVhu.14/9, AAVhu.t 19, AAVrh.2, AAVrh.2R, AAVrh.8, AAVrh.8R, AAVrh.12, AAVrh.13, AAVrh.13R, AAVrh.14, AAVrh.17, AAVrh.18, AAVrh.19, AAVrh.20, AAVrh.21, AAVrh.22, AAVrh.23, AAVrh.24, AAVrh.25, AAVrh.31, AAVrh.32, AAVrh.33, AAVrh.34, AAVrh.35, AAVrh.36, AAVrh.37, AAVrh.37R2, AAVrh.38, AAVrh.39, AAVrh.40, AAVrh.46, AAVrh.48, AAVrh.48.1, AAVrh.48.1.2, AAVrh.48.2, AAVrh.49, AAVrh.51, AAVrh.52, AAVrh.53, AAVrh.54, AAVrh.56, AAVrh.57, AAVrh.58, AAVrh.61, AAVrh.64, AAVrh.64R1, AAVrh.64R2, AAVrh.67, AAVrh.73, AAVrh.74, AAVrh8R, AAVrh8R A586R mutant, AAVrh8R R533A mutant, AAAV, BAAV, caprine AAV, bovine AAV, AAVhE1.1, AAVhEr1.5, AAVhER1.14, AAVhEr1.8, AAVhEr1.16, AAVhEr1.18, AAVhEr1.35, AAVhEr1.7, AAVhEr1.36, AAVhEr2.29, AAVhEr2.4, AAVhEr2.16, AAVhEr2.30, AAVhEr2.31, AAVhEr2.36, AAVhER1.23, AAVhEr3.1, AAV2.5T, AAV-PAEC, AAV-LK01, AAV-LK02, AAV-LK03, AAV-LK04, AAV-LK05, AAV-LK06, AAV-LK07, AAV-LK08, AAV-LK09, AAV-LK10, AAV-LK11, AAV-LK12, AAV-LK13, AAV-LK14, AAV-LK15, AAV-LK16, AAV-LK17, AAV-LK18, AAV-LK19, AAV-PAEC2, AAV-PAEC4, AAV-PAEC6, AAV-PAEC7, AAV-PAEC8, AAV-PAEC11, AAV-PAEC12, AAV-2-pre-miRNA-101, AAV-8h, AAV-8b, AAV-h, AAV-b, AAV SM 10-2, AAV Shuffle 100-1, AAV Shuffle 100-3, AAV Shuffle 100-7, AAV Shuffle 10-2, AAV Shuffle 10-6, AAV Shuffle 10-8, AAV Shuffle 100-2, AAV SM 10-1, AAV SM 10-8, AAV SM 100-3, AAV SM 100-10, BNP61 AAV, BNP62 AAV, BNP63 AAV, AAVrh.50, AAVrh.43, AAVrh.62, AAVrh.48, AAVhu.19, AAVhu.11, AAVhu.53, AAV4-8/rh.64, AAVLG-9/hu.39, AAV54.5/hu.23, AAV54.2/hu.22, AAV54.7/hu.24, AAV54.1/hu.21, AAV54.4R/hu.27, AAV46.2/hu.28, AAV46.6/hu.29, AAV128.1/hu.43, true type AAV (ttAAV), UPENN AAV 10 and/or Japanese AAV 10 serotypes, and variants thereof.

60. The method of claim 58, wherein the administration is at least one route selected from the group consisting of intrathecal (IT) administration, intraparenchymal (IPa) administration, and intracerebroventricular (ICV) administration.

61. The method of claim 60, wherein the first route of administration is IT administration.

62. The method of claim 61, wherein IT administration occurs in at least one location in at least one region of the spine of the subject, and wherein the at least one region of the spine of the subject is selected from the group consisting of cervical, thoracic, lumbar and sacral region.

63. The method of claim 61, wherein IT administration occurs in the cervical region, and wherein IT administration to the cervical region occurs in at least one location selected from the group consisting of C1, C2, C3, C4, C5, C6, and C7.

64. The method of claim 61, wherein IT administration occurs in the thoracic region, and wherein IT administration to the thoracic region occurs in at least one location selected from the group consisting of T1, T2, T3, T3, T4, T5, T6, T7, T8, T9, T10, T11, and T12.

65. The method of claim 61, wherein the IT administration occurs in the lumbar region, and wherein IT administration to the lumbar region occurs in at least one location selected from the group consisting of L1, L2, L3, L4, and L5.

66. The method of claim 61, wherein IT administration occurs in the lumbar region, and wherein IT administration to the sacral region occurs in at least one location selected from the group consisting of S1, S2, S3, S4, and S5.

67. The method of any of claims 62-66, wherein IT administration occurs in one location.

68. The method of claim 67, wherein the location is C1.

69. The method of claim 67, wherein the location is C5.

70. The method of claim 67, wherein the location is T1.

71. The method of claim 67, wherein the location is L1.

72. The method of claim 67, wherein the location is L5.

73. The method of any of claims 62-66, wherein IT administration occurs in three locations.

74. The method of claim 73, wherein the locations are L1, T1 and C5.

75. The method of claim 61, wherein the volume of IT administration is less than 1 mL.

76. The method of claim 61, wherein the volume of IT administration is between about 0.1 mL to about 120 mL.

77. The method of any of claims 61-74, wherein the IT administration is via bolus infusion.

78. The method of any of claims 61-74, wherein the IT administration is via prolonged infusion.

79. The method of claim 78, wherein the prolonged infusion occurs at a volume of more than 1 mL.

80. The method of claim 79, wherein the prolonged infusion occurs at a volume of at least 3 mL.

81. The method of claim 79, wherein the prolonged infusion occurs at a volume of 3 mL.

82. The method of claim 79, wherein the prolonged infusion occurs at a volume of at least 10 mL.

83. The method of claim 79, wherein the prolonged infusion occurs at a volume of 10 mL.

84. The method of claim 78, wherein the prolonged infusion occurs for at least a duration selection from the group consisting of 0.17, 0.33, 0.5, 0.67, 0.83, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, and 36 hour(s).

85. The method of claim 84, wherein the duration is at least one hour.

86. The method of claim 84, wherein the duration is at least 10 hours.

87. The method of claim 78, wherein the prolonged infusion occurs at a constant rate.

88. The method of claim 78, wherein the prolonged infusion occurs at a ramped rate.

89. The method of claim 88, wherein the ramped rate increases over the duration of the prolonged infusion.

90. The method of claim 78, wherein the prolonged infusion occurs at a complex rate alternating between high and low rates over the duration of the prolonged infusion.

91. The method of any one of claims 79-90, wherein the rate of prolonged infusion is between about 0.1 mL/hour and about 25.0 mL/hour.

92. The method of claim 91, wherein the rate of prolonged infusion is selected from the group consisting of 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 10.1, 10.2, 10.3, 10.4, 10.5, 10.6, 10.7, 10.8, 10.9, 11.0, 11.1, 11.2, 11.3, 11.4, 11.5, 11.6, 11.7, 11.8, 11.9, 12.0, 12.1, 12.2, 12.3, 12.4, 12.5, 12.6, 12.7, 12.8, 12.9, 13.0, 13.1, 13.2, 13.3, 13.4, 13.5, 13.6, 13.7, 13.8, 13.9, 14.0, 14.1, 14.2, 14.3, 14.4, 14.5, 14.6, 14.7, 14.8, 14.9, 15.0, 15.1, 15.2, 15.3, 15.4, 15.5, 15.6, 15.7, 15.8, 15.9, 16.0, 16.1, 16.2, 16.3, 16.4, 16.5, 16.6, 16.7, 16.8, 16.9, 17.0, 17.1, 17.2, 17.3, 17.4, 17.5, 17.6, 17.7, 17.8, 17.9, 18.0, 18.1, 18.2, 18.3, 18.4, 18.5, 18.6, 18.7, 18.8, 18.9, 19.0, 19.1, 19.2, 19.3, 19.4, 19.5, 19.6, 19.7, 19.8, 19.9, 20.0, 20.1, 20.2, 20.3, 20.4, 20.5, 20.6, 20.7, 20.8, 20.9, 21.0, 21.1, 21.2, 21.3, 21.4, 21.5, 21.6, 21.7, 21.8, 21.9, 22.0, 22.1, 22.2, 22.3, 22.4, 22.5, 22.6, 22.7, 22.8, 22.9, 23.0, 23.1, 23.2, 23.3, 23.4, 23.5, 23.6, 23.7, 23.8, 23.9, 24.0, 24.1, 24.2, 24.3, 24.4, 24.5, 24.6, 24.7, 24.8, 24.9, and 25.0 mL/hour.

93. The method of claim 92, wherein the rate of prolonged infusion is 1.0 mL/hour.

94. The method of claim 92, wherein the rate of prolonged infusion is 1.5 mL/hour.

95. The method of any one of claims 79-90, wherein the rate of prolonged infusion exceeds the rate of cerebrospinal fluid (CSF) absorption.

96. The method of claim 61, wherein the second route of administration is ICV administration.

97. The method of claim 96, wherein ICV administration is via prolonged infusion to the ventricular system in at least one location selected from the group consisting of right lateral ventricle, left lateral ventricle, third ventricle, and fourth ventricle.

98. The method of claim 96, wherein ICV administration is via prolonged infusion to the ventricular system in at least one location selected from the group consisting of interventricular foramina (also called foramina of Monro), cerebral aqueduct, and central canal.

99. The method of claim 96, wherein ICV administration is via prolonged infusion to the ventricular system in at least one location selected from the group consisting of median aperture, right lateral aperture, and left lateral aperture.

100. The method of claim 96, wherein ICV administration is via prolonged infusion to the ventricular system in the perivascular space in the brain.

101. The method of any one of claims 60-101, wherein the administration comprises a total dose between about 1×10⁶ VG and about 1×10¹⁶ VG.

102. The method of claim 101, wherein the total dose is selected from the group consisting of about 1×10⁶, 2×10⁶, 3×10⁶, 4×10⁶, 5×10⁶, 6×10⁶, 7×10⁶, 8×10⁶, 9×10⁶, 1×10⁷, 2×10⁷, 3×10⁷, 4×10⁷, 5×10⁷, 6×10⁷, 7×10⁷, 8×10⁷, 9×10⁷, 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 2×10¹⁰, 3×10¹⁰, 4×10¹⁰, 5×10¹⁰, 6×10¹⁰, 7×10¹⁰, 8×10¹⁰, 9×10¹⁰, 1×10¹¹, 2×10¹¹, 3×10¹¹, 4×10¹¹, 5×10¹¹, 6×10¹¹, 7×10¹¹, 8×10¹¹, 9×10¹¹, 1×10¹², 2×10¹², 3×10¹², 4×10¹², 5×10¹², 6×10¹², 7×10¹², 8×10¹², 9×10¹², 1×10¹³, 2×10¹³, 3×10¹³, 4×10¹³, 5×10¹³, 6×10¹³, 7×10¹³, 8×10¹³, 9×10¹³, 1×10¹⁴, 2×10¹⁴, 3×10¹⁴, 4×10¹⁴, 5×10¹⁴, 6×10¹⁴, 7×10¹⁴, 8×10¹⁴, 9×10¹⁴, 1×10¹⁵, 2×10¹⁵, 3×10¹⁵, 4×10¹⁵, 5×10¹⁵, 6×10¹⁵, 7×10¹⁵, 8×10¹⁵, 9×10¹⁵, and 1×10¹⁶ VG.

103. The method of any one of claims 60-101, wherein the concentration of the AAV particles in the administration is selected from the group consisting of 1×10⁶, 2×10⁶, 3×10⁶, 4×10⁶, 5×10⁶, 6×10⁶, 7×10⁶, 8×10⁶, 9×10⁶, 1×10⁷, 2×10⁷, 3×10⁷, 4×10⁷, 5×10⁷, 6×10⁷, 7×10⁷, 8×10⁷, 9×10⁷, 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 2×10¹⁰, 3×10¹⁰, 4×10¹⁰, 5×10¹⁰, 6×10¹⁰, 7×10¹⁰, 8×10¹⁰, 9×10¹⁰, 1×10¹¹, 2×10¹¹, 3×10¹¹, 4×10¹¹, 5×10¹¹, 6×10¹¹, 7×10¹¹, 8×10¹¹, 9×10¹¹, 1×10¹², 2×10¹², 3×10¹², 4×10¹², 5×10¹², 6×10¹², 7×10¹², 8×10¹², 9×10¹², 1×10¹³, 2×10¹³, 3×10¹³, 4×10¹³, 5×10¹³, 6×10¹³, 7×10¹³, 8×10¹³, 9×10¹³, 1×10¹⁴, 2×10¹⁴, 3×10¹⁴, 4×10¹⁴, 5×10¹⁴, 6×10¹⁴, 7×10¹⁴, 8×10¹⁴, 9×10¹⁴, 1×10¹⁵, 2×10¹⁵, 3×10¹⁵, 4×10¹⁵, 5×10¹⁵, 6×10¹⁵, 7×10¹⁵, 8×10¹⁵, 9×10¹⁵, and 1×10¹⁶ VG/mL.

104. The method of any one of claims 60-103, wherein the IT administration is by an infusion pump or device, and wherein said infusion pump or devices uses a catheter.

105. The method of claim 104, wherein the catheter is a single port catheter.

106. The method of claim 104, wherein the catheter is a multi-port catheter.

107. The method of claim 104, wherein the catheter is a flexible catheter.

108. The method of claim 104, wherein the catheter is a rigid catheter.

109. The method of claim 104, wherein the catheter is a retractable catheter.

110. The method of any one of claims 60, and 96-109, wherein a device selected from the group consisting of a head trajectory guide, head trajectory frame, and a skull frame is used for ICV administration.

111. The method of claim 110, wherein neuronavigational software is used for ICV administration.

112. The method of any one of claims 58-111, wherein the distribution is increased by a percentage selected from the group consisting of 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 60%, 65%, 70%, 75%, 80%, 85%, 90%, 95% or more than 95%.

113. An AAV particle comprising a vector genome packaged in a capsid, said capsid having a serotype selected from the group consisting of AAVrh.10 (AAVrh10), AAV-DJ (AAVDJ), AAV-DJ8 (AAVDJ8), AAV1, AAV2, AAV2G9, AAV3, AAV3a, AAV3b, AAV3-3, AAV4, AAV4-4, AAV5, AAV6, AAV6.1, AAV6.2, AAV6.1.2, AAV7, AAV7.2, AAV8, AAV9, AAV9.11, AAV9.13, AAV9.16, AAV9.24, AAV9.45, AAV9.47, AAV9.61, AAV9.68, AAV9.84, AAV9.9, AAV10, AAV11, AAV12, AAV16.3, AAV24.1, AAV27.3, AAV42.12, AAV42-1b, AAV42-2, AAV42-3a, AAV42-3b, AAV42-4, AAV42-5a, AAV42-5b, AAV42-6b, AAV42-8, AAV42-10, AAV42-11, AAV42-12, AAV42-13, AAV42-15, AAV42-aa, AAV43-1, AAV43-12, AAV43-20, AAV43-21, AAV43-23, AAV43-25, AAV43-5, AAV44.1, AAV44.2, AAV44.5, AAV223.1, AAV223.2, AAV223.4, AAV223.5, AAV223.6, AAV223.7, AAV1-7/rh.48, AAV1-8/rh.49, AAV2-15/rh.62, AAV2-3/rh.61, AAV2-4/rh.50, AAV2-5/rh.51, AAV3.1/hu.6, AAV3.1/hu.9, AAV3-9/rh.52, AAV3-11/rh.53, AAV4-8/r11.64, AAV4-9/rh.54, AAV4-19/rh.55, AAV5-3/rh.57, AAV5-22/rh.58, AAV7.3/hu.7, AAV16.8/hu.10, AAV16.12/hu.11, AAV29.3/bb.1, AAV29.5/bb.2, AAV106.1/hu.37, AAV114.3/hu.40, AAV127.2/hu.41, AAV127.5/hu.42, AAV128.3/hu.44, AAV130.4/hu.48, AAV145.1/hu.53, AAV145.5/hu.54, AAV145.6/hu.55, AAV161.10/hu.60, AAV161.6/hu.61, AAV33.12/hu.17, AAV33.4/hu.15, AAV33.8/hu.16, AAV52/hu.19, AAV52.1/hu.20, AAV58.2/hu.25, AAVA3.3, AAVA3.4, AAVA3.5, AAVA3.7, AAVC1, AAVC2, AAVC5, AAVF3, AAVF5, AAVH2, AAVrh.72, AAVhu.8, AAVrh.68, AAVrh.70, AAVpi.1, AAVpi.3, AAVpi.2, AAVrh.60, AAVrh.44, AAVrh.65, AAVrh.55, AAVrh.47, AAVrh.69, AAVrh.45, AAVrh.59, AAVhu.12, AAVH6, AAVLK03, AAVH-1/hu.1, AAVH-5/hu.3, AAVLG-10/rh.40, AAVLG-4/rh.38, AAVLG-9/hu.39, AAVN721-8/rh.43, AAVCh.5, AAVCh.5R1, AAVcy.2, AAVcy.3, AAVcy.4, AAVcy.5, AAVCy.5R1, AAVCy.5R2, AAVCy.5R3, AAVCy.5R4, AAVcy.6, AAVhu.1, AAVhu.2, AAVhu.3, AAVhu.4, AAVhu.5, AAVhu.6, AAVhu.7, AAVhu.9, AAVhu.10, AAVhu.11, AAVhu.13, AAVhu.15, AAVhu.16, AAVhu.17, AAVhu.18, AAVhu.20, AAVhu.21, AAVhu.22, AAVhu.23.2, AAVhu.24, AAVhu.25, AAVhu.27, AAVhu.28, AAVhu.29, AAVhu.29R, AAVhu.31, AAVhu.32, AAVhu.34, AAVhu.35, AAVhu.37, AAVhu.39, AAVhu.40, AAVhu.41, AAVhu.42, AAVhu.43, AAVhu.44, AAVhu.44R1, AAVhu.44R2, AAVhu.44R3, AAVhu.45, AAVhu.46, AAVhu.47, AAVhu.48, AAVhu.48R1, AAVhu.48R2, AAVhu.48R3, AAVhu.49, AAVhu.51, AAVhu.52, AAVhu.54, AAVhu.55, AAVhu.56, AAVhu.57, AAVhu.58, AAVhu.60, AAVhu.61, AAVhu.63, AAVhu.64, AAVhu.66, AAVhu.67, AAVhu.14/9, AAVhu.t 19, AAVrh.2, AAVrh.2R, AAVrh.8, AAVrh.8R, AAVrh.12, AAVrh.13, AAVrh.13R, AAVrh.14, AAVrh.17, AAVrh.18, AAVrh.19, AAVrh.20, AAVrh.21, AAVrh.22, AAVrh.23, AAVrh.24, AAVrh.25, AAVrh.31, AAVrh.32, AAVrh.33, AAVrh.34, AAVrh.35, AAVrh.36, AAVrh.37, AAVrh.37R2, AAVrh.38, AAVrh.39, AAVrh.40, AAVrh.46, AAVrh.48, AAVrh.48.1, AAVrh.48.1.2, AAVrh.48.2, AAVrh.49, AAVrh.51, AAVrh.52, AAVrh.53, AAVrh.54, AAVrh.56, AAVrh.57, AAVrh.58, AAVrh.61, AAVrh.64, AAVrh.64R1, AAVrh.64R2, AAVrh.67, AAVrh.73, AAVrh.74, AAVrh8R, AAVrh8R A586R mutant, AAVrh8R R533A mutant, AAAV, BAAV, caprine AAV, bovine AAV, AAVhE1.1, AAVhEr1.5, AAVhER1.14, AAVhEr1.8, AAVhEr1.16, AAVhEr1.18, AAVhEr1.35, AAVhEr1.7, AAVhEr1.36, AAVhEr2.29, AAVhEr2.4, AAVhEr2.16, AAVhEr2.30, AAVhEr2.31, AAVhEr2.36, AAVhER1.23, AAVhEr3.1, AAV2.5T, AAV-PAEC, AAV-LK01, AAV-LK02, AAV-LK03, AAV-LK04, AAV-LK05, AAV-LK06, AAV-LK07, AAV-LK08, AAV-LK09, AAV-LK10, AAV-LK11, AAV-LK12, AAV-LK13, AAV-LK14, AAV-LK15, AAV-LK16, AAV-LK17, AAV-LK18, AAV-LK19, AAV-PAEC2, AAV-PAEC4, AAV-PAEC6, AAV-PAEC7, AAV-PAEC8, AAV-PAEC11, AAV-PAEC12, AAV-2-pre-miRNA-101, AAV-8h, AAV-8b, AAV-h, AAV-b, AAV SM 10-2, AAV Shuffle 100-1, AAV Shuffle 100-3, AAV Shuffle 100-7, AAV Shuffle 10-2, AAV Shuffle 10-6, AAV Shuffle 10-8, AAV Shuffle 100-2, AAV SM 10-1, AAV SM 10-8, AAV SM 100-3, AAV SM 100-10, BNP61 AAV, BNP62 AAV, BNP63 AAV, AAVrh.50, AAVrh.43, AAVrh.62, AAVrh.48, AAVhu.19, AAVhu.11, AAVhu.53, AAV4-8/rh.64, AAVLG-9/hu.39, AAV54.5/hu.23, AAV54.2/hu.22, AAV54.7/hu.24, AAV54.1/hu.21, AAV54.4R/hu.27, AAV46.2/hu.28, AAV46.6/hu.29, AAV128.1/hu.43, true type AAV (ttAAV), UPENN AAV 10 and/or Japanese AAV 10 serotypes, and variants thereof.

114. The AAV particle of claim 113, wherein the capsid is AAVrh10.

115. The AAV particle of claim 113, wherein the capsid is AAV-DJ.

116. The AAV particle of claim 113, wherein the capsid is AAV-DJ8.

117. The AAV particle of claim 113, wherein the vector genome comprises a promoter, and wherein said promoter is selected from the group consisting of CBA, CMV, PGK, FXN, H1, and fragments or variants thereof.

118. The AAV particle of claim 117, wherein the promoter is CBA.

119. The AAV particle of claim 117, wherein the promoter is CMV.

120. The AAV particle of claim 117, wherein the promoter is FXN.

121. The AAV particle of claim 117, wherein the promoter is H1.
